(19) United States
(12) Patent Application Publication
(10) Pub. No.: US 2024/0309399 A1
(43) Pub. Date: Sep. 19, 2024

(54) METHODS AND COMPOSITIONS FOR ADMINISTERING OTOFERLIN DUAL VECTOR SYSTEMS

(71) Applicant: Decibel Therapeutics, Inc., Boston, MA (US)

(72) Inventors: Qi-Ying HU, Needham, MA (US); Orion Paul Keifer, JR., Atlanta, GA (US); Seth Koehler, Brookline, MA (US); John Lee, Arlington, MA (US); Adam Palermo, Somerville, MA (US); Ning Pan, Newton, MA (US); Jonathan B. Sellon, Newton, MA (US); Jonathon Whitton, Cambridge, MA (US)

(21) Appl. No.: 18/444,107

(22) Filed: Feb. 16, 2024

Related U.S. Application Data
(60) Provisional application No. 63/549,204, filed on Feb. 2, 2024.
(60) Provisional application No. 63/618,348, filed on Jan. 7, 2024.
(60) Provisional application No. 63/545,804, filed on Oct. 26, 2023.
(60) Provisional application No. 63/485,656, filed on Feb. 17, 2023.

(51) Int. Cl. C12N 15/86; A61K 38/17; A61K 48/00; A61P 27/16; C07K 14/47

(52) U.S. Cl. CPC C12N 15/86; A61K 38/1738; A61K 48/0066; A61K 48/0083; A61P 27/16; C07K 14/4728; C12N 2750/14143; C12N 2750/14171; C12N 2800/40

(57) ABSTRACT

The disclosure features compositions and methods for the treatment of sensorineural hearing loss and auditory neuropathy, particularly forms of the disease that are associated with a mutation in otoferlin (OTOF), by way of OTOF gene therapy. The disclosure provides a variety of compositions that include a first nucleic acid vector that contains a polynucleotide encoding an N-terminal portion of an OTOF protein (e.g., an OTOF isoform 5 protein) and a second nucleic acid vector that contains a polynucleotide encoding a C-terminal portion of an OTOF protein (e.g., an OTOF isoform 5 protein). These vectors can be used to increase the expression of OTOF in a subject, such as a human subject suffering from sensorineural hearing loss.

### SEQUENCE LISTING

The instant application contains a Sequence Listing which has been submitted electronically in XML file format and is hereby incorporated by reference in its entirety. Said XML copy, created on Feb. 16, 2024, is named 51471-014005_Sequence_Listing_2_16_24.xml and is 188,956 bytes in size.

### FIELD OF THE INVENTION

Described herein are compositions and methods for the treatment of sensorineural hearing loss and auditory neuropathy, particularly forms of the disease that are associated with biallelic mutations in otoferlin (OTOF), by way of OTOF gene therapy. The disclosure provides dual vector systems that include a first nucleic acid vector that contains a polynucleotide encoding an N-terminal portion of an OTOF protein (e.g., an OTOF isoform 5 protein) and a second nucleic acid vector that contains a polynucleotide encoding a C-terminal portion of an OTOF protein (e.g., an OTOF isoform 5 protein). These vectors can be used to increase the expression of or provide wild-type OTOF to a subject, such as a human subject suffering from sensorineural hearing loss.

### BACKGROUND

Sensorineural hearing loss is a type of hearing loss caused by defects in the cells of the inner ear or the neural pathways that project from the inner ear to the brain. Although sensorineural hearing loss is often acquired, and can be caused by noise, infections, head trauma, ototoxic drugs, or aging, there are also congenital forms of sensorineural hearing loss associated with autosomal recessive mutations. One such form of autosomal recessive sensorineural hearing loss is associated with mutation of the otoferlin (OTOF) gene, which is implicated in prelingual nonsyndromic hearing loss. In recent years, efforts to treat hearing loss have increasingly focused on gene therapy as a possible solution; however, OTOF is too large to allow for treatment using standard gene therapy approaches. There is a need for new therapeutics to treat OTOF-related sensorineural hearing loss.

### SUMMARY OF THE INVENTION

The invention provides methods for treating a subject having biallelic otoferlin (OTOF) mutations by administering to an inner ear of the subject an effective amount of an OTOF dual vector system. The OTOF dual vector system may be administered in an amount of 1×10¹³ vector genomes (vg)/mL to 1×10¹⁴ vg/mL in a volume of 200-250 μL. The subject may be, e.g., a pediatric subject with congenital auditory neuropathy and may be determined to have profound sensorineural hearing loss prior to treatment (e.g., based on ABR measurements). The methods described herein may be used to instate hearing and may result in an improvement in ABR measurements and/or behavioral audiometry in the treated subjects.

In a first aspect, the invention provides a method of treating a human subject having biallelic OTOF mutations (e.g., a biallelic likely pathogenic or pathogenic mutation in OTOF), the method including the step of administering to an inner ear of the subject an amount of 1×10¹³ vg/mL to 1×10¹⁴ vg/mL of an OTOF dual vector system (e.g., the combined amount of both vectors is 1×10¹³ vg/mL to 1×10¹⁴ vg/mL) in a volume of 200-250 μL by intracochlear injection, in which the OTOF dual vector system includes: a first adeno-associated virus (AAV) vector including a first inverted terminal repeat (ITR) sequence; a promoter operably linked to a first coding polynucleotide that encodes an N-terminal portion of an OTOF protein; a splice donor sequence positioned 3′ of the first coding polynucleotide; a recombinogenic region positioned 3′ of the splice donor sequence; and a second ITR sequence; and a second AAV vector including a first ITR sequence; a second recombinogenic region; a splice acceptor sequence positioned 3′ of the second recombinogenic region; a second coding polynucleotide that encodes a C-terminal portion of the OTOF protein positioned 3′ of the splice acceptor sequence; a poly(A) sequence positioned 3′ of the second coding polynucleotide; and a second ITR sequence; in which the first coding polynucleotide and the second coding polynucleotide that encode the OTOF protein do not overlap, and in which neither the first nor second AAV vector encodes the full-length OTOF protein. In some embodiments, the first AAV vector and the second AAV vector are administered at a ratio of about 3:1 to about 1:3 (e.g., 3:1 to 1:3, 2:1 to 1:2, or 1:1).

In another aspect, the invention provides a method of improving hearing in a human subject, the method including the steps of selecting a subject having biallelic OTOF mutations (e.g., a biallelic likely pathogenic or pathogenic mutation in OTOF) and administering to an inner ear of the subject an amount of 1×10¹³ vg/mL to 1×10¹⁴ vg/mL of an OTOF dual vector system (e.g., the combined amount of both vectors is 1×10¹³ vg/mL to 1×10¹⁴ vg/mL) in a volume of 200-250 μL by intracochlear injection, in which the OTOF dual vector system includes: a first AAV vector including a first ITR sequence; a promoter operably linked to a first coding polynucleotide that encodes an N-terminal portion of an OTOF protein; a splice donor sequence positioned 3′ of the first coding polynucleotide; a recombinogenic region positioned 3′ of the splice donor sequence; and a second ITR sequence; and a second AAV vector including a first ITR sequence; a second recombinogenic region; a splice acceptor sequence positioned 3′ of the second recombinogenic region; a second coding polynucleotide that encodes a C-terminal portion of the OTOF protein positioned 3′ of the splice acceptor sequence; a poly(A) sequence positioned 3′ of the second coding polynucleotide; and a second ITR sequence; in which the first coding polynucleotide and the second coding polynucleotide that encode the OTOF protein do not overlap, and in which neither the first nor second AAV vector encodes the full-length OTOF protein. In some embodiments, the first AAV vector and the second AAV vector are administered at a ratio of about 3:1 to 1:3 (e.g., 3:1 to 1:3, 2:1 to 1:2, or 1:1). In some embodiments, the subject has or is identified as having profound sensorineural hearing loss (e.g., a subject with absence of an ABR neural signal in response to a click stimulus at or below 85 dB normalized hearing level in the ear(s) to be treated or a subject with ≥90 dB hearing level in the ear(s) to be treated) (e.g., the method includes a step of selecting a subject having biallelic OTOF mutations and profound sensorineural hearing loss). In some embodiments, the subject has or is identified as having present outer hair cell function (e.g., the method includes a step of selecting a subject having biallelic OTOF mutations, profound sensorineural hearing loss, and present outer hair cell function). In some embodiments, the subject is determined to have present outer hair cell function based on detectable otoacoustic emissions (e.g., presence of otoacoustic emissions (≥6 dB signal-to-noise ratio) at ≥3 frequencies from 1 to 8 KHz in the ear(s) to be treated). In some embodiments, the subject is determined to have present outer hair cell function based on a present cochlear microphonic (e.g., in the ear(s) to be treated).

In another aspect, the invention provides an aqueous suspension containing an OTOF dual vector system, 10 mM sodium phosphate or disodium phosphate, 180 mM sodium chloride, 5% (w/v) sucrose, and 0.001% (w/v) poloxamer 188 at a pH of 7.4, in which the OTOF dual vector system includes: a first AAV vector including a first ITR sequence; a promoter operably linked to a first coding polynucleotide that encodes an N-terminal portion of an OTOF protein; a splice donor sequence positioned 3′ of the first coding polynucleotide; a recombinogenic region positioned 3′ of the splice donor sequence; and a second ITR sequence; and a second AAV vector including a first ITR sequence; a second recombinogenic region; a splice acceptor sequence positioned 3′ of the second recombinogenic region; a second coding polynucleotide that encodes a C-terminal portion of the OTOF protein positioned 3′ of the splice acceptor sequence; a poly(A) sequence positioned 3′ of the second coding polynucleotide; and a second ITR sequence; in which the first coding polynucleotide and the second coding polynucleotide that encode the OTOF protein do not overlap, and in which neither the first nor second AAV vector encodes the full-length OTOF protein. In some embodiments, the suspension contains 10 mM sodium phosphate. In some embodiments, the suspension contains 10 mM disodium phosphate. In some embodiments, the ratio of the first vector to the second vector is about 3:1 to about 1:3. In some embodiments, the first vector and second vector are matched in titer and mixed at an approximately equal ratio. In some embodiments, the ratio of the first vector to the second vector is about 1:1. In some embodiments, the suspension has a titer of 1×10¹³ vg/mL to 1×10¹⁴ vg/mL. In some embodiments, the suspension has a titer of 1×10¹³ vg/mL to 5×10¹³ vg/mL. In some embodiments, the suspension has a titer of 3×10¹³ vg/mL. In some embodiments, the suspension has a titer of 5×10¹³ vg/mL to 1×10¹⁴ vg/mL. In some embodiments, the suspension has a titer of 7.3×10¹³ vg/mL. In some embodiments, the suspension is formulated for intracochlear injection.

In some embodiments, the OTOF protein is an OTOF isoform 5 protein. In some embodiments, the protein is an OTOF isoform 1 protein.

In some embodiments, the first AAV vector and the second AAV vector include an AAV1 capsid. In some embodiments, the first AAV vector and the second AAV vector include an Anc80 capsid. In some embodiments, the first AAV vector and the second AAV vector include an AAV9 capsid. In some embodiments, the first AAV vector and the second AAV vector include an AAV8 capsid. In some embodiments, the first AAV vector and the second AAV vector include an AAV2 capsid. In some embodiments, the first AAV vector and the second AAV vector include an AAV2quad(Y-F) capsid. In some embodiments, the first AAV vector and the second AAV vector include an AAV6 capsid. In some embodiments, the first AAV vector and the second AAV vector include an An80L65 capsid. In some embodiments, the first AAV vector and the second AAV vector include a DJ/9 capsid. In some embodiments, the first AAV vector and the second AAV vector include a 7m8 capsid. In some embodiments, the first AAV vector and the second AAV vector include a PHP.B capsid.

In some embodiments, the promoter is a ubiquitous promoter. In some embodiments, the ubiquitous promoter is a CAG promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), the chicken β-actin promoter, a truncated CMV-chicken β-actin promoter (smCBA), the CB7 promoter, the hybrid CMV enhancer/human β-actin promoter, the CASI promoter, the dihydrofolate reductase (DHFR) promoter, the human β-actin promoter, a β-globin promoter (e.g., a minimal β-globin promoter), an HSV promoter (e.g., a minimal HSV ICP0 promoter or a truncated HSV ICP0 promoter), an SV40 promoter (e.g., an SV40 minimal promoter), the EF1α promoter, and the PGK promoter. In some embodiments, the ubiquitous promoter is a CAG promoter. In some embodiments, the ubiquitous promoter is a CMV promoter. In some embodiments, the ubiquitous promoter is the hybrid CMV enhancer/human β-actin promoter. In some embodiments, the ubiquitous promoter is the smCBA promoter. In some embodiments, the smCBA promoter comprises or consists of the sequence of SEQ ID NO: 44.

In some embodiments, the promoter is a Myosin 15 (Myo15) promoter. In some embodiments, the Myo15 promoter comprises a first region having at least 85% sequence identity (e.g., 85%, 90%, 91%, 92%, 93%, 94%, 95%, 96%, 97%, 98%, 99%, or more, sequence identity) to SEQ ID NO: 7 or a functional portion or derivative thereof including the sequence of SEQ ID NO: 9 and/or SEQ ID NO: 10 joined (e.g., operably linked) to a second region having at least 85% sequence identity (e.g., 85%, 90%, 91%, 92%, 93%, 94%, 95%, 96%, 97%, 98%, 99%, or more, sequence identity) to SEQ ID NO: 8 or a functional portion or derivative thereof including the sequence of SEQ ID NO: 14 and/or SEQ ID NO: 15, optionally containing a linker comprising one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region is directly fused to the second region. In some embodiments, the first region comprises or consists of the sequence of SEQ ID NO: 7. In some embodiments, the second region comprises or consists of the sequence of SEQ ID NO: 8. In some embodiments, the Myo15 promoter comprises or consists of the sequence of SEQ ID NO: 19. In some embodiments, the Myo15 promoter comprises or consists of the sequence of SEQ ID NO: 21. In some embodiments, the Myo15 promoter comprises or consists of the sequence of SEQ ID NO: 22. In some embodiments, the Myo15 promoter comprises or consists of the sequence of SEQ ID NO: 36. In some embodiments, the Myo15 promoter comprises or consists of the sequence of SEQ ID NO: 37. In some embodiments, the Myo15 promoter comprises or consists of the sequence of SEQ ID NO: 42. In some embodiments, the Myo15 promoter comprises or consists of the sequence of SEQ ID NO: 43.

In some embodiments, the Myo15 promoter comprises a first region having at least 85% sequence identity (e.g., 85%, 90%, 91%, 92%, 93%, 94%, 95%, 96%, 97%, 98%, 99%, or more, sequence identity) to SEQ ID NO: 8 or a functional portion or derivative thereof including the sequence of SEQ ID NO: 14 and/or SEQ ID NO: 15, joined (e.g., operably linked) to a second region having at least 85% sequence identity (e.g., 85%, 90%, 91%, 92%, 93%, 94%, 95%, 96%, 97%, 98%, 99%, or more, sequence identity) to SEQ ID NO: 7 or a functional portion or derivative thereof including the sequence of SEQ ID NO: 9 and/or SEQ ID NO: 10, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first reaction is directly fused to the second region. In some embodiments, the first region comprises or consists of the sequence of SEQ ID NO: 8. In some embodiments, the second region comprises or consists of the sequence of SEQ ID NO: 7. In some embodiments, the Myo15 promoter comprises or consists of the sequence of SEQ ID NO: 20. In some embodiments, the Myo15 promoter comprises or consists of the sequence of SEQ ID NO: 41.

In some embodiments, the Myo15 promoter comprises a region having at least 85% sequence identity (e.g., 85%, 90%, 91%, 92%, 93%, 94%, 95%, 96%, 97%, 98%, 99%, or more, sequence identity) to SEQ ID NO: 7 or a functional portion or derivative thereof including the sequence of SEQ ID NO: 9 and/or SEQ ID NO: 10. In some embodiments, the region comprises or consists of the sequence of SEQ ID NO: 7.

In some embodiments, the Myo15 promoter comprises a region having at least 85% sequence identity (e.g., 85%, 90%, 91%, 92%, 93%, 94%, 95%, 96%, 97%, 98%, 99%, or more, sequence identity) to SEQ ID NO: 8 or a functional portion or derivative thereof including the sequence of SEQ ID NO: 14 and/or SEQ ID NO: 15. In some embodiments, the region comprises or consists of the sequence of SEQ ID NO: 8.

In some embodiments, the functional portion of SEQ ID NO: 7 contains the sequence of SEQ ID NO: 9. In some embodiments, the functional portion of SEQ ID NO: 7 contains the sequence of SEQ ID NO: 10. In some embodiments, the functional portion of SEQ ID NO: 7 contains the sequence of SEQ ID NO: 9 and the sequence of SEQ ID NO: 10. In some embodiments, the functional portion of SEQ ID NO: 7 contains the sequence of SEQ ID NO: 11. In some embodiments, the functional portion of SEQ ID NO: 7 contains the sequence of SEQ ID NO: 12. In some embodiments, the functional portion of SEQ ID NO: 7 contains the sequence of SEQ ID NO: 13. In some embodiments, the functional portion of SEQ ID NO: 7 contains the sequence of SEQ ID NO: 33.

In some embodiments, the functional portion of SEQ ID NO: 8 contains the sequence of SEQ ID NO: 14. In some embodiments, the functional portion of SEQ ID NO: 8 contains the sequence of SEQ ID NO: 15. In some embodiments, the functional portion of SEQ ID NO: 8 contains the sequence of SEQ ID NO: 34. In some embodiments, the functional portion of SEQ ID NO: 8 contains the sequence of SEQ ID NO: 35. In some embodiments, the functional portion of SEQ ID NO: 8 contains the sequence of SEQ ID NO: 14 and the sequence of SEQ ID NO: 15. In some embodiments, the functional portion of SEQ ID NO: 8 contains the sequence of SEQ ID NO: 16. In some embodiments, the functional portion of SEQ ID NO: 8 contains the sequence of SEQ ID NO: 17. In some embodiments, the functional portion of SEQ ID NO: 8 contains the sequence of SEQ ID NO: 18. In some embodiments, the functional portion of SEQ ID NO: 8 contains the sequence of SEQ ID NO: 38.

In some embodiments, the Myo15 promoter has at least 85% sequence identity (e.g., 85%, 90%, 91%, 92%, 93%, 94%, 95%, 96%, 97%, 98%, 99%, or more, sequence identity) to the nucleic acid sequence of any one of SEQ ID NOs: 33-41. In some embodiments, the Myo15 promoter comprises or consists of the sequence of SEQ ID NO: 33. In some embodiments, the Myo15 promoter comprises or consists of the sequence of SEQ ID NO: 34. In some embodiments, the Myo15 promoter comprises or consists of the sequence of SEQ ID NO: 35. In some embodiments, the Myo15 promoter comprises or consists of the sequence of SEQ ID NO: 36. In some embodiments, the Myo15 promoter comprises or consists of the sequence of SEQ ID NO: 37. In some embodiments, the Myo15 promoter comprises or consists of the sequence of SEQ ID NO: 38. In some embodiments, the Myo15 promoter comprises or consists of the sequence of SEQ ID NO: 39. In some embodiments, the Myo15 promoter comprises or consists of the sequence of SEQ ID NO: 40. In some embodiments, the Myo15 promoter comprises or consists of the sequence of SEQ ID NO: 41.

In some embodiments, the Myo15 promoter comprises a first region having at least 85% sequence identity (e.g., 85%, 90%, 91%, 92%, 93%, 94%, 95%, 96%, 97%, 98%, 99%, or more, sequence identity) to SEQ ID NO: 23 or a functional portion or derivative thereof including the sequence of SEQ ID NO: 25 joined (e.g., operably linked) to a second region having at least 85% sequence identity (e.g., 85%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, optionally containing a linker comprising one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region is directly fused to the second region. In some embodiments, the first region comprises or consists of the sequence of SEQ ID NO: 23. In some embodiments, the second region comprises or consists of the sequence of SEQ ID NO: 24. In some embodiments, the Myo15 promoter comprises or consists of the sequence of SEQ ID NO: 31. In some embodiments, the Myo15 promoter comprises or consists of the sequence of SEQ ID NO: 32.

In some embodiments, the Myo15 promoter comprises a first region having at least 85% sequence identity (e.g., 85%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, joined (e.g., operably linked) to a second region having at least 85% sequence identity (e.g., 85%, 90%, 91%, 92%, 93%, 94%, 95%, 96%, 97%, 98%, 99%, or more, sequence identity) to SEQ ID NO: 23 or a functional portion or derivative thereof including the sequence of SEQ ID NO: 25, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region is directly fused to the second region. In some embodiments, the first region comprises or consists of the sequence of SEQ ID NO: 24. In some embodiments, the second region comprises or consists of the sequence of SEQ ID NO: 23.

In some embodiments, the Myo15 promoter comprises a region having at least 85% sequence identity (e.g., 85%, 90%, 91%, 92%, 93%, 94%, 95%, 96%, 97%, 98%, 99%, or more, sequence identity) to SEQ ID NO: 23 or a functional portion or derivative thereof including the sequence of SEQ ID NO: 25. In some embodiments, the region comprises or consists of the sequence of SEQ ID NO: 23.

In some embodiments, the Myo15 promoter comprises a region having at least 85% sequence identity (e.g., 85%, 90%, 91%, 92%, 93%, 94%, 95%, 96%, 97%, 98%, 99%, or more, sequence identity) to SEQ ID NO: 24 or a functional portion or derivative thereof including the sequence of SEQ ID NO: 26 and/or SEQ ID NO: 27. In some embodiments, the region comprises or consists of the sequence of SEQ ID NO: 24.

In some embodiments, the functional portion of SEQ ID NO: 23 contains the sequence of SEQ ID NO: 25.

In some embodiments, the functional portion of SEQ ID NO: 24 contains the sequence of SEQ ID NO: 26. In some embodiments, the functional portion of SEQ ID NO: 24 contains the sequence of SEQ ID NO: 27. In some embodiments, the functional portion of SEQ ID NO: 24 contains the sequence of SEQ ID NO: 26 and the sequence of SEQ ID NO: 27. In some embodiments, the functional portion of SEQ ID NO: 24 contains the sequence of SEQ ID NO: 28. In some embodiments, the functional portion of SEQ ID NO: 24 contains the sequence of SEQ ID NO: 29. In some embodiments, the functional portion of SEQ ID NO: 24 contains the sequence of SEQ ID NO: 30.

In some embodiments, the first and second recombinogenic regions are the same.

In some embodiments, the first recombinogenic region and/or the second recombinogenic region is an AK recombinogenic region. In some embodiments, the AK recombinogenic region comprises or consists of the sequence of SEQ ID NO: 47.

In some embodiments, the first recombinogenic region and/or the second recombinogenic region is an AP gene fragment. In some embodiments, the AP gene fragment comprises or consists of the sequence of any one of SEQ ID NOs: 48-53. In some embodiments, the AP gene fragment comprises or consists of the sequence of SEQ ID NO: 51.

In some embodiments, each of the first and second coding polynucleotides encode about half of the OTOF isoform 5 protein sequence.

In some embodiments, the first coding polynucleotide encodes amino acids 1-802 of SEQ ID NO: 1. In some embodiments of any of the foregoing aspects, the second coding polynucleotide encodes amino acids 803-1997 of SEQ ID NO: 1.

In some embodiments, the first and second coding polynucleotides are divided at an OTOF exon boundary. In some embodiments, the first and second coding polynucleotides are divided at the boundary between exons 20 and 21 of OTOF. In some embodiments, the first and second coding polynucleotides are divided at the boundary between exons 21 and 22 of OTOF.

In some embodiments, the first coding polynucleotide consists of exons 1-20 of a polynucleotide encoding the OTOF isoform 5 protein and the second coding polynucleotide consists of exons 21-45 and 47 of a polynucleotide encoding the OTOF isoform 5 protein (e.g., a polynucleotide encoding a human OTOF isoform 5 protein).

In some embodiments, the first coding polynucleotide consists of exons 1-21 of a polynucleotide encoding the OTOF isoform 5 protein and the second coding polynucleotide consists of exons 22-45 and 47 of a polynucleotide encoding the OTOF isoform 5 protein (e.g., a polynucleotide encoding a human OTOF isoform 5 protein).

In some embodiments, the first and second coding polynucleotides that encode the OTOF isoform 5 protein do not comprise introns.

In some embodiments, the OTOF isoform 5 protein is a human OTOF isoform 5 protein (e.g., the protein having the sequence of SEQ ID NO: 1).

In some embodiments, the OTOF isoform 5 protein comprises the sequence of SEQ ID NO: 1 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the OTOF isoform 5 protein variant are conservative amino acid substitutions. In some embodiments, the OTOF isoform 5 protein consists of the sequence of SEQ ID NO: 1. In some embodiments, the OTOF isoform 5 protein is encoded by the sequence of SEQ ID NO: 2. In some embodiments, the OTOF isoform 5 protein is encoded by the sequence of SEQ ID NO: 3.

In some embodiments, the N-terminal portion of the OTOF isoform 5 protein consists of the sequence of SEQ ID NO: 58 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the N-terminal portion of the OTOF isoform 5 protein variant are conservative amino acid substitutions. In some embodiments, the N-terminal portion of the OTOF isoform 5 protein consists of the sequence of SEQ ID NO: 58. In some embodiments, the N-terminal portion of the OTOF isoform 5 protein is encoded by the sequence of SEQ ID NO: 56.

In some embodiments, the C-terminal portion of the OTOF isoform 5 protein consists of the sequence of SEQ ID NO: 59 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the C-terminal portion of the OTOF isoform 5 protein variant are conservative amino acid substitutions. In some embodiments, the C-terminal portion of the OTOF isoform 5 protein consists of the sequence of SEQ ID NO: 59. In some embodiments, the C-terminal portion of the OTOF isoform 5 protein is encoded by the sequence of SEQ ID NO: 57.

In some embodiments, the ITRs in the first vector and second vector are AAV2 ITRs. In some embodiments, the ITRs in the first vector and second vector have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.

In some embodiments, the poly(A) sequence is a bovine growth hormone (bGH) poly(A) signal sequence.

In some embodiments, the splice donor sequence in the first vector comprises or consists of the sequence of SEQ ID NO: 54.

In some embodiments, the splice acceptor sequence in the second vector comprises or consists of the sequence of SEQ ID NO: 55.

In some embodiments, the first AAV vector comprises a Kozak sequence 3′ of the promoter and 5′ of the first coding polynucleotide that encodes the N-terminal portion of the OTOF isoform 5 protein.

In some embodiments of, the first AAV vector contains a polynucleotide sequence comprising the sequence of nucleotides 2272 to 6041 of SEQ ID NO: 60. In some embodiments, the first AAV vector contains a polynucleotide sequence comprising or consisting of the sequence of nucleotides 2049 to 6264 of SEQ ID NO: 60.

In some embodiments, the first AAV vector contains a polynucleotide sequence comprising the sequence of nucleotides 182 to 3949 of SEQ ID NO: 62. In some embodiments, the first AAV vector contains a polynucleotide sequence comprising or consisting of the sequence of nucleotides 19 to 4115 of SEQ ID NO: 62.

In some embodiments, the first AAV vector contains a polynucleotide sequence comprising the sequence of positions 2267 to 6014 of SEQ ID NO: 64. In some embodiments, the first AAV vector contains a polynucleotide sequence comprising or consisting of the sequence of positions 2049 to 6237 of SEQ ID NO: 64.

In some embodiments, the first AAV vector contains a polynucleotide sequence comprising the sequence of positions 177 to 3924 of SEQ ID NO: 65. In some embodiments, the first AAV vector contains a polynucleotide sequence comprising or consisting of the sequence of positions 19 to 4090 of SEQ ID NO: 65.

In some embodiments, the second AAV vector contains a polynucleotide sequence comprising the sequence of nucleotides 2267 to 6476 of SEQ ID NO: 61. In some embodiments, the second AAV vector contains a polynucleotide sequence comprising or consisting of the sequence of nucleotides 2049 to 6693 of SEQ ID NO: 61.

In some embodiments, the second AAV vector contains a polynucleotide sequence comprising the sequence of nucleotides 187 to 4396 of SEQ ID NO: 63. In some embodiments, the second AAV vector contains a polynucleotide sequence comprising or consisting of the sequence of nucleotides 19 to 4589 of SEQ ID NO: 63.

In some embodiments, the first AAV vector contains a polynucleotide sequence comprising the sequence of nucleotides 235 to 4004 of SEQ ID NO: 66. In some embodiments, the first AAV vector contains a polynucleotide sequence comprising or consisting of the sequence of nucleotides 12 to 4227 of SEQ ID NO: 66.

In some embodiments, the first AAV vector contains a polynucleotide sequence comprising the sequence of nucleotides 230 to 3977 of SEQ ID NO: 68. In some embodiments, the first AAV vector contains a polynucleotide sequence comprising or consisting of the sequence of nucleotides 12 to 4200 of SEQ ID NO: 68.

In some embodiments, the second AAV vector contains a polynucleotide sequence comprising the sequence of nucleotides 229 to 4438 of SEQ ID NO: 67. In some embodiments, the second AAV vector contains a polynucleotide sequence comprising or consisting of the sequence of nucleotides 12 to 4655 of SEQ ID NO: 67.

In some embodiments, the subject is less than 18 years of age. In some embodiments, the subject is less than 18 years of age and at least 7 years of age. In some embodiments, the subject is older than 24 months of age and younger than 18 years of age. In some embodiments, the subject is older than 24 months of age and younger than 7 years of age (e.g., younger than 6 years of age, younger than 5 years of age, younger than 4 years of age, or younger than 3 years of age). In some embodiments, the subject is 24 months of age or younger (e.g., younger than 24 months of age, younger than 18 months of age, younger than 12 months of age, younger than 6 months of age, or younger than 3 months of age).

In some embodiments, the subject has or is identified as having profound sensorineural hearing loss (e.g., based on ABR measurements, such as ≥90 dB hearing level (HL) or absence of an ABR neural signal in response to a click stimulus at or below 85 decibels normalized Hearing Level (dB nHL), e.g., profound deafness secondary to biallelic OTOF mutations).

In some embodiments, the subject has or is identified as having behavioral open-set word detection scores consistent with the speech criteria listed on a cochlear implant label (e.g., behavioral open-set word detection scores of <30% in the ear(s) to be treated).

In some embodiments, the subject has or is identified as having congenital auditory neuropathy.

In some embodiments, the subject has or is identified as having present outer hair cell function.

In some embodiments, the subject has or is identified as having detectable otoacoustic emissions (OAEs, e.g., ≥6 dB signal-to-noise ratio at ≥3 frequencies in a distortion product (DP) Gram measured from 1 to 8 kHz).

In some embodiments, a cochlear microphonic is present in the ear(s) to be treated.

In some embodiments, the administering is unilateral. In some embodiments, the administering is bilateral.

In some embodiments, the administering to an inner ear includes intracochlear injection via a catheter placed through the round window membrane of the cochlea (e.g., into the inner ear perilymph). In some embodiments, the administering further includes creating a fenestration in the lateral semicircular canal. In some embodiments, the injection is performed using a syringe and syringe pump (e.g., a syringe is attached to the catheter and the syringe and catheter combination are loaded onto the syringe pump). In some embodiments, the administering is at a rate of 0.8-1 mL/hr (e.g., 0.9 mL/hr).

In some embodiments, the administering occurs once per ear.

In some embodiments, the first vector and the second vector are administered at about a 1:1 ratio (e.g., at a 1:1 ratio).

In some embodiments, the dual vector system is formulated as an aqueous suspension. In some embodiments, the first vector and second vector are matched in titer and mixed at an approximately equal ratio (i.e., the titer of 1×10¹³ vg/mL to 1×10¹⁴ vg/mL is produced by combining the first vector and second vector in an approximately 1:1 ratio). In some embodiments, the suspension includes (in addition to the AAV vectors) 10 mM sodium phosphate or disodium phosphate (also called sodium phosphate dibasic and having the formula Na₂HPO₄), 180 mM sodium chloride, 5% (w/v) sucrose, and 0.001% (w/v) poloxamer 188 at a pH of about 7.4. In some embodiments, the suspension includes 10 mM sodium phosphate. In some embodiments, the suspension includes 10 mM disodium phosphate.

In some embodiments, the method further includes administering a corticosteroid (e.g., prednisone) to the subject for the first four weeks after the administering of the OTOF dual vector system. In some embodiments, the method includes administering 1 mg/kg of the corticosteroid daily during the first two weeks after the administering of the OTOF dual vector system, administering 0.5 mg/kg daily during the third week after the administering of the OTOF dual vector system, and administering 0.25 mg/kg daily during the fourth week after the administering of the OTOF dual vector system.

In some embodiments, the OTOF dual vector system is administered in an amount of 1.0×10¹³ vg/mL to 5.0×10¹³ vg/mL in a volume of 200-250 μL. In some embodiments, the OTOF dual vector system is administered in an amount of 2.0×10¹³ vg/mL to 5.0×10¹³ vg/mL in a volume of 200-250 μL. In some embodiments, the OTOF dual vector system is administered in an amount of 3.0×10¹³ vg/mL to 5.0×10¹³ vg/mL in a volume of 200-250 μL. In some embodiments, the OTOF dual vector system is administered in an amount of 4.0×10¹³ vg/mL to 5.0×10¹³ vg/mL in a volume of 200-250 μL.

In some embodiments, the OTOF dual vector system is administered in an amount of 5.0×10¹³ vg/mL to 1.0×10¹⁴ vg/mL in a volume of 200-250 μL. In some embodiments, the OTOF dual vector system is administered in an amount of 5.0×10¹³ vg/mL to 9.0×10¹³ vg/mL in a volume of 200-250 μL. In some embodiments, the OTOF dual vector system is administered in an amount of 5.0×10¹³ vg/mL to 8.0×10¹³ vg/mL in a volume of 200-250 μL. In some embodiments, the OTOF dual vector system is administered in an amount of 5.0×10¹³ vg/mL to 7.0×10¹³ vg/mL in a volume of 200-250 μL. In some embodiments, the OTOF dual vector system is administered in an amount of 5.0×10¹³ vg/mL to 6.0×10¹³ vg/mL in a volume of 200-250 μL.

In some embodiments, the OTOF dual vector system is administered in an amount of 1.0×10¹³ vg/mL to 1.0×10¹⁴ vg/mL in a volume of 210-250 μL. In some embodiments, the OTOF dual vector system is administered in an amount of 1.0×10¹³ vg/mL to 1.0×10¹⁴ vg/mL in a volume of 220-250 μL. In some embodiments, the OTOF dual vector system is administered in an amount of 1.0×10¹³ vg/mL to 1.0×10¹⁴ vg/mL in a volume of 230-250 μL. In some embodiments, the OTOF dual vector system is administered in an amount of 1.0×10¹³ vg/mL to 1.0×10¹⁴ vg/mL in a volume of 240-250 μL. In some embodiments, the OTOF dual vector system is administered in an amount of 2.0×10¹³ vg/mL to 9.0×10¹³ vg/mL in a volume of 240-250 μL. In some embodiments, the OTOF dual vector system is administered in an amount of 3.0×10¹³ vg/mL to 8.0×10¹³ vg/mL in a volume of 240-250 μL. In some embodiments, the OTOF dual vector system is administered in an amount of 3.0×10¹³ vg/mL to 8.0×10¹³ vg/mL in a volume of 240 μL.

In some embodiments, the OTOF dual vector system is administered in an amount of 3.0×10¹³ vg/mL in a volume of 240 μL. In some embodiments, the OTOF dual vector system is administered in an amount of 7.3×10¹³ vg/mL in a volume of 240 μL.

In some embodiments of any of the foregoing aspects, the method further includes identifying the subject as having a mutation in OTOF (e.g., a biallelic likely pathogenic or pathogenic mutation) prior to administering the composition.

In some embodiments of any of the foregoing aspects, the method further includes identifying the subject as having profound sensorineural hearing loss prior to administering the composition.

In some embodiments of any of the foregoing aspects, the subject has or has been identified as having Deafness, Autosomal Recessive 9 (DFNB9).

In some embodiments of any of the foregoing aspects, the method further includes the step of evaluating the hearing of the subject prior to administering the dual vector system.

In some embodiments of any of the foregoing aspects, the method further includes identifying the subject as having present outer hair cell function prior to administering the dual vector system (e.g., based on having detectable otoacoustic emissions (OAEs, e.g., ≥6 dB signal-to-noise ratio at ≥3 frequencies in a distortion product (DP) Gram measured from 1 to 8 kHz and/or a present cochlear microphonic in the ear(s) to be treated).

In some embodiments of any of the foregoing aspects, the method increases OTOF expression in a cochlear hair cell. In some embodiments, the cochlear hair cell is an inner hair cell.

In some embodiments of any of the foregoing aspects, the method further includes evaluating the hearing of the subject after administering the dual vector system.

In some embodiments of any of the foregoing aspects, the method increases OTOF expression in a cell (e.g., a cochlear hair cell), improves hearing (e.g., as assessed by standard tests, such as audiometry, auditory brainstem response (ABR), electrocochleography (ECOG), and otoacoustic emissions), prevents or reduces hearing loss, delays the development of hearing loss, slows the progression of hearing loss, improves speech discrimination, or improves hair cell function.

In some embodiments of any of the foregoing aspects, the method improves one or more parameters selected from the subject's auditory brainstem response (ABR), behavioral audiometry, and score in one or more hearing questionnaires or behavioral tasks. In some embodiments, the hearing questionnaires and behavioral tasks include one or more of the Auditory Skills Checklist, Open & Closed set task, Early Speech Perception test, Pediatric Speech Intelligibility test, Lexical Neighborhood Test Multisyllabic Lexical Neighborhood Test, Consonant-Nucleus-Consonant test, Bamford-Kowal-Bench sentence test, LittIEARS® Auditory Questionnaire, MacArthur-Bates Communicative Development Inventories Words and Gestures, Quality of Life-Cochlear Implant, Pediatric Quality of Life Inventory, Hearing Environments and Reflection on Quality of Life (HEAR-QL)-26, HEAR-QL-28, The Health Utilities Index 3, Vanderbilt Fatigue Scales, and AzBio test. In some embodiments, the method improves the subject's score in the LittIEARS® Auditory Questionnaire. In some embodiments, the method improves hearing thresholds by at least 55 decibels hearing level with air conduction as assessed by behavioral pure tone audiometry. In some embodiments, the method results in a positive ABR wave V amplitude response.

In another aspect, the invention provides a kit including a suspension described herein. In some embodiments, the kit further includes one or more of a syringe, syringe pump, and catheter.

In another aspect, the invention provides a syringe containing a suspension described herein.

In another aspect, the invention provides a method of administering a nucleic acid vector to the inner ear of a human subject by creating an opening in the round widow membrane of the cochlea and an opening in a second location in the inner ear and inserting a catheter through the opening in the round window membrane to infuse the nucleic acid vector into the perilymph. In some embodiments, the opening made in the second location in the inner ear is made in a semicircular canal. In some embodiments, the semicircular canal is a lateral semicircular canal. In some embodiments, the semicircular canal is a posterior semicircular canal. In some embodiments, the semicircular canal is a superior semicircular canal. In some embodiments, a surgical procedure is performed to access the middle ear before creating the opening in the round window membrane and lateral semicircular canal. In some embodiments, the surgical procedure involves a mastoidectomy and opening a facial recess. In some embodiments, the surgical procedure is a standard surgical procedure for cochlear implant surgery. In some embodiments, a syringe and/or a syringe pump are used in combination with the catheter for said administering (e.g., a syringe is attached to the catheter and the syringe and catheter combination are loaded onto the syringe pump). In some embodiments, the nucleic acid vector (e.g., a solution containing the nucleic acid vector) is administered at a rate of 0.5-1 mL/hr (e.g., 0.6-1 mL/hr, 0.7-1 mL/hr, 0.8-1 mL/hr, or 0.9-1 mL/hour, such as 0.9 mL/hr). In some embodiments, the nucleic acid vector is a viral vector. In some embodiments, the viral vector is a lentivirus vector, an adenovirus vector, or an adeno-associated virus (AAV) vector. In some embodiments, the viral vector is an AAV vector. In some embodiments, the human subject has or is at risk of developing sensorineural hearing loss. In some embodiments, the hearing loss is genetic hearing loss. In some embodiments, the genetic hearing loss is autosomal dominant hearing loss, autosomal recessive hearing loss, or X-linked hearing loss. In some embodiments, the nucleic acid vector contains a polynucleotide encoding a protein that is absent or dysfunctional in the subject (e.g., a protein that is not produced or that does not function properly due to a genetic mutation associated with the hearing loss, e.g., the polynucleotide encodes a functional protein that is deficient in the subject due to a genetic mutation) or a polynucleotide encoding an expression product designed to correct a genetic mutation in the subject (e.g., correct a genetic mutation associated with the hearing loss via gene editing). In some embodiments, the hearing loss is acquired hearing loss. In some embodiments, the acquired hearing loss is noise-induced hearing loss, age-related hearing loss, disease or infection-related hearing loss, head trauma-related hearing loss, or ototoxic drug-induced hearing loss. In some embodiments, the nucleic acid vector includes a polynucleotide encoding an expression product that induces or increases cochlear hair cell regeneration, increases cochlear supporting cell numbers, prevents or reduces cochlear hair cell damage, prevents or reduces cochlear hair cell death, increases cochlear hair cell maturation, improves cochlear hair cell function, or promotes or increases cochlear hair cell survival. In some embodiments, the human subject has or is at risk of developing vestibular dysfunction. In some embodiments, the vestibular dysfunction is vertigo, dizziness, imbalance (e.g., loss of balance or a balance disorder), oscillopsia, or bilateral vestibulopathy. In some embodiments, the vestibular dysfunction is associated with a genetic mutation. In some embodiments, the nucleic acid vector contains a polynucleotide encoding a protein that is absent or dysfunctional in the subject (e.g., a protein that is not produced or that does not function properly in the inner ear of the subject due to the genetic mutation associated with the vestibular dysfunction, e.g., the polynucleotide encodes a functional protein that is deficient in the subject due to the genetic mutation) or a polynucleotide encoding an expression product designed to correct a genetic mutation in the subject (e.g., correct a genetic mutation associated with the vestibular dysfunction via gene editing). In some embodiments, the vestibular dysfunction is associated with damage to or loss of vestibular cells (e.g., vestibular hair cells or supporting cells, such as damage to or loss of vestibular cells, related to disease or infection, head trauma, ototoxic drugs (e.g., vestibulotoxic drugs), or aging). In some embodiments, the nucleic acid vector includes a polynucleotide encoding an expression product that induces or increases vestibular hair cell regeneration, increases vestibular supporting cell numbers, prevents or reduces vestibular hair cell damage, prevents or reduces vestibular hair cell death, increases vestibular hair cell maturation, improves vestibular hair cell function, or promotes or increases vestibular hair cell survival.

### Definitions

As used herein, the term “about” refers to a value that is within 10% above or below the value being described.

As used herein, “administration” refers to providing or giving a subject a therapeutic agent (e.g., a composition containing a first nucleic acid vector containing a polynucleotide that encodes an N-terminal portion of an otoferlin protein and a second nucleic acid vector containing a polynucleotide that encodes a C-terminal portion of an otoferlin protein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject in need thereof, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. Note that when a combination of active ingredients is administered, the effective amount of the combination may or may not include amounts of each ingredient that would have been effective if administered individually. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear hair cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear hair cell). Exogenous materials include those that are provided from an external source to an organism or to cultured matter extracted therefrom.

As used herein, the term “hair cell-specific expression” refers to production of an RNA transcript or polypeptide primarily within hair cells (e.g., cochlear hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, Western Blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells (e.g., cochlear hair cells) compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., ABR threshold) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule that can be joined to a second molecule, wherein the molecules are so arranged that the first molecule affects the function of the second molecule. The term “operably linked” includes the juxtaposition of two or more components (e.g., a promoter and another sequence element) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the terms “otoferlin isoform 5” and “OTOF isoform 5” refer to an isoform of the gene associated with nonsyndromic recessive deafness DFNB9. The human isoform of the gene is associated with reference sequence NM_001287489, and the transcript includes exons 1-45 and 47 of human otoferlin but lacks exon 46 of the OTOF gene. The human OTOF isoform 5 protein is also known as Otoferlin isoform e. The terms “otoferlin isoform 5” and “OTOF isoform 5” also refer to variants of the wild-type OTOF isoform 5 protein and polynucleotides encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type OTOF isoform 5 protein (e.g., SEQ ID NO: 1) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the polynucleotide sequence of a wild-type OTOF isoform 5 gene, provided that the OTOF isoform 5 analog encoded retains the therapeutic function of wild-type OTOF isoform 5. OTOF isoform 5 protein variants can have one or more (e.g., 1, 2, 3, 4, 5, 6, 7, 8, 9, 10, 11, 12, 13, 14, 15, 16, 17, 18, 19, 20, or more) conservative amino acid substitutions relative to a wild-type OTOF isoform 5 (e.g., SEQ ID NO: 1), provided that the that the OTOF isoform 5 variant retains the therapeutic function of wild-type OTOF isoform 5 and has no more than 10% amino acid substitutions in an N-terminal portion of the amino acid sequence and no more than 10% amino acid substitutions in a C-terminal portion of the amino acid sequence. As used herein, OTOF isoform 5 may refer to the protein localized to inner hair cells or to the gene encoding this protein, depending upon the context, as will be appreciated by one of skill in the art. OTOF isoform 5 may refer to human OTOF isoform 5 or to a homolog from another mammalian species. Murine otoferlin contains one additional exon relative to human otoferlin (48 exons in murine otoferlin), and the exons of murine otoferlin that correspond to those that encode human OTOF isoform 5 are 1-5, 7-46, and 48. The exon numbering convention used herein is based on the exons currently understood to be present in the consensus transcripts of human OTOF.

As used herein, the term “plasmid” refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

As used herein, the term “promoter” refers to a recognition site on DNA that is bound by an RNA polymerase. The polymerase drives transcription of the transgene. Exemplary promoters suitable for use with the compositions and methods described herein include ubiquitous promoters (e.g., the CAG promoter, cytomegalovirus (CMV) promoter, and smCBA promoter) and cochlear hair cell-specific promoters (e.g., the Myosin 15 (Myo15) promoter).

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full-length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “derivative” as used herein refers to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “recombinogenic region” refers to a region of homology that mediates recombination between two different sequences.

As used herein, the term “regulatory sequence” includes promoters, enhancers, and other expression control elements (e.g., polyadenylation signals) that control the transcription or translation of the polynucleotides that encode OTOF. Such regulatory sequences are described, for example, in Goeddel, Gene Expression Technology: Methods in Enzymology 185 (Academic Press, San Diego, C A, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection and the like.

As used herein, the terms “subject” and “patient” refer to an animal (e.g., a mammal, such as a human), veterinary animals (e.g., cats, dogs, cows, horses, sheep, pigs, etc.) and experimental animal models of diseases (e.g., mice, rats). A subject to be treated according to the methods described herein may be one who has been diagnosed with hearing loss (e.g., hearing loss associated with a mutation in OTOF), or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” of a state, disorder or condition can include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO94/11026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of OTOF as described herein include vectors that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIGS. 1A-1D are a series of graphs of ABR responses measured at Week 6 after a single, unilateral administration of a composition containing an OTOF dual hybrid vector system to the right ear of a subject with biallelic mutations in the OTOF gene who was dosed at 10 months old. ABR responses were measured in response to a click stimulus (FIGS. 1A-1B), 2000 Hz (FIG. 1C), and 500 Hz (FIG. 1D). Neural ABR (Wave V) responses were present at 90 dB nHL in the treated ear and were absent at Baseline.

FIG. 2 is a series of graphs of audiograms (behavioral pure tones) measured at Baseline and at Week 4 and Week 6 after a single, unilateral administration of a composition containing an OTOF dual hybrid vector system to the right ear of the subject with biallelic mutations in the OTOF gene who was dosed at 10 months old. Responses from the right (treated) ear are shown using circles and triangles and responses from the left ear (without the cochlear implant on) are shown using Xs. The treated ear showed profound hearing loss to severe/profound hearing loss from Baseline to Week 4, and the audiogram was essentially stable from Week 4 to Week 6, which may be due to testing difficulties. The untreated ear without the cochlear implant on showed profound hearing loss that did not change from Baseline to Week 6.

FIG. 3 is a series of graphs of ABR responses measured at Baseline in the subject with biallelic mutations in the OTOF gene who was dosed at 10 months old. ABR responses were measured in response to a click stimulus (top) and 2000 Hz (bottom).

FIG. 4 is a graph of a behavioral pure tone audiogram showing responses measured at Baseline, Week 4, and Week 12 in the treated ear of the subject with biallelic mutations in the OTOF gene who was dosed at 10 months old. The treated ear exhibited profound hearing impairment at Baseline and showed responses to multiple pitches at Week 4. At Week 12, the treated ear exhibited responses representative of moderate hearing loss. Arrows indicate no response at the maximum level tested.

FIG. 5 is a drawing depicting the surgical approach and route of administration used for administration of the OTOF dual hybrid vector system. The surgical approach used to access the middle ear was similar to that used for cochlear implant surgery. The OTOF dual hybrid vector system was administered via insertion of a catheter through the round window membrane into the inner ear perilymph for infusion. A fenestration was also made in the lateral semicircular canal. AAV, adeno-associated virus; LSCC, lateral semicircular canal; RWM, round window membrane.

FIG. 6 is a series of graphs of behavioral pure tone audiometry responses measured in the treated ear and the untreated (cochlear implant) ear of the subject with biallelic mutations in the OTOF gene who was dosed at 10 months old. Hearing thresholds for the treated ear improved from profound sensorineural hearing loss to moderate sensorineural hearing loss at Week 12. No improvement was seen in the untreated ear with the cochlear implant turned off. Arrow=no response; HL, hearing level; AC, air conduction.

FIG. 7 is a series of graphs of ABR thresholds measured in the treated ear and the untreated ear of the subject with biallelic mutations in the OTOF gene who was dosed at 10 months old. A positive ABR wave response in hearing intensity from Baseline through Week 12 at thresholds of 40-80 dB was reported in the treated ear; no response was elicited at 100 dB at Baseline. No improvement in hearing intensity was seen in the untreated ear in comparison with the treated ear. Arrow=no response; Week 6: Could not test at 1000 and 4000 Hz due to subject waking up; Week 12: Subject woke up after starting 4000 Hz trial at 60 dB in the treated ear, therefore could not proceed to find threshold. nHL, normalized hearing level.

FIG. 8 is a graph showing the LittIEARS auditory questionnaire score from Baseline to Week 12 from the subject with biallelic mutations in the OTOF gene who was dosed at 10 months old. An improvement in the subject's global auditory skill development was observed at Week 12 from Baseline, according to parental reports and as indicated by the LittIEARS auditory questionnaire. Parents and investigator reported that the subject could hear sounds when the opposite ear cochlear implant was turned off, indicating improved hearing acuity in the treated ear. Parents also reported more natural vocalizations when the opposite ear cochlear implant was turned off.

FIG. 9 is a graph showing measurements of cervical vestibular evoked myogenic potentials (cVEMP) from Baseline to Week 12 from the subject with biallelic mutations in the OTOF gene who was dosed at 10 months old. Vestibular responses were present at Baseline and continued to be present through Week 12 (comparable to Baseline). CI, cochlear implant.

### DETAILED DESCRIPTION

Described herein are compositions and methods for the treatment of sensorineural hearing loss or auditory neuropathy in a subject having biallelic OTOF mutations (such as a mammalian subject, for instance, a human) by administering a first nucleic acid vector (e.g., an AAV vector) containing a promoter and a polynucleotide encoding an N-terminal portion of an otoferlin (OTOF) protein (e.g., a polynucleotide encoding a long isoform of an OTOF protein, such as a wild-type (WT) human OTOF isoform 5 protein or a wild-type human OTOF isoform 1 protein) and a second nucleic acid vector (e.g., an AAV vector) containing a polynucleotide encoding a C-terminal portion of an OTOF protein and a polyadenylation (poly(A)) sequence. When introduced into a mammalian cell, such as a cochlear hair cell, the polynucleotides encoded by the two nucleic acid vectors can combine to form a nucleic acid molecule that encodes the full-length OTOF protein. The compositions and methods described herein can, therefore, be used to induce or increase expression of WT OTOF in cochlear hair cells of a subject who has an OTOF deficiency (e.g., low OTOF expression or an OTOF mutation that impairs OTOF expression or function).

Otoferlin OTOF is a 230 kDa membrane protein that contains at least six C2 domains implicated in calcium, phospholipid, and protein binding. Human OTOF is encoded by a gene that contains 47 exons, and the full-length protein is made up of 1,997 amino acids. OTOF is located at ribbon synapses in inner hair cells, where it is believed to function as a calcium sensor in synaptic vesicle fusion, triggering the fusion of neurotransmitter-containing vesicles with the plasma membrane. It has also been implicated in vesicle replenishment and clathrin-mediated endocytosis, and has been shown to interact with Myosin VI, Rab8b, SNARE proteins, calcium channel Cav1.3, Ergic2, and AP-2. The mechanism by which OTOF mediates exocytosis and the physiological significance of its interactions with its binding partners remain to be determined.

There are multiple long and short isoforms of the Otoferlin gene. Studies of human genetic deafness have suggested that long isoforms are important for inner ear function. However, the role of these individual long isoforms and other protein variants in inner ear function is not understood. To develop effective gene transfer therapies for patients who experience deafness secondary to genetically driven Otoferlin deficiency, a cDNA sequence that encodes functional OTOF isoforms in the ear must be identified.

### Otoferlin-Associated Hearing Loss

OTOF was first identified by a study investigating the genetics of a non-syndromic form of deafness, autosomal recessive deafness-9 (DFNB9). Mutations in OTOF have since been found to cause sensorineural hearing loss in patients throughout the world, with many patients carrying OTOF mutations having auditory neuropathy, a disorder in which the inner ear detects sound, but is unable to properly transmit sound from the ear to the brain. These patients have an abnormal auditory brainstem response (ABR) and impaired speech discrimination with initially normal otoacoustic emissions (OAEs). Patients carrying homozygous or compound heterozygous mutations often develop hearing loss in early childhood, and the severity of hearing impairment has been found to vary with the location and type of mutation in OTOF.

The compositions and methods described herein can be used to treat sensorineural hearing loss or auditory neuropathy by administering a first nucleic acid vector containing a polynucleotide encoding an N-terminal portion of an OTOF protein and a second nucleic acid vector containing a polynucleotide encoding a C-terminal portion of an OTOF protein. The full-length OTOF coding sequence is too large to include in the type of vector that is commonly used for gene therapy (e.g., an adeno-associated virus (AAV) vector, which is thought to have a packaging limit of 5 kb). The compositions and methods described herein overcome this problem by dividing the OTOF coding sequence between two different nucleic acid vectors (e.g., AAV vectors) that can combine in a cell to reconstitute the full-length OTOF sequence. In some embodiments, the OTOF protein encoded by the vectors is an OTOF isoform 5 protein (e.g., a wild-type human OTOF isoform 5 protein). In some embodiments, the OTOF protein encoded by the vectors is an OTOF isoform 1 protein (e.g., a wild-type human OTOF isoform 1 protein). These compositions and methods can be used to treat subjects having one or more mutations in the OTOF gene, e.g., an OTOF mutation that reduces OTOF expression, reduces OTOF function, or is associated with hearing loss (e.g., a frameshift mutation, a nonsense mutation, a deletion, or a missense substitution). When the first and second nucleic acid vectors are administered in a composition, the polynucleotides encoding the N-terminal and C-terminal portions of OTOF can combine within a cell (e.g., a human cell, e.g., a cochlear hair cell) to form a single nucleic acid molecule that contains the full-length OTOF coding sequence (e.g., through homologous recombination and/or splicing).

The nucleic acid vectors (e.g., AAV vectors) used in the compositions and methods described herein include polynucleotide sequences that encode wild-type OTOF isoform 5, or a variant thereof, such as a polynucleotide sequences that, when combined, encode a protein having at least 85% sequence identity (e.g., 85%, 90%, 91%, 92%, 93%, 94%, 95%, 96%, 97%, 98%, 99%, or more, sequence identity) to the amino acid sequence of wild-type mammalian (e.g., human or mouse) OTOF isoform 5. The polynucleotides used in the nucleic acid vectors described herein can encode an N-terminal portion and a C-terminal portion of an OTOF isoform 5 amino acid sequence in Table 2 below (e.g., two portions that, when combined, encode a full-length OTOF isoform 5 amino acid sequence listed in Table 2, e.g., SEQ ID NO: 1).

According to the methods described herein, a subject can be administered a composition containing a first nucleic acid vector and a second nucleic acid vector that contain an N-terminal and C-terminal portion, respectively, of a polynucleotide sequence encoding the amino acid sequence of SEQ ID NO: 1, or a polynucleotide sequence encoding an amino acid sequence having at least 85% sequence identity (e.g., 85%, 90%, 91%, 92%, 93%, 94%, 95%, 96%, 97%, 98%, 99%, or more, sequence identity) to the amino acid sequence of SEQ ID NO: 1, or a polynucleotide sequence encoding an amino acid sequence that contains one or more conservative amino acid substitutions relative to SEQ ID NO: 1 (e.g., 1, 2, 3, 4, 5, 6, 7, 8, 9, 10, 11, 12, 13, 14, 15, 16, 17, 18, 19, 20 or more conservative amino acid substitutions), provided that the OTOF analog encoded retains the therapeutic function of wild-type OTOF isoform 5 (e.g., the ability to regulate exocytosis at ribbon synapses or rescue or improve ABR response in an animal model of hearing loss related to Otoferlin gene deficiency (e.g., OTOF mutation)). No more than 10% of the amino acids in the N-terminal portion of the human OTOF isoform 5 protein and no more than 10% of the amino acids in the C-terminal portion of the human OTOF isoform 5 protein may be replaced with conservative amino acid substitutions. The OTOF isoform 5 protein may be encoded by a polynucleotide having the sequence of SEQ ID NO: 2 or SEQ ID NO: 3. The OTOF isoform 5 protein may also be encoded by a polynucleotide having single nucleotide variants (SNVs) that have been found to be non-pathogenic in human subjects. The OTOF isoform 5 protein may be a human OTOF isoform 5 protein or may be a homolog of the human isoform 5 protein from another mammalian species (e.g., mouse, rat, cow, horse, goat, sheep, donkey, cat, dog, rabbit, guinea pig, or other mammal).

TABLE 2
OTOF Sequences

| SEQ ID | | |
|---|---|---|
| NO. | Sequence Name | Sequence |
| 1 | Human OTOF | MALLIHLKTVSELRGRGDRIAKVTFRGQSFYSRVLENCEDVADFDE |
| | isoform 5 protein | TFRWPVASSIDRNEMLEIQVFNYSKVFSNKLIGTFRMVLQKVVEES |
| | (NP_001274418.1), | HVEVTDTLIDDNNAIIKTSLCVEVRYQATDGTVGSWDDGDFLGDES |
| | also called human | LQEEEKDSQETDGLLPGSRPSSRPPGEKSFRRAGRSVFSAMKLGK |
| | otoferlin isoform e, | NRSHKEEPQRPDEPAVLEMEDLDHLAIRLGDGLDPDSVSLASVTAL |
| | 1997 aa | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLQNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTAFVWFLNPLKSIKYLICTRYKWLIIKIVLAL |
| | | LGLLMLGLFLYSLPGYMVKKLLGA |
| 2 | DNA sequence | ATGGCCTTGCTCATCCACCTCAAGACAGTCTCGGAGCTGCGGG |
| | encoding the human | GCAGGGGCGACCGGATCGCCAAAGTGACTTTCCGAGGGCAATC |
| | otoferlin isoform 5 | CTTCTACTCTCGGGTCCTGGAGAACTGTGAGGATGTGGCTGACT |
| | protein (SEQ ID NO: | TTGATGAGACATTTCGGTGGCCGGTGGCCAGCAGCATCGACAG |
| | 1), 5994 bp, | AAATGAGATGCTGGAGATTCAGGTTTTCAACTACAGCAAAGTCTT |
| | corresponds to the | CAGCAACAAGCTCATCGGGACCTTCCGCATGGTGCTGCAGAAG |
| | coding sequence | GTGGTAGAGGAGAGCCATGTGGAGGTGACTGACACGCTGATTG |
| | documented in | ATGACAACAATGCTATCATCAAGACCAGCCTGTGCGTGGAGGTC |
| | NM_001287489 | CGGTATCAGGCCACTGACGGCACAGTGGGCTCCTGGGACGATG |
| | | GGGACTTCCTGGGAGATGAGTCTCTTCAAGAGGAAGAGAAGGA |
| | | CAGCCAAGAGACGGATGGACTGCTCCCAGGCTCCCGGCCCAGC |
| | | TCCCGGCCCCCAGGAGAGAAGAGCTTCCGGAGAGCCGGGAGG |
| | | AGCGTGTTCTCCGCCATGAAGCTCGGCAAAAACCGGTCTCACAA |
| | | GGAGGAGCCCCAAAGACCAGATGAACCGGCGGTGCTGGAGAT |
| | | GGAAGACCTTGACCATCTGGCCATTCGGCTAGGAGATGGACTG |
| | | GATCCCGACTCGGTGTCTCTAGCCTCAGTCACAGCTCTCACCAC |
| | | TAATGTCTCCAACAAGCGATCTAAGCCAGACATTAAGATGGAGC |
| | | CAAGTGCTGGGCGGCCCATGGATTACCAGGTCAGCATCACGGT |
| | | GATCGAGGCCCGGCAGCTGGTGGGCTTGAACATGGACCCTGTG |
| | | GTGTGCGTGGAGGTGGGTGACGACAAGAAGTACACATCCATGA |
| | | AGGAGTCCACTAACTGCCCCTATTACAACGAGTACTTCGTCTTC |
| | | GACTTCCATGTCTCTCCGGATGTCATGTTTGACAAGATCATCAAG |
| | | ATTTCGGTGATTCACTCCAAGAACCTGCTGCGCAGTGGCACCCT |
| | | GGTGGGCTCCTTCAAAATGGACGTGGGAACCGTGTACTCGCAG |
| | | CCAGAGCACCAGTTCCATCACAAGTGGGCCATCCTGTCTGACCC |
| | | CGATGACATCTCCTCGGGGCTGAAGGGCTACGTGAAGTGTGAC |
| | | GTTGCCGTGGTGGGCAAAGGGGACAACATCAAGACGCCCCACA |
| | | AGGCCAATGAGACCGACGAAGATGACATTGAGGGGAACTTGCT |
| | | GCTCCCCGAGGGGGTGCCCCCCGAACGCCAGTGGGCCCGGTT |
| | | CTATGTGAAAATTTACCGAGCAGAGGGGCTGCCCCGTATGAACA |
| | | CAAGCCTCATGGCCAATGTAAAGAAGGCTTTCATCGGTGAAAAC |
| | | AAGGACCTCGTGGACCCCTACGTGCAAGTCTTCTTTGCTGGCCA |
| | | GAAGGGCAAGACTTCAGTGCAGAAGAGCAGCTATGAGCCCCTG |
| | | TGGAATGAGCAGGTCGTCTTTACAGACCTCTTCCCCCCACTCTG |
| | | CAAACGCATGAAGGTGCAGATCCGAGACTCGGACAAGGTCAAC |
| | | GACGTGGCCATCGGCACCCACTTCATTGACCTGCGCAAGATTTC |
| | | TAATGACGGAGACAAAGGCTTCCTGCCCACACTGGGCCCAGCC |
| | | TGGGTGAACATGTACGGCTCCACACGTAACTACACGCTGCTGGA |
| | | TGAGCATCAGGACCTGAACGAGGGCCTGGGGGAGGGTGTGTC |
| | | CTTCCGGGCCCGGCTCCTGCTGGGCCTGGCTGTGGAGATCGTA |
| | | GACACCTCCAACCCTGAGCTCACCAGCTCCACAGAGGTGCAGG |
| | | TGGAGCAGGCCACGCCCATCTCGGAGAGCTGTGCAGGTAAAAT |
| | | GGAAGAATTCTTTCTCTTTGGAGCCTTCCTGGAGGCCTCAATGA |
| | | TCGACCGGAGAAACGGAGACAAGCCCATCACCTTTGAGGTCAC |
| | | CATAGGCAACTATGGGAACGAAGTTGATGGCCTGTCCCGGCCC |
| | | CAGCGGCCTCGGCCCCGGAAGGAGCCGGGGGATGAGGAAGAA |
| | | GTAGACCTGATTCAGAACGCAAGTGATGACGAGGCCGGTGATG |
| | | CCGGGGACCTGGCCTCAGTCTCCTCCACTCCACCAATGCGGCC |
| | | CCAGGTCACCGACAGGAACTACTTCCATCTGCCCTACCTGGAGC |
| | | GAAAGCCCTGCATCTACATCAAGAGCTGGTGGCCGGACCAGCG |
| | | CCGCCGCCTCTACAATGCCAACATCATGGACCACATTGCCGACA |
| | | AGCTGGAAGAAGGCCTGAACGACATACAGGAGATGATCAAAAC |
| | | GGAGAAGTCCTACCCTGAGCGTCGCCTGCGGGGCGTCCTGGA |
| | | GGAGCTGAGCTGTGGCTGCTGCCGCTTCCTCTCCCTCGCTGAC |
| | | AAGGACCAGGGCCACTCATCCCGCACCAGGCTTGACCGGGAGC |
| | | GCCTCAAGTCCTGCATGAGGGAGCTGGAAAACATGGGGCAGCA |
| | | GGCCAGGATGCTGCGGGCCCAGGTGAAGCGGCACACGGTGCG |
| | | GGACAAGCTGAGGCTGTGCCAGAACTTCCTGCAGAAGCTGCGC |
| | | TTCCTGGCGGACGAGCCCCAGCACAGCATTCCCGACATCTTCAT |
| | | CTGGATGATGAGCAACAACAAGCGTGTCGCCTATGCCCGTGTG |
| | | CCCTCCAAGGACCTGCTCTTCTCCATCGTGGAGGAGGAGACTG |
| | | GCAAGGACTGCGCCAAGGTCAAGACGCTCTTCCTTAAGCTGCC |
| | | AGGGAAGCGGGGCTTCGGCTCGGCAGGCTGGACAGTGCAGGC |
| | | CAAGGTGGAGCTGTACCTGTGGCTGGGCCTCAGCAAACAGCGC |
| | | AAGGAGTTCCTGTGCGGCCTGCCCTGTGGCTTCCAGGAGGTCA |
| | | AGGCAGCCCAGGGCCTGGGCCTGCATGCCTTCCCACCCGTCAG |
| | | CCTGGTCTACACCAAGAAGCAGGCGTTCCAGCTCCGAGCGCAC |
| | | ATGTACCAGGCCCGCAGCCTCTTTGCCGCCGACAGCAGCGGAC |
| | | TCTCAGACCCCTTTGCCCGCGTCTTCTTCATCAATCAGAGTCAG |
| | | TGCACAGAGGTGCTGAATGAGACCCTGTGTCCCACCTGGGACC |
| | | AGATGCTGGTGTTCGACAACCTGGAGCTCTATGGTGAAGCTCAT |
| | | GAGCTGAGGGACGATCCGCCCATCATTGTCATTGAAATCTATGA |
| | | CCAGGATTCCATGGGCAAAGCTGACTTCATGGGCCGGACCTTC |
| | | GCCAAACCCCTGGTGAAGATGGCAGACGAGGCGTACTGCCCAC |
| | | CCCGCTTCCCACCTCAGCTCGAGTACTACCAGATCTACCGTGGC |
| | | AACGCCACAGCTGGAGACCTGCTGGCGGCCTTCGAGCTGCTGC |
| | | AGATTGGACCAGCAGGGAAGGCTGACCTGCCCCCCATCAATGG |
| | | CCCGGTGGACGTGGACCGAGGTCCCATCATGCCCGTGCCCATG |
| | | GGCATCCGGCCCGTGCTCAGCAAGTACCGAGTGGAGGTGCTGT |
| | | TCTGGGGCCTACGGGACCTAAAGCGGGTGAACCTGGCCCAGGT |
| | | GGACCGGCCACGGGTGGACATCGAGTGTGCAGGGAAGGGGGT |
| | | GCAGTCGTCCCTGATCCACAATTATAAGAAGAACCCCAACTTCA |
| | | ACACCCTCGTCAAGTGGTTTGAAGTGGACCTCCCAGAGAACGA |
| | | GCTGCTGCACCCGCCCTTGAACATCCGTGTGGTGGACTGCCGG |
| | | GCCTTCGGTCGCTACACACTGGTGGGCTCCCATGCCGTCAGCT |
| | | CCCTGCGACGCTTCATCTACCGGCCCCCAGACCGCTCGGCCCC |
| | | CAGCTGGAACACCACGGTCAGGCTTCTCCGGCGCTGCCGTGTG |
| | | CTGTGCAATGGGGGCTCCTCCTCTCACTCCACAGGGGAGGTTG |
| | | TGGTGACTATGGAGCCAGAGGTACCCATCAAGAAACTGGAGAC |
| | | CATGGTGAAGCTGGACGCGACTTCTGAAGCTGTTGTCAAGGTG |
| | | GATGTGGCTGAGGAGGAGAAGGAGAAGAAGAAGAAGAAGAAGG |
| | | GCACTGCGGAGGAGCCAGAGGAGGAGGAGCCAGACGAGAGCA |
| | | TGCTGGACTGGTGGTCCAAGTACTTTGCCTCCATTGACACCATG |
| | | AAGGAGCAACTTCGACAACAAGAGCCCTCTGGAATTGACTTGGA |
| | | GGAGAAGGAGGAAGTGGACAATACCGAGGGCCTGAAGGGGTC |
| | | AATGAAGGGCAAGGAGAAGGCAAGGGCTGCCAAAGAGGAGAAG |
| | | AAGAAGAAAACTCAGAGCTCTGGCTCTGGCCAGGGGTCCGAGG |
| | | CCCCCGAGAAGAAGAAACCCAAGATTGATGAGCTTAAGGTATAC |
| | | CCCAAAGAGCTGGAGTCCGAGTTTGATAACTTTGAGGACTGGCT |
| | | GCACACTTTCAACTTGCTTCGGGGCAAGACCGGGGATGATGAG |
| | | GATGGCTCCACCGAGGAGGAGCGCATTGTGGGACGCTTCAAGG |
| | | GCTCCCTCTGCGTGTACAAAGTGCCACTCCCAGAGGACGTGTC |
| | | CCGGGAAGCCGGCTACGACTCCACCTACGGCATGTTCCAGGGC |
| | | ATCCCGAGCAATGACCCCATCAATGTGCTGGTCCGAGTCTATGT |
| | | GGTCCGGGCCACGGACCTGCACCCTGCTGACATCAACGGCAAA |
| | | GCTGACCCCTACATCGCCATCCGGCTAGGCAAGACTGACATCC |
| | | GCGACAAGGAGAACTACATCTCCAAGCAGCTCAACCCTGTCTTT |
| | | GGGAAGTCCTTTGACATCGAGGCCTCCTTCCCCATGGAATCCAT |
| | | GCTGACGGTGGCTGTGTATGACTGGGACCTGGTGGGCACTGAT |
| | | GACCTCATTGGGGAAACCAAGATCGACCTGGAGAACCGCTTCTA |
| | | CAGCAAGCACCGCGCCACCTGCGGCATCGCCCAGACCTACTCC |
| | | ACACATGGCTACAATATCTGGCGGGACCCCATGAAGCCCAGCC |
| | | AGATCCTGACCCGCCTCTGCAAAGACGGCAAAGTGGACGGCCC |
| | | CCACTTTGGGCCCCCTGGGAGAGTGAAGGTGGCCAACCGCGTC |
| | | TTCACTGGGCCCTCTGAGATTGAGGACGAGAACGGTCAGAGGA |
| | | AGCCCACAGACGAGCATGTGGCGCTGTTGGCCCTGAGGCACTG |
| | | GGAGGACATCCCCCGCGCAGGCTGCCGCCTGGTGCCAGAGCA |
| | | TGTGGAGACGAGGCCGCTGCTCAACCCCGACAAGCCGGGCATC |
| | | GAGCAGGGCCGCCTGGAGCTGTGGGTGGACATGTTCCCCATGG |
| | | ACATGCCAGCCCCTGGGACGCCTCTGGACATCTCACCTCGGAA |
| | | GCCCAAGAAGTACGAGCTGCGGGTCATCATCTGGAACACAGAT |
| | | GAGGTGGTCTTGGAGGACGACGACTTCTTCACAGGGGAGAAGT |
| | | CCAGTGACATCTTCGTGAGGGGGTGGCTGAAGGGCCAGCAGGA |
| | | GGACAAGCAGGACACAGACGTCCACTACCACTCCCTCACTGGC |
| | | GAGGGCAACTTCAACTGGCGCTACCTGTTCCCCTTCGACTACCT |
| | | GGCGGCGGAGGAGAAGATCGTCATCTCCAAGAAGGAGTCCATG |
| | | TTCTCCTGGGACGAGACCGAGTACAAGATCCCCGCGCGGCTCA |
| | | CCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGA |
| 3 | Codon-optimized | ATGGCACTGCTGATCCACCTGAAAACCGTCTCCGAACTGAGAGG |
| | (CO) DNA sequence | CAGAGGGGACAGAATCGCTAAAGTCACCTTCCGGGGACAGAGC |
| | encoding the human | TTTTACAGCAGGGTGCTGGAGAACTGCGAGGACGTGGCCGACT |
| | otoferlin isoform 5 | TTGACGAGACATTCAGGTGGCCCGTGGCCAGCTCCATCGATCG |
| | protein (SEQ ID NO: | CAATGAGATGCTGGAGATCCAGGTGTTTAACTATAGCAAGGTGT |
| | 1), 5994 bp | TCTCCAATAAGCTGATCGGCACCTTCCGGATGGTGCTGCAGAAG |
| | | GTGGTGGAGGAGTCCCACGTGGAGGTGACCGACACACTGATCG |
| | | ACGATAACAATGCCATCATCAAGACATCCCTGTGCGTGGAGGTG |
| | | CGCTACCAGGCCACCGATGGCACAGTGGGCTCTTGGGACGATG |
| | | GCGACTTCCTGGGCGATGAGTCCCTGCAGGAGGAGGAGAAGGA |
| | | CTCTCAGGAGACAGATGGCCTGCTGCCTGGCTCCCGGCCATCT |
| | | AGCCGCCCCCCTGGCGAGAAGTCTTTTAGGAGAGCCGGCAGGT |
| | | CCGTGTTCTCTGCCATGAAGCTGGGCAAGAACAGGAGCCACAA |
| | | GGAGGAGCCTCAGAGGCCCGACGAGCCAGCCGTGCTGGAGAT |
| | | GGAGGACCTGGATCACCTGGCCATCAGACTGGGCGATGGCCTG |
| | | GACCCTGATAGCGTGTCCCTGGCCTCCGTGACCGCCCTGACCA |
| | | CAAACGTGTCTAATAAGCGGAGCAAGCCAGACATCAAGATGGAG |
| | | CCATCTGCCGGCAGGCCCATGGATTACCAGGTGAGCATCACAG |
| | | TGATCGAGGCCAGACAGCTGGTGGGCCTGAACATGGACCCCGT |
| | | GGTGTGCGTGGAAGTGGGCGACGATAAGAAGTACACCTCCATG |
| | | AAGGAGTCTACAAACTGTCCATACTACAACGAGTACTTCGTGTTT |
| | | GATTTCCACGTGAGCCCCGACGTGATGTTCGATAAGATCATCAA |
| | | GATCAGCGTGATCCACTCCAAGAATCTGCTGCGGTCTGGCACC |
| | | CTGGTGGGAAGCTTTAAGATGGACGTGGGCACAGTGTACTCTCA |
| | | GCCTGAGCACCAGTTCCACCACAAGTGGGCCATCCTGAGCGAT |
| | | CCAGACGATATCTCCTCTGGCCTGAAGGGCTATGTGAAGTGCGA |
| | | CGTGGCAGTGGTGGGCAAGGGCGATAACATCAAGACCCCACAC |
| | | AAGGCCAATGAGACAGACGAGGACGATATCGAGGGAAACCTGC |
| | | TGCTGCCAGAGGGAGTGCCACCCGAGAGGCAGTGGGCCAGGT |
| | | TCTACGTGAAGATCTATAGGGCAGAGGGCCTGCCTAGGATGAA |
| | | CACCAGCCTGATGGCCAATGTGAAGAAGGCCTTCATCGGCGAG |
| | | AACAAGGACCTGGTGGATCCCTACGTGCAGGTGTTCTTTGCCG |
| | | GCCAGAAGGGCAAGACCTCCGTGCAGAAGAGCTCCTATGAGCC |
| | | TCTGTGGAATGAGCAGGTGGTGTTTACAGACCTGTTCCCTCCAC |
| | | TGTGCAAGAGGATGAAGGTGCAGATCAGAGACTCTGATAAGGT |
| | | GAACGACGTGGCCATCGGCACCCACTTTATCGATCTGAGGAAG |
| | | ATCAGCAATGACGGCGATAAGGGCTTCCTGCCCACCCTGGGCC |
| | | CCGCCTGGGTGAACATGTACGGCAGCACCAGAAATTATACACTG |
| | | CTGGACGAGCACCAGGATCTGAACGAGGGCCTGGGCGAGGGC |
| | | GTGAGCTTTAGAGCCAGGCTGCTGCTGGGCCTGGCCGTGGAGA |
| | | TCGTGGACACCTCCAATCCCGAGCTGACCTCTAGCACAGAGGT |
| | | GCAGGTGGAGCAGGCCACACCTATCTCTGAGAGCTGTGCCGGC |
| | | AAGATGGAGGAGTTCTTTCTGTTTGGCGCCTTCCTGGAGGCCTC |
| | | CATGATCGACCGGCGCAACGGCGATAAGCCTATCACCTTCGAG |
| | | GTGACAATCGGCAACTACGGCAATGAGGTGGACGGCCTGTCTC |
| | | GGCCCCAGCGCCCAAGGCCCAGAAAGGAGCCTGGCGACGAGG |
| | | AGGAGGTGGATCTGATCCAGAACGCCAGCGACGATGAGGCAGG |
| | | CGACGCAGGCGATCTGGCCTCCGTGTCCTCTACCCCCCCTATG |
| | | CGGCCACAGGTGACAGACCGCAATTACTTTCACCTGCCTTATCT |
| | | GGAGCGCAAGCCATGCATCTACATCAAGTCTTGGTGGCCCGAT |
| | | CAGAGGAGACGGCTGTATAACGCCAATATCATGGACCACATCGC |
| | | CGATAAGCTGGAGGAGGGCCTGAATGACATCCAGGAGATGATC |
| | | AAGACCGAGAAGTCCTATCCAGAGCGCAGGCTGAGGGGCGTGC |
| | | TGGAGGAGCTGAGCTGTGGCTGCTGTAGATTCCTGTCCCTGGC |
| | | CGACAAGGATCAGGGGCACTCATCACGGACACGGCTGGACCGG |
| | | GAGCGGCTGAAATCATGTATGCGGGAGCTGGAAAATATGGGAC |
| | | AGCAGGCAAGGATGCTGCGCGCCCAGGTGAAGAGGCACACCG |
| | | TGAGAGACAAGCTGCGGCTGTGCCAGAACTTCCTGCAGAAGCT |
| | | GCGCTTTCTGGCCGATGAGCCACAGCACAGCATCCCCGACATC |
| | | TTCATCTGGATGATGTCCAACAATAAGAGAGTGGCCTACGCCCG |
| | | GGTGCCCTCTAAGGATCTGCTGTTTAGCATCGTGGAGGAGGAG |
| | | ACAGGCAAGGACTGTGCCAAGGTGAAGACCCTGTTCCTGAAGC |
| | | TGCCTGGCAAGAGAGGCTTTGGCAGCGCCGGATGGACCGTGCA |
| | | GGCAAAGGTGGAGCTGTATCTGTGGCTGGGCCTGTCTAAGCAG |
| | | CGGAAGGAGTTCCTGTGCGGCCTGCCCTGTGGCTTTCAGGAGG |
| | | TGAAGGCAGCACAGGGACTGGGACTGCACGCCTTCCCCCCCGT |
| | | GAGCCTGGTGTACACCAAGAAGCAGGCCTTTCAGCTGAGGGCC |
| | | CATATGTACCAGGCCAGGTCTCTGTTCGCCGCCGATAGCTCCG |
| | | GACTGAGCGACCCTTTTGCCAGGGTGTTCTTTATCAATCAGAGC |
| | | CAGTGCACAGAGGTGCTGAACGAGACCCTGTGCCCAACATGGG |
| | | ATCAGATGCTGGTGTTCGACAACCTGGAGCTGTACGGAGAGGC |
| | | ACACGAGCTGAGGGACGATCCACCCATCATCGTGATCGAGATCT |
| | | ATGATCAGGACTCCATGGGCAAGGCCGATTTCATGGGCAGGAC |
| | | CTTTGCCAAGCCCCTGGTGAAGATGGCCGACGAGGCCTACTGC |
| | | CCTCCAAGATTCCCCCCTCAGCTCGAGTACTATCAGATCTATAG |
| | | GGGAAATGCAACCGCCGGAGACCTGCTGGCCGCCTTTGAGCTG |
| | | CTGCAGATCGGCCCCGCCGGAAAGGCAGACCTGCCACCCATCA |
| | | ACGGCCCAGTGGATGTGGACAGAGGCCCCATCATGCCTGTGCC |
| | | AATGGGCATCAGACCAGTGCTGTCCAAGTACAGGGTGGAGGTG |
| | | CTGTTCTGGGGACTGCGCGACCTGAAGAGGGTGAATCTGGCCC |
| | | AGGTGGATAGGCCCAGAGTGGACATCGAGTGCGCCGGAAAGG |
| | | GCGTGCAGTCTAGCCTGATCCACAACTATAAGAAGAACCCAAAT |
| | | TTCAACACCCTGGTGAAGTGGTTTGAGGTGGATCTGCCCGAGAA |
| | | TGAGCTGCTGCACCCTCCACTGAACATCCGGGTGGTGGACTGT |
| | | AGAGCCTTCGGCAGGTACACCCTGGTGGGCAGCCACGCCGTGA |
| | | GCAGCCTGAGGAGGTTCATCTACAGGCCCCCTGACAGGTCCGC |
| | | CCCTTCTTGGAATACCACAGTGAGACTGCTGCGGCGCTGCAGG |
| | | GTGCTGTGCAACGGAGGCAGCTCCTCTCACTCTACCGGCGAGG |
| | | TGGTGGTGACAATGGAGCCTGAGGTACCCATCAAGAAGCTGGA |
| | | GACCATGGTGAAGCTGGATGCCACAAGCGAGGCAGTGGTGAAG |
| | | GTGGACGTGGCAGAGGAGGAGAAGGAGAAGAAGAAGAAGAAG |
| | | AAGGGAACCGCCGAGGAGCCTGAGGAAGAGGAGCCAGATGAG |
| | | AGCATGCTGGACTGGTGGTCCAAGTACTTCGCCTCTATCGACAC |
| | | AATGAAGGAGCAGCTGAGACAGCAGGAGCCTAGCGGCATCGAT |
| | | CTGGAGGAGAAGGAGGAGGTGGACAATACCGAGGGCCTGAAG |
| | | GGCTCCATGAAGGGCAAGGAGAAGGCAAGGGCAGCAAAGGAA |
| | | GAGAAGAAGAAGAAGACCCAGAGCAGCGGCTCTGGACAGGGCA |
| | | GCGAGGCACCAGAGAAGAAGAAGCCTAAGATCGATGAGCTGAA |
| | | GGTGTACCCAAAGGAGCTGGAGTCCGAGTTCGATAATTTTGAGG |
| | | ACTGGCTGCACACCTTCAACCTGCTGCGCGGCAAGACAGGCGA |
| | | CGATGAGGACGGCAGCACCGAGGAGGAGAGAATCGTGGGCCG |
| | | GTTTAAGGGCTCCCTGTGCGTGTACAAGGTGCCACTGCCTGAG |
| | | GACGTGAGCAGGGAGGCCGGATACGACTCTACCTATGGCATGT |
| | | TCCAGGGCATCCCCTCTAATGATCCTATCAACGTGCTGGTGCGC |
| | | GTGTATGTGGTGAGGGCCACAGATCTGCACCCCGCCGACATCA |
| | | ACGGCAAGGCCGACCCTTACATCGCCATCCGCCTGGGCAAGAC |
| | | CGATATCAGGGACAAGGAGAATTATATCTCCAAGCAGCTGAACC |
| | | CCGTGTTCGGCAAGTCTTTTGACATCGAGGCCAGCTTCCCTATG |
| | | GAGTCCATGCTGACCGTGGCCGTGTACGATTGGGACCTGGTGG |
| | | GCACCGACGATCTGATCGGCGAGACAAAGATCGATCTGGAGAA |
| | | TCGCTTTTATTCTAAGCACAGGGCAACCTGCGGAATCGCACAGA |
| | | CCTACAGCACACACGGCTATAACATCTGGCGCGACCCCATGAA |
| | | GCCTAGCCAGATCCTGACAAGGCTGTGCAAGGATGGCAAGGTG |
| | | GACGGACCACACTTCGGACCACCCGGCAGAGTGAAGGTGGCCA |
| | | ATCGGGTGTTTACAGGCCCTTCCGAGATCGAGGATGAGAACGG |
| | | CCAGCGCAAGCCAACCGACGAGCACGTGGCCCTGCTGGCCCT |
| | | GAGGCACTGGGAGGATATCCCAAGGGCCGGATGTAGGCTGGTG |
| | | CCTGAGCACGTGGAGACCAGACCACTGCTGAATCCAGACAAGC |
| | | CAGGAATCGAGCAGGGCAGGCTGGAGCTGTGGGTGGATATGTT |
| | | CCCAATGGACATGCCAGCCCCAGGAACACCCCTGGATATCTCC |
| | | CCTAGAAAGCCAAAGAAGTACGAGCTGAGAGTGATCATCTGGAA |
| | | CACAGACGAGGTGGTGCTGGAGGACGATGACTTCTTTACCGGC |
| | | GAGAAGTCTAGCGATATCTTTGTGCGCGGATGGCTGAAGGGAC |
| | | AGCAGGAGGACAAGCAGGATACAGACGTGCACTACCACTCCCT |
| | | GACCGGCGAGGGCAATTTCAACTGGAGATACCTGTTCCCTTTTG |
| | | ATTATCTGGCCGCCGAGGAGAAGATCGTGATCTCTAAGAAGGAG |
| | | AGCATGTTTTCCTGGGACGAGACAGAGTATAAGATCCCAGCCAG |
| | | ACTGACCCTGCAGATCTGGGATGCCGACCACTTCAGCGCCGAT |
| | | GACTTTCTGGGCGCCATCGAGCTGGACCTGAACCGGTTCCCAA |
| | | GAGGCGCCAAGACCGCCAAGCAGTGCACAATGGAGATGGCAAC |
| | | CGGAGAGGTGGACGTGCCTCTGGTGTCTATCTTCAAGCAGAAG |
| | | CGGGTGAAGGGATGGTGGCCACTGCTGGCCAGGAACGAGAAT |
| | | GATGAGTTTGAGCTGACAGGCAAGGTGGAGGCAGAGCTGCACC |
| | | TGCTGACCGCCGAGGAGGCAGAGAAGAACCCAGTGGGCCTGG |
| | | CCAGGAATGAGCCCGACCCTCTGGAGAAGCCAAACAGGCCCGA |
| | | TACAGCCTTCGTGTGGTTTCTGAATCCTCTGAAGAGCATCAAGT |
| | | ACCTGATCTGTACCAGGTATAAGTGGCTGATCATCAAGATCGTG |
| | | CTGGCCCTGCTGGGACTGCTGATGCTGGGCCTGTTTCTGTACTC |
| | | CCTGCCCGGCTATATGGTGAAGAAGCTGCTGGGCGCCTGA |
| 4 | Human OTOF | MALLIHLKTVSELRGRGDRIAKVTFRGQSFYSRVLENCEDVADFDE |
| | isoform 1 protein | TFRWPVASSIDRNEMLEIQVFNYSKVFSNKLIGTFRMVLQKVVEES |
| | (NP_919224.1), also | HVEVTDTLIDDNNAIIKTSLCVEVRYQATDGTVGSWDDGDFLGDES |
| | called human | LQEEEKDSQETDGLLPGSRPSSRPPGEKSFRRAGRSVFSAMKLGK |
| | otoferlin isoform a, | NRSHKEEPQRPDEPAVLEMEDLDHLAIRLGDGLDPDSVSLASVTAL |
| | 1997 aa | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTSFIWFLNPLKSARYFLWHTYRWLLLKLL |
| | | LLLLLLLLLALFLYSVPGYLVKKILGA |
| 5 | DNA sequence | ATGGCCCTGATTGTTCACCTCAAGACTGTCTCAGAGCTCCGAGG |
| | encoding the human | CAAAGGTGACCGGATTGCCAAAGTCACTTTCCGAGGGCAGTCTT |
| | otoferlin isoform 1 | TCTACTCCCGGGTCCTGGAGAACTGCGAGGGTGTGGCTGACTT |
| | protein (SEQ ID NO: | TGATGAGACGTTCCGGTGGCCAGTGGCCAGCAGCATCGACCGG |
| | 4), 5979 bp, | AATGAAGTGTTGGAGATTCAGATTTTCAACTACAGCAAAGTCTTC |
| | corresponds to the | AGCAACAAGCTGATAGGGACCTTCTGCATGGTGCTGCAGAAAGT |
| | coding sequence | GGTGGAGGAGAATCGGGTAGAGGTGACCGACACGCTGATGGAT |
| | documented in | GACAGCAATGCTATCATCAAGACCAGCCTGAGCATGGAGGTCC |
| | NM_001100395 | GGTATCAGGCCACAGATGGCACTGTGGGCCCCTGGGATGATGG |
| | | AGACTTCCTGGGAGATGAATCCCTCCAGGAGGAGAAGGACAGC |
| | | CAGGAGACAGATGGGCTGCTACCTGGTTCCCGACCCAGCACCC |
| | | GGATATCTGGCGAGAAGAGCTTTCGCAGCAAAGGCAGAGAGAA |
| | | GACCAAGGGAGGCAGAGATGGCGAGCACAAAGCGGGAAGGAG |
| | | TGTGTTCTCGGCCATGAAACTCGGCAAAACTCGGTCCCACAAAG |
| | | AGGAGCCCCAAAGACAAGATGAGCCAGCAGTGCTGGAGATGGA |
| | | GGACCTGGACCACCTAGCCATTCAGCTGGGGGATGGGCTGGAT |
| | | CCTGACTCCGTGTCTCTAGCCTCGGTCACCGCTCTCACCAGCAA |
| | | TGTCTCCAACAAACGGTCTAAGCCAGATATTAAGATGGAGCCCA |
| | | GTGCTGGAAGGCCCATGGATTACCAGGTCAGCATCACAGTGATT |
| | | GAGGCTCGGCAGCTGGTGGGCTTGAACATGGACCCTGTGGTGT |
| | | GTGTGGAGGTGGGTGATGACAAGAAATACACGTCAATGAAGGA |
| | | GTCCACAAACTGCCCTTACTACAACGAGTACTTTGTCTTCGACTT |
| | | CCATGTCTCTCCTGATGTCATGTTTGACAAGATCATCAAGATCTC |
| | | GGTTATCCATTCTAAGAACCTGCTTCGGAGCGGCACCCTGGTGG |
| | | GTTCCTTCAAAATGGATGTGGGGACTGTGTATTCCCAGCCTGAA |
| | | CACCAGTTCCATCACAAATGGGCCATCCTGTCAGACCCCGATGA |
| | | CATCTCTGCTGGGTTGAAGGGTTATGTAAAGTGTGATGTCGCTG |
| | | TGGTGGGCAAGGGAGACAACATCAAGACACCCCACAAGGCCAA |
| | | CGAGACGGATGAGGACGACATTGAAGGGAACTTGCTGCTCCCC |
| | | GAGGGCGTGCCCCCCGAACGGCAGTGGGCACGGTTCTATGTGA |
| | | AAATTTACCGAGCAGAGGGACTGCCCCGGATGAACACAAGCCT |
| | | CATGGCCAACGTGAAGAAGGCGTTCATCGGTGAGAACAAGGAC |
| | | CTCGTCGACCCCTATGTGCAAGTCTTCTTTGCTGGACAAAAGGG |
| | | CAAAACATCAGTGCAGAAGAGCAGCTATGAGCCGCTATGGAATG |
| | | AGCAGGTCGTCTTCACAGACTTGTTCCCCCCACTCTGCAAACGC |
| | | ATGAAGGTGCAGATCCGGGACTCTGACAAGGTCAATGATGTGG |
| | | CCATCGGCACCCACTTCATCGACCTGCGCAAGATTTCCAACGAT |
| | | GGAGACAAAGGCTTCCTGCCTACCCTCGGTCCAGCCTGGGTGA |
| | | ACATGTACGGCTCCACGCGCAACTACACACTGCTGGACGAGCA |
| | | CCAGGACTTGAATGAAGGCCTGGGGGAGGGTGTGTCCTTCCGG |
| | | GCCCGCCTCATGTTGGGACTAGCTGTGGAGATCCTGGACACCT |
| | | CCAACCCAGAGCTCACCAGCTCCACGGAGGTGCAGGTGGAGCA |
| | | GGCCACGCCTGTCTCGGAGAGCTGCACAGGGAGAATGGAAGAA |
| | | TTTTTTCTATTTGGAGCCTTCTTGGAAGCCTCAATGATTGACCGG |
| | | AAAAATGGGGACAAGCCAATTACCTTTGAGGTGACCATAGGAAA |
| | | CTACGGCAATGAAGTCGATGGTATGTCCCGGCCCCTGAGGCCT |
| | | CGGCCCCGGAAAGAGCCTGGGGATGAAGAAGAGGTAGACCTGA |
| | | TTCAGAACTCCAGTGACGATGAAGGTGACGAAGCCGGGGACCT |
| | | GGCCTCGGTGTCCTCCACCCCACCTATGCGGCCCCAGATCACG |
| | | GACAGGAACTATTTCCACCTGCCCTACCTGGAGCGCAAGCCCT |
| | | GCATCTATATCAAGAGCTGGTGGCCTGACCAGAGGCGGCGCCT |
| | | CTACAATGCCAACATCATGGATCACATTGCTGACAAGCTGGAAG |
| | | AAGGCCTGAATGATGTACAGGAGATGATCAAAACGGAGAAGTCC |
| | | TACCCGGAGCGCCGCCTGCGGGGTGTGCTAGAGGAACTCAGCT |
| | | GTGGCTGCCACCGCTTCCTCTCCCTCTCGGACAAGGACCAGGG |
| | | CCGCTCGTCCCGCACCAGGCTGGATCGAGAGCGTCTTAAGTCC |
| | | TGTATGAGGGAGTTGGAGAGCATGGGACAGCAGGCCAAGAGCC |
| | | TGAGGGCTCAGGTGAAGCGGCACACTGTTCGGGACAAGCTGAG |
| | | GTCATGCCAGAACTTTCTGCAGAAGCTACGCTTCCTGGCGGATG |
| | | AGCCCCAGCACAGCATTCCTGATGTGTTCATTTGGATGATGAGC |
| | | AACAACAAACGTATCGCCTATGCCCGCGTGCCTTCCAAAGACCT |
| | | GCTCTTCTCCATCGTGGAGGAGGAACTGGGCAAGGACTGCGCC |
| | | AAAGTCAAGACCCTCTTCCTGAAGCTGCCAGGGAAGAGGGGCT |
| | | TCGGCTCGGCAGGCTGGACAGTACAGGCCAAGCTGGAGCTCTA |
| | | CCTGTGGCTGGGCCTCAGCAAGCAGCGAAAGGACTTCCTGTGT |
| | | GGTCTGCCCTGTGGCTTCGAGGAGGTCAAGGCAGCCCAAGGCC |
| | | TGGGCCTGCATTCCTTTCCGCCCATCAGCCTAGTCTACACCAAG |
| | | AAGCAAGCCTTCCAGCTCCGAGCACACATGTATCAGGCCCGAA |
| | | GCCTCTTTGCTGCTGACAGCAGTGGGCTCTCTGATCCCTTTGCC |
| | | CGTGTCTTCTTCATCAACCAGAGCCAATGCACTGAGGTTCTAAA |
| | | CGAGACACTGTGTCCCACCTGGGACCAGATGCTGGTATTTGACA |
| | | ACCTGGAGCTGTACGGTGAAGCTCACGAGTTACGAGATGATCC |
| | | CCCCATCATTGTCATTGAAATCTACGACCAGGACAGCATGGGCA |
| | | AAGCCGACTTCATGGGCCGGACCTTCGCCAAGCCCCTGGTGAA |
| | | GATGGCAGATGAAGCATACTGCCCACCTCGCTTCCCGCCGCAG |
| | | CTTGAGTACTACCAGATCTACCGAGGCAGTGCCACTGCCGGAG |
| | | ACCTACTGGCTGCCTTCGAGCTGCTGCAGATTGGGCCATCAGG |
| | | GAAGGCTGACCTGCCACCCATCAATGGCCCAGTGGACATGGAC |
| | | AGAGGGCCCATCATGCCTGTGCCCGTGGGAATCCGGCCAGTGC |
| | | TCAGCAAGTACCGAGTGGAGGTGCTGTTCTGGGGCCTGAGGGA |
| | | CCTAAAGAGGGTGAACCTGGCCCAGGTGGACCGACCACGGGTG |
| | | GACATCGAGTGTGCAGGAAAGGGGGTACAATCCTCCCTGATTCA |
| | | CAATTATAAGAAGAACCCCAACTTCAACACGCTGGTCAAGTGGT |
| | | TTGAAGTGGACCTCCCGGAGAATGAGCTCCTGCACCCACCCTT |
| | | GAACATCCGAGTGGTAGATTGCCGGGCCTTTGGACGATACACC |
| | | CTGGTGGGTTCCCACGCAGTCAGCTCACTGAGGCGCTTCATCTA |
| | | CCGACCTCCAGACCGCTCAGCCCCCAACTGGAACACCACAGGG |
| | | GAGGTTGTAGTAAGCATGGAGCCTGAGGAGCCAGTTAAGAAGC |
| | | TGGAGACCATGGTGAAACTGGATGCGACTTCTGATGCTGTGGTC |
| | | AAGGTGGATGTGGCTGAAGATGAGAAGGAAAGGAAGAAGAAGA |
| | | AAAAGAAAGGCCCGTCAGAGGAGCCAGAGGAGGAAGAGCCCG |
| | | ATGAGAGCATGCTGGATTGGTGGTCCAAGTACTTCGCCTCCATC |
| | | GACACAATGAAGGAGCAACTTCGACAACATGAGACCTCTGGAAC |
| | | TGACTTGGAAGAGAAGGAAGAGATGGAAAGCGCTGAGGGCCTG |
| | | AAGGGACCAATGAAGAGCAAGGAGAAGTCCAGAGCTGCAAAGG |
| | | AGGAGAAAAAGAAGAAAAACCAGAGCCCTGGCCCTGGCCAGGG |
| | | ATCGGAGGCTCCTGAGAAGAAGAAAGCCAAGATCGATGAGCTTA |
| | | AGGTGTACCCCAAGGAGCTGGAATCGGAGTTTGACAGCTTTGA |
| | | GGACTGGCTGCACACCTTCAACCTGTTGAGGGGCAAGACGGGA |
| | | GATGATGAGGATGGCTCCACAGAGGAGGAGCGCATAGTAGGCC |
| | | GATTCAAGGGCTCCCTCTGTGTGTACAAAGTGCCACTCCCAGAA |
| | | GATGTATCTCGAGAAGCTGGCTATGATCCCACCTATGGAATGTT |
| | | CCAGGGCATCCCAAGCAATGACCCCATCAATGTGCTGGTCCGA |
| | | ATCTATGTGGTCCGGGCCACAGACCTGCACCCGGCCGACATCA |
| | | ATGGCAAAGCTGACCCCTATATTGCCATCAAGTTAGGCAAGACC |
| | | GACATCCGAGACAAGGAGAACTACATCTCCAAGCAGCTCAACCC |
| | | TGTGTTTGGGAAGTCCTTTGACATTGAGGCCTCCTTCCCCATGG |
| | | AGTCCATGTTGACAGTGGCCGTGTACGACTGGGATCTGGTGGG |
| | | CACTGATGACCTCATCGGAGAAACCAAGATTGACCTGGAAAACC |
| | | GCTTCTACAGCAAGCATCGCGCCACCTGCGGCATCGCACAGAC |
| | | CTATTCCATACATGGCTACAATATCTGGAGGGACCCCATGAAGC |
| | | CCAGCCAGATCCTGACACGCCTCTGTAAAGAGGGCAAAGTGGA |
| | | CGGCCCCCACTTTGGTCCCCATGGGAGAGTGAGGGTTGCCAAC |
| | | CGTGTCTTCACGGGGCCTTCAGAAATAGAGGATGAGAATGGTCA |
| | | GAGGAAGCCCACAGATGAGCACGTGGCACTGTCTGCTCTGAGA |
| | | CACTGGGAGGACATCCCCCGGGTGGGCTGCCGCCTTGTGCCG |
| | | GAACACGTGGAGACCAGGCCGCTGCTCAACCCTGACAAGCCAG |
| | | GCATTGAGCAGGGCCGCCTGGAGCTGTGGGTGGACATGTTCCC |
| | | CATGGACATGCCAGCCCCTGGGACACCTCTGGATATATCCCCCA |
| | | GGAAACCCAAGAAGTACGAGCTGCGGGTCATCGTGTGGAACAC |
| | | AGACGAGGTGGTCCTGGAAGACGATGATTTCTTCACGGGAGAG |
| | | AAGTCCAGTGACATTTTTGTGAGGGGGTGGCTGAAGGGCCAGC |
| | | AGGAGGACAAACAGGACACAGATGTCCACTATCACTCCCTCACG |
| | | GGGGAGGGCAACTTCAACTGGAGATACCTCTTCCCCTTCGACTA |
| | | CCTAGCGGCCGAAGAGAAGATCGTTATGTCCAAAAAGGAGTCTA |
| | | TGTTCTCCTGGGATGAGACGGAGTACAAGATCCCTGCGCGGCT |
| | | CACCCTGCAGATCTGGGACGCTGACCACTTCTCGGCTGACGAC |
| | | TTCCTGGGGGCTATCGAGCTGGACCTGAACCGGTTCCCGAGGG |
| | | GCGCTAAGACAGCCAAGCAGTGCACCATGGAGATGGCCACCGG |
| | | GGAGGTGGACGTACCCCTGGTTTCCATCTTTAAACAGAAACGTG |
| | | TCAAAGGCTGGTGGCCCCTCCTGGCCCGCAATGAGAATGATGA |
| | | GTTTGAGCTCACAGGCAAAGTGGAGGCGGAGCTACACCTACTC |
| | | ACGGCAGAGGAGGCAGAGAAGAACCCTGTGGGCCTGGCTCGC |
| | | AATGAACCTGATCCCCTAGAAAAACCCAACCGGCCTGACACGGC |
| | | ATTCGTCTGGTTCCTGAACCCACTCAAATCTATCAAGTACCTCAT |
| | | CTGCACCCGGTACAAGTGGCTGATCATCAAGATCGTGCTGGCG |
| | | CTGCTGGGGCTGCTCATGCTGGCCCTCTTCCTTTACAGCCTCCC |
| | | AGGCTACATGGTCAAGAAGCTCCTAGGGGCCTGA |
| 6 | Codon-optimized | ATGGCACTGCTGATCCACCTGAAAACCGTCTCCGAACTGAGAGG |
| | (CO) DNA sequence | CAGAGGGGACAGAATCGCTAAAGTCACCTTCCGGGGACAGAGC |
| | encoding the human | TTTTACAGCAGGGTGCTGGAGAACTGCGAGGACGTGGCCGACT |
| | otoferlin isoform 1 | TTGACGAGACATTCAGGTGGCCCGTGGCCAGCTCCATCGATCG |
| | protein (SEQ ID NO: | CAATGAGATGCTGGAGATCCAGGTGTTTAACTATAGCAAGGTGT |
| | 4), 5994 bp | TCTCCAATAAGCTGATCGGCACCTTCCGGATGGTGCTGCAGAAG |
| | | GTGGTGGAGGAGTCCCACGTGGAGGTGACCGACACACTGATCG |
| | | ACGATAACAATGCCATCATCAAGACATCCCTGTGCGTGGAGGTG |
| | | CGCTACCAGGCCACCGATGGCACAGTGGGCTCTTGGGACGATG |
| | | GCGACTTCCTGGGCGATGAGTCCCTGCAGGAGGAGGAGAAGGA |
| | | CTCTCAGGAGACAGATGGCCTGCTGCCTGGCTCCCGGCCATCT |
| | | AGCCGCCCCCCTGGCGAGAAGTCTTTTAGGAGAGCCGGCAGGT |
| | | CCGTGTTCTCTGCCATGAAGCTGGGCAAGAACAGGAGCCACAA |
| | | GGAGGAGCCTCAGAGGCCCGACGAGCCAGCCGTGCTGGAGAT |
| | | GGAGGACCTGGATCACCTGGCCATCAGACTGGGCGATGGCCTG |
| | | GACCCTGATAGCGTGTCCCTGGCCTCCGTGACCGCCCTGACCA |
| | | CAAACGTGTCTAATAAGCGGAGCAAGCCAGACATCAAGATGGAG |
| | | CCATCTGCCGGCAGGCCCATGGATTACCAGGTGAGCATCACAG |
| | | TGATCGAGGCCAGACAGCTGGTGGGCCTGAACATGGACCCCGT |
| | | GGTGTGCGTGGAAGTGGGCGACGATAAGAAGTACACCTCCATG |
| | | AAGGAGTCTACAAACTGTCCATACTACAACGAGTACTTCGTGTTT |
| | | GATTTCCACGTGAGCCCCGACGTGATGTTCGATAAGATCATCAA |
| | | GATCAGCGTGATCCACTCCAAGAATCTGCTGCGGTCTGGCACC |
| | | CTGGTGGGAAGCTTTAAGATGGACGTGGGCACAGTGTACTCTCA |
| | | GCCTGAGCACCAGTTCCACCACAAGTGGGCCATCCTGAGCGAT |
| | | CCAGACGATATCTCCTCTGGCCTGAAGGGCTATGTGAAGTGCGA |
| | | CGTGGCAGTGGTGGGCAAGGGCGATAACATCAAGACCCCACAC |
| | | AAGGCCAATGAGACAGACGAGGACGATATCGAGGGAAACCTGC |
| | | TGCTGCCAGAGGGAGTGCCACCCGAGAGGCAGTGGGCCAGGT |
| | | TCTACGTGAAGATCTATAGGGCAGAGGGCCTGCCTAGGATGAA |
| | | CACCAGCCTGATGGCCAATGTGAAGAAGGCCTTCATCGGCGAG |
| | | AACAAGGACCTGGTGGATCCCTACGTGCAGGTGTTCTTTGCCG |
| | | GCCAGAAGGGCAAGACCTCCGTGCAGAAGAGCTCCTATGAGCC |
| | | TCTGTGGAATGAGCAGGTGGTGTTTACAGACCTGTTCCCTCCAC |
| | | TGTGCAAGAGGATGAAGGTGCAGATCAGAGACTCTGATAAGGT |
| | | GAACGACGTGGCCATCGGCACCCACTTTATCGATCTGAGGAAG |
| | | ATCAGCAATGACGGCGATAAGGGCTTCCTGCCCACCCTGGGCC |
| | | CCGCCTGGGTGAACATGTACGGCAGCACCAGAAATTATACACTG |
| | | CTGGACGAGCACCAGGATCTGAACGAGGGCCTGGGCGAGGGC |
| | | GTGAGCTTTAGAGCCAGGCTGCTGCTGGGCCTGGCCGTGGAGA |
| | | TCGTGGACACCTCCAATCCCGAGCTGACCTCTAGCACAGAGGT |
| | | GCAGGTGGAGCAGGCCACACCTATCTCTGAGAGCTGTGCCGGC |
| | | AAGATGGAGGAGTTCTTTCTGTTTGGCGCCTTCCTGGAGGCCTC |
| | | CATGATCGACCGGCGCAACGGCGATAAGCCTATCACCTTCGAG |
| | | GTGACAATCGGCAACTACGGCAATGAGGTGGACGGCCTGTCTC |
| | | GGCCCCAGCGCCCAAGGCCCAGAAAGGAGCCTGGCGACGAGG |
| | | AGGAGGTGGATCTGATCCAGAACGCCAGCGACGATGAGGCAGG |
| | | CGACGCAGGCGATCTGGCCTCCGTGTCCTCTACCCCCCCTATG |
| | | CGGCCACAGGTGACAGACCGCAATTACTTTCACCTGCCTTATCT |
| | | GGAGCGCAAGCCATGCATCTACATCAAGTCTTGGTGGCCCGAT |
| | | CAGAGGAGACGGCTGTATAACGCCAATATCATGGACCACATCGC |
| | | CGATAAGCTGGAGGAGGGCCTGAATGACATCCAGGAGATGATC |
| | | AAGACCGAGAAGTCCTATCCAGAGCGCAGGCTGAGGGGCGTGC |
| | | TGGAGGAGCTGAGCTGTGGCTGCTGTAGATTCCTGTCCCTGGC |
| | | CGACAAGGATCAGGGGCACTCATCACGGACACGGCTGGACCGG |
| | | GAGCGGCTGAAATCATGTATGCGGGAGCTGGAAAATATGGGAC |
| | | AGCAGGCAAGGATGCTGCGCGCCCAGGTGAAGAGGCACACCG |
| | | TGAGAGACAAGCTGCGGCTGTGCCAGAACTTCCTGCAGAAGCT |
| | | GCGCTTTCTGGCCGATGAGCCACAGCACAGCATCCCCGACATC |
| | | TTCATCTGGATGATGTCCAACAATAAGAGAGTGGCCTACGCCCG |
| | | GGTGCCCTCTAAGGATCTGCTGTTTAGCATCGTGGAGGAGGAG |
| | | ACAGGCAAGGACTGTGCCAAGGTGAAGACCCTGTTCCTGAAGC |
| | | TGCCTGGCAAGAGAGGCTTTGGCAGCGCCGGATGGACCGTGCA |
| | | GGCAAAGGTGGAGCTGTATCTGTGGCTGGGCCTGTCTAAGCAG |
| | | CGGAAGGAGTTCCTGTGCGGCCTGCCCTGTGGCTTTCAGGAGG |
| | | TGAAGGCAGCACAGGGACTGGGACTGCACGCCTTCCCCCCCGT |
| | | GAGCCTGGTGTACACCAAGAAGCAGGCCTTTCAGCTGAGGGCC |
| | | CATATGTACCAGGCCAGGTCTCTGTTCGCCGCCGATAGCTCCG |
| | | GACTGAGCGACCCTTTTGCCAGGGTGTTCTTTATCAATCAGAGC |
| | | CAGTGCACAGAGGTGCTGAACGAGACCCTGTGCCCAACATGGG |
| | | ATCAGATGCTGGTGTTCGACAACCTGGAGCTGTACGGAGAGGC |
| | | ACACGAGCTGAGGGACGATCCACCCATCATCGTGATCGAGATCT |
| | | ATGATCAGGACTCCATGGGCAAGGCCGATTTCATGGGCAGGAC |
| | | CTTTGCCAAGCCCCTGGTGAAGATGGCCGACGAGGCCTACTGC |
| | | CCTCCAAGATTCCCCCCTCAGCTCGAGTACTATCAGATCTATAG |
| | | GGGAAATGCAACCGCCGGAGACCTGCTGGCCGCCTTTGAGCTG |
| | | CTGCAGATCGGCCCCGCCGGAAAGGCAGACCTGCCACCCATCA |
| | | ACGGCCCAGTGGATGTGGACAGAGGCCCCATCATGCCTGTGCC |
| | | AATGGGCATCAGACCAGTGCTGTCCAAGTACAGGGTGGAGGTG |
| | | CTGTTCTGGGGACTGCGCGACCTGAAGAGGGTGAATCTGGCCC |
| | | AGGTGGATAGGCCCAGAGTGGACATCGAGTGCGCCGGAAAGG |
| | | GCGTGCAGTCTAGCCTGATCCACAACTATAAGAAGAACCCAAAT |
| | | TTCAACACCCTGGTGAAGTGGTTTGAGGTGGATCTGCCCGAGAA |
| | | TGAGCTGCTGCACCCTCCACTGAACATCCGGGTGGTGGACTGT |
| | | AGAGCCTTCGGCAGGTACACCCTGGTGGGCAGCCACGCCGTGA |
| | | GCAGCCTGAGGAGGTTCATCTACAGGCCCCCTGACAGGTCCGC |
| | | CCCTTCTTGGAATACCACAGTGAGACTGCTGCGGCGCTGCAGG |
| | | GTGCTGTGCAACGGAGGCAGCTCCTCTCACTCTACCGGCGAGG |
| | | TGGTGGTGACAATGGAGCCTGAGGTACCCATCAAGAAGCTGGA |
| | | GACCATGGTGAAGCTGGATGCCACAAGCGAGGCAGTGGTGAAG |
| | | GTGGACGTGGCAGAGGAGGAGAAGGAGAAGAAGAAGAAGAAG |
| | | AAGGGAACCGCCGAGGAGCCTGAGGAAGAGGAGCCAGATGAG |
| | | AGCATGCTGGACTGGTGGTCCAAGTACTTCGCCTCTATCGACAC |
| | | AATGAAGGAGCAGCTGAGACAGCAGGAGCCTAGCGGCATCGAT |
| | | CTGGAGGAGAAGGAGGAGGTGGACAATACCGAGGGCCTGAAG |
| | | GGCTCCATGAAGGGCAAGGAGAAGGCAAGGGCAGCAAAGGAA |
| | | GAGAAGAAGAAGAAGACCCAGAGCAGCGGCTCTGGACAGGGCA |
| | | GCGAGGCACCAGAGAAGAAGAAGCCTAAGATCGATGAGCTGAA |
| | | GGTGTACCCAAAGGAGCTGGAGTCCGAGTTCGATAATTTTGAGG |
| | | ACTGGCTGCACACCTTCAACCTGCTGCGCGGCAAGACAGGCGA |
| | | CGATGAGGACGGCAGCACCGAGGAGGAGAGAATCGTGGGCCG |
| | | GTTTAAGGGCTCCCTGTGCGTGTACAAGGTGCCACTGCCTGAG |
| | | GACGTGAGCAGGGAGGCCGGATACGACTCTACCTATGGCATGT |
| | | TCCAGGGCATCCCCTCTAATGATCCTATCAACGTGCTGGTGCGC |
| | | GTGTATGTGGTGAGGGCCACAGATCTGCACCCCGCCGACATCA |
| | | ACGGCAAGGCCGACCCTTACATCGCCATCCGCCTGGGCAAGAC |
| | | CGATATCAGGGACAAGGAGAATTATATCTCCAAGCAGCTGAACC |
| | | CCGTGTTCGGCAAGTCTTTTGACATCGAGGCCAGCTTCCCTATG |
| | | GAGTCCATGCTGACCGTGGCCGTGTACGATTGGGACCTGGTGG |
| | | GCACCGACGATCTGATCGGCGAGACAAAGATCGATCTGGAGAA |
| | | TCGCTTTTATTCTAAGCACAGGGCAACCTGCGGAATCGCACAGA |
| | | CCTACAGCACACACGGCTATAACATCTGGCGCGACCCCATGAA |
| | | GCCTAGCCAGATCCTGACAAGGCTGTGCAAGGATGGCAAGGTG |
| | | GACGGACCACACTTCGGACCACCCGGCAGAGTGAAGGTGGCCA |
| | | ATCGGGTGTTTACAGGCCCTTCCGAGATCGAGGATGAGAACGG |
| | | CCAGCGCAAGCCAACCGACGAGCACGTGGCCCTGCTGGCCCT |
| | | GAGGCACTGGGAGGATATCCCAAGGGCCGGATGTAGGCTGGTG |
| | | CCTGAGCACGTGGAGACCAGACCACTGCTGAATCCAGACAAGC |
| | | CAGGAATCGAGCAGGGCAGGCTGGAGCTGTGGGTGGATATGTT |
| | | CCCAATGGACATGCCAGCCCCAGGAACACCCCTGGATATCTCC |
| | | CCTAGAAAGCCAAAGAAGTACGAGCTGAGAGTGATCATCTGGAA |
| | | CACAGACGAGGTGGTGCTGGAGGACGATGACTTCTTTACCGGC |
| | | GAGAAGTCTAGCGATATCTTTGTGCGCGGATGGCTGAAGGGAC |
| | | AGCAGGAGGACAAGCAGGATACAGACGTGCACTACCACTCCCT |
| | | GACCGGCGAGGGCAATTTCAACTGGAGATACCTGTTCCCTTTTG |
| | | ATTATCTGGCCGCCGAGGAGAAGATCGTGATCTCTAAGAAGGAG |
| | | AGCATGTTTTCCTGGGACGAGACAGAGTATAAGATCCCAGCCAG |
| | | ACTGACCCTGCAGATCTGGGATGCCGACCACTTCAGCGCCGAT |
| | | GACTTTCTGGGCGCCATCGAGCTGGACCTGAACCGGTTCCCAA |
| | | GAGGCGCCAAGACCGCCAAGCAGTGCACAATGGAGATGGCAAC |
| | | CGGAGAGGTGGACGTGCCTCTGGTGTCTATCTTCAAGCAGAAG |
| | | AGGGTGAAGGGCTGGTGGCCACTGCTGGCCAGAAACGAGAATG |
| | | ATGAGTTTGAGCTGACAGGCAAGGTGGAGGCAGAGCTGCACCT |
| | | GCTGACCGCCGAGGAGGCAGAGAAGAACCCAGTGGGCCTGGC |
| | | CAGGAATGAGCCCGACCCTCTGGAGAAGCCAAACAGGCCCGAC |
| | | ACCAGCTTCATCTGGTTTCTGAATCCTCTGAAGTCCGCCCGGTA |
| | | CTTCCTGTGGCACACCTATCGCTGGCTGCTGCTGAAGCTGTTAT |
| | | TACTGTTATTACTGCTGCTGCTGCTGGCCCTGTTTCTGTACAGC |
| | | GTGCCCGGCTATCTGGTGAAGAAGATCCTGGGCGCCTGA |

### Expression of OTOF in Mammalian Cells

Mutations in OTOF have been linked to sensorineural hearing loss and auditory neuropathy. The compositions and methods described herein increase the expression of WT OTOF isoform 5 protein by administering a first nucleic acid vector that contains a polynucleotide encoding an N-terminal portion of an OTOF isoform 5 protein and a second nucleic acid vector that contains a polynucleotide encoding a C-terminal portion of an OTOF isoform 5 protein. In order to utilize nucleic acid vectors for therapeutic application in the treatment of sensorineural hearing loss and auditory neuropathy, they can be directed to the interior of the cell, and, in particular, to specific cell types. A wide array of methods has been established for the delivery of proteins to mammalian cells and for the stable expression of genes encoding proteins in mammalian cells.

### Polynucleotides Encoding OTOF

One platform that can be used to achieve therapeutically effective intracellular concentrations of OTOF isoform 5 in mammalian cells is via the stable expression of the gene encoding OTOF isoform 5 (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

OTOF isoform 5 can also be introduced into a mammalian cell by targeting vectors containing portions of a gene encoding an OTOF isoform 5 protein to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding an OTOF isoform 5 protein by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an OTOF protein downstream of a mammalian promoter (e.g., a polynucleotide that encodes an N-terminal portion of an OTOF isoform 5 protein downstream of a mammalian promoter). Promoters that are useful for the expression of an OTOF protein in mammalian cells include ubiquitous promoters and cochlear hair cell-specific promoters. Ubiquitous promoters include the CAG promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), the chicken β-actin promoter, the smCBA promoter, the CB7 promoter, the hybrid CMV enhancer/human β-actin promoter, the CASI promoter, the dihydrofolate reductase (DHFR) promoter, the human β-actin promoter, a β-globin promoter (e.g., a minimal β-globin promoter), an HSV promoter (e.g., a minimal HSV ICP0 promoter or a truncated HSV ICP0 promoter), an SV40 promoter (e.g., an SV40 minimal promoter), the EF1α promoter, and the PGK promoter. Cochlear hair cell-specific promoters include the Myosin 15 (Myo15) promoter. Myo15 promoter sequences for use in the methods and compositions described herein are described below and in Table 3. Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of Moloney virus, Epstein barr virus (EBV) promoter, and the Rous sarcoma virus (RSV) promoter.

### Murine Myosin 15 Promoters

In some embodiments, the Myo15 promoter for use in the compositions and methods described herein includes polynucleotide sequences from regions of the murine Myo15 locus that are capable of expressing a transgene specifically in hair cells, or variants thereof, such as a polynucleotide sequences that have at least 85% sequence identity (e.g., 85%, 90%, 91%, 92%, 93%, 94%, 95%, 96%, 97%, 98%, 99%, or more, sequence identity) to regions of the murine Myo15 locus that are capable of expressing a transgene specifically in hair cells. These regions include polynucleotide sequences immediately preceding the murine Myo15 translation start site and an upstream regulatory element that is located over 5 kb from the murine Myo15 translation start site. The murine Myo15 promoter for use in the compositions and methods described herein can optionally include a linker operably linking the regions of the murine Myo15 locus that are capable of expressing a transgene specifically in hair cells, or the regions of the murine Myo15 locus can be joined directly without an intervening linker.

In some embodiments, the murine Myo15 promoter for use in the compositions and methods described herein contains a first region (an upstream regulatory element) having at least 85% sequence identity (e.g., 85%, 90%, 91%, 92%, 93%, 94%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 7) or a functional portion or derivative thereof joined (e.g., operably linked) to a second region having at least 85% sequence identity (e.g., 85%, 90%, 91%, 92%, 93%, 94%, 95%, 96%, 97%, 98%, 99%, or more, sequence identity) to the polynucleotide sequence immediately preceding the murine Myo15 translation start site (nucleic acids from −1 to −1157 with respect to the murine Myo15 translation start site, the sequence of which is set forth in SEQ ID NO: 8) or a functional portion or derivative thereof. The functional portion of SEQ ID NO: 7 may have the sequence of nucleic acids from −7166 to −7091 with respect to the murine Myo15 translation start site (set forth in SEQ ID NO: 9) and/or the sequence of nucleic acids from −7077 to −6983 with respect to the murine Myo15 translation start site (set forth in SEQ ID NO: 10). The first region may contain the polynucleotide sequence of SEQ ID NO: 9 fused to the polynucleotide sequence of SEQ ID NO: 10 with no intervening nucleic acids, as set forth in SEQ ID NO: 11, or the first region may contain the polynucleotide sequence of SEQ ID NO: 10 fused to the polynucleotide sequence of SEQ ID NO: 9 with no intervening nucleic acids, as set forth in SEQ ID NO: 12. Alternatively, the first region may contain the sequences of SEQ ID NO: 9 and SEQ ID NO: 10 joined by the endogenous intervening polynucleotide sequence (e.g., the first region may have or include the sequence of nucleic acids from −7166 to −6983 with respect to the murine Myo15 translation start site, as set forth in SEQ ID NO: 13 and SEQ ID NO: 33) or a nucleic acid linker. In a murine Myo 15 promoter in which the first region contains both SEQ ID NO: 9 and SEQ ID NO: 10, the two sequences can be included in any order (e.g., SEQ ID NO: 9 may be joined to (e.g., precede) SEQ ID NO: 10, or SEQ ID NO: 10 may be joined to (e.g., precede) SEQ ID NO: 9). The functional portion of SEQ ID NO: 8 may have the sequence of nucleic acids from −590 to −509 with respect to the murine Myo15 translation start site (set forth in SEQ ID NO: 14) and/or the sequence of nucleic acids from −266 to −161 with respect to the murine Myo 15 translation start site (set forth in SEQ ID NO: 15). In some embodiments, the sequence containing SEQ ID NO: 14 has the sequence of SEQ ID NO: 34. In some embodiments, the sequence containing SEQ ID NO: 15 has the sequence of SEQ ID NO: 35. The second region may contain the polynucleotide sequence of SEQ ID NO: 14 fused to the polynucleotide sequence of SEQ ID NO: 15 with no intervening nucleic acids, as set forth in SEQ ID NO: 16, or the second region may contain the polynucleotide sequence of SEQ ID NO: 15 fused to the polynucleotide sequence of SEQ ID NO: 14 with no intervening nucleic acids, as set forth in SEQ ID NO: 17. The second region may contain the nucleic acid sequence of SEQ ID NO: 34 fused to the nucleic acid sequence of SEQ ID NO: 35 with no intervening nucleic acids, as set forth in SEQ ID NO: 38, or the second region may contain the nucleic acid sequence of SEQ ID NO: 35 fused to the nucleic acid sequence of SEQ ID NO: 34 with no intervening nucleic acids. Alternatively, the second region may contain the sequences of SEQ ID NO: 14 and SEQ ID NO: 15 joined by the endogenous intervening polynucleotide sequence (e.g., the second region may have the sequence of nucleic acids from −590 to −161 with respect to the murine Myo15 translation start site, as set forth in SEQ ID NO: 18) or a nucleic acid linker. In a murine Myo15 promoter in which the second region contains both SEQ ID NO: 14 and SEQ ID NO: 15, the two sequences can be included in any order (e.g., SEQ ID NO: 14 may be joined to (e.g., precede) SEQ ID NO: 15, or SEQ ID NO: 15 may be joined to (e.g., precede) SEQ ID NO: 14).

The first region and the second region of the murine Myo15 promoter can be joined directly or can be joined by a nucleic acid linker. For example, the murine Myo15 promoter can contain the sequence of SEQ ID NO: 7 or a functional portion or derivative thereof (e.g., any one or more of SEQ ID NOs: 9-13 and 33, e.g., SEQ ID NOs 9 and 10) fused to the sequence of SEQ ID NO: 8 or a functional portion or derivative thereof (e.g., any one or more of SEQ ID NOs: 14-18, 34, 35, and 38, e.g., SEQ ID NOs 14 and 15) with no intervening nucleic acids. For example, the polynucleotide sequence of the murine Myo15 promoter that results from direct fusion of SEQ ID NO: 7 to SEQ ID NO: 8 is set forth in SEQ ID NO: 19. Alternatively, a linker can be used to join the sequence of SEQ ID NO: 7 or a functional portion or derivative thereof (e.g., any one or more of SEQ ID NOs: 9-13 and 33, e.g., SEQ ID NOs 9 and 10) to the sequence of SEQ ID NO: 8 or a functional portion or derivative thereof (e.g., any one or more of SEQ ID NOs: 14-18, 34, 35, and 38, e.g., SEQ ID NOs 14 and 15). Exemplary Myo15 promoters containing functional portions of both SEQ ID NO: 7 and SEQ ID NO: 8 are provided in SEQ ID NOs: 21, 22, 36, 37, 42, and 43.

The length of a nucleic acid linker for use in a murine Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the murine Myo15 promoter described herein do not disrupt the ability of the murine Myo15 promoter of the invention to induce transgene expression in hair cells.

In some embodiments, the sequence of SEQ ID NO: 7 or a functional portion or derivative thereof (e.g., any one or more of SEQ ID NOs: 9-13 and 33, e.g., SEQ ID NOs 9 and 10) is joined (e.g., operably linked) to the sequence of SEQ ID NO: 8 or a functional portion or derivative thereof (e.g., any one or more of SEQ ID NOs: 14-18, 34, 35, and 38, e.g., SEQ ID NOs 14 and 15), and, in some embodiments, the order of the regions is reversed (e.g., the sequence of SEQ ID NO: 8 or a functional portion or derivative thereof (e.g., any one or more of SEQ ID NOs: 14-18, 34, 35, and 38, e.g., SEQ ID NOs 14 and 15) is joined (e.g., operably linked) to the sequence of SEQ ID NO: 7 or a functional portion or derivative thereof (e.g., any one or more of SEQ ID NOs: 9-13 and 33, e.g., SEQ ID NOs 9 and 10)). For example, the polynucleotide sequence of a murine Myo15 promoter that results from direct fusion of SEQ ID NO: 8 to SEQ ID NO: 7 is set forth in SEQ ID NO: 20. An example of a murine Myo15 promoter in which a functional portion or derivative of SEQ ID NO: 8 precedes a functional portion or derivative of SEQ ID NO: 7 is provided in SEQ ID NO: 41. Regardless of order, the sequence of SEQ ID NO: 7 or a functional portion or derivative thereof and the sequence of SEQ ID NO: 8 or a functional portion or derivative thereof can be joined by direct fusion or a nucleic acid linker, as described above.

In some embodiments, the murine Myo15 promoter for use in the compositions and methods described herein contains a region having at least 85% sequence identity (e.g., 85%, 90%, 91%, 92%, 93%, 94%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 7) or a functional portion or derivative thereof. The functional portion of SEQ ID NO: 7 may have the sequence of nucleic acids from −7166 to −7091 with respect to the murine Myo15 translation start site (set forth in SEQ ID NO: 9) and/or the sequence of nucleic acids from −7077 to −6983 with respect to the murine Myo15 translation start site (set forth in SEQ ID NO: 10). The murine Myo15 promoter may contain the polynucleotide sequence of SEQ ID NO: 9 fused to the polynucleotide sequence of SEQ ID NO: 10 with no intervening nucleic acids, as set forth in SEQ ID NO: 11, or the murine Myo15 promoter may contain the polynucleotide sequence of SEQ ID NO: 10 fused to the polynucleotide sequence of SEQ ID NO: 9 with no intervening nucleic acids, as set forth in SEQ ID NO: 12. Alternatively, the murine Myo15 promoter may contain the sequences of SEQ ID NO: 9 and SEQ ID NO: 10 joined by the endogenous intervening polynucleotide sequence (e.g., the first region may have or include the sequence of nucleic acids from −7166 to −6983 with respect to the murine Myo15 translation start site, as set forth in SEQ ID NO: 13 and SEQ ID NO: 33) or a polynucleotide linker. In a murine Myo15 promoter that contains both SEQ ID NO: 9 and SEQ ID NO: 10, the two sequences can be included in any order (e.g., SEQ ID NO: 9 may be joined to (e.g., precede) SEQ ID NO: 10, or SEQ ID NO: 10 may be joined to (e.g., precede) SEQ ID NO: 9).

In some embodiments, the murine Myo 15 promoter for use in the compositions and methods described herein contains a region having at least 85% sequence identity (e.g., 85%, 90%, 91%, 92%, 93%, 94%, 95%, 96%, 97%, 98%, 99%, or more, sequence identity) to the polynucleotide sequence immediately upstream of the murine Myo15 translation start site (nucleic acids from −1 to −1157 with respect to the murine Myo15 translation start site, the sequence of which is set forth in SEQ ID NO: 8) or a functional portion or derivative thereof. The functional portion of SEQ ID NO: 8 may have the sequence of nucleic acids from −590 to −509 with respect to the murine Myo15 translation start site (set forth in SEQ ID NO: 14) and/or the sequence of nucleic acids from −266 to −161 with respect to the murine Myo15 translation start site (set forth in SEQ ID NO: 15). In some embodiments, the sequence containing SEQ ID NO: 14 has the sequence of SEQ ID NO: 34. In some embodiments, the sequence containing SEQ ID NO: 15 has the sequence of SEQ ID NO: 35. The murine Myo15 promoter may contain the polynucleotide sequence of SEQ ID NO: 14 fused to the polynucleotide sequence of SEQ ID NO: 15 with no intervening nucleic acids, as set forth in SEQ ID NO: 16, or the murine Myo15 promoter may contain the polynucleotide sequence of SEQ ID NO: 15 fused to the polynucleotide sequence of SEQ ID NO: 14 with no intervening nucleic acids, as set forth in SEQ ID NO: 17. The murine Myo15 promoter may contain the nucleic acid sequence of SEQ ID NO: 34 fused to the nucleic acid sequence of SEQ ID NO: 35 with no intervening nucleic acids, as set forth in SEQ ID NO: 38, or the murine Myo15 promoter may contain the nucleic acid sequence of SEQ ID NO: 35 fused to the nucleic acid sequence of SEQ ID NO: 41 with no intervening nucleic acids. Alternatively, the murine Myo15 promoter may contain the sequences of SEQ ID NO: 14 and SEQ ID NO: 15 joined by the endogenous intervening polynucleotide sequence (e.g., the second region may have the sequence of nucleic acids from −590 to −161 with respect to the murine Myo15 translation start site, as set forth in SEQ ID NO: 18) or a nucleic acid linker. In a murine Myo 15 promoter that contains both SEQ ID NO: 14 and SEQ ID NO: 15, the two sequences can be included in any order (e.g., SEQ ID NO: 14 may be joined to (e.g., precede) SEQ ID NO: 15, or SEQ ID NO: 15 may be joined to (e.g., precede) SEQ ID NO: 14).

In some embodiments, the murine Myo15 promoter for use in the compositions and methods described herein contains a functional portion or derivative of a region having at least 85% sequence identity (e.g., 85%, 90%, 91%, 92%, 93%, 94%, 95%, 96%, 97%, 98%, 99%, or more, sequence identity) to a region containing the first non-coding exon of the Myo15 gene (nucleic acids from −6755 to −7209 with respect to the murine Myo15 translation start site, the sequence of which is set forth in SEQ ID NO: 7) flanked on either side by a functional portion or derivative of a region having at least 85% sequence identity (e.g., 85%, 90%, 91%, 92%, 93%, 94%, 95%, 96%, 97%, 98%, 99%, or more, sequence identity) to the nucleic acid sequence immediately upstream of the murine Myo15 translation start site (nucleic acids from −1 to −1157 with respect to the murine Myo15 translation start site, the sequence of which is set forth in SEQ ID NO: 8). For example, a functional portion or derivative of SEQ ID NO: 8, such as SEQ ID NO: 14 or 34 may be directly fused or joined by a nucleic acid linker to a portion of SEQ ID NO: 7, such as any one of SEQ ID NOs: 9-13 and 33, which is directly fused or joined by a nucleic acid linker to a different functional portion of SEQ ID NO: 8, such as SEQ ID NO: 15 or 35. In other embodiments, a functional portion or derivative of SEQ ID NO: 8, such as SEQ ID NO: 15 or 35 may be directly fused or joined by a nucleic acid linker to a portion of SEQ ID NO: 7, such as any one of SEQ ID NOs: 9-13 and 33, which is directly fused or joined by a nucleic acid linker to a different functional portion of SEQ ID NO: 8, such as SEQ ID NO: 14 or 34. For example, polynucleotides having at least 85% sequence identity (e.g., 85%, 90%, 91%, 92%, 93%, 94%, 95%, 96%, 97%, 98%, 99%, or more, sequence identity) to the nucleic acid sequence of SEQ ID NOs: 34, 33, and 35 can be fused to produce a polynucleotide having at least 85% sequence identity (e.g., 85%, 90%, 91%, 92%, 93%, 94%, 95%, 96%, 97%, 98%, 99%, or more, sequence identity) to the nucleic acid sequence of SEQ ID NO: 39. In some embodiments, polynucleotides having at least 85% sequence identity (e.g., 85%, 90%, 91%, 92%, 93%, 94%, 95%, 96%, 97%, 98%, 99%, or more, sequence identity) to the nucleic acid sequence of SEQ ID NOs: 35, 33, and 34 can be fused to produce a polynucleotide having at least 85% sequence identity (e.g., 85%, 90%, 91%, 92%, 93%, 94%, 95%, 96%, 97%, 98%, 99%, or more, sequence identity) to the nucleic acid sequence of SEQ ID NO: 40.

### Human Myosin 15 Promoters

The polynucleotides of the compositions and methods described herein may also include nucleic acid sequences from regions of the human Myo15 locus that are capable of expressing a transgene specifically in hair cells, or variants thereof, such as a nucleic acid sequences that have at least 85% sequence identity (e.g., 85%, 90%, 91%, 92%, 93%, 94%, 95%, 96%, 97%, 98%, 99%, or more, sequence identity) to regions of the human Myo15 locus that are capable of expressing a transgene specifically in hair cells. The polynucleotides of the compositions and methods described herein can optionally include a linker operably linking the regions of the human Myo15 locus that are capable of expressing a transgene specifically in hair cells, or the regions of the human Myo15 locus can be joined directly without an intervening linker.

In some embodiments, the human Myo15 promoter for use in the compositions and methods described herein contains a first region having at least 85% sequence identity (e.g., 85%, 90%, 91%, 92%, 93%, 94%, 95%, 96%, 97%, 98%, 99%, or more, sequence identity) to the sequence set forth in SEQ ID NO: 23 or a functional portion or derivative thereof joined (e.g., operably linked) to a second region having at least 85% sequence identity (e.g., 85%, 90%, 91%, 92%, 93%, 94%, 95%, 96%, 97%, 98%, 99%, or more, sequence identity) to the sequence set forth in SEQ ID NO: 24 or a functional portion or derivative thereof. The functional portion of SEQ ID NO: 23 may have the sequence set forth in SEQ ID NO: 25. The functional portion of SEQ ID NO: 24 may have the sequence set forth in SEQ ID NO: 26 and/or the sequence set forth in SEQ ID NO: 27. The second region may contain the nucleic acid sequence of SEQ ID NO: 26 fused to the nucleic acid sequence of SEQ ID NO: 27 with no intervening nucleic acids, as set forth in SEQ ID NO: 28, or the second region may contain the nucleic acid sequence of SEQ ID NO: 27 fused to the nucleic acid sequence of SEQ ID NO: 26 with no intervening nucleic acids, as set forth in SEQ ID NO: 29. Alternatively, the second region may contain the sequences of SEQ ID NO: 26 and SEQ ID NO: 27 joined by the endogenous intervening nucleic acid sequence (as set forth in SEQ ID NO: 30) or a nucleic acid linker. In a human Myo15 promoter in which the second region contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

The first region and the second region of the human Myo15 promoter can be joined directly or can be joined by a nucleic acid linker. For example, the human Myo15 promoter can contain the sequence of SEQ ID NO: 23 or a functional portion or derivative thereof (e.g., SEQ ID NO: 25) fused to the sequence of SEQ ID NO: 24 or a functional portion or derivative thereof (e.g., any one or more of SEQ ID NOs: 26-30, e.g., SEQ ID NOs: 26 and/or 27) with no intervening nucleic acids. Alternatively, a linker can be used to join the sequence of SEQ ID NO: 23 or a functional portion or derivative thereof (e.g., SEQ ID NO: 25) to the sequence of SEQ ID NO: 24 or a functional portion or derivative thereof (e.g., any one or more of SEQ ID NOs: 26-30, e.g., SEQ ID NOs: 26 and/or 27). Exemplary human Myo15 promoters containing functional portions of both SEQ ID NO: 23 and SEQ ID NO: 24 are provided in SEQ ID NOs: 31 and 32.

In some embodiments, the sequence of SEQ ID NO: 23 or a functional portion or derivative thereof (e.g., SEQ ID NO: 25) is joined (e.g., operably linked) to the sequence of SEQ ID NO: 24 or a functional portion or derivative thereof (e.g., any one or more of SEQ ID NOs: 26-30, e.g., SEQ ID NOs: 26 and 27), and, in some embodiments, the order of the regions is reversed (e.g., the sequence of SEQ ID NO: 24 or a functional portion or derivative thereof (e.g., any one or more of SEQ ID NOs: 26-30, e.g., SEQ ID NOs: 26 and/or 27) is joined (e.g., operably linked) to the sequence of SEQ ID NO: 23 or a functional portion or derivative thereof (e.g., SEQ ID NO: 25)). Regardless of order, the sequence of SEQ ID NO: 23 or a functional portion or derivative thereof and the sequence of SEQ ID NO: 24 or a functional portion or derivative thereof can be joined by direct fusion or a nucleic acid linker, as described above.

In some embodiments, the human Myo15 promoter for use in the compositions and methods described herein contain a region having at least 85% sequence identity (e.g., 85%, 90%, 91%, 92%, 93%, 94%, 95%, 96%, 97%, 98%, 99%, or more, sequence identity) to a region containing the sequence set forth in SEQ ID NO: 23 or a functional portion or derivative thereof. The functional portion of SEQ ID NO: 23 may have the sequence of nucleic acids set forth in SEQ ID NO: 25.

In some embodiments, the human Myo15 promoter for use in the compositions and methods described herein contains a region having at least 85% sequence identity (e.g., 85%, 90%, 91%, 92%, 93%, 94%, 95%, 96%, 97%, 98%, 99%, or more, sequence identity) to the sequence set forth in SEQ ID NO: 18 or a functional portion or derivative thereof. The functional portion of SEQ ID NO: 24 may have the sequence set forth in SEQ ID NO: 26 and/or the sequence set forth in SEQ ID NO: 27. The human Myo15 promoter may contain the nucleic acid sequence of SEQ ID NO: 26 fused to the nucleic acid sequence of SEQ ID NO: 27 with no intervening nucleic acids, as set forth in SEQ ID NO: 28, or the human Myo15 promoter may contain the nucleic acid sequence of SEQ ID NO: 27 fused to the nucleic acid sequence of SEQ ID NO: 26 with no intervening nucleic acids, as set forth in SEQ ID NO: 29. Alternatively, the human Myo15 promoter may contain the sequences of SEQ ID NO: 26 and SEQ ID NO: 27 joined by the endogenous intervening nucleic acid sequence (e.g., as set forth in SEQ ID NO: 30) or a nucleic acid linker. In a human Myo15 promoter that contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

The length of a nucleic acid linker for use in a human Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the human Myo15 promoters described herein do not disrupt the ability of the Myo15 promoter of the invention to induce transgene expression in hair cells.

The foregoing Myo15 promoter sequences are summarized in Table 3, below.

TABLE 3
Exemplary nucleotide sequences for use in the Myo15 promoter described herein

| | Description of | |
|---|---|---|
| SEQ ID | polynucleotide | |
| NO. | sequence | Polynucleotide Sequence |
| 7 | Region containing non- | CTGCAGCTCAGCCTACTACTTGCTTTCCAGGCTGTTCCTAGT |
| | coding exon 1 of | TCCCATGTCAGCTGCTTGTGCTTTCCAGAGACAAAACAGGAA |
| | murine Myo15 (−6755 to | TAATAGATGTCATTAAATATACATTGGGCCCCAGGCGGTCAAT |
| | −7209) | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTAT |
| 8 | Region immediately | GGTCTCACCCAGCATTTTCACTTCTAATAAGTTCAAATGTGAT |
| | preceding the | ACGGCACCTTTCTAAAAATTAGTTTTCAGGGAAATAGGGTTCA |
| | translation start site | AAACTGGTAGTGGTAGGGTCCATTCTCACGACCCCCAGGCCT |
| | of murine Myo15 | GCTAACCCTGACCAAGCTACCTATTACTTACCCTCCTCTTTCT |
| | (−1 to −1157) | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | CCTGATGTATTATTCAGCTCCCTGGAGTTGGCCAGCTCCTGT |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACC |
| 9 | Portion of SEQ ID NO: | CCCATGTCAGCTGCTTGTGCTTTCCAGAGACAAAACAGGAAT |
| | 7 (−7166 to −7091) | AATAGATGTCATTAAATATACATTGGGCCCCAGG |
| 10 | Portion of SEQ ID NO: | AGCCTGAGCCTCCTTTCCATCTCTGTGGAGGCAGACATAGGA |
| | 7 (−7077 to −6983) | CCCCCAACAAACAGCATGCAGGTTGGGAGCCAGCCACAGGA |
| | | CCCAGGTAAGGG |
| 11 | Portion of SEQ ID NO: | CCCATGTCAGCTGCTTGTGCTTTCCAGAGACAAAACAGGAAT |
| | 7 (SEQ ID NO: 9 fused | AATAGATGTCATTAAATATACATTGGGCCCCAGGAGCCTGAG |
| | to SEQ ID NO: 10) | CCTCCTTTCCATCTCTGTGGAGGCAGACATAGGACCCCCAAC |
| | | AAACAGCATGCAGGTTGGGAGCCAGCCACAGGACCCAGGTA |
| | | AGGG |
| 12 | Portion of SEQ ID NO: | AGCCTGAGCCTCCTTTCCATCTCTGTGGAGGCAGACATAGGA |
| | 7 (SEQ ID NO: 10 | CCCCCAACAAACAGCATGCAGGTTGGGAGCCAGCCACAGGA |
| | fused to SEQ ID NO: 9) | CCCAGGTAAGGGCCCATGTCAGCTGCTTGTGCTTTCCAGAGA |
| | | CAAAACAGGAATAATAGATGTCATTAAATATACATTGGGCCCC |
| | | AGG |
| 13 | Portion of SEQ ID NO: | CCCATGTCAGCTGCTTGTGCTTTCCAGAGACAAAACAGGAAT |
| | 7 | AATAGATGTCATTAAATATACATTGGGCCCCAGGCGGTCAAT |
| | (−7166 to −6983) | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGG |
| 14 | Portion of SEQ ID NO: | TGAGGTGGGAGCTGGGCTCTCCCTGATGTATTATTCAGCTCC |
| | 8 (−590 to −509) | CTGGAGTTGGCCAGCTCCTGTTACACTGGCCACAGCCCTG |
| 15 | Portion of SEQ ID NO: | CACAGGCCTCCCAGTCTACCCTCACTAGAAAACAACACCCAG |
| | 8 (−266 to −161) | GCACTTTCCACCACCTCTCAAAGGTGAAACCCAAGGCTGGTC |
| | | TAGAGAATGAATTATGGATCCT |
| 16 | Portion of SEQ ID NO: | TGAGGTGGGAGCTGGGCTCTCCCTGATGTATTATTCAGCTCC |
| | 8 | CTGGAGTTGGCCAGCTCCTGTTACACTGGCCACAGCCCTG |
| | (SEQ ID NO: 14 fused | CACAGGCCTCCCAGTCTACCCTCACTAGAAAACAACACCCAG |
| | to SEQ ID NO: 15) | GCACTTTCCACCACCTCTCAAAGGTGAAACCCAAGGCTGGTC |
| | | TAGAGAATGAATTATGGATCCT |
| 17 | Portion of SEQ ID NO: | CACAGGCCTCCCAGTCTACCCTCACTAGAAAACAACACCCAG |
| | 8 | GCACTTTCCACCACCTCTCAAAGGTGAAACCCAAGGCTGGTC |
| | (SEQ ID NO: 15 fused | TAGAGAATGAATTATGGATCCTTGAGGTGGGAGCTGGGCTCT |
| | to SEQ ID NO: 14) | CCCTGATGTATTATTCAGCTCCCTGGAGTTGGCCAGCTCCTG |
| | | TTACACTGGCCACAGCCCTG |
| 18 | Portion of SEQ ID NO: | TGAGGTGGGAGCTGGGCTCTCCCTGATGTATTATTCAGCTCC |
| | 8 | CTGGAGTTGGCCAGCTCCTGTTACACTGGCCACAGCCCTGG |
| | (−590 to −161) | GCATCCGCTTCTCACTTCTAGTTTCCCCTCCAAGGTAATGTG |
| | | GTGGGTCATGATCATTCTATCCTGGCTTCAGGGACCTGACTC |
| | | CACTTTGGGGCCATTCGAGGGGTCTAGGGTAGATGATGTCC |
| | | CCCTGTGGGGATTAATGTCCTGCTCTGTAAAACTGAGCTAGC |
| | | TGAGATCCAGGAGGGCTTGGCCAGAGACAGCAAGTTGTTGC |
| | | CATGGTGACTTTAAAGCCAGGTTGCTGCCCCAGCACAGGCCT |
| | | CCCAGTCTACCCTCACTAGAAAACAACACCCAGGCACTTTCC |
| | | ACCACCTCTCAAAGGTGAAACCCAAGGCTGGTCTAGAGAATG |
| | | AATTATGGATCCT |
| 19 | SEQ ID NO: 7 fused to | CTGCAGCTCAGCCTACTACTTGCTTTCCAGGCTGTTCCTAGT |
| | SEQ ID NO: 8 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTATGGTC |
| | | TCACCCAGCATTTTCACTTCTAATAAGTTCAAATGTGATACGG |
| | | CACCTTTCTAAAAATTAGTTTTCAGGGAAATAGGGTTCAAAAC |
| | | TGGTAGTGGTAGGGTCCATTCTCACGACCCCCAGGCCTGCT |
| | | AACCCTGACCAAGCTACCTATTACTTACCCTCCTCTTTCTCCT |
| | | CCTCCTCTTTCTCCTTCTCCTGCTTCCCCTCTTCCTTCTCCCT |
| | | CCCTTCCTCTCCCTCCTCCCCCTCCTTGGCTGTGATCAGATC |
| | | CAGAGCCTGAATGAGCCTCCTGACCCCACACCCCCACTAGC |
| | | ATGGGCCTGCAAGTGCCCAGAAGTCCCTCCTGCCTCCTAAAC |
| | | TGCCCAGCCGATCCATTAGCTCTTCCTTCTTCCCAGTGAAAG |
| | | AAGCAGGCACAGCCTGTCCCTCCCGTTCTACAGAAAGGAAG |
| | | CTACAGCACAGGGAGGGCCAAAGGCCTTCCTGGGACTAGAC |
| | | AGTTGATCAACAGCAGGACTGGAGAGCTGGGCTCCATTTTTG |
| | | TTCCTTGGTGCCCTGCCCCTCCCCATGACCTGCAGAGACATT |
| | | CAGCCTGCCAGGCTTTATGAGGTGGGAGCTGGGCTCTCCCT |
| | | GATGTATTATTCAGCTCCCTGGAGTTGGCCAGCTCCTGTTAC |
| | | ACTGGCCACAGCCCTGGGCATCCGCTTCTCACTTCTAGTTTC |
| | | CCCTCCAAGGTAATGTGGTGGGTCATGATCATTCTATCCTGG |
| | | CTTCAGGGACCTGACTCCACTTTGGGGCCATTCGAGGGGTC |
| | | TAGGGTAGATGATGTCCCCCTGTGGGGATTAATGTCCTGCTC |
| | | TGTAAAACTGAGCTAGCTGAGATCCAGGAGGGCTTGGCCAG |
| | | AGACAGCAAGTTGTTGCCATGGTGACTTTAAAGCCAGGTTGC |
| | | TGCCCCAGCACAGGCCTCCCAGTCTACCCTCACTAGAAAACA |
| | | ACACCCAGGCACTTTCCACCACCTCTCAAAGGTGAAACCCAA |
| | | GGCTGGTCTAGAGAATGAATTATGGATCCTCGCTGTCCGTGC |
| | | CACCCAGCTAGTCCCAGCGGCTCAGACACTGAGGAGAGACT |
| | | GTAGGTTCAGCTACAAGCAAAAAGACCTAGCTGGTCTCCAAG |
| | | CAGTGTCTCCAAGTCCCTGAACCTGTGACACCTGCCCCAGG |
| | | CATCATCAGGCACAGAGGGCCACC |
| 20 | SEQ ID NO: 8 fused to | GGTCTCACCCAGCATTTTCACTTCTAATAAGTTCAAATGTGAT |
| | SEQ ID NO: 7 | ACGGCACCTTTCTAAAAATTAGTTTTCAGGGAAATAGGGTTCA |
| | | AAACTGGTAGTGGTAGGGTCCATTCTCACGACCCCCAGGCCT |
| | | GCTAACCCTGACCAAGCTACCTATTACTTACCCTCCTCTTTCT |
| | | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | CCTGATGTATTATTCAGCTCCCTGGAGTTGGCCAGCTCCTGT |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACCCTGCAGCTCAGCCTAC |
| | | TACTTGCTTTCCAGGCTGTTCCTAGTTCCCATGTCAGCTGCTT |
| | | GTGCTTTCCAGAGACAAAACAGGAATAATAGATGTCATTAAAT |
| | | ATACATTGGGCCCCAGGCGGTCAATGTGGCAGCCTGAGCCT |
| | | CCTTTCCATCTCTGTGGAGGCAGACATAGGACCCCCAACAAA |
| | | CAGCATGCAGGTTGGGAGCCAGCCACAGGACCCAGGTAAGG |
| | | GGCCCTGGGTCCTTAAGCTTCTGCCACTGGCTCCGGCATTG |
| | | CAGAGAGAAGAGAAGGGGCGGCAGAGCTGAACCTTAGCCTT |
| | | GCCTTCCTGGGTACCCTTCTGAGCCTCACTGTCTTCTGTGAG |
| | | ATGGGCAAAGTGCGGGTGTGACTCCTTGGCAACGGTGTTAC |
| | | ACCAGGGCAGGTAAAGTTGTAGTTATTTGTGGGGTACACCAG |
| | | GACTGTTAAAGGTGTAACTAT |
| 21 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 14 and SEQ ID NO: 15 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 22 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 14 and SEQ ID NO: 15 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 23 | Region 1 of the human | GTATGCCTTTTGAGATGGATGCAGCAGGTTCTGTGAGGCTGC |
| | Myo15 promoter | CAGGAGGGGTAGAGTTCCCGGGGGCCTCGGGCCCCGCTGG |
| | | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCC |
| 24 | Region 2 of human | TGCCCTGCCTTCTGAGCCGGCAGCCTGGCTCCCCACCCCAT |
| | Myo15 promoter | GTATTATTCAGCTCCTGAGAGCCAGCCAGCTCCTGTTACACT |
| | | GACCGCAGCCCAGCACCTGCTCTGCCCATTCCCCTCCTCCC |
| | | TTGCCTAGGACCTAGAGGGTTCAAAGTTCTCCTCCAAGATGA |
| | | CTTGGTGGGCTTTGGCCATCCCACCCTAGGCCCCACTTCTG |
| | | GCCCAGTGCAGGTGTGCTGGTGATTTAGGGCAGGTGGCATT |
| | | CCATCTCTGTGGCTCAATGTCTTCCTCTGTGAAGCCGAAGTG |
| | | ACCCAAGGGCTCCCTTCATGGGGTTGAGCCAGCTGTGGCCC |
| | | AGGGAGGGCCTAACCAGGATGAGCACTGATGTTGCCATGAC |
| | | GACTCCGAGGCCAGAATGTCTCCCCCAGCACAGGCCTCATA |
| | | GGCAGGCTTCCCCATCCTGGTAAACAACACCCACACACTTTC |
| | | TACTACTGCTCTAGGGTGAAACCCAAGGCGCTCTAGAGGAGA |
| | | TGAATTATGGATCCGCCCTCCCGGAATCCTGGCTCGGCCCTC |
| | | CCCACGCCACCCAGGGCCAGTCGGGTCTGCTCACAGCCCGA |
| | | GGAGGCCGCGTGTCCAGCCGCGGGCAAGAGACAGAGCAGG |
| | | TCCCTGTGTCTCCAAGTCCCTGAGCCCGTGACACCGGCCCC |
| | | AGGCCCTGTAGAGAGCAGGCAGCCACC |
| 25 | Portion of SEQ ID NO: | CCCCTGTCAGCTGCTCACTCCTTTCCAGAGACAAAACAGGAA |
| | 23 | TAATAGACATCATTAAATATACATAGGGCCCCAGG |
| 26 | Portion of SEQ ID NO: | TGAGCCGGCAGCCTGGCTCCCCACCCCATGTATTATTCAGCT |
| | 24 | CCTGAGAGCCAGCCAGCTCCTGTTACACTGACCGCAGCCC |
| 27 | Portion of SEQ ID NO: | CACAGGCCTCATAGGCAGGCTTCCCCATCCTGGTAAACAACA |
| | 24 | CCCACACACTTTCTACTACTGCTCTAGGGTGAAACCCAAGGC |
| | | GCTCTAGAGGAGATGAATTATGGATCC |
| 28 | Portion of SEQ ID NO: | TGAGCCGGCAGCCTGGCTCCCCACCCCATGTATTATTCAGCT |
| | 24 | CCTGAGAGCCAGCCAGCTCCTGTTACACTGACCGCAGCCCC |
| | (SEQ ID NO: 26 fused | ACAGGCCTCATAGGCAGGCTTCCCCATCCTGGTAAACAACAC |
| | to SEQ ID NO: 27) | CCACACACTTTCTACTACTGCTCTAGGGTGAAACCCAAGGCG |
| | | CTCTAGAGGAGATGAATTATGGATCC |
| 29 | Portion of SEQ ID NO: | CACAGGCCTCATAGGCAGGCTTCCCCATCCTGGTAAACAACA |
| | 24 | CCCACACACTTTCTACTACTGCTCTAGGGTGAAACCCAAGGC |
| | (SEQ ID NO: 27 fused | GCTCTAGAGGAGATGAATTATGGATCCTGAGCCGGCAGCCT |
| | to SEQ ID NO: 26) | GGCTCCCCACCCCATGTATTATTCAGCTCCTGAGAGCCAGCC |
| | | AGCTCCTGTTACACTGACCGCAGCCC |
| 30 | Portion of SEQ ID NO: | TGAGCCGGCAGCCTGGCTCCCCACCCCATGTATTATTCAGCT |
| | 24 (contiguous | CCTGAGAGCCAGCCAGCTCCTGTTACACTGACCGCAGCCCA |
| | sequence including | GCACCTGCTCTGCCCATTCCCCTCCTCCCTTGCCTAGGACCT |
| | SEQ ID NO: 26 and | AGAGGGTTCAAAGTTCTCCTCCAAGATGACTTGGTGGGCTTT |
| | SEQ ID NO: 27) | GGCCATCCCACCCTAGGCCCCACTTCTGGCCCAGTGCAGGT |
| | | GTGCTGGTGATTTAGGGCAGGTGGCATTCCATCTCTGTGGCT |
| | | CAATGTCTTCCTCTGTGAAGCCGAAGTGACCCAAGGGCTCCC |
| | | TTCATGGGGTTGAGCCAGCTGTGGCCCAGGGAGGGCCTAAC |
| | | CAGGATGAGCACTGATGTTGCCATGACGACTCCGAGGCCAG |
| | | AATGTCTCCCCCAGCACAGGCCTCATAGGCAGGCTTCCCCAT |
| | | CCTGGTAAACAACACCCACACACTTTCTACTACTGCTCTAGG |
| | | GTGAAACCCAAGGCGCTCTAGAGGAGATGAATTATGGATCC |
| 31 | Polynucleotide | GTATGCCTTTTGAGATGGATGCAGCAGGTTCTGTGAGGCTGC |
| | containing SEQ ID NO: | CAGGAGGGGTAGAGTTCCCGGGGGCCTCGGGCCCCGCTGG |
| | 23 and SEQ ID NO: 24 | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCCCCATAACACTGAG |
| | | CTGCTCTGCTGGGCCAACCGTGCTCCTGGGCCAGCCAGAGG |
| | | ACCCCCATGAGGCGGCATGCAGGCGGGGAGCAGGCCACAG |
| | | AACGCAGGTAAGGAGACCTTAGCCTAGAGTCCTTGGGGTCT |
| | | GTCACTGGCCACCCTCGCATCCCAGGCTGCAGGAAACTGAG |
| | | GCCCAGAGAGGACAAGGACTTTCCTGGACCCACACAGCCAG |
| | | TCAGTGACAGAGCCTAGGGTCTGAGCCAGGCCTGACCCAAC |
| | | CTCCATTTCTGCCTCTCTACCCCTGCCCCCGCCCCAACACAC |
| | | ACACACACACAAGTGGAGTTCCACTGAAACGCCCCTCCTTGC |
| | | CCTGCCTTCTGAGCCGGCAGCCTGGCTCCCCACCCCATGTA |
| | | TTATTCAGCTCCTGAGAGCCAGCCAGCTCCTGTTACACTGAC |
| | | CGCAGCCCAGCACCTGCTCTGCCCATTCCCCTCCTCCCTTGC |
| | | CTAGGACCTAGAGGGTTCAAAGTTCTCCTCCAAGATGACTTG |
| | | GTGGGCTTTGGCCATCCCACCCTAGGCCCCACTTCTGGCCC |
| | | AGTGCAGGTGTGCTGGTGATTTAGGGCAGGTGGCATTCCAT |
| | | CTCTGTGGCTCAATGTCTTCCTCTGTGAAGCCGAAGTGACCC |
| | | AAGGGCTCCCTTCATGGGGTTGAGCCAGCTGTGGCCCAGGG |
| | | AGGGCCTAACCAGGATGAGCACTGATGTTGCCATGACGACT |
| | | CCGAGGCCAGAATGTCTCCCCCAGCACAGGCCTCATAGGCA |
| | | GGCTTCCCCATCCTGGTAAACAACACCCACACACTTTCTACT |
| | | ACTGCTCTAGGGTGAAACCCAAGGCGCTCTAGAGGAGATGA |
| | | ATTATGGATCCGCCCTCCCGGAATCCTGGCTCGGCCCTCCC |
| | | CACGCCACCCAGGGCCAGTCGGGTCTGCTCACAGCCCGAG |
| | | GAGGCCGCGTGTCCAGCCGCGGGCAAGAGACAGAGCAGGT |
| | | CCCTGTGTCTCCAAGTCCCTGAGCCCGTGACACCGGCCCCA |
| | | GGCCCTGTAGAGAGCAGGCAGCCACC |
| 32 | Polynucleotide | GCAGGCCCATGCTCAGCTCTCCAGGCTGTTCGTGGCTCCCC |
| | containing SEQ ID NO: | TGTCAGCTGCTCACTCCTTTCCAGAGACAAAACAGGAATAAT |
| | 25, SEQ ID NO: 26, | AGACATCATTAAATATACATAGGGCCCCAGGCGGTCGGCGTG |
| | and SEQ ID NO: 27 | GTGGGCTGGGCCTCCCTTCCCCATAACACTGAGCTGCTCTG |
| | | CTGGGCCAACCGTGCTCCTGGGCCAGCCAGAGGACCCCCAT |
| | | GAGGCGGCATGCAGGCGGGGAGCAGGCCACAGAACGCAGG |
| | | TAAGGAGACCTTGCCTTCTGAGCCGGCAGCCTGGCTCCCCA |
| | | CCCCATGTATTATTCAGCTCCTGAGAGCCAGCCAGCTCCTGT |
| | | TACACTGACCGCAGCCCAGCACCTGCTCTGCCCATTCCCCTC |
| | | CTCCCTTGCCTAGGACCTAGAGGGTTCAAAGTTCTCCTCCAA |
| | | GATGACTTGGTGGGCTTTGGCCATCGGGCCTAACCAGGATG |
| | | AGCACTGATGTTGCCATGACGACTCCGAGGCCAGAATGTCTC |
| | | CCCCAGCACAGGCCTCATAGGCAGGCTTCCCCATCCTGGTA |
| | | AACAACACCCACACACTTTCTACTACTGCTCTAGGGTGAAAC |
| | | CCAAGGCGCTCTAGAGGAGATGAATTATGGATCCGCCCTCC |
| | | CGGAATCCTGGCTCGGCCCTCCCCACGC |
| 33 | Portion of SEQ ID NO: | CTGCAGCTCAGCCTACTACTTGCTTTCCAGGCTGTTCCTAGT |
| | 7 that contains SEQ ID | TCCCATGTCAGCTGCTTGTGCTTTCCAGAGACAAAACAGGAA |
| | NO: 9 and SEQ ID NO: | TAATAGATGTCATTAAATATACATTGGGCCCCAGGCGGTCAAT |
| | 10 | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTT |
| 34 | Portion of SEQ ID NO: | TTTATGAGGTGGGAGCTGGGCTCTCCCTGATGTATTATTCAG |
| | 8 that contains SEQ ID | CTCCCTGGAGTTGGCCAGCTCCTGTTACACTGGCCACAGCC |
| | NO: 14 | CTGGGCATCCGC |
| 35 | Portion of SEQ ID NO: | TGCCATGGTGACTTTAAAGCCAGGTTGCTGCCCCAGCACAG |
| | 8 that contains SEQ ID | GCCTCCCAGTCTACCCTCACTAGAAAACAACACCCAGGCACT |
| | NO: 15 | TTCCACCACCTCTCAAAGGTGAAACCCAAGGCTGGTCTAGAG |
| | | AATGAATTATGGATCCTCGCTGTCCGTGCCACCCAGCTAGTC |
| | | CCAGCGGCTCAGACACTG |
| 36 | SEQ ID NO: 33 fused | CTGCAGCTCAGCCTACTACTTGCTTTCCAGGCTGTTCCTAGT |
| | to SEQ ID NO: 34 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTTTATGAGGT |
| | | GGGAGCTGGGCTCTCCCTGATGTATTATTCAGCTCCCTGGAG |
| | | TTGGCCAGCTCCTGTTACACTGGCCACAGCCCTGGGCATCC |
| | | GC |
| 37 | SEQ ID NO: 33 fused | CTGCAGCTCAGCCTACTACTTGCTTTCCAGGCTGTTCCTAGT |
| | to SEQ ID NO: 35 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTGCCATGGTG |
| | | ACTTTAAAGCCAGGTTGCTGCCCCAGCACAGGCCTCCCAGTC |
| | | TACCCTCACTAGAAAACAACACCCAGGCACTTTCCACCACCT |
| | | CTCAAAGGTGAAACCCAAGGCTGGTCTAGAGAATGAATTATG |
| | | GATCCTCGCTGTCCGTGCCACCCAGCTAGTCCCAGCGGCTC |
| | | AGACACTG |
| 38 | SEQ ID NO: 34 fused | TTTATGAGGTGGGAGCTGGGCTCTCCCTGATGTATTATTCAG |
| | to SEQ ID NO: 35 | CTCCCTGGAGTTGGCCAGCTCCTGTTACACTGGCCACAGCC |
| | | CTGGGCATCCGCTGCCATGGTGACTTTAAAGCCAGGTTGCTG |
| | | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTG |
| 39 | SEQ ID NO: 34 fused | TTTATGAGGTGGGAGCTGGGCTCTCCCTGATGTATTATTCAG |
| | to SEQ ID NO: 33, | CTCCCTGGAGTTGGCCAGCTCCTGTTACACTGGCCACAGCC |
| | which is fused to SEQ | CTGGGCATCCGCCTGCAGCTCAGCCTACTACTTGCTTTCCAG |
| | ID NO: 35 | GCTGTTCCTAGTTCCCATGTCAGCTGCTTGTGCTTTCCAGAG |
| | | ACAAAACAGGAATAATAGATGTCATTAAATATACATTGGGCCC |
| | | CAGGCGGTCAATGTGGCAGCCTGAGCCTCCTTTCCATCTCTG |
| | | TGGAGGCAGACATAGGACCCCCAACAAACAGCATGCAGGTT |
| | | GGGAGCCAGCCACAGGACCCAGGTAAGGGGCCCTGGGTCC |
| | | TTTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 40 | SEQ ID NO: 35 fused | TGCCATGGTGACTTTAAAGCCAGGTTGCTGCCCCAGCACAG |
| | to SEQ ID NO: 33, | GCCTCCCAGTCTACCCTCACTAGAAAACAACACCCAGGCACT |
| | which is fused to SEQ | TTCCACCACCTCTCAAAGGTGAAACCCAAGGCTGGTCTAGAG |
| | ID NO: 34 | AATGAATTATGGATCCTCGCTGTCCGTGCCACCCAGCTAGTC |
| | | CCAGCGGCTCAGACACTGCTGCAGCTCAGCCTACTACTTGCT |
| | | TTCCAGGCTGTTCCTAGTTCCCATGTCAGCTGCTTGTGCTTTC |
| | | CAGAGACAAAACAGGAATAATAGATGTCATTAAATATACATTG |
| | | GGCCCCAGGCGGTCAATGTGGCAGCCTGAGCCTCCTTTCCA |
| | | TCTCTGTGGAGGCAGACATAGGACCCCCAACAAACAGCATG |
| | | CAGGTTGGGAGCCAGCCACAGGACCCAGGTAAGGGGCCCT |
| | | GGGTCCTTTTTATGAGGTGGGAGCTGGGCTCTCCCTGATGTA |
| | | TTATTCAGCTCCCTGGAGTTGGCCAGCTCCTGTTACACTGGC |
| | | CACAGCCCTGGGCATCCGC |
| 41 | SEQ ID NO: 34 fused | TTTATGAGGTGGGAGCTGGGCTCTCCCTGATGTATTATTCAG |
| | to SEQ ID NO: 35, | CTCCCTGGAGTTGGCCAGCTCCTGTTACACTGGCCACAGCC |
| | which is fused to SEQ | CTGGGCATCCGCTGCCATGGTGACTTTAAAGCCAGGTTGCTG |
| | ID NO: 33 | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTGCTGCAGCTCAGC |
| | | CTACTACTTGCTTTCCAGGCTGTTCCTAGTTCCCATGTCAGCT |
| | | GCTTGTGCTTTCCAGAGACAAAACAGGAATAATAGATGTCATT |
| | | AAATATACATTGGGCCCCAGGCGGTCAATGTGGCAGCCTGA |
| | | GCCTCCTTTCCATCTCTGTGGAGGCAGACATAGGACCCCCAA |
| | | CAAACAGCATGCAGGTTGGGAGCCAGCCACAGGACCCAGGT |
| | | AAGGGGCCCTGGGTCCTT |
| 42 | Portion of SEQ ID NO: | TGCAGCTCAGCCTACTACTTGCTTTCCAGGCTGTTCCTAGTT |
| | 7 that contains SEQ ID | CCCATGTCAGCTGCTTGTGCTTTCCAGAGACAAAACAGGAAT |
| | NO: 9 and SEQ ID NO: | AATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 14 and SEQ ID NO: 15 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 43 | Portion of SEQ ID NO: | TGCAGCTCAGCCTACTACTTGCTTTCCAGGCTGTTCCTAGTT |
| | 7 that contains SEQ ID | CCCATGTCAGCTGCTTGTGCTTTCCAGAGACAAAACAGGAAT |
| | NO: 9 and SEQ ID NO: | AATAGATGTCATTAAATATACATTGGGCCCCAGGCGGTCAAT |
| | 10 fused to portion of | GTGGCAGCCTGAGCCTCCTTTCCATCTCTGTGGAGGCAGAC |
| | SEQ ID NO: 8 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 14 and SEQ ID NO: 15 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |

Additional Myo15 promoters useful in conjunction with the compositions and methods described herein include nucleic acid molecules that have at least 85% sequence identity (e.g., 85%, 90%, 91%, 92%, 93%, 94%, 95%, 96%, 97%, 98%, 99%, or more, sequence identity) to the polynucleotide sequences set forth in Table 3, as well as functional portions or derivatives of the polynucleotide sequences set forth in Table 3. The Myo15 promoters listed in Table 3 are characterized in International Application Publication Nos. WO2019210181A1 and WO2020163761A1, which are incorporated herein by reference.

In embodiments in which an smCBA promoter is included in a dual vector system described herein (e.g., in the first vector in a dual vector system), the smCBA promoter may have the sequence of the smCBA promoter described in U.S. Pat. No. 8,298,818, which is incorporated herein by reference. In some embodiments, the smCBA promoter has the sequence of:

(SEQ ID NO: 44)
GGTACCTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCAC
TCTCCCCATCTCCCCCCCCTCCCCACCCCCAATTTTGTATTTATTTATTTTTTAATTAT
TTTGTGCAGCGATGGGGGGGGGGGGGGGGGGGGGGGGCGCGCCAGGGGGGCG
GGGGGGGGCGAGGGGGGGGGGGGGCGAGGCGGAGAGGTGCGGCGGCAGCCA
ATCAGAGCGGCGCGCTCCGAAAGTTTCCTTTTATGGCGAGGGGGGGGGGGGCG
GCCCTATAAAAAGCGAAGCGCGCGGCGGGCGGGAGTCGCTGCGCGCTGCCTTCG
CCCCGTGCCCCGCTCCGCCGCCGCCTCGCGCCGCCCGCCCCGGCTCTGACTGAC
CGCGTTACTCCCACAGGTGAGCGGGGGGACGGCCCTTCTCCTCCGGGCTGTAAT
TAGCGCTTGGTTTAATGACGGCTTGTTTCTTTTCTGTGGCTGCGTGAAAGCCTTGAG
GGGCTCCGGGAGCTAGAGCCTCTGCTAACCATGTTCATGCCTTCTTCTTTTTCCTAC
AGCTCCTGGGCAACGTGCTGGTTATTGTGCTGTCTCATCATTTTGGCA.

Once a polynucleotide encoding OTOF has been incorporated into the nuclear DNA of a mammalian cell or stabilized in an episomal monomer or concatemer, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in the nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode an OTOF protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv, et al., Nature 297:17 (1982). An enhancer may be spliced into a vector containing a polynucleotide encoding an OTOF protein, for example, at a position 5′ or 3′ to this gene. In a preferred orientation, the enhancer is positioned at the 5′ side of the promoter, which in turn is located 5′ relative to the polynucleotide encoding an OTOF protein.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. The WPRE can be located in the second nucleic acid vector between the polynucleotide encoding a C-terminal portion of an OTOF protein and the poly(A) sequence. In some embodiments of the compositions and methods described herein, the WPRE has the sequence:

(SEQ ID NO: 45)
GATCCAATCAACCTCTGGATTACAAAATTTGTGAAAGATTGACTGGTAT
TCTTAACTATGTTGCTCCTTTTACGCTATGTGGATACGCTGCTTTAATG
CCTTTGTATCATGCTATTGCTTCCCGTATGGCTTTCATTTTCTCCTCCT
TGTATAAATCCTGGTTGCTGTCTCTTTATGAGGAGTTGTGGCCCGTTGT
CAGGCAACGTGGCGTGGTGTGCACTGTGTTTGCTGACGCAACCCCCACT
GGTTGGGGCATTGCCACCACCTGTCAGCTCCTTTCCGGGACTTTCGCTT
TCCCCCTCCCTATTGCCACGGCGGAACTCATCGCCGCCTGCCTTGCCCG
CTGCTGGACAGGGGCTCGGCTGTTGGGCACTGACAATTCCGTGGTGTTG
TCGGGGAAATCATCGTCCTTTCCTTGGCTGCTCGCCTGTGTTGCCACCT
GGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCG
CGTCTTCGA.

In other embodiments, the WPRE has the sequence:

(SEQ ID NO: 46)
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTAGTTCTTGCCACGGCGGAACTCATCGCCGCCTGCCTTG
CCCGCTGCTGGACAGGGGCTCGGCTGTTGGGCACTGACAATTCCGTGGT
GTTTATTTGTGAAATTTGTGATGCTATTGCTTTATTTGTAACCATCTAG
CTTTATTTGTGAAATTTGTGATGCTATTGCTTTATTTGTAACCATTATA
AGCTGCAATAAACAAGTTAACAACAACAATTGCATTCATTTTATGTTTC
AGGTTCAGGGGGAGATGTGGGAGGTTTTTTAAA

### Dual Hybrid Vectors for Expressing OTOF

An OTOF isoform 5 protein (e.g., an OTOF isoform 5 protein having the sequence of SEQ ID NO: 1) can be expressed in mammalian cells using a dual hybrid vector system. This approach uses two nucleic acid vectors (e.g., two adeno-associated virus vectors) to express a single, large protein. Each of the two nucleic acid vectors (e.g., two adeno-associated virus vectors) contains a portion of a polynucleotide that encodes the protein (e.g., one vector contains a polynucleotide encoding an N-terminal portion of the protein and the other vector contains a polynucleotide encoding a C-terminal portion of the protein, and the polynucleotide encoding the N-terminal portion of the protein and the polynucleotide encoding the C-terminal portion of the protein do not overlap). The dual hybrid vectors also feature an overlapping region at which homologous recombination can occur (e.g., a recombinogenic region that is contained within each vector) and splice donor and splice acceptor sequences (e.g., the first vector contains a splice donor sequence and the second vector contains a splice acceptor sequence). The recombinogenic region is 3′ of the splice donor sequence in the first nucleic acid vector and 5′ of the splice acceptor sequence in the second nucleic acid vector. The first and second polynucleotide sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest.

Recombinogenic regions that can be used in the compositions and methods described herein include the F1 phage AK gene having a sequence of: GGGATTTTGCCGATTTCGGCCTATTGGTTAA AAAATGAGCTGATTTAACAAAAATTTAACGCGAATTTTAACAAAAT (SEQ ID NO: 47) and alkaline phosphatase (AP) gene fragments as described in U.S. Pat. No. 8,236,557, which are incorporated herein by reference. In some embodiments, the AP gene fragment has the sequence of:

(SEQ ID NO: 48)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTCCGTATAGG
AGGACCGTGTAGGCCTTCCTGTCCCGGGCCTTGCCAGCGGCCAGCCCGA
TGAAGGAGCTCCCTCGCAGGGGGTAGCCTCCGAAGGAGAAGACGTGGGA
GTGGTCGGCAGTGACGAGGCTCAGCGTGTCCTCCTCGCTGGTGAGCTGG
CCCGCCCTCTCAATGGCGTCGTCGAACATGATCGTCTCAGTCAGTGCCC
GGTAAGCCCTGCTTTCATGATGACCATGGTCGATGCGACCACCCTCCAC
GAAGAGGAAGAAGCCGCGGGGGTGTCTGCTCAGCAGGCGCAGGGCAGCC
TCTGTCATCTCCATCAGGGAGGGGTCCAGTGTGGAGTCTCGGTGGATCT
CGTATTTCATGTCTCCAGGCTCAAAGAGACCCATGAGATGGGTCACAGA
CGGGTCCAGGGAAGCCTGCATGAGCTCAGTGCGGTTCCACACGTACCGG
GCACCCTGGCGTTCGCCGAGCCATTCCTGCACCAGATTCTTCCCGTCCA
GCCTGGTCCCACCTTGGCTGTAGTCATCTGGGTACTCAGGGTCTGGGGT
TCCCATGCGAAACATGTACTTTCGGCCTCCA.

In some embodiments, the AP gene fragment has the sequence of:

(SEQ ID NO: 49)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTCCGTATAGG
AGGACCGTGTAGGCCTTCCTGTCCCGGGCCTTGCCAGCGGCCAGCCCGA
TGAAGGAGCTCCCTCGCAGGGGGTAGCCTCCGAAGGAGAAGACGTGGGA
GTGGTCGGCAGTGACGAGGCTCAGCGTGTCCTCCTCG CTGGTGA.

In some embodiments, the AP gene fragment has the sequence of:

(SEQ ID NO: 50)
GCTGGCCCGCCCTCTCAATGGCGTCGTCGAACATGATCGTCTCAGTCAG
TGCCCGGTAAGCCCTGCTTTCATGATGACCATGGTCGATGCGACCACCC
TCCACGAAGAGGAAGAAGCCGCGGGGGTGTCTGCTCAGCAGGCGCAGGG
CAGCCTCTGTCATCTCCATCAGGGAGGGGTCCAGTGTGGAGTCTCGGTG
GATCTCGTATTTCATGTCTCCAGGCTCAAAGAGACCCATGAGATGGGTC
ACAGACGGGTCCAGGGAAGCCTGCATGAGCTCAGTGCGGTTCCACACGT
ACCGGGCACCCTGGCGTTCGCCGAGCCATTCCTGCACCAGATTCTTCCC
GTCCAGCCTGGTCCCACCTTGGCTGTAGTCATCTGGGTACTCAGGGTCT
GGGGTTCCCATGCGAAACATGTACTTTCGGCCTCCA.

In some embodiments, the AP gene fragment has the sequence of:

(SEQ ID NO: 51)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTC

In some embodiments, the AP gene fragment has the sequence of:

(SEQ ID NO: 52)
CGTATAGGAGGACCGTGTAGGCCTTCCTGTCCCGGGCCTTGCCAGCGGC
CAGCCCGATGAAGGAGCTCCCTCGCAGGGGGTAGCCTCCGAAGGAGAAG
ACGTGGGAGTGGTCGGCAGTGACGAGGCTCAGCGTGTCCTCCTCGCTGG
TGAGCTGGCCCGCCCTCTCAATGGCGTCGTCGAACATGATCGTCTCAGT
CAGTGCCCGGTAAGCCCTGCTTTCATGATGACCATGGTCGATGCGACCA
CCCTCCACGAAGAGGAAGAAGCCGCGGGGGTGTCTGCTCAGCAGG.

In some embodiments, the AP gene fragment has the sequence of:

(SEQ ID NO: 53)
CGCAGGGCAGCCTCTGTCATCTCCATCAGGGAGGGGTCCAGTGTGGAGT
CTCGGTGGATCTCGTATTTCATGTCTCCAGGCTCAAAGAGACCCATGAG
ATGGGTCACAGACGGGTCCAGGGAAGCCTGCATGAGCTCAGTGCGGTTC
CACACGTACCGGGCACCCTGGCGTTCGCCGAGCCATTCCTGCACCAGAT
TCTTCCCGTCCAGCCTGGTCCCACCTTGGCTGTAGTCATCTGGGTACTC
AGGGTCTGGGGTTCCCATGCGAAACATGTACTTTCGGCCTCCA.

An exemplary splice donor sequence for use in the methods and compositions described herein can include the sequence:

(SEQ ID NO: 54)
GTAAGTATCAAGGTTACAAGACAGGTTTAAGGAGACCAATAGAAACTGG
GCTTGTCGAGACAGAGAAGACTCTTGCGTTTCTGA.

An exemplary splice acceptor sequence for use in the methods and compositions described herein can include the sequence:

(SEQ ID NO: 55)
TAGGCACCTATTGGTCTTACTGACATCCACTTTGCCTTTCTCTCCACAG.

Additional examples of splice donor and splice acceptor sequences are known in the art.

Dual hybrid vectors for use in the methods and compositions described herein are designed such that approximately half of the OTOF gene is contained within each vector (e.g., each vector contains a polynucleotide that encodes approximately half of the OTOF isoform 5 protein). The determination of how to split the polynucleotide sequence between the two nucleic acid vectors can be made based on the size of the promoter and the locations of the portions of the polynucleotide that encode the OTOF C2 domains. When a short promoter is used in the dual hybrid vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as CAG, CMV, smCBA, or a Myo15 promoter having a sequence that is 1 kb or shorter (e.g., a Myo15 promoter described hereinabove, e.g., a Myo15 promoter having the sequence of SEQ ID NO: 21 or SEQ ID NO: 42), the OTOF polynucleotide sequence can be divided between the two nucleic acid vectors at an exon boundary that occurs after the portion of the polynucleotide that encodes the C2D domain and before the portion of the polynucleotide that encodes C2E domain, for example, the exon 26/27 boundary. The nucleic acid vectors containing promoters of this size can optionally contain OTOF UTRs (e.g., full-length 5′ and 3′ UTRs). When a long promoter is used in the dual hybrid vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 19), the OTOF polynucleotide sequence can be divided between the two nucleic acid vectors at an exon boundary that occurs after the portion of the polynucleotide that encodes the C2C domain, and either before the portion of the polynucleotide that encodes the C2D domain, such as the exon 19/20 boundary, the exon 20/21 boundary, the exon 21/22 boundary, or within the portion of the polynucleotide that encodes the C2D domain, such as the exon 25/26 boundary. A short promoter (e.g., a CMV promoter, CAG promoter, smCBA promoter, or a Myo15 promoter having a sequence that is 1 kb or shorter, e.g., a Myo15 promoter having the sequence of SEQ ID NO: 21 or SEQ ID NO: 42) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary dual hybrid vector system that uses a short promoter includes a first nucleic acid vector containing a CAG promoter operably linked to exons 1-26 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 27-45 and 47 of a polynucleotide encoding an OTOF isoform 5 protein (e.g., human OTOF isoform 5, e.g., SEQ ID NO: 1), and a poly(A) sequence (e.g., a bGH poly(A) signal sequence). The first and second nucleic acid vectors can also contain full-length 5′ and 3′ OTOF UTRs, respectively (e.g., the 127 bp human OTOF 5′ UTR can be included in the first nucleic acid vector, and the 1035 bp human OTOF 3′ UTR can be included in the second nucleic acid vector). Another exemplary dual hybrid vector system that uses a short promoter includes a first nucleic acid vector containing a smCBA promoter or a Myo 15 promoter that is 1 kb or shorter (e.g., a Myo15 promoter having the sequence of SEQ ID NO: 21 or SEQ ID NO: 42) operably linked to exons 1-20 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 21-45 and 47 of a polynucleotide encoding an OTOF isoform 5 protein (e.g., human OTOF, e.g., SEQ ID NO: 1), and a poly(A) sequence (e.g., a bGH poly(A) signal sequence). The first nucleic acid vector can also contain the full-length 5′ OTOF UTRs (e.g., the 127 bp human OTOF 5′ UTR can be included in the first nucleic acid vector). The CMV promoter can be used in place of the CAG, smCBA, or Myo15 promoter in either of the foregoing dual vector systems.

An exemplary dual hybrid vector system that uses a long promoter includes a first nucleic acid vector containing a Myo15 promoter that is longer than 1 kb (e.g., SEQ ID NO: 19) operably linked to exons 1-19 or exons 1-20 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 20-45 and 47 (when the first nucleic acid vector contains exons 1-19 of the polynucleotide) or exons 21-45 and 47 (when the first nucleic acid vector contains exons 1-20 of the polynucleotide) of a polynucleotide encoding an OTOF isoform 5 protein (e.g., human OTOF isoform 5, e.g., SEQ ID NO: 1), and a poly(A) sequence (e.g., a bGH poly(A) signal sequence). Neither the first nor the second nucleic acid vector in the foregoing Myo15 promoter dual hybrid vector system contains an OTOF UTR. A short promoter (e.g., a CMV promoter, CAG promoter, smCBA promoter, or a Myo15 promoter having a sequence that is 1 kb or shorter, e.g., a Myo15 promoter having the sequence of SEQ ID NO: 21 or SEQ ID NO: 42) can also be used in the foregoing dual vector systems designed for large promoters. If these dual vector systems contain a short promoter, they may also include a 5′ OTOF UTR in the first vector.

To accommodate an OTOF UTR, the OTOF coding sequence can be divided in a different position. For example, in a dual hybrid vector system in which the first nucleic acid vector contains a Myo15 promoter that is longer than 1 kb (e.g., SEQ ID NO: 19) operably linked to exons 1-25 of a polynucleotide encoding an OTOF isoform 5 protein (e.g., human OTOF isoform 5, e.g., SEQ ID NO: 1), a splice donor sequence 3′ of the polynucleotide sequence, and a recombinogenic region 3′ of the splice donor sequence; and in which the second nucleic acid vector contains a recombinogenic region, a splice acceptor sequence 3′ of the recombinogenic region, a polynucleotide 3′ of the splice acceptor sequence that contains exons 26-45 and 47 of a polynucleotide encoding an OTOF protein (e.g., human OTOF isoform 5, e.g., SEQ ID NO: 1), and a poly(A) sequence (e.g., a bGH poly(A) signal sequence), the second nucleic acid can also contain a full-length OTOF 3′ UTR (e.g., the 1035 bp human OTOF UTR). A short promoter (e.g., a CMV promoter, CAG promoter, smCBA promoter, or a Myo15 promoter having a sequence that is 1 kb or shorter, e.g., a Myo15 promoter having the sequence of SEQ ID NO: 21 or SEQ ID NO: 42) can also be used in the foregoing dual vector system designed for large promoters. If these dual vector systems contain a short promoter, they may also include a 5′ OTOF UTR in the first vector.

The polynucleotide sequence encoding an OTOF isoform 5 protein can be a cDNA sequence (e.g., a sequence that does not include introns). In some embodiments, the first and/or the second nucleic acid vector in the dual vector system can include intronic sequence. The intronic sequence may be included between one or more exons in the OTOF coding sequence, or the intronic sequence can be included between an exon of the coding sequence and another component of the nucleic acid vector (e.g., between an exon of the OTOF coding sequence and the splice donor sequence in the first nucleic acid vector or between an exon of the OTOF coding sequence and the splice acceptor sequence in the second nucleic acid vector).

In some embodiments, the polynucleotide encoding OTOF isoform 5 is divided between the first and second nucleic acid vectors (e.g., AAV vectors) in the dual vector system at the exon 20/21 boundary. In some embodiments, the polynucleotide encoding OTOF isoform 5 is divided between the first and second nucleic acid vectors (e.g., AAV vectors) in the dual vector system at the exon 21/22 boundary.

When the polynucleotide encoding OTOF isoform 5 is divided between the first and second nucleic acid vectors (e.g., AAV vectors) at the exon 20/21 boundary, the polynucleotide sequence encoding the N-terminal portion of OTOF has the sequence of:

(SEQ ID NO: 56)
ATGGCCTTGCTCATCCACCTCAAGACAGTCTCGGAGCTGCGGGGCAGGG
GCGACCGGATCGCCAAAGTGACTTTCCGAGGGCAATCCTTCTACTCTCG
GGTCCTGGAGAACTGTGAGGATGTGGCTGACTTTGATGAGACATTTCGG
TGGCCGGTGGCCAGCAGCATCGACAGAAATGAGATGCTGGAGATTCAGG
TTTTCAACTACAGCAAAGTCTTCAGCAACAAGCTCATCGGGACCTTCCG
CATGGTGCTGCAGAAGGTGGTAGAGGAGAGCCATGTGGAGGTGACTGAC
ACGCTGATTGATGACAACAATGCTATCATCAAGACCAGCCTGTGCGTGG
AGGTCCGGTATCAGGCCACTGACGGCACAGTGGGCTCCTGGGACGATGG
GGACTTCCTGGGAGATGAGTCTCTTCAAGAGGAAGAGAAGGACAGCCAA
GAGACGGATGGACTGCTCCCAGGCTCCCGGCCCAGCTCCCGGCCCCCAG
GAGAGAAGAGCTTCCGGAGAGCCGGGAGGAGCGTGTTCTCCGCCATGAA
GCTCGGCAAAAACCGGTCTCACAAGGAGGAGCCCCAAAGACCAGATGAA
CCGGCGGTGCTGGAGATGGAAGACCTTGACCATCTGGCCATTCGGCTAG
GAGATGGACTGGATCCCGACTCGGTGTCTCTAGCCTCAGTCACAGCTCT
CACCACTAATGTCTCCAACAAGCGATCTAAGCCAGACATTAAGATGGAG
CCAAGTGCTGGGCGGCCCATGGATTACCAGGTCAGCATCACGGTGATCG
AGGCCCGGCAGCTGGTGGGCTTGAACATGGACCCTGTGGTGTGCGTGGA
GGTGGGTGACGACAAGAAGTACACATCCATGAAGGAGTCCACTAACTGC
CCCTATTACAACGAGTACTTCGTCTTCGACTTCCATGTCTCTCCGGATG
TCATGTTTGACAAGATCATCAAGATTTCGGTGATTCACTCCAAGAACCT
GCTGCGCAGTGGCACCCTGGTGGGCTCCTTCAAAATGGACGTGGGAACC
GTGTACTCGCAGCCAGAGCACCAGTTCCATCACAAGTGGGCCATCCTGT
CTGACCCCGATGACATCTCCTCGGGGCTGAAGGGCTACGTGAAGTGTGA
CGTTGCCGTGGTGGGCAAAGGGGACAACATCAAGACGCCCCACAAGGCC
AATGAGACCGACGAAGATGACATTGAGGGGAACTTGCTGCTCCCCGAGG
GGGTGCCCCCCGAACGCCAGTGGGCCCGGTTCTATGTGAAAATTTACCG
AGCAGAGGGGCTGCCCCGTATGAACACAAGCCTCATGGCCAATGTAAAG
AAGGCTTTCATCGGTGAAAACAAGGACCTCGTGGACCCCTACGTGCAAG
TCTTCTTTGCTGGCCAGAAGGGCAAGACTTCAGTGCAGAAGAGCAGCTA
TGAGCCCCTGTGGAATGAGCAGGTCGTCTTTACAGACCTCTTCCCCCCA
CTCTGCAAACGCATGAAGGTGCAGATCCGAGACTCGGACAAGGTCAACG
ACGTGGCCATCGGCACCCACTTCATTGACCTGCGCAAGATTTCTAATGA
CGGAGACAAAGGCTTCCTGCCCACACTGGGCCCAGCCTGGGTGAACATG
TACGGCTCCACACGTAACTACACGCTGCTGGATGAGCATCAGGACCTGA
ACGAGGGCCTGGGGGAGGGTGTGTCCTTCCGGGCCCGGCTCCTGCTGGG
CCTGGCTGTGGAGATCGTAGACACCTCCAACCCTGAGCTCACCAGCTCC
ACAGAGGTGCAGGTGGAGCAGGCCACGCCCATCTCGGAGAGCTGTGCAG
GTAAAATGGAAGAATTCTTTCTCTTTGGAGCCTTCCTGGAGGCCTCAAT
GATCGACCGGAGAAACGGAGACAAGCCCATCACCTTTGAGGTCACCATA
GGCAACTATGGGAACGAAGTTGATGGCCTGTCCCGGCCCCAGCGGCCTC
GGCCCCGGAAGGAGCCGGGGGATGAGGAAGAAGTAGACCTGATTCAGAA
CGCAAGTGATGACGAGGCCGGTGATGCCGGGGACCTGGCCTCAGTCTCC
TCCACTCCACCAATGCGGCCCCAGGTCACCGACAGGAACTACTTCCATC
TGCCCTACCTGGAGCGAAAGCCCTGCATCTACATCAAGAGCTGGTGGCC
GGACCAGCGCCGCCGCCTCTACAATGCCAACATCATGGACCACATTGCC
GACAAGCTGGAAGAAGGCCTGAACGACATACAGGAGATGATCAAAACGG
AGAAGTCCTACCCTGAGCGTCGCCTGCGGGGCGTCCTGGAGGAGCTGAG
CTGTGGCTGCTGCCGCTTCCTCTCCCTCGCTGACAAGGACCAGGGCCAC
TCATCCCGCACCAGGCTTGACCGGGAGCGCCTCAAGTCCTGCATGAGGG
AGCTG.

When the polynucleotide encoding OTOF isoform 5 is divided between the first and second nucleic acid vectors (e.g., AAV vectors) at the exon 20/21 boundary, the polynucleotide sequence encoding the C-terminal portion of OTOF has the sequence of:

(SEQ ID NO: 57)
GAAAACATGGGGCAGCAGGCCAGGATGCTGCGGGCCCAGGTGAAGCGGC
ACACGGTGCGGGACAAGCTGAGGCTGTGCCAGAACTTCCTGCAGAAGCT
GCGCTTCCTGGCGGACGAGCCCCAGCACAGCATTCCCGACATCTTCATC
TGGATGATGAGCAACAACAAGCGTGTCGCCTATGCCCGTGTGCCCTCCA
AGGACCTGCTCTTCTCCATCGTGGAGGAGGAGACTGGCAAGGACTGCGC
CAAGGTCAAGACGCTCTTCCTTAAGCTGCCAGGGAAGCGGGGCTTCGGC
TCGGCAGGCTGGACAGTGCAGGCCAAGGTGGAGCTGTACCTGTGGCTGG
GCCTCAGCAAACAGCGCAAGGAGTTCCTGTGCGGCCTGCCCTGTGGCTT
CCAGGAGGTCAAGGCAGCCCAGGGCCTGGGCCTGCATGCCTTCCCACCC
GTCAGCCTGGTCTACACCAAGAAGCAGGCGTTCCAGCTCCGAGCGCACA
TGTACCAGGCCCGCAGCCTCTTTGCCGCCGACAGCAGCGGACTCTCAGA
CCCCTTTGCCCGCGTCTTCTTCATCAATCAGAGTCAGTGCACAGAGGTG
CTGAATGAGACCCTGTGTCCCACCTGGGACCAGATGCTGGTGTTCGACA
ACCTGGAGCTCTATGGTGAAGCTCATGAGCTGAGGGACGATCCGCCCAT
CATTGTCATTGAAATCTATGACCAGGATTCCATGGGCAAAGCTGACTTC
ATGGGCCGGACCTTCGCCAAACCCCTGGTGAAGATGGCAGACGAGGCGT
ACTGCCCACCCCGCTTCCCACCTCAGCTCGAGTACTACCAGATCTACCG
TGGCAACGCCACAGCTGGAGACCTGCTGGCGGCCTTCGAGCTGCTGCAG
ATTGGACCAGCAGGGAAGGCTGACCTGCCCCCCATCAATGGCCCGGTGG
ACGTGGACCGAGGTCCCATCATGCCCGTGCCCATGGGCATCCGGCCCGT
GCTCAGCAAGTACCGAGTGGAGGTGCTGTTCTGGGGCCTACGGGACCTA
AAGCGGGTGAACCTGGCCCAGGTGGACCGGCCACGGGTGGACATCGAGT
GTGCAGGGAAGGGGGTGCAGTCGTCCCTGATCCACAATTATAAGAAGAA
CCCCAACTTCAACACCCTCGTCAAGTGGTTTGAAGTGGACCTCCCAGAG
AACGAGCTGCTGCACCCGCCCTTGAACATCCGTGTGGTGGACTGCCGGG
CCTTCGGTCGCTACACACTGGTGGGCTCCCATGCCGTCAGCTCCCTGCG
ACGCTTCATCTACCGGCCCCCAGACCGCTCGGCCCCCAGCTGGAACACC
ACGGTCAGGCTTCTCCGGCGCTGCCGTGTGCTGTGCAATGGGGGCTCCT
CCTCTCACTCCACAGGGGAGGTTGTGGTGACTATGGAGCCAGAGGTACC
CATCAAGAAACTGGAGACCATGGTGAAGCTGGACGCGACTTCTGAAGCT
GTTGTCAAGGTGGATGTGGCTGAGGAGGAGAAGGAGAAGAAGAAGAAGA
AGAAGGGCACTGCGGAGGAGCCAGAGGAGGAGGAGCCAGACGAGAGCAT
GCTGGACTGGTGGTCCAAGTACTTTGCCTCCATTGACACCATGAAGGAG
CAACTTCGACAACAAGAGCCCTCTGGAATTGACTTGGAGGAGAAGGAGG
AAGTGGACAATACCGAGGGCCTGAAGGGGTCAATGAAGGGCAAGGAGAA
GGCAAGGGCTGCCAAAGAGGAGAAGAAGAAGAAAACTCAGAGCTCTGGC
TCTGGCCAGGGGTCCGAGGCCCCCGAGAAGAAGAAACCCAAGATTGATG
AGCTTAAGGTATACCCCAAAGAGCTGGAGTCCGAGTTTGATAACTTTGA
GGACTGGCTGCACACTTTCAACTTGCTTCGGGGCAAGACCGGGGATGAT
GAGGATGGCTCCACCGAGGAGGAGCGCATTGTGGGACGCTTCAAGGGCT
CCCTCTGCGTGTACAAAGTGCCACTCCCAGAGGACGTGTCCCGGGAAGC
CGGCTACGACTCCACCTACGGCATGTTCCAGGGCATCCCGAGCAATGAC
CCCATCAATGTGCTGGTCCGAGTCTATGTGGTCCGGGCCACGGACCTGC
ACCCTGCTGACATCAACGGCAAAGCTGACCCCTACATCGCCATCCGGCT
AGGCAAGACTGACATCCGCGACAAGGAGAACTACATCTCCAAGCAGCTC
AACCCTGTCTTTGGGAAGTCCTTTGACATCGAGGCCTCCTTCCCCATGG
AATCCATGCTGACGGTGGCTGTGTATGACTGGGACCTGGTGGGCACTGA
TGACCTCATTGGGGAAACCAAGATCGACCTGGAGAACCGCTTCTACAGC
AAGCACCGCGCCACCTGCGGCATCGCCCAGACCTACTCCACACATGGCT
ACAATATCTGGCGGGACCCCATGAAGCCCAGCCAGATCCTGACCCGCCT
CTGCAAAGACGGCAAAGTGGACGGCCCCCACTTTGGGCCCCCTGGGAGA
GTGAAGGTGGCCAACCGCGTCTTCACTGGGCCCTCTGAGATTGAGGACG
AGAACGGTCAGAGGAAGCCCACAGACGAGCATGTGGCGCTGTTGGCCCT
GAGGCACTGGGAGGACATCCCCCGCGCAGGCTGCCGCCTGGTGCCAGAG
CATGTGGAGACGAGGCCGCTGCTCAACCCCGACAAGCCGGGCATCGAGC
AGGGCCGCCTGGAGCTGTGGGTGGACATGTTCCCCATGGACATGCCAGC
CCCTGGGACGCCTCTGGACATCTCACCTCGGAAGCCCAAGAAGTACGAG
CTGCGGGTCATCATCTGGAACACAGATGAGGTGGTCTTGGAGGACGACG
ACTTCTTCACAGGGGAGAAGTCCAGTGACATCTTCGTGAGGGGGTGGCT
GAAGGGCCAGCAGGAGGACAAGCAGGACACAGACGTCCACTACCACTCC
CTCACTGGCGAGGGCAACTTCAACTGGCGCTACCTGTTCCCCTTCGACT
ACCTGGCGGCGGAGGAGAAGATCGTCATCTCCAAGAAGGAGTCCATGTT
CTCCTGGGACGAGACCGAGTACAAGATCCCCGCGCGGCTCACCCTGCAG
ATCTGGGATGCGGACCACTTCTCCGCTGACGACTTCCTGGGGGCCATCG
AGCTGGACCTGAACCGGTTCCCGCGGGGCGCAAAGACAGCCAAGCAGTG
CACCATGGAGATGGCCACCGGGGAGGTGGACGTGCCCCTCGTGTCCATC
TTCAAGCAAAAGCGCGTCAAAGGCTGGTGGCCCCTCCTGGCCCGCAATG
AGAACGATGAGTTTGAGCTCACGGGCAAGGTGGAGGCTGAGCTGCATTT
ACTGACAGCAGAGGAGGCAGAGAAGAACCCAGTGGGCCTGGCCCGCAAT
GAACCTGACCCCCTAGAGAAACCCAACCGGCCCGACACGGCCTTCGTCT
GGTTCCTCAACCCTCTCAAGTCCATCAAGTACCTCATCTGCACCCGGTA
CAAGTGGCTCATCATCAAGATCGTGCTGGCGCTGTTGGGGCTGCTCATG
TTGGGGCTCTTCCTCTACAGCCTCCCTGGCTACATGGTCAAAAAGCTCC
TTGGGGCATGA.

In embodiments in which the polynucleotide encoding OTOF isoform 5 is divided between the first and second nucleic acid vectors (e.g., AAV vectors) at the exon 20/21 boundary, the N-terminal portion of the OTOF polypeptide has the sequence of:

(SEQ ID NO: 58)
MALLIHLKTVSELRGRGDRIAKVTFRGQSFYSRVLENCEDVADFDETFR
WPVASSIDRNEMLEIQVFNYSKVFSNKLIGTFRMVLQKVVEESHVEVTD
TLIDDNNAIIKTSLCVEVRYQATDGTVGSWDDGDFLGDESLQEEEKDSQ
ETDGLLPGSRPSSRPPGEKSFRRAGRSVFSAMKLGKNRSHKEEPQRPDE
PAVLEMEDLDHLAIRLGDGLDPDSVSLASVTALTTNVSNKRSKPDIKME
PSAGRPMDYQVSITVIEARQLVGLNMDPVVCVEVGDDKKYTSMKESTNC
PYYNEYFVFDFHVSPDVMFDKIIKISVIHSKNLLRSGTLVGSFKMDVGT
VYSQPEHQFHHKWAILSDPDDISSGLKGYVKCDVAVVGKGDNIKTPHKA
NETDEDDIEGNLLLPEGVPPERQWARFYVKIYRAEGLPRMNTSLMANVK
KAFIGENKDLVDPYVQVFFAGQKGKTSVQKSSYEPLWNEQVVFTDLFPP
LCKRMKVQIRDSDKVNDVAIGTHFIDLRKISNDGDKGFLPTLGPAWVNM
YGSTRNYTLLDEHQDLNEGLGEGVSFRARLLLGLAVEIVDTSNPELTSS
TEVQVEQATPISESCAGKMEEFFLFGAFLEASMIDRRNGDKPITFEVTI
GNYGNEVDGLSRPQRPRPRKEPGDEEEVDLIQNASDDEAGDAGDLASVS
STPPMRPQVTDRNYFHLPYLERKPCIYIKSWWPDQRRRLYNANIMDHIA
DKLEEGLNDIQEMIKTEKSYPERRLRGVLEELSCGCCRFLSLADKDQGH
SSRTRLDRERLKSCMREL.

In embodiments in which the polynucleotide encoding OTOF isoform 5 is divided between the first and second nucleic acid vectors (e.g., AAV vectors) at the exon 20/21 boundary, the C-terminal portion of the OTOF polypeptide has the sequence of:

(SEQ ID NO: 59)
ENMGQQARMLRAQVKRHTVRDKLRLCQNFLQKLRFLADEPQHSIPDIFI
WMMSNNKRVAYARVPSKDLLFSIVEEETGKDCAKVKTLFLKLPGKRGFG
SAGWTVQAKVELYLWLGLSKQRKEFLCGLPCGFQEVKAAQGLGLHAFPP
VSLVYTKKQAFQLRAHMYQARSLFAADSSGLSDPFARVFFINQSQCTEV
LNETLCPTWDQMLVFDNLELYGEAHELRDDPPIIVIEIYDQDSMGKADF
MGRTFAKPLVKMADEAYCPPRFPPQLEYYQIYRGNATAGDLLAAFELLQ
IGPAGKADLPPINGPVDVDRGPIMPVPMGIRPVLSKYRVEVLFWGLRDL
KRVNLAQVDRPRVDIECAGKGVQSSLIHNYKKNPNFNTLVKWFEVDLPE
NELLHPPLNIRVVDCRAFGRYTLVGSHAVSSLRRFIYRPPDRSAPSWNT
TVRLLRRCRVLQNGGSSSHSTGEVVVTMEPEVPIKKLETMVKLDATSEA
VVKVDVAEEEKEKKKKKKGTAEEPEEEEPDESMLDWWSKYFASIDTMKE
QLRQQEPSGIDLEEKEEVDNTEGLKGSMKGKEKARAAKEEKKKKTQSSG
SGQGSEAPEKKKPKIDELKVYPKELESEFDNFEDWLHTFNLLRGKTGDD
EDGSTEEERIVGRFKGSLCVYKVPLPEDVSREAGYDSTYGMFQGIPSND
PINVLVRVYVVRATDLHPADINGKADPYIAIRLGKTDIRDKENYISKQL
NPVFGKSFDIEASFPMESMLTVAVYDWDLVGTDDLIGETKIDLENRFYS
KHRATCGIAQTYSTHGYNIWRDPMKPSQILTRLCKDGKVDGPHFGPPGR
VKVANRVFTGPSEIEDENGQRKPTDEHVALLALRHWEDIPRAGCRLVPE
HVETRPLLNPDKPGIEQGRLELWVDMFPMDMPAPGTPLDISPRKPKKYE
LRVIIWNTDEVVLEDDDFFTGEKSSDIFVRGWLKGQQEDKQDTDVHYHS
LTGEGNFNWRYLFPFDYLAAEEKIVISKKESMFSWDETEYKIPARLTLQ
IWDADHFSADDFLGAIELDLNRFPRGAKTAKQCTMEMATGEVDVPLVSI
FKQKRVKGWWPLLARNENDEFELTGKVEAELHLLTAEEAEKNPVGLARN
EPDPLEKPNRPDTAFVWFLNPLKSIKYLICTRYKWLIIKIVLALLGLLM
LGLFLYSLPGYMVKKLLGA.

Transfer plasmids that may be used to produce the nucleic acid vectors for use in the compositions and methods described herein are provided in Table 4. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. Nucleic acid vectors (e.g., a nucleic acid vector (e.g., an AAV vector) containing a polynucleotide encoding an N-terminal portion of OTOF isoform 5 and a nucleic acid vector (e.g., an AAV vector) containing a polynucleotide encoding a C-terminal portion of OTOF isoform 5) can be combined (e.g., in a single formulation) prior to administration. The following transfer plasmids are designed to produce nucleic acid vectors (e.g., AAV vectors) for co-formulation or co-administration (e.g., administration simultaneously or sequentially) in a dual hybrid vector system: SEQ ID NO: 60 and SEQ ID NO: 61; SEQ ID NO: 62 and SEQ ID NO: 63; SEQ ID NO: 64 and SEQ ID NO: 61; SEQ ID NO: 65 and SEQ ID NO: 63; SEQ ID NO: 66 and SEQ ID NO: 67; and SEQ ID NO: 68 and SEQ ID NO: 67.

TABLE 4
Transfer plasmids for the production of dual hybrid vector systems

| SEQ | | |
|---|---|---|
| ID | | |
| NO. | Description | Plasmid Sequence |
| 60 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda (Biologically inert and | TTTCCATCATTCCAGTCGAACTCACACACAACACCAAA |
| | inactivated DNA derived from | TGCATTTAAGTCGCTTGAAATTGCTATAAGCAGAGCAT |
| | bacteriophage lambda to | GTTGCGCCAGCATGATTAATACAGCATTTAATACAGAG |
| | reduce off-target DNA | CCGTGTTTATTGAGTCGGTATTCAGAGTCTGACCAGAA |
| | encapsidation) at positions | ATTATTAATCTGGTGAAGTTATTCCTCTGTCATTACGTC |
| | 53-2027 | ATGGTCGATTTCAATTTCTATTGATGCTTTCCAGTCGTA |
| | ITR at positions 2049-2178 | ATCAATGATGTATTTTTTGATGTTTGACCTCTGTTCATA |
| | Myo15 promoter at positions | TCCTCACAGATAAAAAATCGCCCTCACACTGGAGGGC |
| | 2272-3236 | AAAGAAGATTTCCAATAATCAGAACAAGTCGGCTCCTG |
| | Kozak sequence (Site to initiate | TTTAGTTACGAGCGACATTGCTCCGTGTATTCACTCGT |
| | protein translation) at positions | TGGAATGAATACACAGTGCAGTGTTTATTCTGTTATTTA |
| | 3253-3262 | TGCCAAAAATTAAGGCCACTATCAGGCAGCTTTGTTGT |
| | N-terminal portion of human | TCTGTTTACCAAGTTCTCTGGCAATCATTGCCGTCGTT |
| | OTOF isoform 5 at positions | CGTATTGCCCATTTATCGACATATTTCCCATCTTCCTAT |
| | 3259-5664 | ACAGGAAACATTTCTTCAGGCTTAACCATGCATTCCGA |
| | Splice donor (APSD) sequence | TTGCAGCTTGCATCCATTGCATCGCTTGAATTGTCCAC |
| | at positions 5665-5748 | ACCATTGATTTTTATCAATAGTCGTAGTTTAACGGATAG |
| | AP head sequence | TCCTGGTATTGTTCCATCACATCCTGAGGATGCCCTTC |
| | (recombinogenic region) at | GAACTCTTCAAATTCTTCTTCCTAATATCACCTTAAATA |
| | positions 5755-6041 | GTGGATTGCGGTAGTAAAGATTGTGCCTGTCTTTTAAC |
| | ITR at positions 6135-6264 | CACATCAGGCTCGGTGGTTCTCGTGTACCCCTACAGC |
| | Lambda at positions 6275-8287 | GAGAAATCGGATAAACTATTACAACCCCTACAGTTTGT |
| | Ori (origin of replication) at | AGAGTATAGAAAATGATCCACTCGTTATTCTCGGACGA |
| | positions 8344-8932 | GTGTTCAGTAATGAACCTCTGGAGAGAACCATCTATAT |
| | KanR (antibiotic resistance | GATCGTTATCTGGGTTTGACTTCTGCTTTTAAGCCCAG |
| | gene) at positions 9110-9919 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6264 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCAATTC |
| | | TGCAGCTCAGCCTACTACTTGCTTTCCAGGCTGTTCCT |
| | | AGTTCCCATGTCAGCTGCTTGTGCTTTCCAGAGACAAA |
| | | ACAGGAATAATAGATGTCATTAAATATACATTGGGCCC |
| | | CAGGCGGTCAATGTGGCAGCCTGAGCCTCCTTTCCAT |
| | | CTCTGTGGAGGCAGACATAGGACCCCCAACAAACAGC |
| | | ATGCAGGTTGGGAGCCAGCCACAGGACCCAGGTAAG |
| | | GGGCCCTGGGTCCTTAAGCTTCTGCCACTGGCTCCGG |
| | | CATTGCAGAGAGAAGAGAAGGGGCGGCAGACTGGAG |
| | | AGCTGGGCTCCATTTTTGTTCCTTGGTGCCCTGCCCCT |
| | | CCCCATGACCTGCAGAGACATTCAGCCTGCCAGGCTT |
| | | TATGAGGTGGGAGCTGGGCTCTCCCTGATGTATTATTC |
| | | AGCTCCCTGGAGTTGGCCAGCTCCTGTTACACTGGCC |
| | | ACAGCCCTGGGCATCCGCTTCTCACTTCTAGTTTCCCC |
| | | TCCAAGGTAATGTGGTGGGTCATGATCATTCTATCCTG |
| | | GCTTCAGGGACCTGACTCCACTTTGGGGCCATTCGAG |
| | | GGGTCTAGGGTAGATGATGTCCCCCTGTGGGGATTAA |
| | | TGTCCTGCTCTGTAAAACTGAGCTAGCTGAGATCCAG |
| | | GAGGGCTTGGCCAGAGACAGCAAGTTGTTGCCATGGT |
| | | GACTTTAAAGCCAGGTTGCTGCCCCAGCACAGGCCTC |
| | | CCAGTCTACCCTCACTAGAAAACAACACCCAGGCACTT |
| | | TCCACCACCTCTCAAAGGTGAAACCCAAGGCTGGTCT |
| | | AGAGAATGAATTATGGATCCTCGCTGTCCGTGCCACC |
| | | CAGCTAGTCCCAGCGGCTCAGACACTGAGGAGAGACT |
| | | GTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCAAGA |
| | | ATTCTAGCGGCCGCCACCATGGCCTTGCTCATCCACC |
| | | TCAAGACAGTCTCGGAGCTGCGGGGCAGGGGCGACC |
| | | GGATCGCCAAAGTGACTTTCCGAGGGCAATCCTTCTA |
| | | CTCTCGGGTCCTGGAGAACTGTGAGGATGTGGCTGAC |
| | | TTTGATGAGACATTTCGGTGGCCGGTGGCCAGCAGCA |
| | | TCGACAGAAATGAGATGCTGGAGATTCAGGTTTTCAAC |
| | | TACAGCAAAGTCTTCAGCAACAAGCTCATCGGGACCTT |
| | | CCGCATGGTGCTGCAGAAGGTGGTAGAGGAGAGCCA |
| | | TGTGGAGGTGACTGACACGCTGATTGATGACAACAAT |
| | | GCTATCATCAAGACCAGCCTGTGCGTGGAGGTCCGGT |
| | | ATCAGGCCACTGACGGCACAGTGGGCTCCTGGGACG |
| | | ATGGGGACTTCCTGGGAGATGAGTCTCTTCAAGAGGA |
| | | AGAGAAGGACAGCCAAGAGACGGATGGACTGCTCCCA |
| | | GGCTCCCGGCCCAGCTCCCGGCCCCCAGGAGAGAAG |
| | | AGCTTCCGGAGAGCCGGGAGGAGCGTGTTCTCCGCC |
| | | ATGAAGCTCGGCAAAAACCGGTCTCACAAGGAGGAGC |
| | | CCCAAAGACCAGATGAACCGGCGGTGCTGGAGATGG |
| | | AAGACCTTGACCATCTGGCCATTCGGCTAGGAGATGG |
| | | ACTGGATCCCGACTCGGTGTCTCTAGCCTCAGTCACA |
| | | GCTCTCACCACTAATGTCTCCAACAAGCGATCTAAGCC |
| | | AGACATTAAGATGGAGCCAAGTGCTGGGCGGCCCATG |
| | | GATTACCAGGTCAGCATCACGGTGATCGAGGCCCGGC |
| | | AGCTGGTGGGCTTGAACATGGACCCTGTGGTGTGCGT |
| | | GGAGGTGGGTGACGACAAGAAGTACACATCCATGAAG |
| | | GAGTCCACTAACTGCCCCTATTACAACGAGTACTTCGT |
| | | CTTCGACTTCCATGTCTCTCCGGATGTCATGTTTGACA |
| | | AGATCATCAAGATTTCGGTGATTCACTCCAAGAACCTG |
| | | CTGCGCAGTGGCACCCTGGTGGGCTCCTTCAAAATGG |
| | | ACGTGGGAACCGTGTACTCGCAGCCAGAGCACCAGTT |
| | | CCATCACAAGTGGGCCATCCTGTCTGACCCCGATGAC |
| | | ATCTCCTCGGGGCTGAAGGGCTACGTGAAGTGTGACG |
| | | TTGCCGTGGTGGGCAAAGGGGACAACATCAAGACGCC |
| | | CCACAAGGCCAATGAGACCGACGAAGATGACATTGAG |
| | | GGGAACTTGCTGCTCCCCGAGGGGGTGCCCCCCGAA |
| | | CGCCAGTGGGCCCGGTTCTATGTGAAAATTTACCGAG |
| | | CAGAGGGGCTGCCCCGTATGAACACAAGCCTCATGGC |
| | | CAATGTAAAGAAGGCTTTCATCGGTGAAAACAAGGACC |
| | | TCGTGGACCCCTACGTGCAAGTCTTCTTTGCTGGCCA |
| | | GAAGGGCAAGACTTCAGTGCAGAAGAGCAGCTATGAG |
| | | CCCCTGTGGAATGAGCAGGTCGTCTTTACAGACCTCTT |
| | | CCCCCCACTCTGCAAACGCATGAAGGTGCAGATCCGA |
| | | GACTCGGACAAGGTCAACGACGTGGCCATCGGCACC |
| | | CACTTCATTGACCTGCGCAAGATTTCTAATGACGGAGA |
| | | CAAAGGCTTCCTGCCCACACTGGGCCCAGCCTGGGTG |
| | | AACATGTACGGCTCCACACGTAACTACACGCTGCTGG |
| | | ATGAGCATCAGGACCTGAACGAGGGCCTGGGGGAGG |
| | | GTGTGTCCTTCCGGGCCCGGCTCCTGCTGGGCCTGG |
| | | CTGTGGAGATCGTAGACACCTCCAACCCTGAGCTCAC |
| | | CAGCTCCACAGAGGTGCAGGTGGAGCAGGCCACGCC |
| | | CATCTCGGAGAGCTGTGCAGGTAAAATGGAAGAATTC |
| | | TTTCTCTTTGGAGCCTTCCTGGAGGCCTCAATGATCGA |
| | | CCGGAGAAACGGAGACAAGCCCATCACCTTTGAGGTC |
| | | ACCATAGGCAACTATGGGAACGAAGTTGATGGCCTGT |
| | | CCCGGCCCCAGCGGCCTCGGCCCCGGAAGGAGCCG |
| | | GGGGATGAGGAAGAAGTAGACCTGATTCAGAACGCAA |
| | | GTGATGACGAGGCCGGTGATGCCGGGGACCTGGCCT |
| | | CAGTCTCCTCCACTCCACCAATGCGGCCCCAGGTCAC |
| | | CGACAGGAACTACTTCCATCTGCCCTACCTGGAGCGA |
| | | AAGCCCTGCATCTACATCAAGAGCTGGTGGCCGGACC |
| | | AGCGCCGCCGCCTCTACAATGCCAACATCATGGACCA |
| | | CATTGCCGACAAGCTGGAAGAAGGCCTGAACGACATA |
| | | CAGGAGATGATCAAAACGGAGAAGTCCTACCCTGAGC |
| | | GTCGCCTGCGGGGCGTCCTGGAGGAGCTGAGCTGTG |
| | | GCTGCTGCCGCTTCCTCTCCCTCGCTGACAAGGACCA |
| | | GGGCCACTCATCCCGCACCAGGCTTGACCGGGAGCG |
| | | CCTCAAGTCCTGCATGAGGGAGCTGGTAAGTATCAAG |
| | | GTTACAAGACAGGTTTAAGGAGACCAATAGAAACTGG |
| | | GCTTGTCGAGACAGAGAAGACTCTTGCGTTTCTGAGC |
| | | TAGCCCCCGGGTGCGCGGCGTCGGTGGTGCCGGCG |
| | | GGGGGCGCCAGGTCGCAGGCGGTGTAGGGCTCCAG |
| | | GCAGGCGGCGAAGGCCATGACGTGCGCTATGAAGGT |
| | | CTGCTCCTGCACGCCGTGAACCAGGTGCGCCTGCGG |
| | | GCCGCGCGCGAACACCGCCACGTCCTCGCCTGCGTG |
| | | GGTCTCTTCGTCCAGGGGCACTGCTGACTGCTGCCGA |
| | | TACTCGGGGCTCCCGCTCTCGCTCTCGGTAACATCCG |
| | | GCCGGGCGCCGTCCTTGAGCACATAGCCTGGACCGTT |
| | | TCGTCGACCTCGAGTTAAGGGCGAATTCCCGATAAGG |
| | | ATCTTCCTAGAGCATGGCTACGTAGATAAGTAGCATGG |
| | | CGGGTTAATCATTAACTACAAGGAACCCCTAGTGATGG |
| | | AGTTGGCCACTCCCTCTCTGCGCGCTCGCTCGCTCAC |
| | | TGAGGCCGGGCGACCAAAGGTCGCCCGACGCCCGGG |
| | | CTTTGCCCGGGCGGCCTCAGTGAGCGAGCGAGCGCG |
| | | CAGCCTTAATTAAATCCACATCTGTATGTTTTTTATATT |
| | | AATTTATTTTTTGCAGGGGGGCATTGTTTGGTAGGTGA |
| | | GAGTTCTGAATTGCTATGTTTAGTGAGTTGTATCTATTT |
| | | ATTTTTCAATAAATACAATTAGTTATGTGTTTTGGGGGC |
| | | GATCGTGAGGCAAAGAAAACCCGGCGCTGAGGCCGG |
| | | GTTATTCTTGTTCTCTGGTCAAATTATATAGTTGGAAAA |
| | | CAAGGATGCATATATGAATGAACGATGCAGAGGCAAT |
| | | GCCGATGGCGATAGTGGGTATCAGGTAGCCGCTTATG |
| | | CTGGAAAGAAGCAATAACCCGCAGAAAAACAAAGCTC |
| | | CAAGCTCAACAAAACTAAGGGCATAGACAATAACTACC |
| | | TATGTCATATACCCATACTCTCTAATCTTGGCCAGTCG |
| | | GCGCGTTCTGCTTCCGATTAGAAACGTCAAGGCAGCA |
| | | ATCAGGATTGCAATCTTGGTTCCTGCATAGGATGACAA |
| | | TGTCGCCCCAAGACCATCTCTATGAGCTGAAAAAGAAA |
| | | CACAAGGAATGTAGTGGCGGAAAAGGAGATAGCAAAT |
| | | GCTTACGATAACGTAAGGAATTATTACTATGTAAACAC |
| | | CAGGCAAGATTCTGTTCCGTATAATTACTCCTGATAATT |
| | | AATCCTTAACTTTGCCCACCTGCCTTTTAAAACATTCCA |
| | | GTATATCACTTTTCATTCTTGCGTAGCAATATGCCCTCT |
| | | CTTCAGCTATCTCAGCATTGGTGACCTTGTTCAGAGGC |
| | | GCTGAGAGATGGCCTTTTTCTGATAGATAATGTTCTGT |
| | | TAAAATATCTCCGGCCTCATCTTTTGCCCGCAGGCTAA |
| | | TGTCTGAAAATTGAGGTGACGGGTTAAAAATAATATCC |
| | | TTGGCAACCTTTTTTATATCCCTTTTAAATTTTGGOTTA |
| | | ATGACTATATCCAATGAGTCAAAAAGCTCCCCTTCAAT |
| | | ATCTGTTGCCCCTAAGACCTTTAATATATCGCCAAATA |
| | | CAGGTAGCTTGGCTTCTACCTTCACCGTTGTTCTGCCG |
| | | ATGAAATGCTAATGCATAACATCGTCTTTGGTGGTTCC |
| | | CCTCATCAGTGGCTCTATCTGAACGCGCTCTCCACTG |
| | | CTTAATGACATTCCTTTCCCGATTAAAAAATCTGTCAGA |
| | | TCGGATGTGGTCGGCCCGAAAACAGTTCTGGCAAAAC |
| | | CAATGGTGTCGCCTTCAACAAACAAAAAAGATGGGAAT |
| | | CCCAATGATTCGTCATCTGCGAGGCTGTTCTTAATATC |
| | | TTCAACTGTAGCTTTAGAGCGATTTATCTTCTGAACCA |
| | | GACTCTTGTCATTTGTTTTGGTAAAGAGAAAAGTTTTTC |
| | | CATCGATTTTATGAATATACAAATAATTGGAGCCAACCT |
| | | TCAGGTGATGATTATCAGCCAGCAGAGAATTAAGGAAA |
| | | ACAGACAGGTTTATTGAGCACTTATCTTTCCCTTTATTT |
| | | TTGCTGCGGTAAGTCGCATAAAAACCATTCTTCACAAT |
| | | TCAATCCATTTACTATGTTATGTTCTGAGGGGAGTGAA |
| | | AATTCCCCTAATTCGATGAAGATTCTTGCTAAATTGTTA |
| | | TCAGCTATGCGCCGACCAGAACACCTTGCCGATCAGC |
| | | CAAACGTCTAATCAGGCCACTGACTAGCGATAACTTTC |
| | | CCCACAACGGAACAACTCTCATTGCATGGGATAATTGG |
| | | GTACTGTGGGTTTAGTGGTTGTAAAAACACCTGACCGC |
| | | TATCCCTGATCAGTTTCTTGAAGGTAAACTCATCACCC |
| | | CCAAGTCTGGCTATACAGAAATCACCTGGCTCAACAG |
| | | CCTGCTCAGGGTCAACGAGAATTTACATTCCGTCAGG |
| | | ATAGCTTGGCTTGGAGCCTGTTGGTGCGGTCACGGAA |
| | | TTACCTTCAACCTCAAGCCAGAATGCAGAATCACTGGC |
| | | TTTTTTGGTTGTGCTTACCCATCTCTCCGCATCACCTTT |
| | | GGTAAAGGTTCTAAGCTAAGGTGAGAACATCCCTGCC |
| | | TGAACATGAGAAAAAACAGGGTACTCATACTCACTTAT |
| | | TAGTGACGGCTATGAGCAAAAGGCCAGCAAAAGGCCA |
| | | GGAACCGTAAAAAGGCCGCGTTGCTGGOGTTTTTCCA |
| | | TAGGCTCCGCCCCCCTGACGAGCATCACAAAAATCGA |
| | | CGCTCAAGTCAGAGGTGGCGAAACCCGACAGGACTAT |
| | | AAAGATACCAGGCGTTTCCCCCTGGAAGCTCCCTCGT |
| | | GCGCTCTCCTGTTCCGACCCTGCCGCTTACCGGATAC |
| | | CTGTCCGCCTTTCTCCCTTCGGGAAGCGTGGCGCTTT |
| | | CTCATAGCTCACGCTGTAGGTATCTCAGTTCGGTGTAG |
| | | GTCGTTCGCTCCAAGCTGGGCTGTGTGCACGAACCCC |
| | | CCGTTCAGCCCGACCGCTGCGCCTTATCCGGTAACTA |
| | | TCGTCTTGAGTCCAACCCGGTAAGACACGACTTATCG |
| | | CCACTGGCAGCAGCCACTGGTAACAGGATTAGCAGAG |
| | | CGAGGTATGTAGGCGGTGCTACAGAGTTCTTGAAGTG |
| | | GTGGCCTAACTACGGCTACACTAGAAGAACAGTATTTG |
| | | GTATCTGCGCTCTGCTGAAGCCAGTTACCTTCGGAAAA |
| | | AGAGTTGGTAGCTCTTGATCCGGCAAACAAACCACCG |
| | | CTGGTAGCGGTGGTTTTTTTGTTTGCAAGCAGCAGATT |
| | | ACGCGCAGAAAAAAAGGATCTCAAGAAGATCCTTTGAT |
| | | CTTTTCTACGGGGTCTGACGCTCAGTGGAACGAAAAC |
| | | TCACGTTAAGGGATTTTGGTCATGAGATTATCAAAAAG |
| | | GATCTTCACCTAGATCCTTTTAAATTAAAAATGAAGTTT |
| | | TAAATCAAGCCCAATCTGAATAATGTTACAACCAATTAA |
| | | CCAATTCTGATTAGAAAAACTCATCGAGCATCAAATGA |
| | | AACTGCAATTTATTCATATCAGGATTATCAATACCATAT |
| | | TTTTGAAAAAGCCGTTTCTGTAATGAAGGAGAAAACTC |
| | | ACCGAGGCAGTTCCATAGGATGGCAAGATCCTGGTAT |
| | | CGGTCTGCGATTCCGACTCGTCCAACATCAATACAACC |
| | | TATTAATTTCCCCTCGTCAAAAATAAGGTTATCAAGTGA |
| | | GAAATCACCATGAGTGACGACTGAATCCGGTGAGAAT |
| | | GGCAAAAGTTTATGCATTTCTTTCCAGACTTGTTCAACA |
| | | GGCCAGCCATTACGCTCGTCATCAAAATCACTCGCATC |
| | | AACCAAACCGTTATTCATTCGTGATTGCGCCTGAGCAA |
| | | GACGAAATACGCGATCGCTGTTAAAAGGACAATTACAA |
| | | ACAGGAATCGAATGCAACCGGCGCAGGAACACTGCCA |
| | | GCGCATCAACAATATTTTCACCTGAATCAGGATATTCTT |
| | | CTAATACCTGGAATGCTGTTTTTCCGGGGATCGCAGTG |
| | | GTGAGTAACCATGCATCATCAGGAGTACGGATAAAATG |
| | | CTTGATGGTCGGAAGAGGCATAAATTCCGTCAGCCAG |
| | | TTTAGTCTGACCATCTCATCTGTAACATCATTGGCAAC |
| | | GCTACCTTTGCCATGTTTCAGAAACAACTCTGGCGCAT |
| | | CGGGCTTCCCATACAAGCGATAGATTGTCGCACCTGA |
| | | TTGCCCGACATTATCGCGAGCCCATTTATACCCATATA |
| | | AATCAGCATCCATGTTGGAATTTAATCGCGGCCTCGAC |
| | | GTTTCCCGTTGAATATGGCTCATAACACCCCTTGTATT |
| | | ACTGTTTATGTAAGCAGACAGTTTTATTGTTCATGATGA |
| | | TATATTTTTATCTTGTGCAATGTAACATCAGAGATTTTG |
| | | AGACACGGGCCAGAGCTGCA |
| 61 | 3′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | AP head sequence at positions | GTTGCGCCAGCATGATTAATACAGCATTTAATACAGAG |
| | 2267-2553 | CCGTGTTTATTGAGTCGGTATTCAGAGTCTGACCAGAA |
| | Splice acceptor sequence at | ATTATTAATCTGGTGAAGTTATTCCTCTGTCATTACGTC |
| | positions 2576-2624 | ATGGTCGATTTCAATTTCTATTGATGCTTTCCAGTCGTA |
| | C-terminal portion of human | ATCAATGATGTATTTTTTGATGTTTGACCTCTGTTCATA |
| | OTOF isoform 5 at positions | TCCTCACAGATAAAAAATCGCCCTCACACTGGAGGGC |
| | 2625-6212 | AAAGAAGATTTCCAATAATCAGAACAAGTCGGCTCCTG |
| | bGH poly(A) sequence at | TTTAGTTACGAGCGACATTGCTCCGTGTATTCACTCGT |
| | positions 6255-6476 | TGGAATGAATACACAGTGCAGTGTTTATTCTGTTATTTA |
| | ITR at positions 6564-6693 | TGCCAAAAATTAAGGCCACTATCAGGCAGCTTTGTTGT |
| | Lambda at positions 6704-8716 | TCTGTTTACCAAGTTCTCTGGCAATCATTGCCGTCGTT |
| | Ori at positions 8773-9361 | CGTATTGCCCATTTATCGACATATTTCCCATCTTCCTAT |
| | KanR at positions 9539-10, 348 | ACAGGAAACATTTCTTCAGGCTTAACCATGCATTCCGA |
| | Transgene to be transferred | TTGCAGCTTGCATCCATTGCATCGCTTGAATTGTCCAC |
| | into vector in dual vector | ACCATTGATTTTTATCAATAGTCGTAGTTTAACGGATAG |
| | system at positions 2049-6693 | TCCTGGTATTGTTCCATCACATCCTGAGGATGCCCTTC |
| | | GAACTCTTCAAATTCTTCTTCCTAATATCACCTTAAATA |
| | | GTGGATTGCGGTAGTAAAGATTGTGCCTGTCTTTTAAC |
| | | CACATCAGGCTCGGTGGTTCTCGTGTACCCCTACAGC |
| | | GAGAAATCGGATAAACTATTACAACCCCTACAGTTTGT |
| | | AGAGTATAGAAAATGATCCACTCGTTATTCTCGGACGA |
| | | GTGTTCAGTAATGAACCTCTGGAGAGAACCATCTATAT |
| | | GATCGTTATCTGGGTTTGACTTCTGCTTTTAAGCCCAG |
| | | ATAACTTGCCTGAATATGTTAATGAGAGAATCGGTATT |
| | | CCTCATGTGTGGCATGTTTTCGTCTTTGCTCTTGCATTT |
| | | TCACTAGCAATTAATGTGCATCGATTATCAGCTATTGC |
| | | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGCGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCCCCCG |
| | | GGTGCGCGGCGTCGGTGGTGCCGGGGGGGGCGCC |
| | | AGGTCGCAGGCGGTGTAGGGCTCCAGGCAGGCGGCG |
| | | AAGGCCATGACGTGCGCTATGAAGGTCTGCTCCTGCA |
| | | CGCCGTGAACCAGGTGCGCCTGCGGGCCGCGCGCGA |
| | | ACACCGCCACGTCCTCGCCTGCGTGGGTCTCTTCGTC |
| | | CAGGGGCACTGCTGACTGCTGCCGATACTCGGGGCT |
| | | CCCGCTCTCGCTCTCGGTAACATCCGGCCGGGCGCC |
| | | GTCCTTGAGCACATAGCCTGGACCGTTTCCTTAAGCG |
| | | ACGCATGCTCGCGATAGGCACCTATTGGTCTTACTGA |
| | | CATCCACTTTGCCTTTCTCTCCACAGGAAAACATGGGG |
| | | CAGCAGGCCAGGATGCTGCGGGCCCAGGTGAAGCGG |
| | | CACACGGTGCGGGACAAGCTGAGGCTGTGCCAGAAC |
| | | TTCCTGCAGAAGCTGCGCTTCCTGGCGGACGAGCCCC |
| | | AGCACAGCATTCCCGACATCTTCATCTGGATGATGAGC |
| | | AACAACAAGCGTGTCGCCTATGCCCGTGTGCCCTCCA |
| | | AGGACCTGCTCTTCTCCATCGTGGAGGAGGAGACTGG |
| | | CAAGGACTGCGCCAAGGTCAAGACGCTCTTCCTTAAG |
| | | CTGCCAGGGAAGCGGGGCTTCGGCTCGGCAGGCTGG |
| | | ACAGTGCAGGCCAAGGTGGAGCTGTACCTGTGGCTG |
| | | GGCCTCAGCAAACAGCGCAAGGAGTTCCTGTGCGGC |
| | | CTGCCCTGTGGCTTCCAGGAGGTCAAGGCAGCCCAG |
| | | GGCCTGGGCCTGCATGCCTTCCCACCCGTCAGCCTG |
| | | GTCTACACCAAGAAGCAGGCGTTCCAGCTCCGAGCGC |
| | | ACATGTACCAGGCCCGCAGCCTCTTTGCCGCCGACAG |
| | | CAGCGGACTCTCAGACCCCTTTGCCCGCGTCTTCTTC |
| | | ATCAATCAGAGTCAGTGCACAGAGGTGCTGAATGAGA |
| | | CCCTGTGTCCCACCTGGGACCAGATGCTGGTGTTCGA |
| | | CAACCTGGAGCTCTATGGTGAAGCTCATGAGCTGAGG |
| | | GACGATCCGCCCATCATTGTCATTGAAATCTATGACCA |
| | | GGATTCCATGGGCAAAGCTGACTTCATGGGCCGGACC |
| | | TTCGCCAAACCCCTGGTGAAGATGGCAGACGAGGCGT |
| | | ACTGCCCACCCCGOTTCCCACCTCAGCTCGAGTACTA |
| | | CCAGATCTACCGTGGCAACGCCACAGCTGGAGACCTG |
| | | CTGGCGGCCTTCGAGCTGCTGCAGATTGGACCAGCAG |
| | | GGAAGGCTGACCTGCCCCCCATCAATGGCCCGGTGG |
| | | ACGTGGACCGAGGTCCCATCATGCCCGTGCCCATGG |
| | | GCATCCGGCCCGTGCTCAGCAAGTACCGAGTGGAGG |
| | | TGCTGTTCTGGGGCCTACGGGACCTAAAGCGGGTGAA |
| | | CCTGGCCCAGGTGGACCGGCCACGGGTGGACATCGA |
| | | GTGTGCAGGGAAGGGGGTGCAGTCGTCCCTGATCCA |
| | | CAATTATAAGAAGAACCCCAACTTCAACACCCTCGTCA |
| | | AGTGGTTTGAAGTGGACCTCCCAGAGAACGAGCTGCT |
| | | GCACCCGCCCTTGAACATCCGTGTGGTGGACTGCCGG |
| | | GCCTTCGGTCGCTACACACTGGTGGGCTCCCATGCCG |
| | | TCAGCTCCCTGCGACGCTTCATCTACCGGCCCCCAGA |
| | | CCGCTCGGCCCCCAGCTGGAACACCACGGTCAGGCT |
| | | TCTCCGGCGCTGCCGTGTGCTGTGCAATGGGGGCTC |
| | | CTCCTCTCACTCCACAGGGGAGGTTGTGGTGACTATG |
| | | GAGCCAGAGGTACCCATCAAGAAACTGGAGACCATGG |
| | | TGAAGCTGGACGCGACTTCTGAAGCTGTTGTCAAGGT |
| | | GGATGTGGCTGAGGAGGAGAAGGAGAAGAAGAAGAA |
| | | GAAGAAGGGCACTGCGGAGGAGCCAGAGGAGGAGGA |
| | | GCCAGACGAGAGCATGCTGGACTGGTGGTCCAAGTAC |
| | | TTTGCCTCCATTGACACCATGAAGGAGCAACTTCGACA |
| | | ACAAGAGCCCTCTGGAATTGACTTGGAGGAGAAGGAG |
| | | GAAGTGGACAATACCGAGGGCCTGAAGGGGTCAATGA |
| | | AGGGCAAGGAGAAGGCAAGGGCTGCCAAAGAGGAGA |
| | | AGAAGAAGAAAACTCAGAGCTCTGGCTCTGGCCAGGG |
| | | GTCCGAGGCCCCCGAGAAGAAGAAACCCAAGATTGAT |
| | | GAGCTTAAGGTATACCCCAAAGAGCTGGAGTCCGAGT |
| | | TTGATAACTTTGAGGACTGGCTGCACACTTTCAACTTG |
| | | CTTCGGGGCAAGACCGGGGATGATGAGGATGGCTCC |
| | | ACCGAGGAGGAGCGCATTGTGGGACGCTTCAAGGGC |
| | | TCCCTCTGCGTGTACAAAGTGCCACTCCCAGAGGACG |
| | | TGTCCCGGGAAGCCGGCTACGACTCCACCTACGGCAT |
| | | GTTCCAGGGCATCCCGAGCAATGACCCCATCAATGTG |
| | | CTGGTCCGAGTCTATGTGGTCCGGGCCACGGACCTGC |
| | | ACCCTGCTGACATCAACGGCAAAGCTGACCCCTACAT |
| | | CGCCATCCGGCTAGGCAAGACTGACATCCGCGACAAG |
| | | GAGAACTACATCTCCAAGCAGCTCAACCCTGTCTTTGG |
| | | GAAGTCCTTTGACATCGAGGCCTCCTTCCCCATGGAAT |
| | | CCATGCTGACGGTGGCTGTGTATGACTGGGACCTGGT |
| | | GGGCACTGATGACCTCATTGGGGAAACCAAGATCGAC |
| | | CTGGAGAACCGCTTCTACAGCAAGCACCGCGCCACCT |
| | | GCGGCATCGCCCAGACCTACTCCACACATGGCTACAA |
| | | TATCTGGCGGGACCCCATGAAGCCCAGCCAGATCCTG |
| | | ACCCGCCTCTGCAAAGACGGCAAAGTGGACGGCCCC |
| | | CACTTTGGGCCCCCTGGGAGAGTGAAGGTGGCCAAC |
| | | CGCGTCTTCACTGGGCCCTCTGAGATTGAGGACGAGA |
| | | ACGGTCAGAGGAAGCCCACAGACGAGCATGTGGCGC |
| | | TGTTGGCCCTGAGGCACTGGGAGGACATCCCCCGCG |
| | | CAGGCTGCCGCCTGGTGCCAGAGCATGTGGAGACGA |
| | | GGCCGCTGCTCAACCCCGACAAGCCGGGCATCGAGC |
| | | AGGGCCGCCTGGAGCTGTGGGTGGACATGTTCCCCAT |
| | | GGACATGCCAGCCCCTGGGACGCCTCTGGACATCTCA |
| | | CCTCGGAAGCCCAAGAAGTACGAGCTGCGGGTCATCA |
| | | TCTGGAACACAGATGAGGTGGTCTTGGAGGACGACGA |
| | | CTTCTTCACAGGGGAGAAGTCCAGTGACATCTTCGTG |
| | | AGGGGGTGGCTGAAGGGCCAGCAGGAGGACAAGCAG |
| | | GACACAGACGTCCACTACCACTCCCTCACTGGCGAGG |
| | | GCAACTTCAACTGGCGCTACCTGTTCCCCTTCGACTAC |
| | | CTGGCGGCGGAGGAGAAGATCGTCATCTCCAAGAAG |
| | | GAGTCCATGTTCTCCTGGGACGAGACCGAGTACAAGA |
| | | TCCCCGCGCGGCTCACCCTGCAGATCTGGGATGCGG |
| | | ACCACTTCTCCGCTGACGACTTCCTGGGGGCCATCGA |
| | | GCTGGACCTGAACCGGTTCCCGCGGGGCGCAAAGAC |
| | | AGCCAAGCAGTGCACCATGGAGATGGCCACCGGGGA |
| | | GGTGGACGTGCCCCTCGTGTCCATCTTCAAGCAAAAG |
| | | CGCGTCAAAGGCTGGTGGCCCCTCCTGGCCCGCAAT |
| | | GAGAACGATGAGTTTGAGCTCACGGGCAAGGTGGAG |
| | | GCTGAGCTGCATTTACTGACAGCAGAGGAGGCAGAGA |
| | | AGAACCCAGTGGGCCTGGCCCGCAATGAACCTGACCC |
| | | CCTAGAGAAACCCAACCGGCCCGACACGGCCTTCGTC |
| | | TGGTTCCTCAACCCTCTCAAGTCCATCAAGTACCTCAT |
| | | CTGCACCCGGTACAAGTGGCTCATCATCAAGATCGTG |
| | | CTGGCGCTGTTGGGGCTGCTCATGTTGGGGCTCTTCC |
| | | TCTACAGCCTCCCTGGCTACATGGTCAAAAAGCTCCTT |
| | | GGGGCATGAACGGCCGCTATGCTAGCTTGGTACCAAG |
| | | GGCGGATCCTGCATAGAGCTCGCTGATCAGCCTCGAC |
| | | TGTGCCTTCTAGTTGCCAGCCATCTGTTGTTTGCCCCT |
| | | CCCCCGTGCCTTCCTTGACCCTGGAAGGTGCCACTCC |
| | | CACTGTCCTTTCCTAATAAAATGAGGAAATTGCATCGC |
| | | ATTGTCTGAGTAGGTGTCATTCTATTCTGGGGGGTGG |
| | | GGTGGGGCAGGACAGCAAGGGGGAGGATTGGGAAGA |
| | | CAATAGCAGGCATCTCGAGTTAAGGGCGAATTCCCGA |
| | | TAAGGATCTTCCTAGAGCATGGCTACGTAGATAAGTAG |
| | | CATGGGGGGTTAATCATTAACTACAAGGAACCCCTAGT |
| | | GATGGAGTTGGCCACTCCCTCTCTGCGCGCTCGCTCG |
| | | CTCACTGAGGCCGGGCGACCAAAGGTCGCCCGACGC |
| | | CCGGGCTTTGCCCGGGGGGCCTCAGTGAGCGAGCGA |
| | | GCGCGCAGCCTTAATTAAATCCACATCTGTATGTTTTTT |
| | | ATATTAATTTATTTTTTGCAGGGGGGCATTGTTTGGTAG |
| | | GTGAGAGTTCTGAATTGCTATGTTTAGTGAGTTGTATC |
| | | TATTTATTTTTCAATAAATACAATTAGTTATGTGTTTTGG |
| | | GGGCGATCGTGAGGCAAAGAAAACCCGGCGCTGAGG |
| | | CCGGGTTATTCTTGTTCTCTGGTCAAATTATATAGTTG |
| | | GAAAACAAGGATGCATATATGAATGAACGATGCAGAG |
| | | GCAATGCCGATGGCGATAGTGGGTATCAGGTAGCCGC |
| | | TTATGCTGGAAAGAAGCAATAACCCGCAGAAAAACAAA |
| | | GCTCCAAGCTCAACAAAACTAAGGGCATAGACAATAAC |
| | | TACCTATGTCATATACCCATACTCTCTAATCTTGGCCA |
| | | GTCGGCGCGTTCTGCTTCCGATTAGAAACGTCAAGGC |
| | | AGCAATCAGGATTGCAATCTTGGTTCCTGCATAGGATG |
| | | ACAATGTCGCCCCAAGACCATCTCTATGAGCTGAAAAA |
| | | GAAACACAAGGAATGTAGTGGCGGAAAAGGAGATAGC |
| | | AAATGCTTACGATAACGTAAGGAATTATTACTATGTAAA |
| | | CACCAGGCAAGATTCTGTTCCGTATAATTACTCCTGAT |
| | | AATTAATCCTTAACTTTGCCCACCTGCCTTTTAAAACAT |
| | | TCCAGTATATCACTTTTCATTCTTGCGTAGCAATATGCC |
| | | CTCTCTTCAGCTATCTCAGCATTGGTGACCTTGTTCAG |
| | | AGGCGCTGAGAGATGGCCTTTTTCTGATAGATAATGTT |
| | | CTGTTAAAATATCTCCGGCCTCATCTTTTGCCCGCAGG |
| | | CTAATGTCTGAAAATTGAGGTGACGGGTTAAAAATAAT |
| | | ATCCTTGGCAACCTTTTTTATATCCCTTTTAAATTTTGG |
| | | CTTAATGACTATATCCAATGAGTCAAAAAGCTCCCCTT |
| | | CAATATCTGTTGCCCCTAAGACCTTTAATATATCGCCA |
| | | AATACAGGTAGCTTGGCTTCTACCTTCACCGTTGTTCT |
| | | GCCGATGAAATGCTAATGCATAACATCGTCTTTGGTGG |
| | | TTCCCCTCATCAGTGGCTCTATCTGAACGCGCTCTCCA |
| | | CTGCTTAATGACATTCCTTTCCCGATTAAAAAATCTGTC |
| | | AGATCGGATGTGGTCGGCCCGAAAACAGTTCTGGCAA |
| | | AACCAATGGTGTCGCCTTCAACAAACAAAAAAGATGGG |
| | | AATCCCAATGATTCGTCATCTGCGAGGCTGTTCTTAAT |
| | | ATCTTCAACTGTAGCTTTAGAGCGATTTATCTTCTGAAC |
| | | CAGACTCTTGTCATTTGTTTTGGTAAAGAGAAAAGTTTT |
| | | TCCATCGATTTTATGAATATACAAATAATTGGAGCCAAC |
| | | CTTCAGGTGATGATTATCAGCCAGCAGAGAATTAAGGA |
| | | AAACAGACAGGTTTATTGAGCACTTATCTTTCCCTTTAT |
| | | TTTTGCTGCGGTAAGTCGCATAAAAACCATTCTTCACA |
| | | ATTCAATCCATTTACTATGTTATGTTCTGAGGGGAGTG |
| | | AAAATTCCCCTAATTCGATGAAGATTCTTGCTAAATTGT |
| | | TATCAGCTATGCGCCGACCAGAACACCTTGCCGATCA |
| | | GCCAAACGTCTAATCAGGCCACTGACTAGCGATAACTT |
| | | TCCCCACAACGGAACAACTCTCATTGCATGGGATAATT |
| | | GGGTACTGTGGGTTTAGTGGTTGTAAAAACACCTGAC |
| | | CGCTATCCCTGATCAGTTTCTTGAAGGTAAACTCATCA |
| | | CCCCCAAGTCTGGCTATACAGAAATCACCTGGCTCAA |
| | | CAGCCTGCTCAGGGTCAACGAGAATTTACATTCCGTCA |
| | | GGATAGCTTGGCTTGGAGCCTGTTGGTGCGGTCACGG |
| | | AATTACCTTCAACCTCAAGCCAGAATGCAGAATCACTG |
| | | GCTTTTTTGGTTGTGCTTACCCATCTCTCCGCATCACC |
| | | TTTGGTAAAGGTTCTAAGCTAAGGTGAGAACATCCCTG |
| | | CCTGAACATGAGAAAAAACAGGGTACTCATACTCACTT |
| | | ATTAGTGACGGCTATGAGCAAAAGGCCAGCAAAAGGC |
| | | CAGGAACCGTAAAAAGGCCGCGTTGCTGGCGTTTTTC |
| | | CATAGGCTCCGCCCCCCTGACGAGCATCACAAAAATC |
| | | GACGCTCAAGTCAGAGGTGGCGAAACCCGACAGGACT |
| | | ATAAAGATACCAGGCGTTTCCCCCTGGAAGCTCCCTC |
| | | GTGCGCTCTCCTGTTCCGACCCTGCCGCTTACCGGAT |
| | | ACCTGTCCGCCTTTCTCCCTTCGGGAAGCGTGGCGCT |
| | | TTCTCATAGCTCACGCTGTAGGTATCTCAGTTCGGTGT |
| | | AGGTCGTTCGCTCCAAGCTGGGCTGTGTGCACGAACC |
| | | CCCCGTTCAGCCCGACCGCTGCGCCTTATCCGGTAAC |
| | | TATCGTCTTGAGTCCAACCCGGTAAGACACGACTTATC |
| | | GCCACTGGCAGCAGCCACTGGTAACAGGATTAGCAGA |
| | | GCGAGGTATGTAGGCGGTGCTACAGAGTTCTTGAAGT |
| | | GGTGGCCTAACTACGGCTACACTAGAAGAACAGTATTT |
| | | GGTATCTGCGCTCTGCTGAAGCCAGTTACCTTCGGAA |
| | | AAAGAGTTGGTAGCTCTTGATCCGGCAAACAAACCAC |
| | | CGCTGGTAGCGGTGGTTTTTTTGTTTGCAAGCAGCAG |
| | | ATTACGCGCAGAAAAAAAGGATCTCAAGAAGATCCTTT |
| | | GATCTTTTCTACGGGGTCTGACGCTCAGTGGAACGAA |
| | | AACTCACGTTAAGGGATTTTGGTCATGAGATTATCAAA |
| | | AAGGATCTTCACCTAGATCCTTTTAAATTAAAAATGAAG |
| | | TTTTAAATCAAGCCCAATCTGAATAATGTTACAACCAAT |
| | | TAACCAATTCTGATTAGAAAAACTCATCGAGCATCAAAT |
| | | GAAACTGCAATTTATTCATATCAGGATTATCAATACCAT |
| | | ATTTTTGAAAAAGCCGTTTCTGTAATGAAGGAGAAAAC |
| | | TCACCGAGGCAGTTCCATAGGATGGCAAGATCCTGGT |
| | | ATCGGTCTGCGATTCCGACTCGTCCAACATCAATACAA |
| | | CCTATTAATTTCCCCTCGTCAAAAATAAGGTTATCAAGT |
| | | GAGAAATCACCATGAGTGACGACTGAATCCGGTGAGA |
| | | ATGGCAAAAGTTTATGCATTTCTTTCCAGACTTGTTCAA |
| | | CAGGCCAGCCATTACGCTCGTCATCAAAATCACTCGC |
| | | ATCAACCAAACCGTTATTCATTCGTGATTGCGCCTGAG |
| | | CAAGACGAAATACGCGATCGCTGTTAAAAGGACAATTA |
| | | CAAACAGGAATCGAATGCAACCGGCGCAGGAACACTG |
| | | CCAGCGCATCAACAATATTTTCACCTGAATCAGGATAT |
| | | TCTTCTAATACCTGGAATGCTGTTTTTCCGGGGATCGC |
| | | AGTGGTGAGTAACCATGCATCATCAGGAGTACGGATA |
| | | AAATGCTTGATGGTCGGAAGAGGCATAAATTCCGTCA |
| | | GCCAGTTTAGTCTGACCATCTCATCTGTAACATCATTG |
| | | GCAACGCTACCTTTGCCATGTTTCAGAAACAACTCTGG |
| | | CGCATCGGGCTTCCCATACAAGCGATAGATTGTCGCA |
| | | CCTGATTGCCCGACATTATCGCGAGCCCATTTATACCC |
| | | ATATAAATCAGCATCCATGTTGGAATTTAATCGCGGCC |
| | | TCGACGTTTCCCGTTGAATATGGCTCATAACACCCCTT |
| | | GTATTACTGTTTATGTAAGCAGACAGTTTTATTGTTCAT |
| | | GATGATATATTTTTATCTTGTGCAATGTAACATCAGAGA |
| | | TTTTGAGACACGGGCCAGAGCTGCA |
| 62 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGGGGCCTC |
| | Myo15 promoter at positions | AGTGAGCGAGCGAGCGCGCAGAGAGGGAGTGGCCAA |
| | 182-1146 | CTCCATCACTAGGGGTTCCTCAGATCTGAATTCGGTAC |
| | N-terminal portion of human | CTGCAGCTCAGCCTACTACTTGCTTTCCAGGCTGTTCC |
| | OTOF isoform 5 at positions | TAGTTCCCATGTCAGCTGCTTGTGCTTTCCAGAGACAA |
| | 1167-3572 | AACAGGAATAATAGATGTCATTAAATATACATTGGGCC |
| | Splice donor sequence at | CCAGGCGGTCAATGTGGCAGCCTGAGCCTCCTTTCCA |
| | positions 3573-3656 | TCTCTGTGGAGGCAGACATAGGACCCCCAACAAACAG |
| | AP head sequence at positions | CATGCAGGTTGGGAGCCAGCCACAGGACCCAGGTAA |
| | 3663-3949 | GGGGCCCTGGGTCCTTAAGCTTCTGCCACTGGCTCCG |
| | ITR at positions 3973-4115 | GCATTGCAGAGAGAAGAGAAGGGGCGGCAGACTGGA |
| | KanR at positions 4641-5435 | GAGCTGGGCTCCATTTTTGTTCCTTGGTGCCCTGCCC |
| | pUC ori at positions 5821-6491 | CTCCCCATGACCTGCAGAGACATTCAGCCTGCCAGGC |
| | Transgene to be transferred | TTTATGAGGTGGGAGCTGGGCTCTCCCTGATGTATTAT |
| | into vector in dual vector | TCAGCTCCCTGGAGTTGGCCAGCTCCTGTTACACTGG |
| | system at positions 19-4115 | CCACAGCCCTGGGCATCCGCTTCTCACTTCTAGTTTCC |
| | | CCTCCAAGGTAATGTGGTGGGTCATGATCATTCTATCC |
| | | TGGCTTCAGGGACCTGACTCCACTTTGGGGCCATTCG |
| | | AGGGGTCTAGGGTAGATGATGTCCCCCTGTGGGGATT |
| | | AATGTCCTGCTCTGTAAAACTGAGCTAGCTGAGATCCA |
| | | GGAGGGCTTGGCCAGAGACAGCAAGTTGTTGCCATGG |
| | | TGACTTTAAAGCCAGGTTGCTGCCCCAGCACAGGCCT |
| | | CCCAGTCTACCCTCACTAGAAAACAACACCCAGGCAC |
| | | TTTCCACCACCTCTCAAAGGTGAAACCCAAGGCTGGT |
| | | CTAGAGAATGAATTATGGATCCTCGCTGTCCGTGCCAC |
| | | CCAGCTAGTCCCAGCGGCTCAGACACTGAGGAGAGAC |
| | | TGTAGGTTCAGCTACAAGCAAAAAGACCTAGCTGGTCT |
| | | CCAAGCAGTGTCTCCAAGTCCCTGAACCTGTGACACC |
| | | TGCCCCAGGCATCATCAGGCACAGAGGGCCACCGAAT |
| | | TCTAGCGGCCGCCACCATGGCCTTGCTCATCCACCTC |
| | | AAGACAGTCTCGGAGCTGCGGGGCAGGGGCGACCGG |
| | | ATCGCCAAAGTGACTTTCCGAGGGCAATCCTTCTACTC |
| | | TCGGGTCCTGGAGAACTGTGAGGATGTGGCTGACTTT |
| | | GATGAGACATTTCGGTGGCCGGTGGCCAGCAGCATCG |
| | | ACAGAAATGAGATGCTGGAGATTCAGGTTTTCAACTAC |
| | | AGCAAAGTCTTCAGCAACAAGCTCATCGGGACCTTCC |
| | | GCATGGTGCTGCAGAAGGTGGTAGAGGAGAGCCATGT |
| | | GGAGGTGACTGACACGCTGATTGATGACAACAATGCT |
| | | ATCATCAAGACCAGCCTGTGCGTGGAGGTCCGGTATC |
| | | AGGCCACTGACGGCACAGTGGGCTCCTGGGACGATG |
| | | GGGACTTCCTGGGAGATGAGTCTCTTCAAGAGGAAGA |
| | | GAAGGACAGCCAAGAGACGGATGGACTGCTCCCAGG |
| | | CTCCCGGCCCAGCTCCCGGCCCCCAGGAGAGAAGAG |
| | | CTTCCGGAGAGCCGGGAGGAGCGTGTTCTCCGCCAT |
| | | GAAGCTCGGCAAAAACCGGTCTCACAAGGAGGAGCCC |
| | | CAAAGACCAGATGAACCGGCGGTGCTGGAGATGGAA |
| | | GACCTTGACCATCTGGCCATTCGGCTAGGAGATGGAC |
| | | TGGATCCCGACTCGGTGTCTCTAGCCTCAGTCACAGC |
| | | TCTCACCACTAATGTCTCCAACAAGCGATCTAAGCCAG |
| | | ACATTAAGATGGAGCCAAGTGCTGGGCGGCCCATGGA |
| | | TTACCAGGTCAGCATCACGGTGATCGAGGCCCGGCAG |
| | | CTGGTGGGCTTGAACATGGACCCTGTGGTGTGCGTGG |
| | | AGGTGGGTGACGACAAGAAGTACACATCCATGAAGGA |
| | | GTCCACTAACTGCCCCTATTACAACGAGTACTTCGTCT |
| | | TCGACTTCCATGTCTCTCCGGATGTCATGTTTGACAAG |
| | | ATCATCAAGATTTCGGTGATTCACTCCAAGAACCTGCT |
| | | GCGCAGTGGCACCCTGGTGGGCTCCTTCAAAATGGAC |
| | | GTGGGAACCGTGTACTCGCAGCCAGAGCACCAGTTCC |
| | | ATCACAAGTGGGCCATCCTGTCTGACCCCGATGACAT |
| | | CTCCTCGGGGCTGAAGGGCTACGTGAAGTGTGACGTT |
| | | GCCGTGGTGGGCAAAGGGGACAACATCAAGACGCCC |
| | | CACAAGGCCAATGAGACCGACGAAGATGACATTGAGG |
| | | GGAACTTGCTGCTCCCCGAGGGGGTGCCCCCCGAAC |
| | | GCCAGTGGGCCCGGTTCTATGTGAAAATTTACCGAGC |
| | | AGAGGGGCTGCCCCGTATGAACACAAGCCTCATGGCC |
| | | AATGTAAAGAAGGCTTTCATCGGTGAAAACAAGGACCT |
| | | CGTGGACCCCTACGTGCAAGTCTTCTTTGCTGGCCAG |
| | | AAGGGCAAGACTTCAGTGCAGAAGAGCAGCTATGAGC |
| | | CCCTGTGGAATGAGCAGGTCGTCTTTACAGACCTCTTC |
| | | CCCCCACTCTGCAAACGCATGAAGGTGCAGATCCGAG |
| | | ACTCGGACAAGGTCAACGACGTGGCCATCGGCACCCA |
| | | CTTCATTGACCTGCGCAAGATTTCTAATGACGGAGACA |
| | | AAGGCTTCCTGCCCACACTGGGCCCAGCCTGGGTGAA |
| | | CATGTACGGCTCCACACGTAACTACACGCTGCTGGAT |
| | | GAGCATCAGGACCTGAACGAGGGCCTGGGGGAGGGT |
| | | GTGTCCTTCCGGGCCCGGCTCCTGCTGGGCCTGGCT |
| | | GTGGAGATCGTAGACACCTCCAACCCTGAGCTCACCA |
| | | GCTCCACAGAGGTGCAGGTGGAGCAGGCCACGCCCA |
| | | TCTCGGAGAGCTGTGCAGGTAAAATGGAAGAATTCTTT |
| | | CTCTTTGGAGCCTTCCTGGAGGCCTCAATGATCGACC |
| | | GGAGAAACGGAGACAAGCCCATCACCTTTGAGGTCAC |
| | | CATAGGCAACTATGGGAACGAAGTTGATGGCCTGTCC |
| | | CGGCCCCAGCGGCCTCGGCCCCGGAAGGAGCCGGG |
| | | GGATGAGGAAGAAGTAGACCTGATTCAGAACGCAAGT |
| | | GATGACGAGGCCGGTGATGCCGGGGACCTGGCCTCA |
| | | GTCTCCTCCACTCCACCAATGCGGCCCCAGGTCACCG |
| | | ACAGGAACTACTTCCATCTGCCCTACCTGGAGCGAAA |
| | | GCCCTGCATCTACATCAAGAGCTGGTGGCCGGACCAG |
| | | CGCCGCCGCCTCTACAATGCCAACATCATGGACCACA |
| | | TTGCCGACAAGCTGGAAGAAGGCCTGAACGACATACA |
| | | GGAGATGATCAAAACGGAGAAGTCCTACCCTGAGCGT |
| | | CGCCTGCGGGGCGTCCTGGAGGAGCTGAGCTGTGGC |
| | | TGCTGCCGCTTCCTCTCCCTCGCTGACAAGGACCAGG |
| | | GCCACTCATCCCGCACCAGGCTTGACCGGGAGCGCC |
| | | TCAAGTCCTGCATGAGGGAGCTGGTAAGTATCAAGGT |
| | | TACAAGACAGGTTTAAGGAGACCAATAGAAACTGGGC |
| | | TTGTCGAGACAGAGAAGACTCTTGCGTTTCTGAGCTAG |
| | | CCCCCGGGTGCGCGGCGTCGGTGGTGCCGGCGGGG |
| | | GGCGCCAGGTCGCAGGCGGTGTAGGGCTCCAGGCAG |
| | | GCGGCGAAGGCCATGACGTGCGCTATGAAGGTCTGCT |
| | | CCTGCACGCCGTGAACCAGGTGCGCCTGCGGGCCGC |
| | | GCGCGAACACCGCCACGTCCTCGCCTGCGTGGGTCT |
| | | CTTCGTCCAGGGGCACTGCTGACTGCTGCCGATACTC |
| | | GGGGCTCCCGCTCTCGCTCTCGGTAACATCCGGCCG |
| | | GGCGCCGTCCTTGAGCACATAGCCTGGACCGTTTCGT |
| | | CGACTGGGGAGAGATCTGAGGAACCCCTAGTGATGGA |
| | | GTTGGCCACTCCCTCTCTGCGCGCTCGCTCGCTCACT |
| | | GAGGCCGCCCGGGCAAAGCCCGGGCGTCGGGCGAC |
| | | CTTTGGTCGCCCGGCCTCAGTGAGCGAGCGAGCGCG |
| | | CAGAGAGGGAGTGGCCAACCCCCCCCCCCCCCCCCC |
| | | TGCAGCCTGGCGTAATAGCGAAGAGGCCCGCACCGAT |
| | | CGCCCTTCCCAACAGTTGCGTAGCCTGAATGGCGAAT |
| | | GGCGCGACGCGCCCTGTAGCGGCGCATTAAGCGCGG |
| | | CGGGTGTGGTGGTTACGCGCAGCGTGACCGCTACACT |
| | | TGCCAGCGCCCTAGCGCCCGCTCCTTTCGCTTTCTTC |
| | | CCTTCCTTTCTCGCCACGTTCGCCGGCTTTCCCCGTCA |
| | | AGCTCTAAATCGGGGGCTCCCTTTAGGGTTCCGATTTA |
| | | GTGCTTTACGGCACCTCGACCCCAAAAAACTTGATTAG |
| | | GGTGATGGTTCACGTAGTGGGCCATCGCCCTGATAGA |
| | | CGGTTTTTCGCCCTTTGACGTTGGAGTCCACGTTCTTT |
| | | AATAGTGGACTCTTGTTCCAAACTGGAACAACACTCAA |
| | | CCCTATCGCGGTCTATTCTTTTGATTTATAAGGGATGTT |
| | | GCCGATTTCGGCCTATTGGTTAAAAAATGAGCTGATTT |
| | | AACAAAAATTTTAACAAAATTCAGAAGAACTCGTCAAGA |
| | | AGGCGATAGAAGGCGATGCGCTGCGAATCGGGAGCG |
| | | GCGATACCGTAAAGCACGAGGAAGCGGTCAGCCCATT |
| | | CGCCGCCAAGCTCTTCAGCAATATCACGGGTAGCCAA |
| | | CGCTATGTCCTGATAGCGGTCCGCCACACCCAGCCGG |
| | | CCACAGTCGATGAATCCAGAAAAGCGGCCATTTTCCA |
| | | CCATGATATTCGGCAAGCAGGCATCGCCATGGGTCAC |
| | | GACGAGATCCTCGCCGTCGGGCATGCTCGCCTTGAGC |
| | | CTGGCGAACAGTTCGGCTGGCGCGAGCCCCTGATGC |
| | | TCTTCGTCCAGATCATCCTGATCGACAAGACCGGCTTC |
| | | CATCCGAGTACGTGCTCGCTCGATGCGATGTTTCGCT |
| | | TGGTGGTCGAATGGGCAGGTAGCCGGATCAAGCGTAT |
| | | GCAGCCGCCGCATTGCATCAGCCATGATGGATACTTT |
| | | CTCGGCAGGAGCAAGGTGAGATGACAGGAGATCCTG |
| | | CCCCGGCACTTCGCCCAATAGCAGCCAGTCCCTTCCC |
| | | GCTTCAGTGACAACGTCGAGCACAGCTGCGCAAGGAA |
| | | CGCCCGTCGTGGCCAGCCACGATAGCCGCGCTGCCT |
| | | CGTCTTGCAGTTCATTCAGGGCACCGGACAGGTCGGT |
| | | CTTGACAAAAAGAACCGGGCGCCCCTGCGCTGACAGC |
| | | CGGAACACGGCGGCATCAGAGCAGCCGATTGTCTGTT |
| | | GTGCCCAGTCATAGCCGAATAGCCTCTCCACCCAAGC |
| | | GGCCGGAGAACCTGCGTGCAATCCATCTTGTTCAATC |
| | | ATGCGAAACGATCCTCATCCTGTCTCTTGATCAGATCT |
| | | TGATCCCCTGCGCCATCAGATCCTTGGCGGCGAGAAA |
| | | GCCATCCAGTTTACTTTGCAGGGCTTCCCAACCTTACC |
| | | AGAGGGCGCCCCAGCTGGCAATTCCGGTTCGCTTGCT |
| | | GTCCATAAAACCGCCCAGTCTAGCTATCGCCATGTAAG |
| | | CCCACTGCAAGCTACCTGCTTTCTCTTTGCGCTTGCGT |
| | | TTTCCCTTGTCCAGATAGCCCAGTAGCTGACATTCATC |
| | | CGGGGTCAGCACCGTTTCTGCGGACTGGCTTTCTACG |
| | | TGAAAAGGATCTAGGTGAAGATCCTTTTTGATAATCTC |
| | | ATGACCAAAATCCCTTAACGTGAGTTTTCGTTCCACTG |
| | | AGCGTCAGACCCCGTAGAAAAGATCAAAGGATCTTCTT |
| | | GAGATCCTTTTTTTCTGCGCGTAATCTGCTGCTTGCAA |
| | | ACAAAAAAACCACCGCTACCAGCGGTGGTTTGTTTGC |
| | | CGGATCAAGAGCTACCAACTCTTTTTCCGAAGGTAACT |
| | | GGCTTCAGCAGAGCGCAGATACCAAATACTGTCCTTCT |
| | | AGTGTAGCCGTAGTTAGGCCACCACTTCAAGAACTCT |
| | | GTAGCACCGCCTACATACCTCGCTCTGCTAATCCTGTT |
| | | ACCAGTGGCTGCTGCCAGTGGCGATAAGTCGTGTCTT |
| | | ACCGGGTTGGACTCAAGACGATAGTTACCGGATAAGG |
| | | CGCAGCGGTCGGGCTGAACGGGGGGTTCGTGCACAC |
| | | AGCCCAGCTTGGAGCGAACGACCTACACCGAACTGAG |
| | | ATACCTACAGCGTGAGCTATGAGAAAGCGCCACGCTT |
| | | CCCGAAGGGAGAAAGGCGGACAGGTATCCGGTAAGC |
| | | GGCAGGGTCGGAACAGGAGAGCGCACGAGGGAGCTT |
| | | CCAGGGGGAAACGCCTGGTATCTTTATAGTCCTGTCG |
| | | GGTTTCGCCACCTCTGACTTGAGCGTCGATTTTTGTGA |
| | | TGCTCGTCAGGGGGGCGGAGCCTATGGAAAAACGCC |
| | | AGCAACGCGGCCTTTTTACGGTTCCTGGGCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGGGCTGCA |
| 63 | 3′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | AP head sequence at positions | AGTGAGCGAGCGAGCGCGCAGAGAGGGAGTGGCCAA |
| | 187-473 | CTCCATCACTAGGGGTTCCTCAGATCTGAATTCTAGCG |
| | Splice acceptor sequence at | GCCGCCCCCGGGTGCGCGGCGTCGGTGGTGCCGGC |
| | positions 496-544 | GGGGGGCGCCAGGTCGCAGGCGGTGTAGGGCTCCA |
| | C-terminal portion of human | GGCAGGCGGCGAAGGCCATGACGTGCGCTATGAAGG |
| | OTOF isoform 5 at positions | TCTGCTCCTGCACGCCGTGAACCAGGTGCGCCTGCG |
| | 545-4132 | GGCCGCGCGCGAACACCGCCACGTCCTCGCCTGCGT |
| | bGH poly(A) sequence at | GGGTCTCTTCGTCCAGGGGCACTGCTGACTGCTGCCG |
| | positions 4175-4396 | ATACTCGGGGCTCCCGCTCTCGCTCTCGGTAACATCC |
| | ITR at positions 4447-4589 | GGCCGGGCGCCGTCCTTGAGCACATAGCCTGGACCG |
| | KanR at positions 5115-5909 | TTTCCTTAAGCGACGCATGCTCGCGATAGGCACCTATT |
| | pUC ori at positions 6295-6965 | GGTCTTACTGACATCCACTTTGCCTTTCTCTCCACAGG |
| | Transgene to be transferred | AAAACATGGGGCAGCAGGCCAGGATGCTGCGGGCCC |
| | into vector in dual vector | AGGTGAAGCGGCACACGGTGCGGGACAAGCTGAGGC |
| | system at positions 19-4589 | TGTGCCAGAACTTCCTGCAGAAGCTGCGCTTCCTGGC |
| | | GGACGAGCCCCAGCACAGCATTCCCGACATCTTCATC |
| | | TGGATGATGAGCAACAACAAGCGTGTCGCCTATGCCC |
| | | GTGTGCCCTCCAAGGACCTGCTCTTCTCCATCGTGGA |
| | | GGAGGAGACTGGCAAGGACTGCGCCAAGGTCAAGAC |
| | | GCTCTTCCTTAAGCTGCCAGGGAAGCGGGGCTTCGGC |
| | | TCGGCAGGCTGGACAGTGCAGGCCAAGGTGGAGCTG |
| | | TACCTGTGGCTGGGCCTCAGCAAACAGCGCAAGGAGT |
| | | TCCTGTGCGGCCTGCCCTGTGGCTTCCAGGAGGTCAA |
| | | GGCAGCCCAGGGCCTGGGCCTGCATGCCTTCCCACC |
| | | CGTCAGCCTGGTCTACACCAAGAAGCAGGCGTTCCAG |
| | | CTCCGAGCGCACATGTACCAGGCCCGCAGCCTCTTTG |
| | | CCGCCGACAGCAGCGGACTCTCAGACCCCTTTGCCCG |
| | | CGTCTTCTTCATCAATCAGAGTCAGTGCACAGAGGTGC |
| | | TGAATGAGACCCTGTGTCCCACCTGGGACCAGATGCT |
| | | GGTGTTCGACAACCTGGAGCTCTATGGTGAAGCTCAT |
| | | GAGCTGAGGGACGATCCGCCCATCATTGTCATTGAAA |
| | | TCTATGACCAGGATTCCATGGGCAAAGCTGACTTCATG |
| | | GGCCGGACCTTCGCCAAACCCCTGGTGAAGATGGCA |
| | | GACGAGGCGTACTGCCCACCCCGOTTCCCACCTCAGC |
| | | TCGAGTACTACCAGATCTACCGTGGCAACGCCACAGC |
| | | TGGAGACCTGCTGGCGGCCTTCGAGCTGCTGCAGATT |
| | | GGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGGGGGGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGTGGGGGGGGCAGGACAGCAAGGGG |
| | | GAGGATTGGGAAGACAATAGCAGGCATGCTGGGGAG |
| | | AGATCTGAGGACTAGTCCGTCGACTGGGGAGAGATCT |
| | | GAGGAACCCCTAGTGATGGAGTTGGCCACTCCCTCTC |
| | | TGCGCGCTCGCTCGCTCACTGAGGCCGCCCGGGCAA |
| | | AGCCCGGGCGTCGGGCGACCTTTGGTCGCCCGGCCT |
| | | CAGTGAGCGAGCGAGCGCGCAGAGAGGGAGTGGCCA |
| | | ACCCCCCCCCCCCCCCCCCTGCAGCCTGGCGTAATAG |
| | | CGAAGAGGCCCGCACCGATCGCCCTTCCCAACAGTTG |
| | | CGTAGCCTGAATGGCGAATGGCGCGACGCGCCCTGT |
| | | AGCGGCGCATTAAGCGCGGCGGGTGTGGTGGTTACG |
| | | CGCAGCGTGACCGCTACACTTGCCAGCGCCCTAGCG |
| | | CCCGCTCCTTTCGCTTTCTTCCCTTCCTTTCTCGCCAC |
| | | GTTCGCCGGCTTTCCCCGTCAAGCTCTAAATCGGGGG |
| | | CTCCCTTTAGGGTTCCGATTTAGTGCTTTACGGCACCT |
| | | CGACCCCAAAAAACTTGATTAGGGTGATGGTTCACGTA |
| | | GTGGGCCATCGCCCTGATAGACGGTTTTTCGCCCTTT |
| | | GACGTTGGAGTCCACGTTCTTTAATAGTGGACTCTTGT |
| | | TCCAAACTGGAACAACACTCAACCCTATCGCGGTCTAT |
| | | TCTTTTGATTTATAAGGGATGTTGCCGATTTCGGCCTA |
| | | TTGGTTAAAAAATGAGCTGATTTAACAAAAATTTTAACA |
| | | AAATTCAGAAGAACTCGTCAAGAAGGCGATAGAAGGC |
| | | GATGCGCTGCGAATCGGGAGCGGCGATACCGTAAAG |
| | | CACGAGGAAGCGGTCAGCCCATTCGCCGCCAAGCTCT |
| | | TCAGCAATATCACGGGTAGCCAACGCTATGTCCTGATA |
| | | GCGGTCCGCCACACCCAGCCGGCCACAGTCGATGAA |
| | | TCCAGAAAAGCGGCCATTTTCCACCATGATATTCGGCA |
| | | AGCAGGCATCGCCATGGGTCACGACGAGATCCTCGCC |
| | | GTCGGGCATGCTCGCCTTGAGCCTGGCGAACAGTTCG |
| | | GCTGGCGCGAGCCCCTGATGCTCTTCGTCCAGATCAT |
| | | CCTGATCGACAAGACCGGCTTCCATCCGAGTACGTGC |
| | | TCGCTCGATGCGATGTTTCGCTTGGTGGTCGAATGGG |
| | | CAGGTAGCCGGATCAAGCGTATGCAGCCGCCGCATTG |
| | | CATCAGCCATGATGGATACTTTCTCGGCAGGAGCAAG |
| | | GTGAGATGACAGGAGATCCTGCCCCGGCACTTCGCCC |
| | | AATAGCAGCCAGTCCCTTCCCGCTTCAGTGACAACGT |
| | | CGAGCACAGCTGCGCAAGGAACGCCCGTCGTGGCCA |
| | | GCCACGATAGCCGCGCTGCCTCGTCTTGCAGTTCATT |
| | | CAGGGCACCGGACAGGTCGGTCTTGACAAAAAGAACC |
| | | GGGCGCCCCTGCGCTGACAGCCGGAACACGGCGGCA |
| | | TCAGAGCAGCCGATTGTCTGTTGTGCCCAGTCATAGC |
| | | CGAATAGCCTCTCCACCCAAGCGGCCGGAGAACCTGC |
| | | GTGCAATCCATCTTGTTCAATCATGCGAAACGATCCTC |
| | | ATCCTGTCTCTTGATCAGATCTTGATCCCCTGCGCCAT |
| | | CAGATCCTTGGCGGCGAGAAAGCCATCCAGTTTACTTT |
| | | GCAGGGCTTCCCAACCTTACCAGAGGGCGCCCCAGCT |
| | | GGCAATTCCGGTTCGCTTGCTGTCCATAAAACCGCCC |
| | | AGTCTAGCTATCGCCATGTAAGCCCACTGCAAGCTAC |
| | | CTGCTTTCTCTTTGCGCTTGCGTTTTCCCTTGTCCAGA |
| | | TAGCCCAGTAGCTGACATTCATCCGGGGTCAGCACCG |
| | | TTTCTGCGGACTGGCTTTCTACGTGAAAAGGATCTAGG |
| | | TGAAGATCCTTTTTGATAATCTCATGACCAAAATCCCTT |
| | | AACGTGAGTTTTCGTTCCACTGAGCGTCAGACCCCGT |
| | | AGAAAAGATCAAAGGATCTTCTTGAGATCCTTTTTTTCT |
| | | GCGCGTAATCTGCTGCTTGCAAACAAAAAAACCACCG |
| | | CTACCAGCGGTGGTTTGTTTGCCGGATCAAGAGCTAC |
| | | CAACTCTTTTTCCGAAGGTAACTGGCTTCAGCAGAGCG |
| | | CAGATACCAAATACTGTCCTTCTAGTGTAGCCGTAGTT |
| | | AGGCCACCACTTCAAGAACTCTGTAGCACCGCCTACA |
| | | TACCTCGCTCTGCTAATCCTGTTACCAGTGGCTGCTGC |
| | | CAGTGGCGATAAGTCGTGTCTTACCGGGTTGGACTCA |
| | | AGACGATAGTTACCGGATAAGGCGCAGCGGTCGGGCT |
| | | GAACGGGGGGTTCGTGCACACAGCCCAGCTTGGAGC |
| | | GAACGACCTACACCGAACTGAGATACCTACAGCGTGA |
| | | GCTATGAGAAAGCGCCACGCTTCCCGAAGGGAGAAAG |
| | | GCGGACAGGTATCCGGTAAGCGGCAGGGTCGGAACA |
| | | GGAGAGCGCACGAGGGAGCTTCCAGGGGGAAACGCC |
| | | TGGTATCTTTATAGTCCTGTCGGGTTTCGCCACCTCTG |
| | | ACTTGAGCGTCGATTTTTGTGATGCTCGTCAGGGGGG |
| | | CGGAGCCTATGGAAAAACGCCAGCAACGCGGCCTTTT |
| | | TACGGTTCCTGGGCTTTTGCTGGCCTTTTGCTCACATG |
| | | TTCTTTCCTGCGTTATCCCCTGATTCTGTGGATAACCG |
| | | TATTACCGCCTTTGAGTGAGCTGATACCGCTCGCCGC |
| | | AGCCGAACGACCGAGCGCAGCGAGTCAGTGAGCGAG |
| | | GAAGCGGAAGAGCGCCCAATACGCAAACCGCCTCTCC |
| | | CCGCGCGTTGGCCGATTCATTAATGCAGGGCTGCA |
| 64 | 5′ transgene plasmid containing | TCGCGCGTTTCGGTGATGACGGTGAAAACCTCTGACA |
| | the following features: | CATGCAGCTCCCGGATAGAGGTCATCCTTCCTGACCA |
| | Lambda at positions 53-2027 | TTTCCATCATTCCAGTCGAACTCACACACAACACCAAA |
| | ITR at positions 2049-2178 | TGCATTTAAGTCGCTTGAAATTGCTATAAGCAGAGCAT |
| | CMV i.e enhancer at positions | GTTGCGCCAGCATGATTAATACAGCATTTAATACAGAG |
| | 2267-2636 (part of smCBA | CCGTGTTTATTGAGTCGGTATTCAGAGTCTGACCAGAA |
| | promoter) | ATTATTAATCTGGTGAAGTTATTCCTCTGTCATTACGTC |
| | Chicken ß-actin promoter at | ATGGTCGATTTCAATTTCTATTGATGCTTTCCAGTCGTA |
| | positions 2633-2915 (part of | ATCAATGATGTATTTTTTGATGTTTGACCTCTGTTCATA |
| | smCBA promoter) | TCCTCACAGATAAAAAATCGCCCTCACACTGGAGGGC |
| | Exon1 at positions 2916-3008 | AAAGAAGATTTCCAATAATCAGAACAAGTCGGCTCCTG |
| | (part of smCBA promoter) | TTTAGTTACGAGCGACATTGCTCCGTGTATTCACTCGT |
| | Chimeric intron at positions | TGGAATGAATACACAGTGCAGTGTTTATTCTGTTATTTA |
| | 3008-3209 (part of smCBA | TGCCAAAAATTAAGGCCACTATCAGGCAGCTTTGTTGT |
| | promoter) | TCTGTTTACCAAGTTCTCTGGCAATCATTGCCGTCGTT |
| | Kozak sequence at positions | CGTATTGCCCATTTATCGACATATTTCCCATCTTCCTAT |
| | 3226-3235 | ACAGGAAACATTTCTTCAGGCTTAACCATGCATTCCGA |
| | N-terminal portion of human | TTGCAGCTTGCATCCATTGCATCGCTTGAATTGTCCAC |
| | OTOF isoform 5 at positions | ACCATTGATTTTTATCAATAGTCGTAGTTTAACGGATAG |
| | 3232-5637 | TCCTGGTATTGTTCCATCACATCCTGAGGATGCCCTTC |
| | Splice donor sequence at | GAACTCTTCAAATTCTTCTTCCTAATATCACCTTAAATA |
| | positions 5638-5721 | GTGGATTGCGGTAGTAAAGATTGTGCCTGTCTTTTAAC |
| | AP head sequence at positions | CACATCAGGCTCGGTGGTTCTCGTGTACCCCTACAGC |
| | 5728-6014 | GAGAAATCGGATAAACTATTACAACCCCTACAGTTTGT |
| | ITR at positions 6108-6237 | AGAGTATAGAAAATGATCCACTCGTTATTCTCGGACGA |
| | Lambda at positions 6248-8260 | GTGTTCAGTAATGAACCTCTGGAGAGAACCATCTATAT |
| | Ori at positions 8317-8905 | GATCGTTATCTGGGTTTGACTTCTGCTTTTAAGCCCAG |
| | KanR at positions 9083-9892 | ATAACTTGCCTGAATATGTTAATGAGAGAATCGGTATT |
| | Transgene to be transferred | CCTCATGTGTGGCATGTTTTCGTCTTTGCTCTTGCATTT |
| | into vector in dual vector | TCACTAGCAATTAATGTGCATCGATTATCAGCTATTGC |
| | system at positions 2049-6237 | CAGCGCCAGATATAAGCGATTTAAGCTAAGAAAACGCA |
| | | TTAAGGTGCAAAACGATAAAGTGCGATCAGTAATTCAA |
| | | AACCTTACAGGAGAGCAATCTATGGTTTTGTGCTCAGC |
| | | CCTTAATGAAGGCAGGTAGTATGTGGTTACATCAAAAC |
| | | AATTCCCATACATTAGTGAGTTGATTGAGCTTGGTGTG |
| | | TTGAACAAAACTTTTTCCCGATGGAATGGAAAGCATAT |
| | | ATTATTCCCTATTGAGGATATTTACTGGACTGAATTAGT |
| | | TGCCAGCTATGATCCATATAATATTGAGATAAAGCCAA |
| | | GGCCAATATCTAAGTAACTAGATAAGAGGAATCGATTT |
| | | TCCCTTAATTTTCTGGCGTCCACTGCATGTTATGCCGC |
| | | GTTCGCCAGGCTTGCTGTACCATGTGCGCTGATTCTT |
| | | GCGCTCAATACGTTGCAGGTTGCTTTCAATCTGTTTGT |
| | | GGTATTCAGCCAGCACTGTAAGGTCTATCGGATTTAGT |
| | | GCGCTTTCTACTCGTGATTTCGGTTTGCGATTCAGCGA |
| | | GAGAATAGGGGGGTTAACTGGTTTTGCGCTTACCCCA |
| | | ACCAACAGGGGATTTGCTGCTTTCCATTGAGCCTGTTA |
| | | CTCTGCGCGACGTTCGCGGCGGCGTGTTTGTGCATCC |
| | | ATCTGGATTCTCCTGTCAGTTAGCTTTGGTGGTGTGTG |
| | | GCAGTTGTAGTCCTGAACGAAAACCCCCCGCGATTGG |
| | | CACGTTGGCAGCTAATCCGGAATCGCACTTACGGCCA |
| | | ATGCTTCGTTTCGTATCACACACCCCAAAGCCTTCTGC |
| | | TTTGAATGCTGCCCTTCTTCAGGGCTTAATTTTTAAGA |
| | | GCGTCACCTTCATGGTGGTCAGTGCGTCCTGCTGATG |
| | | TGCTCAGGCACGATTTAATTAAGGCCTTAATTAGGCTG |
| | | CGCGCTCGCTCGCTCACTGAGGCCGCCCGGGCAAAG |
| | | CCCGGGCGTCGGGCGACCTTTGGTCGCCCGGCCTCA |
| | | GTGAGCGAGCGAGCGCGCAGAGAGGGAGTGGCCAAC |
| | | TCCATCACTAGGGGTTCCTTGTAGTTAATGATTAACCC |
| | | GCCATGCTACTTATCTACGTAGCCATGCTCTAGGAAGA |
| | | TCGGAATTCGCCCTTAAGCTAGCGGCGCGCCGGTACC |
| | | TAGTTATTAATAGTAATCAATTACGGGGTCATTAGTTCA |
| | | TAGCCCATATATGGAGTTCCGCGTTACATAACTTACGG |
| | | TAAATGGCCCGCCTGGCTGACCGCCCAACGACCCCC |
| | | GCCCATTGACGTCAATAATGACGTATGTTCCCATAGTA |
| | | ACGCCAATAGGGACTTTCCATTGACGTCAATGGGTGG |
| | | AGTATTTACGGTAAACTGCCCACTTGGCAGTACATCAA |
| | | GTGTATCATATGCCAAGTACGCCCCCTATTGACGTCAA |
| | | TGACGGTAAATGGCCCGCCTGGCATTATGCCCAGTAC |
| | | ATGACCTTATGGGACTTTCCTACTTGGCAGTACATCTA |
| | | CGTATTAGTCATCGCTATTACCATGGTCGAGGTGAGCC |
| | | CCACGTTCTGCTTCACTCTCCCCATCTCCCCCCCCTCC |
| | | CCACCCCCAATTTTGTATTTATTTATTTTTTAATTATTTT |
| | | GTGCAGCGATGGGGGGGGGGGGGGGGGGGGGGGG |
| | | CGCGCCAGGGGGGGGGGGGCGGGGCGAGGGGCGG |
| | | GGCGGGGCGAGGCGGAGAGGTGCGGCGGCAGCCAA |
| | | TCAGAGCGGCGCGCTCCGAAAGTTTCCTTTTATGGCG |
| | | AGGCGGCGGCGGCGGCGGCCCTATAAAAAGCGAAGC |
| | | GCGCGGCGGGGGGAGTCGCTGCGCGCTGCCTTCGC |
| | | CCCGTGCCCCGCTCCGCCGCCGCCTCGCGCCGCCCG |
| | | CCCCGGCTCTGACTGACCGCGTTACTCCCACAGGTGA |
| | | GCGGGCGGGACGGCCCTTCTCCTCCGGGCTGTAATTA |
| | | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGGGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGGGGCCTCAGTGA |
| | | GCGAGCGAGCGCGCAGCCTTAATTAAATCCACATCTG |
| | | TATGTTTTTTATATTAATTTATTTTTTGCAGGGGGGCAT |
| | | TGTTTGGTAGGTGAGAGTTCTGAATTGCTATGTTTAGT |
| | | GAGTTGTATCTATTTATTTTTCAATAAATACAATTAGTTA |
| | | TGTGTTTTGGGGGCGATCGTGAGGCAAAGAAAACCCG |
| | | GCGCTGAGGCCGGGTTATTCTTGTTCTCTGGTCAAATT |
| | | ATATAGTTGGAAAACAAGGATGCATATATGAATGAACG |
| | | ATGCAGAGGCAATGCCGATGGCGATAGTGGGTATCAG |
| | | GTAGCCGCTTATGCTGGAAAGAAGCAATAACCCGCAG |
| | | AAAAACAAAGCTCCAAGCTCAACAAAACTAAGGGCATA |
| | | GACAATAACTACCTATGTCATATACCCATACTCTCTAAT |
| | | CTTGGCCAGTCGGCGCGTTCTGCTTCCGATTAGAAAC |
| | | GTCAAGGCAGCAATCAGGATTGCAATCTTGGTTCCTG |
| | | CATAGGATGACAATGTCGCCCCAAGACCATCTCTATGA |
| | | GCTGAAAAAGAAACACAAGGAATGTAGTGGCGGAAAA |
| | | GGAGATAGCAAATGCTTACGATAACGTAAGGAATTATT |
| | | ACTATGTAAACACCAGGCAAGATTCTGTTCCGTATAAT |
| | | TACTCCTGATAATTAATCCTTAACTTTGCCCACCTGCCT |
| | | TTTAAAACATTCCAGTATATCACTTTTCATTCTTGCGTA |
| | | GCAATATGCCCTCTCTTCAGCTATCTCAGCATTGGTGA |
| | | CCTTGTTCAGAGGCGCTGAGAGATGGCCTTTTTCTGAT |
| | | AGATAATGTTCTGTTAAAATATCTCCGGCCTCATCTTTT |
| | | GCCCGCAGGCTAATGTCTGAAAATTGAGGTGACGGGT |
| | | TAAAAATAATATCCTTGGCAACCTTTTTTATATCCCTTTT |
| | | AAATTTTGGCTTAATGACTATATCCAATGAGTCAAAAAG |
| | | CTCCCCTTCAATATCTGTTGCCCCTAAGACCTTTAATAT |
| | | ATCGCCAAATACAGGTAGCTTGGCTTCTACCTTCACCG |
| | | TTGTTCTGCCGATGAAATGCTAATGCATAACATCGTCT |
| | | TTGGTGGTTCCCCTCATCAGTGGCTCTATCTGAACGC |
| | | GCTCTCCACTGCTTAATGACATTCCTTTCCCGATTAAA |
| | | AAATCTGTCAGATCGGATGTGGTCGGCCCGAAAACAG |
| | | TTCTGGCAAAACCAATGGTGTCGCCTTCAACAAACAAA |
| | | AAAGATGGGAATCCCAATGATTCGTCATCTGCGAGGC |
| | | TGTTCTTAATATCTTCAACTGTAGCTTTAGAGCGATTTA |
| | | TCTTCTGAACCAGACTCTTGTCATTTGTTTTGGTAAAGA |
| | | GAAAAGTTTTTCCATCGATTTTATGAATATACAAATAAT |
| | | TGGAGCCAACCTTCAGGTGATGATTATCAGCCAGCAG |
| | | AGAATTAAGGAAAACAGACAGGTTTATTGAGCACTTAT |
| | | CTTTCCCTTTATTTTTGCTGCGGTAAGTCGCATAAAAAC |
| | | CATTCTTCACAATTCAATCCATTTACTATGTTATGTTCT |
| | | GAGGGGAGTGAAAATTCCCCTAATTCGATGAAGATTCT |
| | | TGCTAAATTGTTATCAGCTATGCGCCGACCAGAACACC |
| | | TTGCCGATCAGCCAAACGTCTAATCAGGCCACTGACTA |
| | | GCGATAACTTTCCCCACAACGGAACAACTCTCATTGCA |
| | | TGGGATAATTGGGTACTGTGGGTTTAGTGGTTGTAAAA |
| | | ACACCTGACCGCTATCCCTGATCAGTTTCTTGAAGGTA |
| | | AACTCATCACCCCCAAGTCTGGCTATACAGAAATCACC |
| | | TGGCTCAACAGCCTGCTCAGGGTCAACGAGAATTTAC |
| | | ATTCCGTCAGGATAGCTTGGCTTGGAGCCTGTTGGTG |
| | | CGGTCACGGAATTACCTTCAACCTCAAGCCAGAATGC |
| | | AGAATCACTGGCTTTTTTGGTTGTGCTTACCCATCTCT |
| | | CCGCATCACCTTTGGTAAAGGTTCTAAGCTAAGGTGAG |
| | | AACATCCCTGCCTGAACATGAGAAAAAACAGGGTACTC |
| | | ATACTCACTTATTAGTGACGGCTATGAGCAAAAGGCCA |
| | | GCAAAAGGCCAGGAACCGTAAAAAGGCCGCGTTGCTG |
| | | GCGTTTTTCCATAGGCTCCGCCCCCCTGACGAGCATC |
| | | ACAAAAATCGACGCTCAAGTCAGAGGTGGCGAAACCC |
| | | GACAGGACTATAAAGATACCAGGCGTTTCCCCCTGGA |
| | | AGCTCCCTCGTGCGCTCTCCTGTTCCGACCCTGCCGC |
| | | TTACCGGATACCTGTCCGCCTTTCTCCCTTCGGGAAG |
| | | CGTGGCGCTTTCTCATAGCTCACGCTGTAGGTATCTCA |
| | | GTTCGGTGTAGGTCGTTCGCTCCAAGCTGGGCTGTGT |
| | | GCACGAACCCCCCGTTCAGCCCGACCGCTGCGCCTTA |
| | | TCCGGTAACTATCGTCTTGAGTCCAACCCGGTAAGACA |
| | | CGACTTATCGCCACTGGCAGCAGCCACTGGTAACAGG |
| | | ATTAGCAGAGCGAGGTATGTAGGCGGTGCTACAGAGT |
| | | TCTTGAAGTGGTGGCCTAACTACGGCTACACTAGAAG |
| | | AACAGTATTTGGTATCTGCGCTCTGCTGAAGCCAGTTA |
| | | CCTTCGGAAAAAGAGTTGGTAGCTCTTGATCCGGCAA |
| | | ACAAACCACCGCTGGTAGCGGTGGTTTTTTTGTTTGCA |
| | | AGCAGCAGATTACGCGCAGAAAAAAAGGATCTCAAGA |
| | | AGATCCTTTGATCTTTTCTACGGGGTCTGACGCTCAGT |
| | | GGAACGAAAACTCACGTTAAGGGATTTTGGTCATGAGA |
| | | TTATCAAAAAGGATCTTCACCTAGATCCTTTTAAATTAA |
| | | AAATGAAGTTTTAAATCAAGCCCAATCTGAATAATGTTA |
| | | CAACCAATTAACCAATTCTGATTAGAAAAACTCATCGA |
| | | GCATCAAATGAAACTGCAATTTATTCATATCAGGATTAT |
| | | CAATACCATATTTTTGAAAAAGCCGTTTCTGTAATGAAG |
| | | GAGAAAACTCACCGAGGCAGTTCCATAGGATGGCAAG |
| | | ATCCTGGTATCGGTCTGCGATTCCGACTCGTCCAACAT |
| | | CAATACAACCTATTAATTTCCCCTCGTCAAAAATAAGGT |
| | | TATCAAGTGAGAAATCACCATGAGTGACGACTGAATCC |
| | | GGTGAGAATGGCAAAAGTTTATGCATTTCTTTCCAGAC |
| | | TTGTTCAACAGGCCAGCCATTACGCTCGTCATCAAAAT |
| | | CACTCGCATCAACCAAACCGTTATTCATTCGTGATTGC |
| | | GCCTGAGCAAGACGAAATACGCGATCGCTGTTAAAAG |
| | | GACAATTACAAACAGGAATCGAATGCAACCGGCGCAG |
| | | GAACACTGCCAGCGCATCAACAATATTTTCACCTGAAT |
| | | CAGGATATTCTTCTAATACCTGGAATGCTGTTTTTCCG |
| | | GGGATCGCAGTGGTGAGTAACCATGCATCATCAGGAG |
| | | TACGGATAAAATGCTTGATGGTCGGAAGAGGCATAAAT |
| | | TCCGTCAGCCAGTTTAGTCTGACCATCTCATCTGTAAC |
| | | ATCATTGGCAACGCTACCTTTGCCATGTTTCAGAAACA |
| | | ACTCTGGCGCATCGGGCTTCCCATACAAGCGATAGAT |
| | | TGTCGCACCTGATTGCCCGACATTATCGCGAGCCCAT |
| | | TTATACCCATATAAATCAGCATCCATGTTGGAATTTAAT |
| | | CGCGGCCTCGACGTTTCCCGTTGAATATGGCTCATAA |
| | | CACCCCTTGTATTACTGTTTATGTAAGCAGACAGTTTTA |
| | | TTGTTCATGATGATATATTTTTATCTTGTGCAATGTAAC |
| | | ATCAGAGATTTTGAGACACGGGCCAGAGCTGCA |
| 65 | 5′ transgene plasmid containing | GGGGGGGGGGGGGGGGGGTTGGCCACTCCCTCTCT |
| | the following features: | GCGCGCTCGCTCGCTCACTGAGGCCGGGCGACCAAA |
| | ITR at positions 19-161 | GGTCGCCCGACGCCCGGGCTTTGCCCGGGCGGCCTC |
| | CMV enhancer at positions | AGTGAGCGAGCGAGCGCGCAGAGAGGGAGTGGCCAA |
| | 177-546 (part of smCBA | CTCCATCACTAGGGGTTCCTCAGATCTGAATTCGGTAC |
| | promoter) | CTAGTTATTAATAGTAATCAATTACGGGGTCATTAGTTC |
| | Chicken β-actin promoter at | ATAGCCCATATATGGAGTTCCGCGTTACATAACTTACG |
| | positions 548-825 (part of | GTAAATGGCCCGCCTGGCTGACCGCCCAACGACCCC |
| | smCBA promoter) | CGCCCATTGACGTCAATAATGACGTATGTTOCCATAGT |
| | Exon1 at positions 826-918 | AACGCCAATAGGGACTTTCCATTGACGTCAATGGGTG |
| | (part of smCBA promoter) | GAGTATTTACGGTAAACTGCCCACTTGGCAGTACATCA |
| | Chimeric intron at positions | AGTGTATCATATGCCAAGTACGCCCCCTATTGACGTCA |
| | 918-1119 (part of smCBA | ATGACGGTAAATGGCCCGCCTGGCATTATGCCCAGTA |
| | promoter) | CATGACCTTATGGGACTTTCCTACTTGGCAGTACATCT |
| | N-terminal portion of human | ACGTATTAGTCATCGCTATTACCATGGTCGAGGTGAGC |
| | OTOF isoform 5 at positions | CCCACGTTCTGCTTCACTCTCCCCATCTCCCCCCCCTC |
| | 1142-3547 | CCCACCCCCAATTTTGTATTTATTTATTTTTTAATTATTT |
| | Splice donor sequence at | TGTGCAGCGATGGGGGGGGGGGGGGGGGGGGGCG |
| | positions 3548-3631 | CGCGCCAGGCGGGGGGGGGGGGGGCGAGGGGCGG |
| | AP head sequence at positions | GGCGGGGCGAGGCGGAGAGGTGCGGCGGCAGCCAA |
| | 3638-3924 | TCAGAGCGGCGCGCTCCGAAAGTTTCCTTTTATGGCG |
| | ITR at positions 3948-4090 | AGGCGGCGGCGGGGGGGGCCCTATAAAAAGCGAAGC |
| | KanR at positions 4616-5410 | GCGCGGGGGGCGGGAGTCGCTGCGCGCTGCCTTCGC |
| | pUC ori at positions 5796-6466 | CCCGTGCCCCGCTCCGCCGCCGCCTCGCGCCGCCCG |
| | Transgene to be transferred | CCCCGGCTCTGACTGACCGCGTTACTCCCACAGGTGA |
| | into vector in dual vector | GCGGGGGGGACGGCCCTTCTCCTCCGGGCTGTAATTA |
| | system at positions 19-4090 | GCGCTTGGTTTAATGACGGCTTGTTTCTTTTCTGTGGC |
| | | TGCGTGAAAGCCTTGAGGGGCTCCGGGAGCTAGAGC |
| | | CTCTGCTAACCATGTTCATGCCTTCTTCTTTTTCCTACA |
| | | GCTCCTGGGCAACGTGCTGGTTATTGTGCTGTCTCAT |
| | | CATTTTGGCAAAGAATTCTAGCGGCCGCCACCATGGC |
| | | CTTGCTCATCCACCTCAAGACAGTCTCGGAGCTGCGG |
| | | GGCAGGGGCGACCGGATCGCCAAAGTGACTTTCCGA |
| | | GGGCAATCCTTCTACTCTCGGGTCCTGGAGAACTGTG |
| | | AGGATGTGGCTGACTTTGATGAGACATTTCGGTGGCC |
| | | GGTGGCCAGCAGCATCGACAGAAATGAGATGCTGGAG |
| | | ATTCAGGTTTTCAACTACAGCAAAGTCTTCAGCAACAA |
| | | GCTCATCGGGACCTTCCGCATGGTGCTGCAGAAGGTG |
| | | GTAGAGGAGAGCCATGTGGAGGTGACTGACACGCTGA |
| | | TTGATGACAACAATGCTATCATCAAGACCAGCCTGTGC |
| | | GTGGAGGTCCGGTATCAGGCCACTGACGGCACAGTG |
| | | GGCTCCTGGGACGATGGGGACTTCCTGGGAGATGAG |
| | | TCTCTTCAAGAGGAAGAGAAGGACAGCCAAGAGACGG |
| | | ATGGACTGCTCCCAGGCTCCCGGCCCAGCTCCCGGC |
| | | CCCCAGGAGAGAAGAGCTTCCGGAGAGCCGGGAGGA |
| | | GCGTGTTCTCCGCCATGAAGCTCGGCAAAAACCGGTC |
| | | TCACAAGGAGGAGCCCCAAAGACCAGATGAACCGGC |
| | | GGTGCTGGAGATGGAAGACCTTGACCATCTGGCCATT |
| | | CGGCTAGGAGATGGACTGGATCCCGACTCGGTGTCTC |
| | | TAGCCTCAGTCACAGCTCTCACCACTAATGTCTCCAAC |
| | | AAGCGATCTAAGCCAGACATTAAGATGGAGCCAAGTG |
| | | CTGGGCGGCCCATGGATTACCAGGTCAGCATCACGGT |
| | | GATCGAGGCCCGGCAGCTGGTGGGCTTGAACATGGA |
| | | CCCTGTGGTGTGCGTGGAGGTGGGTGACGACAAGAA |
| | | GTACACATCCATGAAGGAGTCCACTAACTGCCCCTATT |
| | | ACAACGAGTACTTCGTCTTCGACTTCCATGTCTCTCCG |
| | | GATGTCATGTTTGACAAGATCATCAAGATTTCGGTGAT |
| | | TCACTCCAAGAACCTGCTGCGCAGTGGCACCCTGGTG |
| | | GGCTCCTTCAAAATGGACGTGGGAACCGTGTACTCGC |
| | | AGCCAGAGCACCAGTTCCATCACAAGTGGGCCATCCT |
| | | GTCTGACCCCGATGACATCTCCTCGGGGCTGAAGGGC |
| | | TACGTGAAGTGTGACGTTGCCGTGGTGGGCAAAGGG |
| | | GACAACATCAAGACGCCCCACAAGGCCAATGAGACCG |
| | | ACGAAGATGACATTGAGGGGAACTTGCTGCTCCCCGA |
| | | GGGGGTGCCCCCCGAACGCCAGTGGGCCCGGTTCTA |
| | | TGTGAAAATTTACCGAGCAGAGGGGCTGCCCCGTATG |
| | | AACACAAGCCTCATGGCCAATGTAAAGAAGGCTTTCAT |
| | | CGGTGAAAACAAGGACCTCGTGGACCCCTACGTGCAA |
| | | GTCTTCTTTGCTGGCCAGAAGGGCAAGACTTCAGTGC |
| | | AGAAGAGCAGCTATGAGCCCCTGTGGAATGAGCAGGT |
| | | CGTCTTTACAGACCTCTTCCCCCCACTCTGCAAACGCA |
| | | TGAAGGTGCAGATCCGAGACTCGGACAAGGTCAACGA |
| | | CGTGGCCATCGGCACCCACTTCATTGACCTGCGCAAG |
| | | ATTTCTAATGACGGAGACAAAGGCTTCCTGCCCACACT |
| | | GGGCCCAGCCTGGGTGAACATGTACGGCTCCACACGT |
| | | AACTACACGCTGCTGGATGAGCATCAGGACCTGAACG |
| | | AGGGCCTGGGGGAGGGTGTGTCCTTCCGGGCCCGGC |
| | | TCCTGCTGGGCCTGGCTGTGGAGATCGTAGACACCTC |
| | | CAACCCTGAGCTCACCAGCTCCACAGAGGTGCAGGTG |
| | | GAGCAGGCCACGCCCATCTCGGAGAGCTGTGCAGGT |
| | | AAAATGGAAGAATTCTTTCTCTTTGGAGCCTTCCTGGA |
| | | GGCCTCAATGATCGACCGGAGAAACGGAGACAAGCCC |
| | | ATCACCTTTGAGGTCACCATAGGCAACTATGGGAACG |
| | | AAGTTGATGGCCTGTCCCGGCCCCAGCGGCCTCGGC |
| | | CCCGGAAGGAGCCGGGGGATGAGGAAGAAGTAGACC |
| | | TGATTCAGAACGCAAGTGATGACGAGGCCGGTGATGC |
| | | CGGGGACCTGGCCTCAGTCTCCTCCACTCCACCAATG |
| | | CGGCCCCAGGTCACCGACAGGAACTACTTCCATCTGC |
| | | CCTACCTGGAGCGAAAGCCCTGCATCTACATCAAGAG |
| | | CTGGTGGCCGGACCAGCGCCGCCGCCTCTACAATGC |
| | | CAACATCATGGACCACATTGCCGACAAGCTGGAAGAA |
| | | GGCCTGAACGACATACAGGAGATGATCAAAACGGAGA |
| | | AGTCCTACCCTGAGCGTCGCCTGCGGGGCGTCCTGG |
| | | AGGAGCTGAGCTGTGGCTGCTGCCGCTTCCTCTCCCT |
| | | CGCTGACAAGGACCAGGGCCACTCATCCCGCACCAG |
| | | GCTTGACCGGGAGCGCCTCAAGTCCTGCATGAGGGA |
| | | GCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGCGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACTGGGGAGAGATCTGAG |
| | | GAACCCCTAGTGATGGAGTTGGCCACTCCCTCTCTGC |
| | | GCGCTCGCTCGCTCACTGAGGCCGCCCGGGCAAAGC |
| | | CCGGGCGTCGGGCGACCTTTGGTCGCCCGGCCTCAG |
| | | TGAGCGAGCGAGCGCGCAGAGAGGGAGTGGCCAACC |
| | | CCCCCCCCCCCCCCCCTGCAGCCTGGCGTAATAGCG |
| | | AAGAGGCCCGCACCGATCGCCCTTCCCAACAGTTGCG |
| | | TAGCCTGAATGGCGAATGGCGCGACGCGCCCTGTAG |
| | | CGGCGCATTAAGCGCGGCGGGTGTGGTGGTTACGCG |
| | | CAGCGTGACCGCTACACTTGCCAGCGCCCTAGCGCCC |
| | | GCTCCTTTCGCTTTCTTCCCTTCCTTTCTCGCCACGTT |
| | | CGCCGGCTTTCCCCGTCAAGCTCTAAATCGGGGGCTC |
| | | CCTTTAGGGTTCCGATTTAGTGCTTTACGGCACCTCGA |
| | | CCCCAAAAAACTTGATTAGGGTGATGGTTCACGTAGTG |
| | | GGCCATCGCCCTGATAGACGGTTTTTCGCCCTTTGAC |
| | | GTTGGAGTCCACGTTCTTTAATAGTGGACTCTTGTTCC |
| | | AAACTGGAACAACACTCAACCCTATCGCGGTCTATTCT |
| | | TTTGATTTATAAGGGATGTTGCCGATTTCGGCCTATTG |
| | | GTTAAAAAATGAGCTGATTTAACAAAAATTTTAACAAAA |
| | | TTCAGAAGAACTCGTCAAGAAGGCGATAGAAGGCGAT |
| | | GCGCTGCGAATCGGGAGCGGCGATACCGTAAAGCAC |
| | | GAGGAAGCGGTCAGCCCATTCGCCGCCAAGCTCTTCA |
| | | GCAATATCACGGGTAGCCAACGCTATGTCCTGATAGC |
| | | GGTCCGCCACACCCAGCCGGCCACAGTCGATGAATCC |
| | | AGAAAAGCGGCCATTTTCCACCATGATATTCGGCAAGC |
| | | AGGCATCGCCATGGGTCACGACGAGATCCTCGCCGTC |
| | | GGGCATGCTCGCCTTGAGCCTGGCGAACAGTTCGGCT |
| | | GGCGCGAGCCCCTGATGCTCTTCGTCCAGATCATCCT |
| | | GATCGACAAGACCGGCTTCCATCCGAGTACGTGCTCG |
| | | CTCGATGCGATGTTTCGCTTGGTGGTCGAATGGGCAG |
| | | GTAGCCGGATCAAGCGTATGCAGCCGCCGCATTGCAT |
| | | CAGCCATGATGGATACTTTCTCGGCAGGAGCAAGGTG |
| | | AGATGACAGGAGATCCTGCCCCGGCACTTCGCCCAAT |
| | | AGCAGCCAGTCCCTTCCCGCTTCAGTGACAACGTCGA |
| | | GCACAGCTGCGCAAGGAACGCCCGTCGTGGCCAGCC |
| | | ACGATAGCCGCGCTGCCTCGTCTTGCAGTTCATTCAG |
| | | GGCACCGGACAGGTCGGTCTTGACAAAAAGAACCGG |
| | | GCGCCCCTGCGCTGACAGCCGGAACACGGCGGCATC |
| | | AGAGCAGCCGATTGTCTGTTGTGCCCAGTCATAGCCG |
| | | AATAGCCTCTCCACCCAAGCGGCCGGAGAACCTGCGT |
| | | GCAATCCATCTTGTTCAATCATGCGAAACGATCCTCAT |
| | | CCTGTCTCTTGATCAGATCTTGATCCCCTGCGCCATCA |
| | | GATCCTTGGCGGCGAGAAAGCCATCCAGTTTACTTTG |
| | | CAGGGCTTCCCAACCTTACCAGAGGGCGCCCCAGCTG |
| | | GCAATTCCGGTTCGCTTGCTGTCCATAAAACCGCCCA |
| | | GTCTAGCTATCGCCATGTAAGCCCACTGCAAGCTACCT |
| | | GCTTTCTCTTTGCGCTTGCGTTTTCCCTTGTCCAGATA |
| | | GCCCAGTAGCTGACATTCATCCGGGGTCAGCACCGTT |
| | | TCTGCGGACTGGCTTTCTACGTGAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTCCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGGCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGGGCTGCA |
| 66 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | Myo15 promoter at positions | GGGAGTGGCCAACTCCATCACTAGGGGTTCCTTGTAG |
| | 235-1199 | TTAATGATTAACCCGCCATGCTACTTATCTACGTAGCC |
| | Kozak sequence at positions | ATGCTCTAGGAAGATCGGAATTCGCCCTTAAGCTAGC |
| | 1216-1225 | GGCGCGCCCAATTCTGCAGCTCAGCCTACTACTTGCT |
| | N-terminal portion of human | TTCCAGGCTGTTCCTAGTTCCCATGTCAGCTGCTTGTG |
| | OTOF isoform 5 at positions | CTTTCCAGAGACAAAACAGGAATAATAGATGTCATTAA |
| | 1222-3627 | ATATACATTGGGCCCCAGGCGGTCAATGTGGCAGCCT |
| | Splice donor sequence at | GAGCCTCCTTTCCATCTCTGTGGAGGCAGACATAGGA |
| | positions 3628-3711 | CCCCCAACAAACAGCATGCAGGTTGGGAGCCAGCCAC |
| | AP head sequence at positions | AGGACCCAGGTAAGGGGCCCTGGGTCCTTAAGCTTCT |
| | 3718-4004 | GCCACTGGCTCCGGCATTGCAGAGAGAAGAGAAGGG |
| | ITR at positions 4098-4227 | GCGGCAGACTGGAGAGCTGGGCTCCATTTTTGTTCCT |
| | M13 fwd at positions 4246-4262 | TGGTGCCCTGCCCCTCCCCATGACCTGCAGAGACATT |
| | f1 ori at positions 4404-4859 | CAGCCTGCCAGGCTTTATGAGGTGGGAGCTGGGCTCT |
| | AmpR promoter at positions | CCCTGATGTATTATTCAGCTCCCTGGAGTTGGCCAGCT |
| | 4885-4989 | CCTGTTACACTGGCCACAGCCCTGGGCATCCGCTTCT |
| | KanR at positions 4990-5799 | CACTTCTAGTTTCCCCTCCAAGGTAATGTGGTGGGTCA |
| | mutBsmBI at positions | TGATCATTCTATCCTGGCTTCAGGGACCTGACTCCACT |
| | 55430-430 | TTGGGGCCATTCGAGGGGTCTAGGGTAGATGATGTCC |
| | ori at positions 5970-6558 | CCCTGTGGGGATTAATGTCCTGCTCTGTAAAACTGAGC |
| | CAP binding site at positions | TAGCTGAGATCCAGGAGGGCTTGGCCAGAGACAGCAA |
| | 6846-6867 | GTTGTTGCCATGGTGACTTTAAAGCCAGGTTGCTGCC |
| | lac promoter at positions | CCAGCACAGGCCTCCCAGTCTACCCTCACTAGAAAAC |
| | 6882-6912 | AACACCCAGGCACTTTCCACCACCTCTCAAAGGTGAAA |
| | lac operator at positions | CCCAAGGCTGGTCTAGAGAATGAATTATGGATCCTCG |
| | 6920-6936 | CTGTCCGTGCCACCCAGCTAGTCCCAGCGGCTCAGAC |
| | M13 rev at positions 6944-6960 | ACTGAGGAGAGACTGTAGGTTCAGCTACAAGCAAAAA |
| | Transgene to be transferred | GACCTAGCTGGTCTCCAAGCAGTGTCTCCAAGTCCCT |
| | into vector in dual vector | GAACCTGTGACACCTGCCCCAGGCATCATCAGGCACA |
| | system at positions 12-4227 | GAGGGCCACCAAGAATTCTAGCGGCCGCCACCATGG |
| | | CCTTGCTCATCCACCTCAAGACAGTCTCGGAGCTGCG |
| | | GGGCAGGGGCGACCGGATCGCCAAAGTGACTTTCCG |
| | | AGGGCAATCCTTCTACTCTCGGGTCCTGGAGAACTGT |
| | | GAGGATGTGGCTGACTTTGATGAGACATTTCGGTGGC |
| | | CGGTGGCCAGCAGCATCGACAGAAATGAGATGCTGGA |
| | | GATTCAGGTTTTCAACTACAGCAAAGTCTTCAGCAACA |
| | | AGCTCATCGGGACCTTCCGCATGGTGCTGCAGAAGGT |
| | | GGTAGAGGAGAGCCATGTGGAGGTGACTGACACGCT |
| | | GATTGATGACAACAATGCTATCATCAAGACCAGCCTGT |
| | | GCGTGGAGGTCCGGTATCAGGCCACTGACGGCACAG |
| | | TGGGCTCCTGGGACGATGGGGACTTCCTGGGAGATG |
| | | AGTCTCTTCAAGAGGAAGAGAAGGACAGCCAAGAGAC |
| | | GGATGGACTGCTCCCAGGCTCCCGGCCCAGCTCCCG |
| | | GCCCCCAGGAGAGAAGAGCTTCCGGAGAGCCGGGAG |
| | | GAGCGTGTTCTCCGCCATGAAGCTCGGCAAAAACCGG |
| | | TCTCACAAGGAGGAGCCCCAAAGACCAGATGAACCGG |
| | | CGGTGCTGGAGATGGAAGACCTTGACCATCTGGCCAT |
| | | TCGGCTAGGAGATGGACTGGATCCCGACTCGGTGTCT |
| | | CTAGCCTCAGTCACAGCTCTCACCACTAATGTCTCCAA |
| | | CAAGCGATCTAAGCCAGACATTAAGATGGAGCCAAGT |
| | | GCTGGGCGGCCCATGGATTACCAGGTCAGCATCACG |
| | | GTGATCGAGGCCCGGCAGCTGGTGGGCTTGAACATG |
| | | GACCCTGTGGTGTGCGTGGAGGTGGGTGACGACAAG |
| | | AAGTACACATCCATGAAGGAGTCCACTAACTGCCCCTA |
| | | TTACAACGAGTACTTCGTCTTCGACTTCCATGTCTCTC |
| | | CGGATGTCATGTTTGACAAGATCATCAAGATTTCGGTG |
| | | ATTCACTCCAAGAACCTGCTGCGCAGTGGCACCCTGG |
| | | TGGGCTCCTTCAAAATGGACGTGGGAACCGTGTACTC |
| | | GCAGCCAGAGCACCAGTTCCATCACAAGTGGGCCATC |
| | | CTGTCTGACCCCGATGACATCTCCTCGGGGCTGAAGG |
| | | GCTACGTGAAGTGTGACGTTGCCGTGGTGGGCAAAGG |
| | | GGACAACATCAAGACGCCCCACAAGGCCAATGAGACC |
| | | GACGAAGATGACATTGAGGGGAACTTGCTGCTCCCCG |
| | | AGGGGGTGCCCCCCGAACGCCAGTGGGCCCGGTTCT |
| | | ATGTGAAAATTTACCGAGCAGAGGGGCTGCCCCGTAT |
| | | GAACACAAGCCTCATGGCCAATGTAAAGAAGGCTTTCA |
| | | TCGGTGAAAACAAGGACCTCGTGGACCCCTACGTGCA |
| | | AGTCTTCTTTGCTGGCCAGAAGGGCAAGACTTCAGTG |
| | | CAGAAGAGCAGCTATGAGCCCCTGTGGAATGAGCAGG |
| | | TCGTCTTTACAGACCTCTTCCCCCCACTCTGCAAACGC |
| | | ATGAAGGTGCAGATCCGAGACTCGGACAAGGTCAACG |
| | | ACGTGGCCATCGGCACCCACTTCATTGACCTGCGCAA |
| | | GATTTCTAATGACGGAGACAAAGGCTTCCTGCCCACA |
| | | CTGGGCCCAGCCTGGGTGAACATGTACGGCTCCACAC |
| | | GTAACTACACGCTGCTGGATGAGCATCAGGACCTGAA |
| | | CGAGGGCCTGGGGGAGGGTGTGTCCTTCCGGGCCCG |
| | | GCTCCTGCTGGGCCTGGCTGTGGAGATCGTAGACACC |
| | | TCCAACCCTGAGCTCACCAGCTCCACAGAGGTGCAGG |
| | | TGGAGCAGGCCACGCCCATCTCGGAGAGCTGTGCAG |
| | | GTAAAATGGAAGAATTCTTTCTCTTTGGAGCCTTCCTG |
| | | GAGGCCTCAATGATCGACCGGAGAAACGGAGACAAGC |
| | | CCATCACCTTTGAGGTCACCATAGGCAACTATGGGAA |
| | | CGAAGTTGATGGCCTGTCCCGGCCCCAGCGGCCTCG |
| | | GCCCCGGAAGGAGCCGGGGGATGAGGAAGAAGTAGA |
| | | CCTGATTCAGAACGCAAGTGATGACGAGGCCGGTGAT |
| | | GCCGGGGACCTGGCCTCAGTCTCCTCCACTCCACCAA |
| | | TGCGGCCCCAGGTCACCGACAGGAACTACTTCCATCT |
| | | GCCCTACCTGGAGCGAAAGCCCTGCATCTACATCAAG |
| | | AGCTGGTGGCCGGACCAGCGCCGCCGCCTCTACAAT |
| | | GCCAACATCATGGACCACATTGCCGACAAGCTGGAAG |
| | | AAGGCCTGAACGACATACAGGAGATGATCAAAACGGA |
| | | GAAGTCCTACCCTGAGCGTCGCCTGCGGGGCGTCCT |
| | | GGAGGAGCTGAGCTGTGGCTGCTGCCGCTTCCTCTCC |
| | | CTCGCTGACAAGGACCAGGGCCACTCATCCCGCACCA |
| | | GGCTTGACCGGGAGCGCCTCAAGTCCTGCATGAGGG |
| | | AGCTGGTAAGTATCAAGGTTACAAGACAGGTTTAAGGA |
| | | GACCAATAGAAACTGGGCTTGTCGAGACAGAGAAGAC |
| | | TCTTGCGTTTCTGAGCTAGCCCCCGGGTGCGCGGCGT |
| | | CGGTGGTGCCGGCGGGGGGGGCCAGGTCGCAGGCG |
| | | GTGTAGGGCTCCAGGCAGGCGGCGAAGGCCATGACG |
| | | TGCGCTATGAAGGTCTGCTCCTGCACGCCGTGAACCA |
| | | GGTGCGCCTGCGGGCCGCGCGCGAACACCGCCACGT |
| | | CCTCGCCTGCGTGGGTCTCTTCGTCCAGGGGCACTGC |
| | | TGACTGCTGCCGATACTCGGGGCTCCCGCTCTCGCTC |
| | | TCGGTAACATCCGGCCGGGCGCCGTCCTTGAGCACAT |
| | | AGCCTGGACCGTTTCGTCGACCTCGAGTTAAGGGCGA |
| | | ATTCCCGATAAGGATCTTCCTAGAGCATGGCTACGTAG |
| | | ATAAGTAGCATGGGGGGTTAATCATTAACTACAAGGAA |
| | | CCCCTAGTGATGGAGTTGGCCACTCCCTCTCTGCGCG |
| | | CTCGCTCGCTCACTGAGGCCGGGCGACCAAAGGTCG |
| | | CCCGACGCCCGGGCTTTGCCCGGGGGGCCTCAGTGA |
| | | GCGAGCGAGCGCGCAGCCTTAATTAACCTAATTCACT |
| | | GGCCGTCGTTTTACAACGTCGTGACTGGGAAAACCCT |
| | | GGCGTTACCCAACTTAATCGCCTTGCAGCACATCCCC |
| | | CTTTCGCCAGCTGGCGTAATAGCGAAGAGGCCCGCAC |
| | | CGATCGCCCTTCCCAACAGTTGCGCAGCCTGAATGGC |
| | | GAATGGGACGCGCCCTGTAGCGGCGCATTAAGCGCG |
| | | GCGGGTGTGGTGGTTACGCGCAGCGTGACCGCTACA |
| | | CTTGCCAGCGCCCTAGCGCCCGCTCCTTTCGCTTTCT |
| | | TCCCTTCCTTTCTCGCCACGTTCGCCGGCTTTCCCCGT |
| | | CAAGCTCTAAATCGGGGGCTCCCTTTAGGGTTCCGAT |
| | | TTAGTGCTTTACGGCACCTCGACCCCAAAAAACTTGAT |
| | | TAGGGTGATGGTTCACGTAGTGGGCCATCGCCCTGAT |
| | | AGACGGTTTTTCGCCCTTTGACGTTGGAGTCCACGTTC |
| | | TTTAATAGTGGACTCTTGTTCCAAACTGGAACAACACT |
| | | CAACCCTATCTCGGTCTATTCTTTTGATTTATAAGGGAT |
| | | TTTGCCGATTTCGGCCTATTGGTTAAAAAATGAGCTGA |
| | | TTTAACAAAAATTTAACGCGAATTTTAACAAAATATTAA |
| | | CGCTTACAATTTAGGTGGCACTTTTCGGGGAAATGTGC |
| | | GCGGAACCCCTATTTGTTTATTTTTCTAAATACATTCAA |
| | | ATATGTATCCGCTCATGAGACAATAACCCTGATAAATG |
| | | CTTCAATAATATTGAAAAAGGAAGAGTATGAGCCATAT |
| | | TCAACGGGAAACGTCGAGGCCGCGATTAAATTCCAAC |
| | | ATGGATGCTGATTTATATGGGTATAAATGGGCTCGCGA |
| | | TAATGTCGGGCAATCAGGTGCGACAATCTATCGCTTGT |
| | | ATGGGAAGCCCGATGCGCCAGAGTTGTTTCTGAAACA |
| | | TGGCAAAGGTAGCGTTGCCAATGATGTTACAGATGAG |
| | | ATGGTCAGACTAAACTGGCTGACGGAATTTATGCCTCT |
| | | TCCGACCATCAAGCATTTTATCCGTACTCCTGATGATG |
| | | CATGGTTACTCACCACTGCGATCCCCGGAAAAACAGC |
| | | ATTCCAGGTATTAGAAGAATATCCTGATTCAGGTGAAA |
| | | ATATTGTTGATGCGCTGGCAGTGTTCCTGCGCCGGTT |
| | | GCATTCGATTCCTGTTTGTAATTGTCCTTTTAACAGCGA |
| | | TCGCGTATTTCGTCTTGCTCAGGCGCAATCACGAATGA |
| | | ATAACGGTTTGGTTGATGCGAGTGATTTTGATGACGAG |
| | | CGTAATGGCTGGCCTGTTGAACAAGTCTGGAAAGAAA |
| | | TGCATAAACTTTTGCCATTCTCACCGGATTCAGTCGTC |
| | | ACTCATGGTGATTTCTCACTTGATAACCTTATTTTTGAC |
| | | GAGGGGAAATTAATAGGTTGTATTGATGTTGGACGAGT |
| | | CGGAATCGCAGACCGATACCAGGATCTTGCCATCCTA |
| | | TGGAACTGCCTCGGTGAGTTTTCTCCTTCATTACAGAA |
| | | ACGGCTTTTTCAAAAATATGGTATTGATAATCCTGATAT |
| | | GAATAAATTGCAGTTTCATTTGATGCTCGATGAGTTTTT |
| | | CTAACTGTCAGACCAAGTTTACTCATATATACTTTAGAT |
| | | TGATTTAAAACTTCATTTTTAATTTAAAAGGATCTAGGT |
| | | GAAGATCCTTTTTGATAATCTCATGACCAAAATCCCTTA |
| | | ACGTGAGTTTTCGTTCCACTGAGCGTCAGACCCCGTA |
| | | GAAAAGATCAAAGGATCTTCTTGAGATCCTTTTTTTCTG |
| | | CGCGTAATCTGCTGCTTGCAAACAAAAAAACCACCGCT |
| | | ACCAGCGGTGGTTTGTTTGCCGGATCAAGAGCTACCA |
| | | ACTCTTTTTCCGAAGGTAACTGGCTTCAGCAGAGCGCA |
| | | GATACCAAATACTGTTCTTCTAGTGTAGCCGTAGTTAG |
| | | GCCACCACTTCAAGAACTCTGTAGCACCGCCTACATAC |
| | | CTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGA |
| | | ACGACCTACACCGAACTGAGATACCTACAGCGTGAGC |
| | | TATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGCCTTTTGCTGGCCTTTTGCTCACATGTT |
| | | CTTTCCTGCGTTATCCCCTGATTCTGTGGATAACCGTA |
| | | TTACCGCCTTTGAGTGAGCTGATACCGCTCGCCGCAG |
| | | CCGAACGACCGAGCGCAGCGAGTCAGTGAGCGAGGA |
| | | AGCGGAAGAGCGCCCAATACGCAAACCGCCTCTCCCC |
| | | GCGCGTTGGCCGATTCATTAATGCAGCTGGCACGACA |
| | | GGTTTCCCGACTGGAAAGCGGGCAGTGAGCGCAACG |
| | | CAATTAATGTGAGTTAGCTCACTCATTAGGCACCCCAG |
| | | GCTTTACACTTTATGCTTCCGGCTCGTATGTTGTGTGG |
| | | AATTGTGAGCGGATAACAATTTCACACAGGAAACAGCT |
| | | ATGACCATGATTACGCCAGATTTAATTAAGG |
| 67 | 3′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | AP head sequence at positions | GGGAGTGGCCAACTCCATCACTAGGGGTTCCTTGTAG |
| | 229-515 | TTAATGATTAACCCGCCATGCTACTTATCTACGTAGCC |
| | Splice acceptor sequence at | ATGCTCTAGGAAGATCGGAATTCGCCCTTAAGCTAGC |
| | positions 538-586 | GGCGCGCCCCCGGGTGCGCGGCGTCGGTGGTGCCG |
| | C-terminal portion of human | GCGGGGGGCGCCAGGTCGCAGGCGGTGTAGGGCTC |
| | OTOF isoform 5 at positions | CAGGCAGGCGGCGAAGGCCATGACGTGCGCTATGAA |
| | 587-4174 | GGTCTGCTCCTGCACGCCGTGAACCAGGTGCGCCTG |
| | bGH poly(A) sequence at | CGGGCCGCGCGCGAACACCGCCACGTCCTCGCCTGC |
| | positions 4217-4438 | GTGGGTCTCTTCGTCCAGGGGCACTGCTGACTGCTGC |
| | ITR at positions 4526-4655 | CGATACTCGGGGCTCCCGCTCTCGCTCTCGGTAACAT |
| | M13 fwd at positions 4674-4690 | CCGGCCGGGCGCCGTCCTTGAGCACATAGCCTGGAC |
| | f1 ori at positions 4832-5287 | CGTTTCCTTAAGCGACGCATGCTCGCGATAGGCACCT |
| | AmpR promoter at positions | ATTGGTCTTACTGACATCCACTTTGCCTTTCTCTCCACA |
| | 5313-5417 | GGAAAACATGGGGCAGCAGGCCAGGATGCTGCGGGC |
| | KanR at positions 5418-6227 | CCAGGTGAAGCGGCACACGGTGCGGGACAAGCTGAG |
| | mutBsmBI at positions 5858- | GCTGTGCCAGAACTTCCTGCAGAAGCTGCGCTTCCTG |
| | 5858 | GCGGACGAGCCCCAGCACAGCATTCCCGACATCTTCA |
| | ori at positions 6398-6986 | TCTGGATGATGAGCAACAACAAGCGTGTCGCCTATGC |
| | CAP binding site at positions | CCGTGTGCCCTCCAAGGACCTGCTCTTCTCCATCGTG |
| | 7274-7295 | GAGGAGGAGACTGGCAAGGACTGCGCCAAGGTCAAG |
| | lac promoter at positions | ACGCTCTTCCTTAAGCTGCCAGGGAAGCGGGGCTTCG |
| | 7310-7340 | GCTCGGCAGGCTGGACAGTGCAGGCCAAGGTGGAGC |
| | lac operator at positions | TGTACCTGTGGCTGGGCCTCAGCAAACAGCGCAAGGA |
| | 7348-7364 | GTTCCTGTGCGGCCTGCCCTGTGGCTTCCAGGAGGTC |
| | M13 rev at positions 7372-7388 | AAGGCAGCCCAGGGCCTGGGCCTGCATGCCTTCCCA |
| | Transgene to be transferred | CCCGTCAGCCTGGTCTACACCAAGAAGCAGGCGTTCC |
| | into vector in dual vector | AGCTCCGAGCGCACATGTACCAGGCCCGCAGCCTCTT |
| | system at positions 12-4655 | TGCCGCCGACAGCAGCGGACTCTCAGACCCCTTTGCC |
| | | CGCGTCTTCTTCATCAATCAGAGTCAGTGCACAGAGGT |
| | | GCTGAATGAGACCCTGTGTCCCACCTGGGACCAGATG |
| | | CTGGTGTTCGACAACCTGGAGCTCTATGGTGAAGCTC |
| | | ATGAGCTGAGGGACGATCCGCCCATCATTGTCATTGA |
| | | AATCTATGACCAGGATTCCATGGGCAAAGCTGACTTCA |
| | | TGGGCCGGACCTTCGCCAAACCCCTGGTGAAGATGGC |
| | | AGACGAGGCGTACTGCCCACCCCGOTTCCCACCTCAG |
| | | CTCGAGTACTACCAGATCTACCGTGGCAACGCCACAG |
| | | CTGGAGACCTGCTGGCGGCCTTCGAGCTGCTGCAGAT |
| | | TGGACCAGCAGGGAAGGCTGACCTGCCCCCCATCAAT |
| | | GGCCCGGTGGACGTGGACCGAGGTCCCATCATGCCC |
| | | GTGCCCATGGGCATCCGGCCCGTGCTCAGCAAGTACC |
| | | GAGTGGAGGTGCTGTTCTGGGGCCTACGGGACCTAAA |
| | | GCGGGTGAACCTGGCCCAGGTGGACCGGCCACGGGT |
| | | GGACATCGAGTGTGCAGGGAAGGGGGTGCAGTCGTC |
| | | CCTGATCCACAATTATAAGAAGAACCCCAACTTCAACA |
| | | CCCTCGTCAAGTGGTTTGAAGTGGACCTCCCAGAGAA |
| | | CGAGCTGCTGCACCCGCCCTTGAACATCCGTGTGGTG |
| | | GACTGCCGGGCCTTCGGTCGCTACACACTGGTGGGCT |
| | | CCCATGCCGTCAGCTCCCTGCGACGCTTCATCTACCG |
| | | GCCCCCAGACCGCTCGGCCCCCAGCTGGAACACCAC |
| | | GGTCAGGCTTCTCCGGCGCTGCCGTGTGCTGTGCAAT |
| | | GGGGGCTCCTCCTCTCACTCCACAGGGGAGGTTGTG |
| | | GTGACTATGGAGCCAGAGGTACCCATCAAGAAACTGG |
| | | AGACCATGGTGAAGCTGGACGCGACTTCTGAAGCTGT |
| | | TGTCAAGGTGGATGTGGCTGAGGAGGAGAAGGAGAA |
| | | GAAGAAGAAGAAGAAGGGCACTGCGGAGGAGCCAGA |
| | | GGAGGAGGAGCCAGACGAGAGCATGCTGGACTGGTG |
| | | GTCCAAGTACTTTGCCTCCATTGACACCATGAAGGAGC |
| | | AACTTCGACAACAAGAGCCCTCTGGAATTGACTTGGA |
| | | GGAGAAGGAGGAAGTGGACAATACCGAGGGCCTGAA |
| | | GGGGTCAATGAAGGGCAAGGAGAAGGCAAGGGCTGC |
| | | CAAAGAGGAGAAGAAGAAGAAAACTCAGAGCTCTGGC |
| | | TCTGGCCAGGGGTCCGAGGCCCCCGAGAAGAAGAAA |
| | | CCCAAGATTGATGAGCTTAAGGTATACCCCAAAGAGCT |
| | | GGAGTCCGAGTTTGATAACTTTGAGGACTGGCTGCAC |
| | | ACTTTCAACTTGCTTCGGGGCAAGACCGGGGATGATG |
| | | AGGATGGCTCCACCGAGGAGGAGCGCATTGTGGGAC |
| | | GCTTCAAGGGCTCCCTCTGCGTGTACAAAGTGCCACT |
| | | CCCAGAGGACGTGTCCCGGGAAGCCGGCTACGACTC |
| | | CACCTACGGCATGTTCCAGGGCATCCCGAGCAATGAC |
| | | CCCATCAATGTGCTGGTCCGAGTCTATGTGGTCCGGG |
| | | CCACGGACCTGCACCCTGCTGACATCAACGGCAAAGC |
| | | TGACCCCTACATCGCCATCCGGCTAGGCAAGACTGAC |
| | | ATCCGCGACAAGGAGAACTACATCTCCAAGCAGCTCA |
| | | ACCCTGTCTTTGGGAAGTCCTTTGACATCGAGGCCTC |
| | | CTTCCCCATGGAATCCATGCTGACGGTGGCTGTGTAT |
| | | GACTGGGACCTGGTGGGCACTGATGACCTCATTGGGG |
| | | AAACCAAGATCGACCTGGAGAACCGCTTCTACAGCAA |
| | | GCACCGCGCCACCTGCGGCATCGCCCAGACCTACTC |
| | | CACACATGGCTACAATATCTGGCGGGACCCCATGAAG |
| | | CCCAGCCAGATCCTGACCCGCCTCTGCAAAGACGGCA |
| | | AAGTGGACGGCCCCCACTTTGGGCCCCCTGGGAGAG |
| | | TGAAGGTGGCCAACCGCGTCTTCACTGGGCCCTCTGA |
| | | GATTGAGGACGAGAACGGTCAGAGGAAGCCCACAGA |
| | | CGAGCATGTGGCGCTGTTGGCCCTGAGGCACTGGGA |
| | | GGACATCCCCCGCGCAGGCTGCCGCCTGGTGCCAGA |
| | | GCATGTGGAGACGAGGCCGCTGCTCAACCCCGACAA |
| | | GCCGGGCATCGAGCAGGGCCGCCTGGAGCTGTGGGT |
| | | GGACATGTTCCCCATGGACATGCCAGCCCCTGGGACG |
| | | CCTCTGGACATCTCACCTCGGAAGCCCAAGAAGTACG |
| | | AGCTGCGGGTCATCATCTGGAACACAGATGAGGTGGT |
| | | CTTGGAGGACGACGACTTCTTCACAGGGGAGAAGTCC |
| | | AGTGACATCTTCGTGAGGGGGTGGCTGAAGGGCCAG |
| | | CAGGAGGACAAGCAGGACACAGACGTCCACTACCACT |
| | | CCCTCACTGGCGAGGGCAACTTCAACTGGCGCTACCT |
| | | GTTCCCCTTCGACTACCTGGGGGGGGAGGAGAAGATC |
| | | GTCATCTCCAAGAAGGAGTCCATGTTCTCCTGGGACG |
| | | AGACCGAGTACAAGATCCCCGCGCGGCTCACCCTGCA |
| | | GATCTGGGATGCGGACCACTTCTCCGCTGACGACTTC |
| | | CTGGGGGCCATCGAGCTGGACCTGAACCGGTTCCCG |
| | | CGGGGCGCAAAGACAGCCAAGCAGTGCACCATGGAG |
| | | ATGGCCACCGGGGAGGTGGACGTGCCCCTCGTGTCC |
| | | ATCTTCAAGCAAAAGCGCGTCAAAGGCTGGTGGCCCC |
| | | TCCTGGCCCGCAATGAGAACGATGAGTTTGAGCTCAC |
| | | GGGCAAGGTGGAGGCTGAGCTGCATTTACTGACAGCA |
| | | GAGGAGGCAGAGAAGAACCCAGTGGGCCTGGCCCGC |
| | | AATGAACCTGACCCCCTAGAGAAACCCAACCGGCCCG |
| | | ACACGGCCTTCGTCTGGTTCCTCAACCCTCTCAAGTCC |
| | | ATCAAGTACCTCATCTGCACCCGGTACAAGTGGCTCAT |
| | | CATCAAGATCGTGCTGGCGCTGTTGGGGCTGCTCATG |
| | | TTGGGGCTCTTCCTCTACAGCCTCCCTGGCTACATGG |
| | | TCAAAAAGCTCCTTGGGGCATGAACGGCCGCTATGCT |
| | | AGCTTGGTACCAAGGGCGGATCCTGCATAGAGCTCGC |
| | | TGATCAGCCTCGACTGTGCCTTCTAGTTGCCAGCCATC |
| | | TGTTGTTTGCCCCTCCCCCGTGCCTTCCTTGACCCTG |
| | | GAAGGTGCCACTCCCACTGTCCTTTCCTAATAAAATGA |
| | | GGAAATTGCATCGCATTGTCTGAGTAGGTGTCATTCTA |
| | | TTCTGGGGGGGGGGTGGGGCAGGACAGCAAGGGG |
| | | GAGGATTGGGAAGACAATAGCAGGCATCTCGAGTTAA |
| | | GGGCGAATTCCCGATAAGGATCTTCCTAGAGCATGGC |
| | | TACGTAGATAAGTAGCATGGGGGGTTAATCATTAACTA |
| | | CAAGGAACCCCTAGTGATGGAGTTGGCCACTCCCTCT |
| | | CTGCGCGCTCGCTCGCTCACTGAGGCCGGGCGACCA |
| | | AAGGTCGCCCGACGCCCGGGCTTTGCCCGGGGGGCC |
| | | TCAGTGAGCGAGCGAGCGCGCAGCCTTAATTAACCTA |
| | | ATTCACTGGCCGTCGTTTTACAACGTCGTGACTGGGAA |
| | | AACCCTGGCGTTACCCAACTTAATCGCCTTGCAGCACA |
| | | TCCCCCTTTCGCCAGCTGGCGTAATAGCGAAGAGGCC |
| | | CGCACCGATCGCCCTTCCCAACAGTTGCGCAGCCTGA |
| | | ATGGCGAATGGGACGCGCCCTGTAGCGGCGCATTAA |
| | | GCGCGGCGGGTGTGGTGGTTACGCGCAGCGTGACCG |
| | | CTACACTTGCCAGCGCCCTAGCGCCCGCTCCTTTCGC |
| | | TTTCTTCCCTTCCTTTCTCGCCACGTTCGCCGGCTTTC |
| | | CCCGTCAAGCTCTAAATCGGGGGCTCCCTTTAGGGTT |
| | | CCGATTTAGTGCTTTACGGCACCTCGACCCCAAAAAAC |
| | | TTGATTAGGGTGATGGTTCACGTAGTGGGCCATCGCC |
| | | CTGATAGACGGTTTTTCGCCCTTTGACGTTGGAGTCCA |
| | | CGTTCTTTAATAGTGGACTCTTGTTCCAAACTGGAACA |
| | | ACACTCAACCCTATCTCGGTCTATTCTTTTGATTTATAA |
| | | GGGATTTTGCCGATTTCGGCCTATTGGTTAAAAAATGA |
| | | GCTGATTTAACAAAAATTTAACGCGAATTTTAACAAAAT |
| | | ATTAACGCTTACAATTTAGGTGGCACTTTTCGGGGAAA |
| | | TGTGCGCGGAACCCCTATTTGTTTATTTTTCTAAATACA |
| | | TTCAAATATGTATCCGCTCATGAGACAATAACCCTGAT |
| | | AAATGCTTCAATAATATTGAAAAAGGAAGAGTATGAGC |
| | | CATATTCAACGGGAAACGTCGAGGCCGCGATTAAATT |
| | | CCAACATGGATGCTGATTTATATGGGTATAAATGGGCT |
| | | CGCGATAATGTCGGGCAATCAGGTGCGACAATCTATC |
| | | GCTTGTATGGGAAGCCCGATGCGCCAGAGTTGTTTCT |
| | | GAAACATGGCAAAGGTAGCGTTGCCAATGATGTTACA |
| | | GATGAGATGGTCAGACTAAACTGGCTGACGGAATTTAT |
| | | GCCTCTTCCGACCATCAAGCATTTTATCCGTACTCCTG |
| | | ATGATGCATGGTTACTCACCACTGCGATCCCCGGAAA |
| | | AACAGCATTCCAGGTATTAGAAGAATATCCTGATTCAG |
| | | GTGAAAATATTGTTGATGCGCTGGCAGTGTTCCTGCG |
| | | CCGGTTGCATTCGATTCCTGTTTGTAATTGTCCTTTTAA |
| | | CAGCGATCGCGTATTTCGTCTTGCTCAGGCGCAATCA |
| | | CGAATGAATAACGGTTTGGTTGATGCGAGTGATTTTGA |
| | | TGACGAGCGTAATGGCTGGCCTGTTGAACAAGTCTGG |
| | | AAAGAAATGCATAAACTTTTGCCATTCTCACCGGATTC |
| | | AGTCGTCACTCATGGTGATTTCTCACTTGATAACCTTAT |
| | | TTTTGACGAGGGGAAATTAATAGGTTGTATTGATGTTG |
| | | GACGAGTCGGAATCGCAGACCGATACCAGGATCTTGC |
| | | CATCCTATGGAACTGCCTCGGTGAGTTTTCTCCTTCAT |
| | | TACAGAAACGGCTTTTTCAAAAATATGGTATTGATAATC |
| | | CTGATATGAATAAATTGCAGTTTCATTTGATGCTCGATG |
| | | AGTTTTTCTAACTGTCAGACCAAGTTTACTCATATATAC |
| | | TTTAGATTGATTTAAAACTTCATTTTTAATTTAAAAGGAT |
| | | CTAGGTGAAGATCCTTTTTGATAATCTCATGACCAAAAT |
| | | CCCTTAACGTGAGTTTTCGTTCCACTGAGCGTCAGACC |
| | | CCGTAGAAAAGATCAAAGGATCTTCTTGAGATCCTTTT |
| | | TTTCTGCGCGTAATCTGCTGCTTGCAAACAAAAAAACC |
| | | ACCGCTACCAGCGGTGGTTTGTTTGCCGGATCAAGAG |
| | | CTACCAACTCTTTTTCCGAAGGTAACTGGCTTCAGCAG |
| | | AGCGCAGATACCAAATACTGTTCTTCTAGTGTAGCCGT |
| | | AGTTAGGCCACCACTTCAAGAACTCTGTAGCACCGCC |
| | | TACATACCTCGCTCTGCTAATCCTGTTACCAGTGGCTG |
| | | CTGCCAGTGGCGATAAGTCGTGTCTTACCGGGTTGGA |
| | | CTCAAGACGATAGTTACCGGATAAGGCGCAGCGGTCG |
| | | GGCTGAACGGGGGGTTCGTGCACACAGCCCAGCTTG |
| | | GAGCGAACGACCTACACCGAACTGAGATACCTACAGC |
| | | GTGAGCTATGAGAAAGCGCCACGCTTCCCGAAGGGAG |
| | | AAAGGCGGACAGGTATCCGGTAAGCGGCAGGGTCGG |
| | | AACAGGAGAGCGCACGAGGGAGCTTCCAGGGGGAAA |
| | | CGCCTGGTATCTTTATAGTCCTGTCGGGTTTCGCCACC |
| | | TCTGACTTGAGCGTCGATTTTTGTGATGCTCGTCAGGG |
| | | GGGCGGAGCCTATGGAAAAACGCCAGCAACGCGGCC |
| | | TTTTTACGGTTCCTGGCCTTTTGCTGGCCTTTTGCTCA |
| | | CATGTTCTTTCCTGCGTTATCCCCTGATTCTGTGGATA |
| | | ACCGTATTACCGCCTTTGAGTGAGCTGATACCGCTCG |
| | | CCGCAGCCGAACGACCGAGCGCAGCGAGTCAGTGAG |
| | | CGAGGAAGCGGAAGAGCGCCCAATACGCAAACCGCC |
| | | TCTCCCCGCGCGTTGGCCGATTCATTAATGCAGCTGG |
| | | CACGACAGGTTTCCCGACTGGAAAGCGGGCAGTGAG |
| | | CGCAACGCAATTAATGTGAGTTAGCTCACTCATTAGGC |
| | | ACCCCAGGCTTTACACTTTATGCTTCCGGCTCGTATGT |
| | | TGTGTGGAATTGTGAGCGGATAACAATTTCACACAGGA |
| | | AACAGCTATGACCATGATTACGCCAGATTTAATTAAGG |
| 68 | 5′ transgene plasmid containing | CCTTAATTAGGCTGCGCGCTCGCTCGCTCACTGAGGC |
| | the following features: | CGCCCGGGCAAAGCCCGGGCGTCGGGCGACCTTTGG |
| | ITR at positions 12-141 | TCGCCCGGCCTCAGTGAGCGAGCGAGCGCGCAGAGA |
| | CMV i.e enhancer at positions | GGGAGTGGCCAACTCCATCACTAGGGGTTCCTTGTAG |
| | 230-594 | TTAATGATTAACCCGCCATGCTACTTATCTACGTAGCC |
| | CMV enhancer at positions | ATGCTCTAGGAAGATCGGAATTCGCCCTTAAGCTAGC |
| | 296-599 | GGCGCGCCGGTACCTAGTTATTAATAGTAATCAATTAC |
| | Chicken ß-actin promoter at | GGGGTCATTAGTTCATAGCCCATATATGGAGTTCCGC |
| | positions 596-878 | GTTACATAACTTACGGTAAATGGCCCGCCTGGCTGAC |
| | Exon 1 at positions 879-971 | CGCCCAACGACCCCCGCCCATTGACGTCAATAATGAC |
| | Chimeric intron at positions | GTATGTTCCCATAGTAACGCCAATAGGGACTTTCCATT |
| | 971-1172 | GACGTCAATGGGTGGAGTATTTACGGTAAACTGCCCA |
| | Kozak sequence at positions | CTTGGCAGTACATCAAGTGTATCATATGCCAAGTACGC |
| | 1189-1198 | CCCCTATTGACGTCAATGACGGTAAATGGCCCGCCTG |
| | N-terminal portion of human | GCATTATGCCCAGTACATGACCTTATGGGACTTTCCTA |
| | OTOF isoform 5 at positions | CTTGGCAGTACATCTACGTATTAGTCATCGCTATTACC |
| | 1195-3600 | ATGGTCGAGGTGAGCCCCACGTTCTGCTTCACTCTCC |
| | Splice donor sequence at | CCATCTCCCCCCCCTCCCCACCCCCAATTTTGTATTTA |
| | positions 3601-3684 | TTTATTTTTTAATTATTTTGTGCAGCGATGGGGGGGGG |
| | AP head sequence at positions | GGGGGGGGGGGGGCGCGCGCCAGGCGGGGGGGG |
| | 3691-3977 | CGGGGCGAGGGGGGGGGGGGGGCGAGGCGGAGAG |
| | ITR at positions 4071-4200 | GTGCGGCGGCAGCCAATCAGAGCGGCGCGCTCCGAA |
| | M13 fwd at positions 4219-4235 | AGTTTCCTTTTATGGCGAGGGGGGGGCGGCGGCGGC |
| | f1 ori at positions 4377-4832 | CCTATAAAAAGCGAAGCGCGGGGGGGGGGGGAGTCG |
| | AmpR promoter at positions | CTGCGCGCTGCCTTCGCCCCGTGCCCCGCTCCGCCG |
| | 4858-4962 | CCGCCTCGCGCCGCCCGCCCCGGCTCTGACTGACCG |
| | KanR at positions 4963-5772 | CGTTACTCCCACAGGTGAGCGGGCGGGACGGCCCTT |
| | mutBsmBI at positions 4963- | CTCCTCCGGGCTGTAATTAGCGCTTGGTTTAATGACG |
| | 5772 | GCTTGTTTCTTTTCTGTGGCTGCGTGAAAGCCTTGAGG |
| | ori at positions 5943-6531 | GGCTCCGGGAGCTAGAGCCTCTGCTAACCATGTTCAT |
| | CAP binding site at positions | GCCTTCTTCTTTTTCCTACAGCTCCTGGGCAACGTGCT |
| | 6819-6840 | GGTTATTGTGCTGTCTCATCATTTTGGCAAAGAATTCTA |
| | lac promoter at positions | GCGGCCGCCACCATGGCCTTGCTCATCCACCTCAAGA |
| | 6855-6885 | CAGTCTCGGAGCTGCGGGGCAGGGGCGACCGGATCG |
| | lac operator at positions | CCAAAGTGACTTTCCGAGGGCAATCCTTCTACTCTCGG |
| | 6835-6909 | GTCCTGGAGAACTGTGAGGATGTGGCTGACTTTGATG |
| | M13 rev at positions 6917-6933 | AGACATTTCGGTGGCCGGTGGCCAGCAGCATCGACAG |
| | Transgene to be transferred | AAATGAGATGCTGGAGATTCAGGTTTTCAACTACAGCA |
| | into vector in dual vector | AAGTCTTCAGCAACAAGCTCATCGGGACCTTCCGCAT |
| | system at positions 12-4200 | GGTGCTGCAGAAGGTGGTAGAGGAGAGCCATGTGGA |
| | | GGTGACTGACACGCTGATTGATGACAACAATGCTATCA |
| | | TCAAGACCAGCCTGTGCGTGGAGGTCCGGTATCAGGC |
| | | CACTGACGGCACAGTGGGCTCCTGGGACGATGGGGA |
| | | CTTCCTGGGAGATGAGTCTCTTCAAGAGGAAGAGAAG |
| | | GACAGCCAAGAGACGGATGGACTGCTCCCAGGCTCC |
| | | CGGCCCAGCTCCCGGCCCCCAGGAGAGAAGAGCTTC |
| | | CGGAGAGCCGGGAGGAGCGTGTTCTCCGCCATGAAG |
| | | CTCGGCAAAAACCGGTCTCACAAGGAGGAGCCCCAAA |
| | | GACCAGATGAACCGGCGGTGCTGGAGATGGAAGACC |
| | | TTGACCATCTGGCCATTCGGCTAGGAGATGGACTGGA |
| | | TCCCGACTCGGTGTCTCTAGCCTCAGTCACAGCTCTC |
| | | ACCACTAATGTCTCCAACAAGCGATCTAAGCCAGACAT |
| | | TAAGATGGAGCCAAGTGCTGGGCGGCCCATGGATTAC |
| | | CAGGTCAGCATCACGGTGATCGAGGCCCGGCAGCTG |
| | | GTGGGCTTGAACATGGACCCTGTGGTGTGCGTGGAG |
| | | GTGGGTGACGACAAGAAGTACACATCCATGAAGGAGT |
| | | CCACTAACTGCCCCTATTACAACGAGTACTTCGTCTTC |
| | | GACTTCCATGTCTCTCCGGATGTCATGTTTGACAAGAT |
| | | CATCAAGATTTCGGTGATTCACTCCAAGAACCTGCTGC |
| | | GCAGTGGCACCCTGGTGGGCTCCTTCAAAATGGACGT |
| | | GGGAACCGTGTACTCGCAGCCAGAGCACCAGTTCCAT |
| | | CACAAGTGGGCCATCCTGTCTGACCCCGATGACATCT |
| | | CCTCGGGGCTGAAGGGCTACGTGAAGTGTGACGTTGC |
| | | CGTGGTGGGCAAAGGGGACAACATCAAGACGCCCCA |
| | | CAAGGCCAATGAGACCGACGAAGATGACATTGAGGGG |
| | | AACTTGCTGCTCCCCGAGGGGGTGCCCCCCGAACGC |
| | | CAGTGGGCCCGGTTCTATGTGAAAATTTACCGAGCAG |
| | | AGGGGCTGCCCCGTATGAACACAAGCCTCATGGCCAA |
| | | TGTAAAGAAGGCTTTCATCGGTGAAAACAAGGACCTC |
| | | GTGGACCCCTACGTGCAAGTCTTCTTTGCTGGCCAGA |
| | | AGGGCAAGACTTCAGTGCAGAAGAGCAGCTATGAGCC |
| | | CCTGTGGAATGAGCAGGTCGTCTTTACAGACCTCTTCC |
| | | CCCCACTCTGCAAACGCATGAAGGTGCAGATCCGAGA |
| | | CTCGGACAAGGTCAACGACGTGGCCATCGGCACCCAC |
| | | TTCATTGACCTGCGCAAGATTTCTAATGACGGAGACAA |
| | | AGGCTTCCTGCCCACACTGGGCCCAGCCTGGGTGAAC |
| | | ATGTACGGCTCCACACGTAACTACACGCTGCTGGATG |
| | | AGCATCAGGACCTGAACGAGGGCCTGGGGGAGGGTG |
| | | TGTCCTTCCGGGCCCGGCTCCTGCTGGGCCTGGCTGT |
| | | GGAGATCGTAGACACCTCCAACCCTGAGCTCACCAGC |
| | | TCCACAGAGGTGCAGGTGGAGCAGGCCACGCCCATC |
| | | TCGGAGAGCTGTGCAGGTAAAATGGAAGAATTCTTTCT |
| | | CTTTGGAGCCTTCCTGGAGGCCTCAATGATCGACCGG |
| | | AGAAACGGAGACAAGCCCATCACCTTTGAGGTCACCA |
| | | TAGGCAACTATGGGAACGAAGTTGATGGCCTGTCCCG |
| | | GCCCCAGCGGCCTCGGCCCCGGAAGGAGCCGGGGG |
| | | ATGAGGAAGAAGTAGACCTGATTCAGAACGCAAGTGA |
| | | TGACGAGGCCGGTGATGCCGGGGACCTGGCCTCAGT |
| | | CTCCTCCACTCCACCAATGCGGCCCCAGGTCACCGAC |
| | | AGGAACTACTTCCATCTGCCCTACCTGGAGCGAAAGC |
| | | CCTGCATCTACATCAAGAGCTGGTGGCCGGACCAGCG |
| | | CCGCCGCCTCTACAATGCCAACATCATGGACCACATT |
| | | GCCGACAAGCTGGAAGAAGGCCTGAACGACATACAGG |
| | | AGATGATCAAAACGGAGAAGTCCTACCCTGAGCGTCG |
| | | CCTGCGGGGCGTCCTGGAGGAGCTGAGCTGTGGCTG |
| | | CTGCCGCTTCCTCTCCCTCGCTGACAAGGACCAGGGC |
| | | CACTCATCCCGCACCAGGCTTGACCGGGAGCGCCTCA |
| | | AGTCCTGCATGAGGGAGCTGGTAAGTATCAAGGTTAC |
| | | AAGACAGGTTTAAGGAGACCAATAGAAACTGGGCTTG |
| | | TCGAGACAGAGAAGACTCTTGCGTTTCTGAGCTAGCC |
| | | CCCGGGTGCGCGGCGTCGGTGGTGCCGGGGGGGG |
| | | CGCCAGGTCGCAGGCGGTGTAGGGCTCCAGGCAGGC |
| | | GGCGAAGGCCATGACGTGCGCTATGAAGGTCTGCTCC |
| | | TGCACGCCGTGAACCAGGTGCGCCTGCGGGCCGCGC |
| | | GCGAACACCGCCACGTCCTCGCCTGCGTGGGTCTCTT |
| | | CGTCCAGGGGCACTGCTGACTGCTGCCGATACTCGG |
| | | GGCTCCCGCTCTCGCTCTCGGTAACATCCGGCCGGG |
| | | CGCCGTCCTTGAGCACATAGCCTGGACCGTTTCGTCG |
| | | ACCTCGAGTTAAGGGCGAATTCCCGATAAGGATOTTC |
| | | CTAGAGCATGGCTACGTAGATAAGTAGCATGGCGGGT |
| | | TAATCATTAACTACAAGGAACCCCTAGTGATGGAGTTG |
| | | GCCACTCCCTCTCTGCGCGCTCGCTCGCTCACTGAGG |
| | | CCGGGCGACCAAAGGTCGCCCGACGCCCGGGCTTTG |
| | | CCCGGGCGGCCTCAGTGAGCGAGCGAGCGCGCAGCC |
| | | TTAATTAACCTAATTCACTGGCCGTCGTTTTACAACGTC |
| | | GTGACTGGGAAAACCCTGGCGTTACCCAACTTAATCG |
| | | CCTTGCAGCACATCCCCCTTTCGCCAGCTGGCGTAAT |
| | | AGCGAAGAGGCCCGCACCGATCGCCCTTCCCAACAGT |
| | | TGCGCAGCCTGAATGGCGAATGGGACGCGCCCTGTA |
| | | GCGGCGCATTAAGCGCGGCGGGTGTGGTGGTTACGC |
| | | GCAGCGTGACCGCTACACTTGCCAGCGCCCTAGCGC |
| | | CCGCTCCTTTCGCTTTCTTCCCTTCCTTTCTCGCCACG |
| | | TTCGCCGGCTTTCCCCGTCAAGCTCTAAATCGGGGGC |
| | | TCCCTTTAGGGTTCCGATTTAGTGCTTTACGGCACCTC |
| | | GACCCCAAAAAACTTGATTAGGGTGATGGTTCACGTAG |
| | | TGGGCCATCGCCCTGATAGACGGTTTTTCGCCCTTTG |
| | | ACGTTGGAGTCCACGTTCTTTAATAGTGGACTCTTGTT |
| | | CCAAACTGGAACAACACTCAACCCTATCTCGGTCTATT |
| | | CTTTTGATTTATAAGGGATTTTGCCGATTTCGGCCTATT |
| | | GGTTAAAAAATGAGCTGATTTAACAAAAATTTAACGCG |
| | | AATTTTAACAAAATATTAACGCTTACAATTTAGGTGGCA |
| | | CTTTTCGGGGAAATGTGCGCGGAACCCCTATTTGTTTA |
| | | TTTTTCTAAATACATTCAAATATGTATCCGCTCATGAGA |
| | | CAATAACCCTGATAAATGCTTCAATAATATTGAAAAAGG |
| | | AAGAGTATGAGCCATATTCAACGGGAAACGTCGAGGC |
| | | CGCGATTAAATTCCAACATGGATGCTGATTTATATGGG |
| | | TATAAATGGGCTCGCGATAATGTCGGGCAATCAGGTG |
| | | CGACAATCTATCGCTTGTATGGGAAGCCCGATGCGCC |
| | | AGAGTTGTTTCTGAAACATGGCAAAGGTAGCGTTGCCA |
| | | ATGATGTTACAGATGAGATGGTCAGACTAAACTGGCTG |
| | | ACGGAATTTATGCCTCTTCCGACCATCAAGCATTTTAT |
| | | CCGTACTCCTGATGATGCATGGTTACTCACCACTGCGA |
| | | TCCCCGGAAAAACAGCATTCCAGGTATTAGAAGAATAT |
| | | CCTGATTCAGGTGAAAATATTGTTGATGCGCTGGCAGT |
| | | GTTCCTGCGCCGGTTGCATTCGATTCCTGTTTGTAATT |
| | | GTCCTTTTAACAGCGATCGCGTATTTCGTCTTGCTCAG |
| | | GCGCAATCACGAATGAATAACGGTTTGGTTGATGCGA |
| | | GTGATTTTGATGACGAGCGTAATGGCTGGCCTGTTGA |
| | | ACAAGTCTGGAAAGAAATGCATAAACTTTTGCCATTCT |
| | | CACCGGATTCAGTCGTCACTCATGGTGATTTCTCACTT |
| | | GATAACCTTATTTTTGACGAGGGGAAATTAATAGGTTG |
| | | TATTGATGTTGGACGAGTCGGAATCGCAGACCGATAC |
| | | CAGGATCTTGCCATCCTATGGAACTGCCTCGGTGAGT |
| | | TTTCTCCTTCATTACAGAAACGGCTTTTTCAAAAATATG |
| | | GTATTGATAATCCTGATATGAATAAATTGCAGTTTCATT |
| | | TGATGCTCGATGAGTTTTTCTAACTGTCAGACCAAGTT |
| | | TACTCATATATACTTTAGATTGATTTAAAACTTCATTTTT |
| | | AATTTAAAAGGATCTAGGTGAAGATCCTTTTTGATAATC |
| | | TCATGACCAAAATCCCTTAACGTGAGTTTTCGTTCCAC |
| | | TGAGCGTCAGACCCCGTAGAAAAGATCAAAGGATCTT |
| | | CTTGAGATCCTTTTTTTCTGCGCGTAATCTGCTGCTTG |
| | | CAAACAAAAAAACCACCGCTACCAGCGGTGGTTTGTTT |
| | | GCCGGATCAAGAGCTACCAACTCTTTTTCCGAAGGTAA |
| | | CTGGCTTCAGCAGAGCGCAGATACCAAATACTGTTCTT |
| | | CTAGTGTAGCCGTAGTTAGGCCACCACTTCAAGAACTC |
| | | TGTAGCACCGCCTACATACCTCGCTCTGCTAATCCTGT |
| | | TACCAGTGGCTGCTGCCAGTGGCGATAAGTCGTGTCT |
| | | TACCGGGTTGGACTCAAGACGATAGTTACCGGATAAG |
| | | GCGCAGCGGTCGGGCTGAACGGGGGGTTCGTGCACA |
| | | CAGCCCAGCTTGGAGCGAACGACCTACACCGAACTGA |
| | | GATACCTACAGCGTGAGCTATGAGAAAGCGCCACGCT |
| | | TCCCGAAGGGAGAAAGGCGGACAGGTATCCGGTAAG |
| | | CGGCAGGGTCGGAACAGGAGAGCGCACGAGGGAGCT |
| | | TCCAGGGGGAAACGCCTGGTATCTTTATAGTCCTGTC |
| | | GGGTTTCGCCACCTCTGACTTGAGCGTCGATTTTTGTG |
| | | ATGCTCGTCAGGGGGGCGGAGCCTATGGAAAAACGC |
| | | CAGCAACGCGGCCTTTTTACGGTTCCTGGCCTTTTGCT |
| | | GGCCTTTTGCTCACATGTTCTTTCCTGCGTTATCCCCT |
| | | GATTCTGTGGATAACCGTATTACCGCCTTTGAGTGAGC |
| | | TGATACCGCTCGCCGCAGCCGAACGACCGAGCGCAG |
| | | CGAGTCAGTGAGCGAGGAAGCGGAAGAGCGCCCAAT |
| | | ACGCAAACCGCCTCTCCCCGCGCGTTGGCCGATTCAT |
| | | TAATGCAGCTGGCACGACAGGTTTCCCGACTGGAAAG |
| | | CGGGCAGTGAGCGCAACGCAATTAATGTGAGTTAGCT |
| | | CACTCATTAGGCACCCCAGGCTTTACACTTTATGCTTC |
| | | CGGCTCGTATGTTGTGTGGAATTGTGAGCGGATAACA |
| | | ATTTCACACAGGAAACAGCTATGACCATGATTACGCCA |
| | | GATTTAATTAAGG |

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an N-terminal or C-terminal portion of an OTOF protein) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. For targeting cochlear hair cells, AAV1, AAV2, AAV6, AAV9, Anc80, Anc80L65, DJ/9, 7m8, and PHP.B may be particularly useful. Serotypes evolved for transduction of the retina may also be used in the methods and compositions described herein. The first and second nucleic acid vectors (e.g., AAV vectors) in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the use of AAV vectors for delivering a functional OTOF isoform 5 protein requires the use of a dual vector system, in in which the first member of the dual vector system encodes an N-terminal portion of an OTOF isoform 5 protein and the second member encodes a C-terminal portion of an OTOF isoform 5 protein such that, upon administration of the dual vector system to a cell, the polynucleotide sequences contained within the two vectors can join to form a single sequence that results in the production of a full-length OTOF isoform 5 protein.

In some embodiments, the first member of the dual vector system will also include, in 5′ to 3′ order, a first inverted terminal repeat (“ITR”); a promoter (e.g., a Myo15 promoter); a Kozak sequence; an N-terminal portion of an OTOF isoform 5 coding sequence; a splice donor sequence; an AP gene fragment (e.g., an AP head sequence); and a second ITR; and the second member of the dual vector system will include, in 5′ to 3′ order, a first ITR; an AP gene fragment (e.g., an AP head sequence); a splice acceptor sequence; a C-terminal portion of an OTOF isoform 5 coding sequence; a polyA sequence; and a second ITR. In some embodiments, the N-terminal portion of the OTOF isoform 5 coding sequence and the C-terminal portion of the OTOF isoform 5 coding sequence do not overlap and are joined in a cell (e.g., by recombination at the overlapping region (the AP gene fragment), or by concatemerization of the ITRs) to produce the full-length OTOF isoform 5 amino sequence as set forth in SEQ ID NO:1. In particular embodiments, the N-terminal portion of the OTOF isoform 5 coding sequence encodes amino acids 1-802 of SEQ ID NO:1 (SEQ ID NO: 58) and the C-terminal portion of the OTOF isoform 5 coding sequence encodes amino acids 803-1997 of SEQ ID NO:1 (SEQ ID NO: 59).

In some embodiments, the first member of the dual vector system includes the Myo15 promoter of SEQ ID NO:21 (also represented by nucleotides 235-1199 of SEQ ID NO:66) operably linked to nucleotides that encode the N-terminal 802 amino acids of the OTOF isoform 5 protein (amino acids 1-802 of SEQ ID NO:1), which are encoded by exons 1-20 of the native polynucleotide sequence encoding that protein. In certain embodiments, the nucleotide sequence that encodes the N-terminal amino acids of the OTOF isoform 5 protein is nucleotides 1222-3627 of SEQ ID NO:66. In some embodiments, the nucleotide sequence that encodes the N-terminal amino acids of the OTOF isoform 5 protein is any nucleotide sequence that, by redundancy of the genetic code, encodes amino acids 1-802 of SEQ ID NO:1. The nucleotide sequences that encode the OTOF isoform 5 protein can be partially or fully codon-optimized for expression. In some embodiments, the first member of the dual vector system includes the Kozak sequence corresponding to nucleotides 1216-1225 of SEQ ID NO:66. In some embodiments, the first member of the dual vector system includes the splice donor sequence corresponding to nucleotides 3628-3711 of SEQ ID NO:66. In some embodiments, the first member of the dual vector system includes the AP head sequence corresponding to nucleotides 3718-4004 of SEQ ID NO:66. In particular embodiments, the first member of the dual vector system includes nucleotides 235-4004 of SEQ ID NO:66 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO:66 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4098-4227 of SEQ ID NO:66 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 60, 62, 64, 65, 66, or 68), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4227 of SEQ ID NO:66.

In some embodiments, the second member of the dual vector system includes nucleotides that encode the C-terminal 1195 amino acids of the OTOF isoform 5 protein (amino acids 803-1997 of SEQ ID NO:1) immediately followed by a stop codon. In certain embodiments, the nucleotide sequence that encodes the C-terminal amino acids of the OTOF isoform 5 protein is nucleotides 587-4174 of SEQ ID NO:67. In some embodiments, the nucleotide sequence that encodes the C-terminal amino acids of the OTOF isoform 5 protein is any nucleotide sequence that, by redundancy of the genetic code, encodes amino acids 803-1997 of SEQ ID NO:1. The nucleotide sequences that encode the OTOF isoform 5 protein can be partially or fully codon-optimized for expression. In some embodiments, the second member of the dual vector system includes the splice acceptor sequence corresponding to nucleotides 538-586 of SEQ ID NO:67. In some embodiments, the second member of the dual vector system includes the AP head sequence corresponding to nucleotides 229-515 of SEQ ID NO:67. In some embodiments, the second member of the dual vector system includes the poly(A) sequence corresponding to nucleotides 4217-4438 of SEQ ID NO:67. In particular embodiments, the second member of the dual vector system includes nucleotides 229-4438 of SEQ ID NO:67 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO:67 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4526-4655 of SEQ ID NO:67 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., any of SEQ ID NOs: 61, 63, or 67), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-4655 of SEQ ID NO:67. In some embodiments, the dual vector system is an AAV1 dual vector system. In some embodiments, the dual vector system is an AAV9 dual vector system. In some embodiments, the dual vector system is an Anc80 dual vector system.

### Pharmaceutical Compositions

The AAV vectors described herein may be incorporated into a vehicle for administration into a patient, such as a human patient suffering from sensorineural hearing loss or auditory neuropathy, as described herein. Pharmaceutical compositions containing vectors, such as viral vectors, that contain a polynucleotide encoding a portion of an OTOF isoform 5 protein can be prepared using methods known in the art.

Mixtures of the AAV vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

In some embodiments, both vectors in the OTOF dual vector system (e.g., the first or 5′ vector and the second or 3′ vector) are mixed in a single formulation. The formulation may be a sterile, aqueous suspension. In some embodiments, both vectors are present in the formulation in an about 3:1 to about 1:3 ratio (e.g., 3:1 to 1:3, 2:1 to 1:2, or 1:1). In some embodiments, both vectors are present in the formulation in an approximately 1:1 ratio (vector genomes (vg)). For example, a suspension containing a titer of 4×10¹³ total vg/mL can contain an approximately equimolar ratio of 2×10¹³ vg/mL of the first (5′) vector and 2×10¹³ vg/mL of the second (3′) vector. In another example, a suspension containing a titer of 3×10¹³ total vg/mL can contain an approximately equimolar ratio of 1.5×10¹³ vg/mL of the first (5′) vector and 1.5×10¹³ vg/mL of the second (3′) vector, or a suspension containing a titer of 7.3×10¹³ total vg/mL can contain an approximately equimolar ratio of 3.65×10¹³ vg/mL of the first (5′) vector and 3.65×10¹³ vg/mL of the second (3′) vector. In some embodiments, in addition to the AAV vectors, the formulation contains 10 mM sodium phosphate or disodium phosphate, 180 mM sodium chloride, 5% (w/v) sucrose, and 0.001% (w/v) poloxamer 188 and has a target of pH 7.3-7.4. In some embodiments, in addition to the AAV vectors, the formulation contains 10 mM sodium phosphate, 180 mM sodium chloride, 5% (w/v) sucrose, and 0.001% (w/v) poloxamer 188 and has a pH of about 7.4. In some embodiments, in addition to the AAV vectors, the formulation contains 10 mM disodium phosphate, 180 mM sodium chloride, 5% (w/v) sucrose, and 0.001% (w/v) poloxamer 188 and has a pH of about 7.4. The formulation can be frozen prior to administration (e.g., at or below −65° C.). If needed, the formulation can be diluted to the desired titer on the day of administration using a diluent that contains the same excipients at the same target concentration (and lacks the AAV vectors).

### Methods of Treatment

The compositions described herein may be administered to a subject with sensorineural hearing loss or auditory neuropathy by local administration to the inner ear (e.g., administration into the perilymph or endolymph, e.g., by injection or catheter insertion through the round window membrane, injection into a semicircular canal, by canalostomy, or by intratympanic or transtympanic injection, e.g., administration to a cochlear hair cell). If the compositions are administered by direct delivery to the inner ear, a second fenestration or vent hole may be added elsewhere in the inner ear (e.g., in a semicircular canal). In some embodiments, the composition is administered as an intracochlear injection via a catheter placed through the round window membrane of the cochlea. The intracochlear injection may also utilize a syringe and syringe pump in combination with the catheter (e.g., a syringe is attached to the catheter and the syringe and catheter combination are loaded onto the syringe pump). For intracochlear injection, a site of egress will be created by fenestrating the lateral semicircular canal. The surgical approach may be similar to that used to place cochlear implants. In some embodiments, the intracochlear injection is at a rate of 0.8-1 mL/hr (e.g., 0.9 mL/hr). The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once (e.g., once per ear to be treated). In some embodiments, the first and second nucleic acid vectors (e.g., AAV vectors) are administered simultaneously (e.g., in a single composition). The first and second nucleic acid vector can have the same serotype or different serotypes (e.g., AAV serotypes). For example, the first and second nucleic acid vectors can both have an AAV1 capsid, an Anc80 capsid, an AAV2 capsid, or an AAV9 capsid.

The surgical approach and administration method described herein can also be used for the administration of other nucleic acid vectors (e.g., AAV vectors) to the inner ear (e.g., into the perilymph) of a human subject (e.g., for treatment of sensorineural hearing loss, such as genetic hearing loss or acquired hearing loss, or for the treatment of vestibular dysfunction, such as vertigo, dizziness, imbalance (e.g., loss of balance or a balance disorder), oscillopsia, or bilateral vestibulopathy). For example, the traditional surgical procedure for cochlear implant surgery can be used to access the middle ear, which involves a mastoidectomy and opening the facial recess. Openings can then be made in the round window membrane of the cochlea (e.g., for insertion of a catheter) and in a second location in the inner ear (e.g., a fenestration or vent hole to serve as a site of egress, such as a fenestration in a semicircular canal) and the nucleic acid vector can be infused into the perilymph via a catheter placed through the round window membrane. In some embodiments, the second opening (i.e., fenestration or vent hole) is made in the lateral semicircular canal. In some embodiments, the second opening (i.e., fenestration or vent hole) is made in the posterior semicircular canal. In some embodiments, the second opening (i.e., fenestration or vent hole) is made in the superior semicircular canal. A syringe and/or syringe pump can be used in combination with the catheter for the administration (e.g., a syringe is attached to the catheter and the syringe and catheter combination are loaded onto the syringe pump). The nucleic acid vector (e.g., a solution containing the nucleic acid vector) can be administered at a rate of 0.5-1 mL/hr (e.g., 0.6-1 mL/hr, 0.7-1 mL/hr, 0.8-1 mL/hr, or 0.9-1 mL/hour, such as 0.9 mL/hr). For the treatment of genetic hearing loss or vestibular dysfunction (i.e., hearing loss or vestibular dysfunction associated with a genetic mutation), the nucleic acid vector may contain a polynucleotide encoding a functional protein that is deficient in the subject (e.g., a polynucleotide encoding a functional (e.g., wild-type) protein that is absent or that does not function properly in the inner ear of the subject due to a genetic mutation associated with the hearing loss or vestibular dysfunction) or a polynucleotide encoding an expression product designed to correct a genetic mutation in the subject (e.g., correct a genetic mutation associated with the hearing loss or vestibular dysfunction via gene editing). For treatment of acquired hearing loss (e.g., noise-induced hearing loss, age-related hearing loss, disease or infection-related hearing loss, head trauma-related hearing loss, or ototoxic drug-induced hearing loss; e.g., hearing loss associated with damage to or loss of cochlear hair cells) the nucleic acid vector may contain a polynucleotide encoding an expression product that induces or increases cochlear hair cell regeneration, increases cochlear supporting cell numbers, prevents or reduces cochlear hair cell damage, prevents or reduces cochlear hair cell death, increases cochlear hair cell maturation, improves cochlear hair cell function, or promotes or increases cochlear hair cell survival. For treatment of vestibular dysfunction associated with damage to or loss of vestibular cells (e.g., damage to or loss of vestibular cells, such as vestibular hair cells or supporting cells, related to disease or infection, head trauma, ototoxic drugs (e.g., vestibulotoxic drugs), or aging) the nucleic acid vector may contain a polynucleotide encoding an expression product that induces or increases vestibular hair cell regeneration, increases vestibular supporting cell numbers, prevents or reduces vestibular hair cell damage, prevents or reduces vestibular hair cell death, increases vestibular hair cell maturation, improves vestibular hair cell function, or promotes or increases vestibular hair cell survival.

Subjects that may be treated as described herein are subjects having or at risk of developing sensorineural hearing loss or auditory neuropathy. The compositions and methods described herein can be used to treat subjects having a mutation in OTOF (e.g., a mutation that reduces OTOF function or expression, or an OTOF mutation associated with sensorineural hearing loss), subjects having a family history of autosomal recessive sensorineural hearing loss or deafness (e.g., a family history of OTOF-related hearing loss), or subjects whose OTOF mutational status and/or OTOF activity level is unknown. In some embodiments, a subject to be treated as described herein has or has been diagnosed as having a biallelic mutation in OTOF (e.g., a likely pathogenic or pathogenic mutation). The methods described herein may include a step of screening a subject for a mutation in OTOF (e.g., a biallelic likely pathogenic or pathogenic mutation) prior to treatment with or administration of the compositions described herein. In some embodiments, prior to treatment with a composition described herein, the subject is also screened for mutations in other hearing loss-related genes. A subject can be screened for an OTOF mutation or a mutation in another hearing loss-related gene using standard methods known to those of skill in the art (e.g., genetic testing).

Subjects that may be treated as described herein include those for whom it has been determined that minimal benefit has been provided by amplification of the ear to be treated with the OTOF dual vector system. In some embodiments, the subject is deemed to meet the criteria for cochlear implantation in one or both ears prior to treatment initiation. In some embodiments, the subject has not previously received a cochlear implant in the ear(s) to be treated with an OTOF dual vector system. In some embodiments, the subject is diagnosed as having profound sensorineural hearing loss prior to treatment. The subject may be diagnosed has having profound sensorineural hearing loss based on behavioral and physiologic measurements of inner ear function, such as having an absence of an ABR neural signal in response to a click stimulus at or below 85 decibels normalized Hearing Level (dB nHL) in the ear(s) to be treated with an OTOF dual vector system or having ≥90 dB HL as assessed by ABR. In some embodiments, outer hair cell presence is confirmed prior to treatment based on detectable otoacoustic emissions (OAEs). For example, the subject may have distortion product otoacoustic emission (DPOAE) present with ≥6 dB signal-to-noise ratio at ≥3 frequencies in a distortion product (DP) Gram measured from 1 to 8 kHz in the ear(s) to be treated with an OTOF dual vector system. In some embodiments, outer hair cell presence is confirmed via presence of the cochlear microphonic in the ear(s) to be treated with an OTOF dual vector system (e.g., in subjects older than 24 months of age). In some embodiments, the subject has behavioral open-set word detection scores of <30% in the ear(s) to be treated with an OTOF dual vector system or has behavioral open-set word detection scores consistent with the speech criteria from the recommended cochlear implant label. In some embodiments, the subject has no evidence from measures of hearing loss that show dependence on body temperature. In some embodiments, the subject does not have a history or presence of other permanent or untreatable hearing loss conditions. In some embodiments, the subject's inner ear anatomy is compatible with the route of administration (e.g., for intracochlear injection, the subject does not have an enlarged cochlear or vestibular aqueduct). In some embodiments, the subject does not have a history or presence of treatment with ototoxic drugs. In some embodiments, the subject does not have a history of risk factor(s) for auditory neuropathy not caused by biallelic OTOF mutations (e.g., prematurity, low birth weight, hyperbilirubinemia, neonatal intensive care unit admission, and/or low Apgar scores).

In some embodiments, the subject to be treated with an OTOF dual vector system is a pediatric subject (a subject under 18 years of age). In some embodiments, the pediatric subject is at least 7 years of age (i.e., at least 7 years of age and under 18 years of age). In some embodiments, the subject is older than 24 months of age and under 18 years of age. In some embodiments, the subject is older than 24 months of age and younger than 7 years of age. In some embodiments, the subject is 24 months of age or younger (e.g., 24 months of age or younger, 18 months of age or younger, 12 months of age or younger, or 6 months of age or younger).

The methods described herein may also include a step of assessing hearing in a subject prior to treatment with or administration of the compositions described herein. Hearing can be assessed using standard tests, such as audiometry, ABR, electrocochleography (ECOG), and otoacoustic emissions. In some embodiments, the method further includes performing ABR measurements prior to treatment and after treatment initiation (e.g., prior to treatment and 1 day, 1 week, 2 weeks, 4 weeks, 6 weeks, 12 weeks, 24 weeks, 36 weeks, 48 weeks, one year, 72 weeks, 2 years, 3 years, 4 years, and/or 5 years after administration of an OTOF dual vector system). In some embodiments, the method further includes performing behavioral audiometry prior to treatment and after treatment initiation (e.g., prior to treatment and 1 day, 1 week, 2 weeks, 4 weeks, 6 weeks, 12 weeks, 24 weeks, 36 weeks, 48 weeks, one year, 72 weeks, 2 years, 3 years, 4 years, and/or 5 years after administration of an OTOF dual vector system), such as SAT/Speech Detection Threshold (SDT) audiometry, SRT/Speech Reception Threshold (SRT) audiometry, Pure tone air- and bone-conduction audiometry, visual reinforcement audiometry (VRA), and/or conditioned play audiometry (CPA). In some embodiments, the method further includes performing one or more assessments or questionnaires prior to treatment and after treatment initiation (e.g., prior to treatment and 1 day, 1 week, 2 weeks, 4 weeks, 6 weeks, 12 weeks, 24 weeks, 36 weeks, 48 weeks, one year, 72 weeks, 2 years, 3 years, 4 years, and/or 5 years after administration of an OTOF dual vector system), such as the Auditory Skills Checklist (ASC), Open & Closed (O&C) set task, Early Speech Perception (ESP) test, Pediatric Speech Intelligibility (PSI) test, Lexical Neighborhood Test (LNT)/Multisyllabic Lexical Neighborhood Test (MLNT), Consonant-Nucleus-Consonant (CNC) test, Bamford-Kowal-Bench (BKB) sentence test, LittIEARS® Auditory Questionnaire, MacArthur-Bates Communicative Development Inventories (MB-CDI) Words and Gestures, Quality of Life-Cochlear Implant (QoL-CI), Pediatric Quality of Life Inventory (PedsQL™), Hearing Environments and Reflection on Quality of Life (HEAR-QL)-26, HEAR-QL-28, The Health Utilities Index 3 (HUI-3), Vanderbilt Fatigue Scales (VFS), or AzBio test (e.g., a pediatric AzBio test). The method may also include performing hearing assessments (e.g., DPOAE assessment, otoscopy, and tympanometry) and/or vestibular assessments (e.g., cervical vestibular-evoked myogenic potential, dizziness handicap inventory, pediatric vestibular symptom questionnaire, age and stages questionnaire, and video head impulse test) prior to treatment and/or after treatment initiation (e.g., prior to treatment and 1 day, 1 week, 2 weeks, 4 weeks, 6 weeks, 12 weeks, 24 weeks, 36 weeks, 48 weeks, one year, 72 weeks, 2 years, 3 years, 4 years, and/or 5 years after administration of an OTOF dual vector system). The compositions and methods described herein may also be administered as a preventative treatment to patients at risk of developing hearing loss or auditory neuropathy, e.g., patients who have a family history of inherited hearing loss or patients carrying an OTOF mutation who do not yet exhibit hearing loss or impairment.

Treatment may include administration of a composition containing the nucleic acid vectors (e.g., AAV vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the cochlea. In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the AAV vectors may have a titer of, for example, from about 1×10¹³ vector genomes (vg)/mL to about 1×10¹⁴ vg/mL (e.g., 1×10¹³ vg/mL, 2×10¹³ VG/mL, 3×10¹³ vg/mL, 4×10¹³ vg/mL, 5×10¹³ VG/mL, 6×10¹³ vg/mL, 7×10¹³ vg/mL, 8×10¹³ vg/mL, 9×10¹³ vg/mL, 1×10¹⁴ vg/mL, e.g., 1.0×10¹³ vg/mL to 5.0×10¹³ vg/mL, 2.0×10¹³ vg/mL to 5.0×10¹³ vg/mL, 3.0×10¹³ vg/mL to 5.0×10¹³ vg/mL, 4.0×10¹³ vg/mL to 5.0×10¹³ vg/mL, 5.0×10¹³ vg/mL to 1.0×10¹⁴ vg/mL, 5.0×10¹³ vg/mL to 9.0×10¹³ vg/mL, 5.0×10¹³ vg/mL to 8.0×10¹³ vg/mL, 5.0×10¹³ vg/mL to 7.0×10¹³ vg/mL, or 5.0×10¹³ vg/mL to 6.0×10¹³ vg/mL) in a volume of 200 μL to 250 μL (e.g., 200 μL, 210 μL, 220 μL, 230 μL, 240 μL, or 250 μL, e.g., 200-225 μL, 225-250 UL, 200-210 μL, 210-220 UL, 220-230 μL, 230-240 μL, or 240-250 μL). In some embodiments, the AAV vectors have a titer of about 3×10¹³ vg/mL to about 8×10¹³ vg/mL (e.g., 3.0×10¹³ vg/mL to 8.0×10¹³ vg/mL, 3.0×10¹³ vg/mL to 7.5×10¹³ vg/mL, 3.0×10¹³ vg/mL to 7.0×10¹³ vg/mL, 3.0×10¹³ vg/mL to 6.5×10¹³ vg/mL, 3.0×10¹³ vg/mL to 6.0×10¹³ vg/mL, 3.0×10¹³ vg/mL to 5.5×10¹³ vg/mL, 3.0×10¹³ vg/mL to 5.0×10¹³ vg/mL, 3.0×10¹³ vg/mL to 4.5×10¹³ vg/mL, 3.0×10¹³ vg/mL to 4.0×10¹³ vg/mL, 3.5×10¹³ vg/mL to 8.0×10¹³ vg/mL, 4.0×10¹³ vg/mL to 8.0×10¹³ vg/mL, 4.5×10¹³ vg/mL to 8.0×10¹³ vg/mL, 5.0×10¹³ vg/mL to 8.0×10¹³ vg/mL, 5.5×10¹³ vg/mL to 8.0×10¹³ vg/mL, 6.0×10¹³ vg/mL to 8.0×10¹³ vg/mL, 6.5×10¹³ vg/mL to 8.0×10¹³ vg/mL, or 7.0×10¹³ vg/mL to 8.0×10¹³ vg/mL) in a volume of 225 μL to 250 μL (e.g., 225 μL, 230 μL, 235 μL, 240 μL, 245 μL, or 250 μL, e.g., 225-235 μL, 230-240 μL, 235-245 μL, or 240-250 μL). In some embodiments, the AAV vectors have a titer of about 3×10¹³ VG/mL or 7.3×10¹³ VG/mL in a volume of 240 L. The AAV vectors may be administered to the subject at a dose of about 2×10¹² VG/ear to about 2.5×10¹³ VG/ear (e.g., 2×10¹² VG/ear to 5×10¹² VG/ear, 5×10¹² VG/ear to 8×10¹² VG/ear, 7×10¹² VG/ear to 2×10¹³ VG/ear, 8×10¹² VG/ear to 2.5×10¹³ VG/ear, e.g., 2×10¹² VG/ear, 2.25×10¹² VG/ear, 2.5×10¹² VG/ear, 2.75×10¹² VG/ear, 3×10¹² VG/ear, 3.25×10¹² VG/ear, 3.5×10¹² VG/ear, 3.75×10¹² VG/ear, 4×10¹² VG/ear, 4.25×10¹² VG/ear, 4.5×10¹² VG/ear, 4.75×10¹² VG/ear, 5×10¹² VG/ear, 5.25×10¹² VG/ear, 5.5×10¹² VG/ear, 5.75×10¹² VG/ear, 6×10¹² VG/ear, 6.25×10¹² VG/ear, 6.5×10¹² VG/ear, 6.75×10¹² VG/ear, 7×10¹² VG/ear, 7.2×10¹² VG/ear, 7.5×10¹² VG/ear, 7.75×10¹² VG/ear, 8×10¹² VG/ear, 8.25×10¹² VG/ear, 8.5×10¹² VG/ear, 8.75×10¹² VG/ear, 9×10¹² VG/ear, 9.25×10¹² VG/ear, 9.5×10¹² VG/ear, 9.75×10¹² VG/ear, 1×10¹³ VG/ear, 1.25×10¹³ VG/ear, 1.5×10¹³ VG/ear, 1.75×10¹³ VG/ear, 2×10¹³ VG/ear, 2.25×10¹³ VG/ear, or 2.5×10¹³ VG/ear). In some embodiments, the AAV vectors are administered to the subject at a dose of about 7.2×10¹² VG/ear to about 1.75×10¹³ VG/ear (e.g., a dose of 7.2×10¹² VG/ear or 1.75×10¹³ VG/ear).

The methods described herein may also include a step of administering antibiotics for prophylaxis for post-operative infection (e.g., before administration of the OTOF dual vector system) and/or administering a corticosteroid as prophylaxis against inflammatory and immunological responses (e.g., after administration of the OTOF dual vector system). For example, the day after administration of the dual vector system, a subject may begin a four-week treatment with a corticosteroid (e.g., prednisone or equivalent). In one embodiment, the subject may receive 1 mg/kg corticosteroid daily for the first two weeks post-treatment, 0.5 mg/kg corticosteroid daily for the third week post-treatment, and 0.25 mg/kg daily for the fourth week post-treatment. The total dose of corticosteroid is not to exceed 60 mg/day and may be weight adjusted as needed. If a pre-operative corticosteroid is administered (IV) as part of standard of care for cochlear implant surgery, the prophylactic corticosteroid dose on Day 0 may be adjusted so the daily corticosteroid dose does not exceed 1 mg/kg of prednisone equivalent dose; the Day 0 prophylactic corticosteroid may not be required if the pre-operative corticosteroid dose is ≥1 mg/kg of prednisone equivalent dose. If a pre-operative corticosteroid is not administered, the initial prophylactic corticosteroid dose may be spaced appropriately after the surgery and may be initiated the night before surgery.

The compositions described herein are administered in an amount sufficient to improve hearing, increase WT OTOF expression (e.g., expression of OTOF isoform 5 in a cochlear hair cell, e.g., an inner hair cell), or increase OTOF function. In some embodiments, the methods described herein instate hearing (e.g., instate more natural hearing and a more natural developmental trajectory) in profoundly deaf pediatric subjects by restoring the natural physiology of IHC transduction. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech. In some embodiments, the methods described herein result in a change in intensity threshold (decibels normalized hearing level (dB nHL)) across frequency domains as assessed using ABR (e.g., a change indicating improved hearing). In some embodiments, the methods described herein result in a change in intensity thresholds (decibel hearing level (dB HL)) across frequency domains and/or a change in speech awareness threshold (SAT) in the treated ear(s) (e.g., changes indicating improved hearing, e.g., assessed using behavioral audiometry such as SAT/SDT audiometry, SRT audiometry, pure tone air- and bone-conduction audiometry, VRA, or CPA). In some embodiments, methods described herein result in a change (e.g., an improvement) in score in one or more assessments or questionnaires after OTOF dual vector administration, such as a change (e.g., a change indicative of improved hearing) in score in the ASC, O & C set task, ESP test, PSI test, LNT/MLNT, CNC test, BKB sentence test, LittIEARS® Auditory Questionnaire, MB-CDI Words and Gestures, QoL-CI, PedsQL™, HEAR-QL-26, HEAR-QL-28, HUI-3, VFS, or AzBio test (e.g., a pediatric AzBio test). The compositions described herein may also be administered in an amount sufficient to slow or prevent the development or progression of sensorineural hearing loss or auditory neuropathy (e.g., in subjects who carry a mutation in OTOF or have a family history of autosomal recessive hearing loss but do not exhibit hearing impairment, or in subjects exhibiting mild to moderate hearing loss). OTOF expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to OTOF expression prior to administration of the compositions described herein. OTOF expression may also be assessed by detecting antibodies to the therapeutic OTOF transgene in serum from treated subjects. OTOF function may be evaluated directly (e.g., using electrophysiological methods or imaging methods to assess exocytosis) or indirectly based on hearing tests, and may be increased by 5% or more (e.g., 5%, 10%, 15%, 20%, 30%, 40%, 50%, 60%, 70%, 80%, 90%, 100% or more) compared to OTOF function prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 12 weeks, 15 weeks, 20 weeks, 24 weeks, 25 weeks, 36 weeks, 48 weeks, one year, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating sensorineural hearing loss or auditory neuropathy (e.g., hearing loss associated with a biallelic mutation in OTOF). Compositions may include nucleic acid vectors (e.g., AAV vectors) described herein (e.g., a first nucleic acid vector containing a polynucleotide that encodes and N-terminal portion of an OTOF isoform 5 protein and a second nucleic acid vector containing a polynucleotide that encodes a C-terminal portion of an OTOF isoform 5 protein), optionally packaged in an AAV virus capsid (e.g., an AAV1 capsid, an Anc80 capsid, an AAV2 capsid, or an AAV9 capsid). The kit can further include a package insert that instructs a user of the kit, such as a physician, to perform the methods described herein. The kit may optionally include a syringe, a syringe pump, a catheter, or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Administration of a Composition Containing a Dual Hybrid Vector System that Expresses OTOF Isoform 5 to a Subject Under Two Years of Age with Profound Genetic Hearing Loss

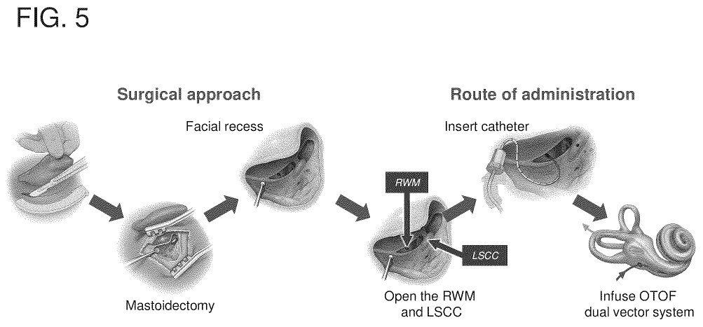

An eight-month-old female subject with profound congenital bilateral hearing loss and auditory neuropathy with present OAEs (i.e., the subject was identified as having detectable otoacoustic emissions) and biallelic mutations in the OTOF gene (pathogenic OTOF variant c.2887C>T (p.Arg963Ter)/c.2676+1G>T) was screened, qualified, and enrolled in a Phase 1/2 study. The subject was 10 months old at dosing and received an intracochlear injection of a composition containing an OTOF dual hybrid vector system in one ear. The composition contained a first AAV1 vector containing a Myo15 promoter (SEQ ID NO: 21) operably linked to exons 1-20 of a polynucleotide encoding a human OTOF isoform 5 protein (SEQ ID NO: 56), a splice donor sequence 3′ of the polynucleotide sequence, and an AP recombinogenic region (SEQ ID NO: 51) 3′ of the splice donor sequence, and a second AAV1 vector containing an AP recombinogenic region (SEQ ID NO: 51), a splice acceptor sequence 3′ of the recombinogenic region, a polynucleotide 3′ of the splice acceptor sequence that contains exons 21-45 and 47 of a polynucleotide encoding a human OTOF isoform 5 protein (SEQ ID NO: 57), and a bGH poly(A) sequence. The composition containing the OTOF dual hybrid AAV vector system was administered by a single, unilateral intracochlear injection in the right ear at an amount of 7.2×10¹² VG (3×10¹³ vg/mL in a volume of 240 μL). The surgical approach used to access the middle ear was similar to that used for cochlear implant surgery and involved a mastoidectomy and opening the facial recess. The composition was administered via insertion of a catheter through the round window membrane into the inner ear perilymph for direct infusion. A fenestration was also made in the lateral semicircular canal (FIG. 5). A cochlear implant was placed in the contralateral (left) ear during the same procedure. The subject received intraoperative antibiotics. The subject also received perioperative steroids and oral steroids (1 mg/kg/d) for two weeks that were tapered over two weeks. The perioperative period was uneventful, and the subject was discharged on day 1 post-operation. Following administration of the composition containing the OTOF dual hybrid vector system, the subject was assessed for safety and tolerability (including adverse events (AEs) and effects on balance and vestibular function (assessed by cervical vestibular evoked myogenic potential)) and efficacy (pure tone audiometry, auditory brainstem response, and hearing questionnaires) from Baseline (prior to dosing) to Week 12.

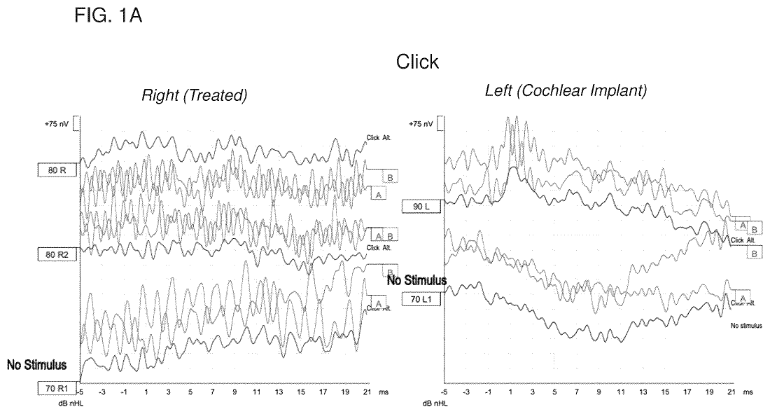

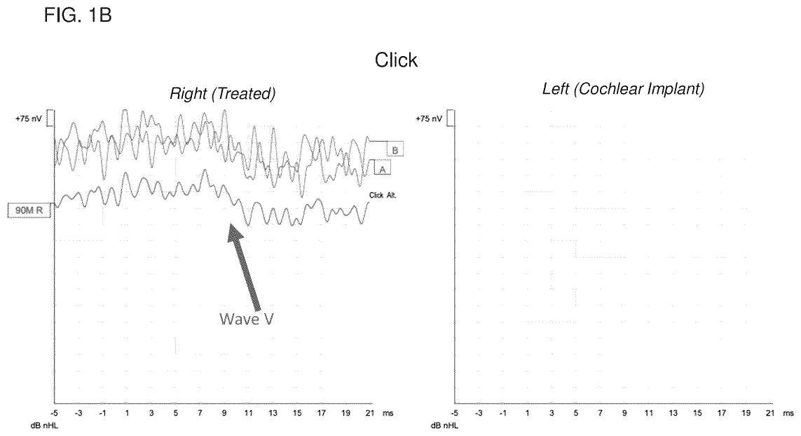

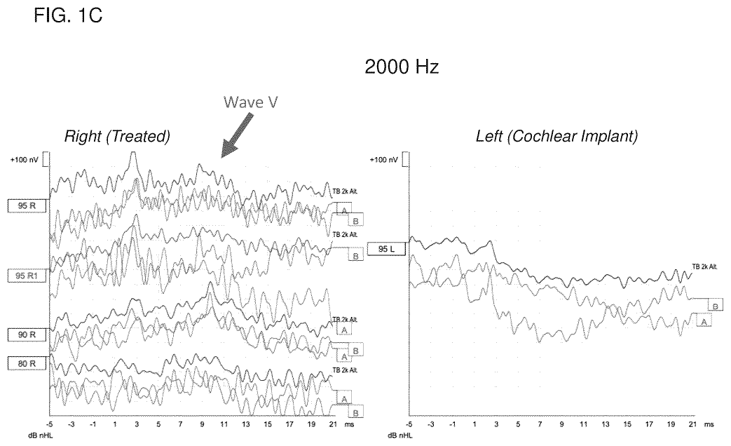

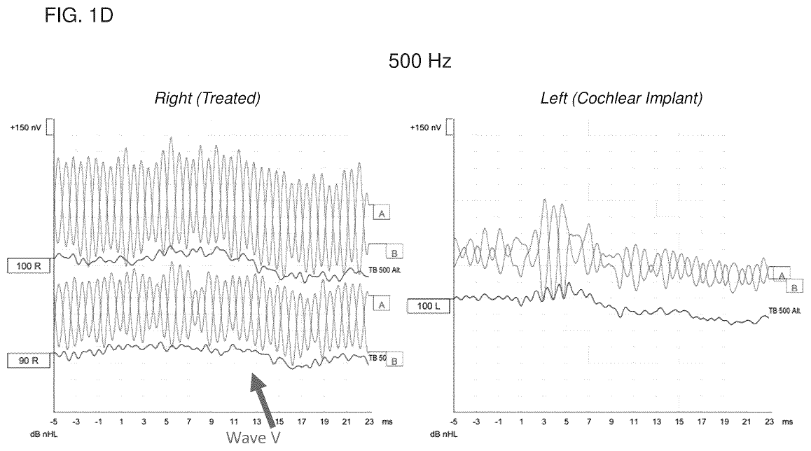

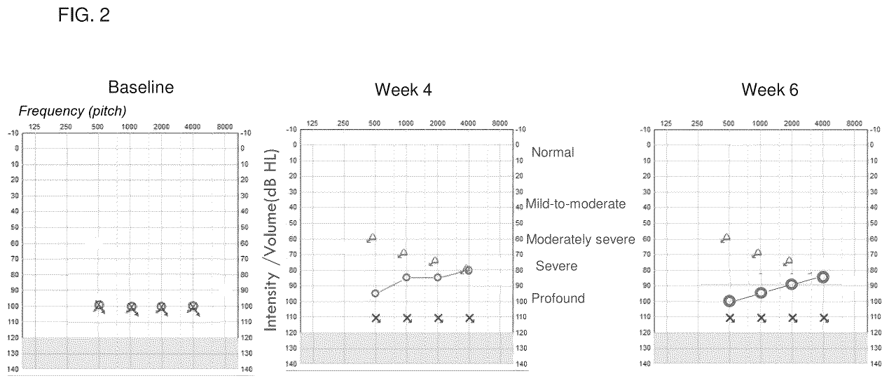

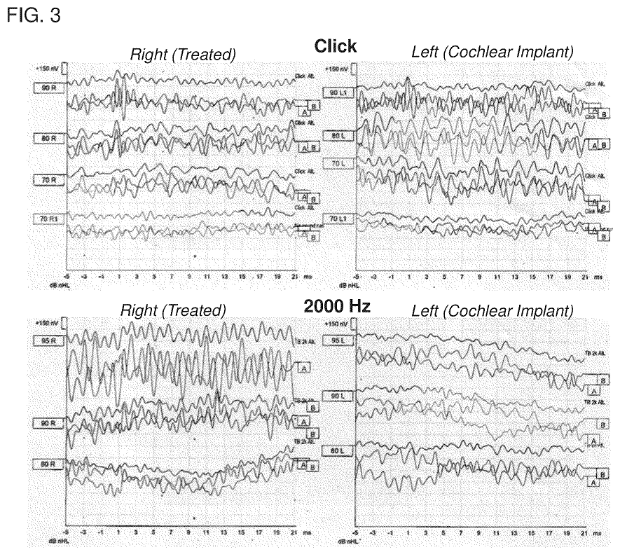

At planned follow-ups, the subject experienced improvements in auditory responses through Week 6 compared to Baseline, per auditory brainstem response (ABR) and behavioral (pure tone) audiometry. ABR, a clinically accepted physiologic measure of hearing sensitivity, is often absent in those with classic otoferlin-related hearing loss and was absent in both ears of the subject at Baseline. The subject also had no detectable hearing by behavioral pure tone audiogram at Baseline. At Week 6, ABR showed a response between 80-90 dB in the treated ear in an incomplete test (the subject woke up while the test was ongoing) despite the presence of some electric interference. Behavioral testing at Week 6 showed similar results consistent with Week 4 showing thresholds in the 80 dB range; however, the results are considered less reliable than the previous results due to lack of cooperation of the subject, potential learning effect or fatigue from repeated testing, and an ongoing upper airway infection. The subject's parents reported possible response to vocalization and clear response to loud sounds in keeping with Week 4. DPOAEs (a measure of outer hair cell function) were similar to Week 4 but testing conditions were not ideal (abnormal tympanogram due to subject's nasal congestion). Results from Week 4 and Week 6 are early indicators of a positive auditory response. There were no concerning safety signals through Week 6 following treatment and notably no treatment-emergent adverse events related to the OTOF dual hybrid vector system. ABR and behavioral (pure tone) audiometry results are shown in FIGS. 1A-1D, FIG. 2, and FIG. 3. Behavioral audiometry results are also provided below in Table 5.

TABLE 5
Audiogram (behavioral pure tone testing)
Baseline

| Right Ear - Audiogram (air) | | | | | | Left Ear - Audiogram | | | |
|---|---|---|---|---|---|---|---|---|---|
| | | 500 | 1000 | 2000 | 4000 | 500 | 1000 | 2000 | 4000 |
| | 250 | Hz | Hz | Hz | Hz | Hz | Hz | Hz | Hz |
| Threshold (dBHL)- Air | | NR | NR | NR | NR | | | | |
| Max stimulation (air) | | 100 | 100 | 100 | 95 | | | | |
| Bone | | | | | | | | | |

| Right Ear - Audiogram (air) | | | | | Left Ear - Audiogram | | | |
|---|---|---|---|---|---|---|---|---|
| | 500 | 1000 | 2000 | 4000 | 500 | 1000 | 2000 | 4000 |
| | Hz | Hz | Hz | Hz | Hz | Hz | Hz | Hz |

Week 4

| | | | | | | | | |
|---|---|---|---|---|---|---|---|---|
| Threshold | 95 | 85 | 85 | 80 | NR110 | NR110 | NR110 | NR110 |
| (dBHL)- | | | | | | | | |
| Air | | | | | | | | |
| Bone | | | | | | | | |

Week 6

| | | | | | | | | |
|---|---|---|---|---|---|---|---|---|
| Threshold | 100 | 95 | 90 | 85 | NR 110 | NR 110 | NR 110 | NR 110 |
| (dBHL)- | | | | | | | | |
| Air | | | | | | | | |
| Bone | NR 60 | NR 70 | NR 75 | NR 80 | DNT | DNT | DNT | DNT |

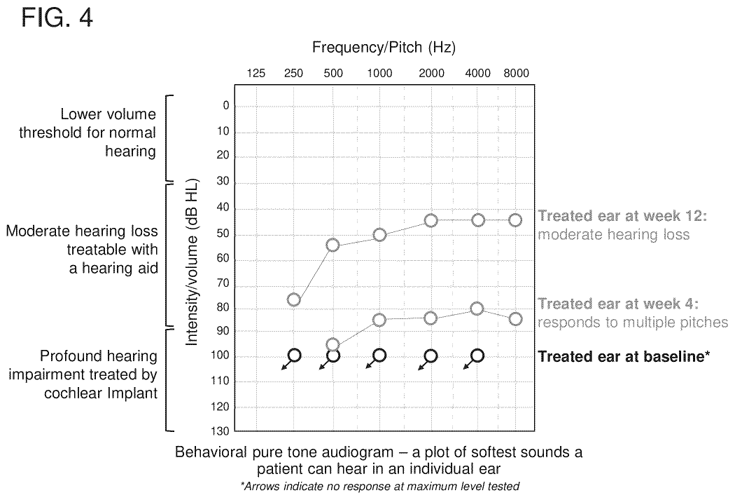

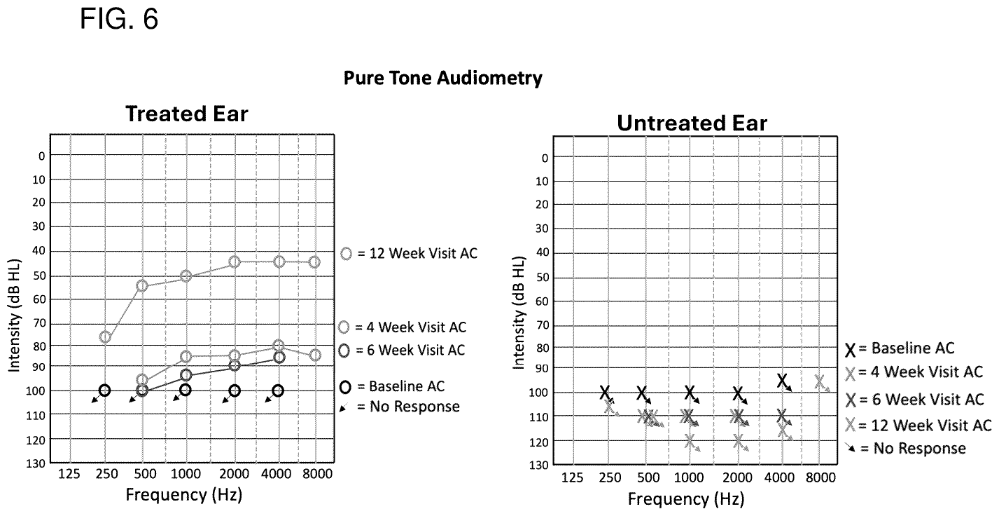

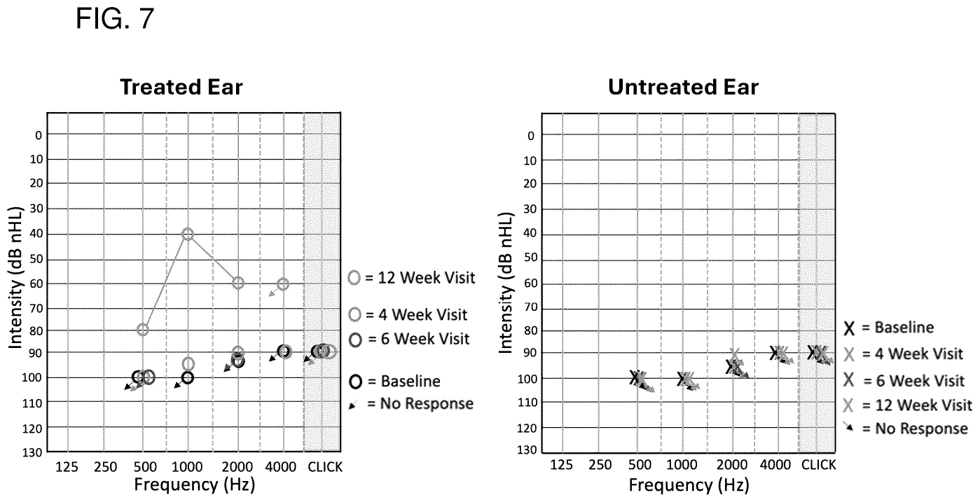

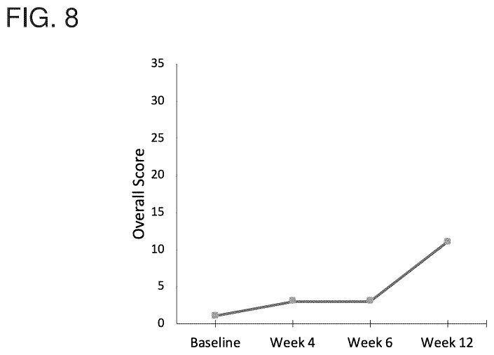

The subject was evaluated again at Week 12 and continued to show improvements in auditory responses compared to Baseline. Results from a behavioral pure tone audiogram are shown in FIG. 4. As shown in FIG. 4, the subject had profound hearing impairment at Baseline in the treated ear. At Week 4, the treated ear responded to multiple pitches. At Week 12, the treated ear exhibited responses in the range of moderate sensorineural hearing loss, indicating that the subject's hearing had markedly improved from initial profound sensorineural hearing loss. At Week 12 from Baseline, an improvement of 55 dB HL in hearing thresholds with air conduction versus Baseline (absent thresholds at 100 dB HL, maximum air conduction intensity tested, 250-4000 Hz) was observed by pure tone audiometry in the treated ear (FIG. 6). No improvement was seen in the untreated ear with cochlear implant with the cochlear implant turned off (FIG. 6). A positive ABR wave V amplitude response (thresholds of 40-80 dB, no response was elicited at 100 dB at Baseline) at Week 12 was also reported in the treated ear (FIG. 7). No improvement in hearing intensity was observed in the untreated ear when compared with the treated ear (FIG. 7). An improvement in the subject's global auditory skill development was observed at Week 12 from Baseline, according to parental reports and as indicated by the LittIEARS auditory questionnaire outcomes (FIG. 8). This is in line with normative data for patients with cochlear implants. The development of auditory skills was also observed, and parents reported more natural vocalizations (subject's voice less “screechy”) when the cochlear implant was turned off. The parents and investigator indicated that the subject could hear sounds when the opposite ear cochlear implant was turned off, indicating improved hearing acuity in the treated ear.

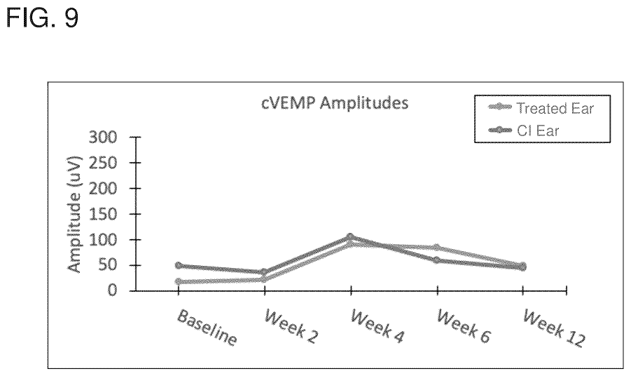

Through Week 12 after treatment, the study drug was well-tolerated and no dose-limiting toxicities and no adverse events (AEs) related to the OTOF dual hybrid vector system were reported, including absence of vestibular manifestations. Seven Grade 1 AEs were reported after dosing (upper respiratory tract infection, hand/foot/mouth rash, white blood cells in urine, reduced distortion product otoacoustic emissions (considered not related to study drug), vomiting, and elevated alanine transaminase), all of which were unrelated to treatment with the OTOF dual hybrid vector system. One serious AE (possible mastoiditis in ear with cochlear implant; Grade 2) was also reported and was considered unrelated to the OTOF dual hybrid vector system. Cervical vestibular evoked myogenic potential (cVEMP) responses were present and were similar to Baseline (FIG. 9). No observable nystagmus or overt motor behavioral changes were reported.

### Example 2. Administration of a Composition Containing Dual Hybrid Vectors that Express OTOF Isoform 5 to a Subject with Sensorineural Hearing Loss

According to the methods disclosed herein, a physician of skill in the art can treat a patient, such as a human patient, with sensorineural hearing loss (e.g., sensorineural hearing loss associated with a biallelic mutation in OTOF) so as to improve or restore hearing. In one example, a physician of skill in the art can administer to the human patient a composition containing a first AAV vector (e.g., an AAV1 vector) containing a Myo15 promoter (e.g., SEQ ID NO: 19, 21, 22, 31, or 32) operably linked to exons 1-20 of a polynucleotide encoding an OTOF isoform 5 protein (e.g., human OTOF isoform 5, e.g., SEQ ID NO: 1, e.g., a polynucleotide having the sequence of SEQ ID NO: 56), a splice donor sequence 3′ of the polynucleotide sequence, and an AP recombinogenic region (e.g., an AP gene fragment, any one of SEQ ID NOs: 48-53, e.g., SEQ ID NO: 51) 3′ of the splice donor sequence, and a second AAV vector (e.g., an AAV1 vector) containing an AP recombinogenic region (an AP gene fragment, any one of SEQ ID NOs: 48-53, e.g., SEQ ID NO: 51), a splice acceptor sequence 3′ of the recombinogenic region, a polynucleotide 3′ of the splice acceptor sequence that contains exons 21-45 and 47 of a polynucleotide encoding an OTOF isoform 5 protein (e.g., human OTOF isoform 5, e.g., SEQ ID NO: 1, e.g., a polynucleotide having the sequence of SEQ ID NO: 57), and a bGH poly(A) sequence. The composition containing the dual hybrid AAV vectors may be administered to the patient, for example, by intracochlear injection of an amount of 1×10¹³ vg/mL to 1×10¹⁴ vg/mL of the OTOF dual vector system in a volume of 200-250 μL (e.g., an amount of 3×10¹³ vg/mL or 7.3×10¹³ vg/mL in a volume of 240 μL), to treat sensorineural hearing loss.

Following administration of the composition to a patient, a practitioner of skill in the art can monitor the expression of OTOF, and the patient's improvement in response to the therapy, by a variety of methods. For example, a physician can monitor the patient's hearing by performing standard tests, such as audiometry, ABR, and measuring otoacoustic emissions following administration of the composition. A finding that the patient exhibits improved hearing (e.g., improved ABR and/or pure tone audiometry) in one or more of the tests following administration of the composition compared to hearing test results prior to administration of the composition indicates that the patient is responding favorably to the treatment.

Exemplary embodiments of the invention are described in the enumerated paragraphs below.
E1. A method of treating a human subject having biallelic otoferlin (OTOF) mutations, the method comprising administering to an inner ear of the subject an amount of 1×10¹³ vg/mL to 1×10¹⁴ vg/mL of an OTOF dual vector system in a volume of 200-250 μL by intracochlear injection, wherein the OTOF dual vector system comprises:
   a first adeno-associated virus (AAV) vector comprising a Myo15 promoter operably linked to a first coding polynucleotide that encodes an N-terminal portion of an Otoferlin (OTOF) isoform 5 protein, a splice donor sequence positioned 3′ of the first coding polynucleotide, and a first recombinogenic region positioned 3′ of the splice donor sequence; and a second AAV vector comprising a second recombinogenic region, a splice acceptor sequence positioned 3′ of the second recombinogenic region, a second coding polynucleotide that encodes a C-terminal portion of the OTOF isoform 5 protein positioned 3′ of the splice acceptor sequence, and a poly(A) sequence positioned 3′ of the second coding polynucleotide;
   wherein the first coding polynucleotide and the second coding polynucleotide that encode the OTOF isoform 5 protein do not overlap, wherein neither the first nor second AAV vector encodes the full-length OTOF isoform 5 protein, and wherein the first AAV vector and the second AAV vector are administered at a ratio of about 3:1 to about 1:3 (e.g., 3:1 to 1:3, 2:1 to 1:2, or 1:1).
E2. The method of E1, wherein the first AAV vector and the second AAV vector comprise an AAV1 capsid.
E3. The method of E1 or E2, wherein the Myo15 promoter comprises a first region having at least 85% sequence identity to SEQ ID NO: 7 or a functional portion or derivative thereof comprising the sequence of SEQ ID NO: 9 and/or SEQ ID NO: 10 operably linked to a second region having at least 85% sequence identity to SEQ ID NO: 8 or a functional portion or derivative thereof comprising the sequence of SEQ ID NO: 14 and/or SEQ ID NO: 15, wherein the first region and the second region are directly fused or joined by a linker comprising one to one hundred nucleotides.
E4. The method of E3, wherein the first region comprises the sequence of SEQ ID NO: 7.
E5. The method of E4, wherein the first region consists of the sequence of SEQ ID NO: 7.
E6. The method of E3, wherein the functional portion of SEQ ID NO: 7 comprises the sequence of SEQ ID NO: 9.
E7. The method of E3, wherein the functional portion of SEQ ID NO: 7 comprises the sequence of SEQ ID NO: 10.
E8. The method of E3, wherein the functional portion of SEQ ID NO: 7 comprises the sequence of SEQ ID NO: 9 and the sequence of SEQ ID NO: 10.
E9. The method of E8, wherein the functional portion of SEQ ID NO: 7 comprises the sequence of SEQ ID NO: 11.
E10. The method of E8, wherein the functional portion of SEQ ID NO: 7 comprises the sequence of SEQ ID NO: 12.
E11. The method of E8, wherein the functional portion of SEQ ID NO: 7 comprises the sequence of SEQ ID NO: 13.
E12. The method of E8, wherein the functional portion of SEQ ID NO: 7 comprises the sequence of SEQ ID NO: 33.
E13. The method of any one of E3-E12, wherein the second region comprises the sequence of SEQ ID NO: 8.
E14. The method of E13, wherein the second region consists of the sequence of SEQ ID NO: 8.
E15. The method of any one of E3-E12, wherein the functional portion of SEQ ID NO: 8 comprises the sequence of SEQ ID NO: 14.
E16. The method of any one of E3-E12, wherein the functional portion of SEQ ID NO: 8 comprises the sequence of SEQ ID NO: 15.
E17. The method of any one of E3-E12, wherein the functional portion of SEQ ID NO: 8 comprises the sequence of SEQ ID NO: 14 and the sequence of SEQ ID NO: 15.
E18. The method of E17, wherein the functional portion of SEQ ID NO: 8 comprises the sequence of SEQ ID NO: 16.
E19. The method of E17, wherein the functional portion of SEQ ID NO: 8 comprises the sequence of SEQ ID NO: 17.
E20. The method of E17, wherein the functional portion of SEQ ID NO: 8 comprises the sequence of SEQ ID NO: 18.
E21. The method of any one of E3-E12, wherein the functional portion of SEQ ID NO: 8 comprises the sequence of SEQ ID NO: 34.
E22. The method of any one of E3-E12, wherein the functional portion of SEQ ID NO: 8 comprises the sequence of SEQ ID NO: 35.
E23. The method of any one of E3-E12, wherein the functional portion of SEQ ID NO: 8 comprises the sequence of SEQ ID NO: 34 and the sequence of SEQ ID NO: 35.
E24. The method of E23, wherein the functional portion of SEQ ID NO: 8 comprises the sequence of SEQ ID NO: 38.
E25. The method of E3, wherein the Myo15 promoter comprises the sequence of SEQ ID NO: 19.
E26. The method of E25, wherein the Myo15 promoter consists of the sequence of SEQ ID NO: 19
E27. The method of E3, wherein the Myo15 promoter comprises the sequence of SEQ ID NO: 21.
E28. The method of E27, wherein the Myo15 promoter consists of the sequence of SEQ ID NO: 21.
E29. The method of E3, wherein the Myo15 promoter comprises the sequence of SEQ ID NO: 22.
E30. The method of E29, wherein the Myo15 promoter consists of the sequence of SEQ ID NO: 22.
E31. The method of E3, wherein the Myo15 promoter comprises the sequence of SEQ ID NO: 42. E31.
E32. The method of E31, wherein the Myo15 promoter consists of the sequence of SEQ ID NO: 42.
E33. The method of E3, wherein the Myo15 promoter comprises the sequence of SEQ ID NO: 43.
E34. The method of E33, wherein the Myo15 promoter consists of the sequence of SEQ ID NO: 43.
E35. The method of E1 or E2, wherein the Myo15 promoter comprises a first region having at least 85% sequence identity to SEQ ID NO: 23 or a functional portion or derivative thereof comprising the sequence of SEQ ID NO: 25 operably linked to a second region having at least 85% sequence identity to SEQ ID NO: 24 or a functional portion or derivative thereof comprising the sequence of SEQ ID NO: 26 and/or SEQ ID NO: 27, wherein the first region and the second region are directly fused or joined by linker comprising one to four hundred nucleotides between the first region and the second region.
E36. The method of E35, wherein the first region comprises the sequence of SEQ ID NO: 23.
E37. The method of E36, wherein the first region consists of the sequence of SEQ ID NO: 23.
E38. The method of E35, wherein the functional portion of SEQ ID NO: 23 comprises the sequence of SEQ ID NO: 25.
E39. The method of any one of E35-E38, wherein the second region comprises the sequence of SEQ ID NO: 24.
E40. The method of E39, wherein the second region consists of the sequence of SEQ ID NO: 24.
E41. The method of any one of E35-E38, wherein the functional portion of SEQ ID NO: 24 comprises the sequence of SEQ ID NO: 26.
E42. The method of any one of E35-E38, wherein the functional portion of SEQ ID NO: 24 comprises the sequence of SEQ ID NO: 27.
E43. The method of any one of E35-E38, wherein the functional portion of SEQ ID NO: 24 comprises the sequence of SEQ ID NO: 26 and the sequence of SEQ ID NO: 27.
E44. The method of E43, wherein the functional portion of SEQ ID NO: 24 comprises the sequence of SEQ ID NO: 28.
E45. The method of E43, wherein the functional portion of SEQ ID NO: 24 comprises the sequence of SEQ ID NO: 29.
E46. The method of E43, wherein the functional portion of SEQ ID NO: 24 comprises the sequence of SEQ ID NO: 30.
E47. The method of E35, wherein the Myo15 promoter comprises the sequence of SEQ ID NO: 31.
E48. The method of E47, wherein the Myo15 promoter consists of the sequence of SEQ ID NO: 31.
E49. The method of E35, wherein the Myo15 promoter comprises the sequence of SEQ ID NO: 32.
E50. The method of E49, wherein the Myo15 promoter consists of the sequence of SEQ ID NO: 32.
E51. The method of E1 or E2, wherein the Myo15 promoter comprises a sequence having at least 85% sequence identity to SEQ ID NO: 34.
E52. The method of E1 or E2, wherein the Myo15 promoter comprises a sequence having at least 85% sequence identity to SEQ ID NO: 38.
E53. The method of E1 or E2, wherein the Myo15 promoter comprises a sequence having at least 85% sequence identity to SEQ ID NO: 39.
E54. The method of E1 or E2, wherein the Myo15 promoter comprises a sequence having at least 85% sequence identity to SEQ ID NO: 40.
E55. A method of treating a human subject having biallelic OTOF mutations (e.g., a biallelic likely pathogenic or pathogenic OTOF mutation), the method comprising administering to an inner ear of the subject an amount of 1×10¹³ vg/mL to 1×10¹⁴ vg/mL of an OTOF dual vector system in a volume of 200-250 μL by intracochlear injection, wherein the OTOF dual vector system comprises:
   a first AAV vector comprising a ubiquitous promoter operably linked to a first coding polynucleotide that encodes an N-terminal portion of an OTOF isoform 5 protein, a splice donor sequence positioned 3′ of the first coding polynucleotide, and a first recombinogenic region positioned 3′ of the splice donor sequence; and a second AAV vector comprising a second recombinogenic region, a splice acceptor sequence positioned 3′ of the second recombinogenic region, a second coding polynucleotide that encodes a C-terminal portion of the OTOF isoform 5 protein positioned 3′ of the splice acceptor sequence, and a poly(A) sequence positioned 3′ of the second coding polynucleotide;
   wherein the first coding polynucleotide and the second coding polynucleotide do not overlap, wherein neither the first nor second AAV vector encodes the full-length OTOF isoform 5 protein, and wherein the first AAV vector and the second AAV vector are administered at a ratio of about 3:1 to about 1:3 (e.g., 3:1 to 1:3, 2:1 to 1:2, or 1:1).
E56. The method of E55, wherein the ubiquitous promoter is selected from the group consisting of a CAG promoter, a cytomegalovirus (CMV) promoter, a truncated CMV-chicken β-actin promoter (smCBA promoter), a chicken β-actin promoter, a CB7 promoter, a hybrid CMV enhancer/human β-actin promoter, or a human β-actin promoter.
E57. The method of E56, wherein the ubiquitous promoter is the smCBA promoter.
E58. The method of E57, wherein the smCBA promoter comprises the sequence of SEQ ID NO: 44.
E59. The method of E58, wherein the smCBA promoter consists of the sequence of SEQ ID NO: 44.
E60. The method of any one of E55-E59, wherein the first AAV vector and the second AAV vector comprise an Anc80 capsid, an AAV2 capsid, an AAVquad(Y-F) capsid, an AAV8 capsid, an AAV9 capsid, an AAV1 capsid, an AAV6 capsid, a DJ/9 capsid, a 7m8 capsid, an Anc80L65 capsid, or a PHP.B capsid.
E61. A method of improving hearing in a human subject, the method comprising selecting a subject having biallelic OTOF mutations (e.g., a biallelic likely pathogenic or pathogenic OTOF mutation) and administering to an inner ear of the subject an amount of 1×10¹³ vg/mL to 1×10¹⁴ vg/mL of an OTOF dual vector system in a volume of 200-250 μL by intracochlear injection, wherein the OTOF dual vector system comprises:
   a first AAV vector comprising a first ITR sequence; a promoter operably linked to a first coding polynucleotide that encodes an N-terminal portion of an OTOF isoform 5 protein; a splice donor sequence positioned 3′ of the first coding polynucleotide; a recombinogenic region positioned 3′ of the splice donor sequence; and a second ITR sequence; and a second AAV vector comprising a first ITR sequence; a second recombinogenic region; a splice acceptor sequence positioned 3′ of the second recombinogenic region; a second coding polynucleotide that encodes a C-terminal portion of the OTOF isoform 5 protein positioned 3′ of the splice acceptor sequence; a poly(A) sequence positioned 3′ of the second coding polynucleotide; and a second ITR sequence;
   wherein the first coding polynucleotide and the second coding polynucleotide that encode the OTOF isoform 5 protein do not overlap, wherein neither the first nor second AAV vector encodes the full-length OTOF isoform 5 protein, and wherein the first AAV vector and the second AAV vector are administered at a ratio of about 3:1 to 1:3 (e.g., 3:1 to 1:3, 2:1 to 1:2, or 1:1).
E62. The method of E61, wherein the promoter is a Myo15 promoter.
E63. The method of E62, wherein the Myo15 promoter comprises a first region having at least 85% sequence identity to SEQ ID NO: 7 or a functional portion or derivative thereof comprising the sequence of SEQ ID NO: 9 and/or SEQ ID NO: 10 operably linked to a second region having at least 85% sequence identity to SEQ ID NO: 8 or a functional portion or derivative thereof comprising the sequence of SEQ ID NO: 14 and/or SEQ ID NO: 15, wherein the first region and the second region are directly fused or joined by a linker comprising one to one hundred nucleotides.
E64. The method of E63, wherein the first region comprises the sequence of SEQ ID NO: 7.
E65. The method of E64, wherein the first region consists of the sequence of SEQ ID NO: 7.
E66. The method of E63, wherein the functional portion of SEQ ID NO: 7 comprises the sequence of SEQ ID NO: 9.
E67. The method of E63, wherein the functional portion of SEQ ID NO: 7 comprises the sequence of SEQ ID NO: 10.
E68. The method of E63, wherein the functional portion of SEQ ID NO: 7 comprises the sequence of SEQ ID NO: 9 and the sequence of SEQ ID NO: 10.
E69. The method of E68, wherein the functional portion of SEQ ID NO: 7 comprises the sequence of SEQ ID NO: 11.
E70. The method of E68, wherein the functional portion of SEQ ID NO: 7 comprises the sequence of SEQ ID NO: 12.
E71. The method of E68, wherein the functional portion of SEQ ID NO: 7 comprises the sequence of SEQ ID NO: 13.
E72. The method of E68, wherein the functional portion of SEQ ID NO: 7 comprises the sequence of SEQ ID NO: 33.
E73. The method of any one of E63-E72, wherein the second region comprises the sequence of SEQ ID NO: 8.
E74. The method of E73, wherein the second region consists of the sequence of SEQ ID NO: 8.
E75. The method of any one of E63-E72, wherein the functional portion of SEQ ID NO: 8 comprises the sequence of SEQ ID NO: 14.
E76. The method of any one of E63-E72, wherein the functional portion of SEQ ID NO: 8 comprises the sequence of SEQ ID NO: 15.
E77. The method of any one of E63-E72, wherein the functional portion of SEQ ID NO: 8 comprises the sequence of SEQ ID NO: 14 and the sequence of SEQ ID NO: 15.
E78. The method of E77, wherein the functional portion of SEQ ID NO: 8 comprises the sequence of SEQ ID NO: 16.
E79. The method of E77, wherein the functional portion of SEQ ID NO: 8 comprises the sequence of SEQ ID NO: 17.
E80. The method of E77, wherein the functional portion of SEQ ID NO: 8 comprises the sequence of SEQ ID NO: 18.
E81. The method of any one of E63-E72, wherein the functional portion of SEQ ID NO: 8 comprises the sequence of SEQ ID NO: 34.
E82. The method of any one of E63-E72, wherein the functional portion of SEQ ID NO: 8 comprises the sequence of SEQ ID NO: 35.
E83. The method of any one of E63-E72, wherein the functional portion of SEQ ID NO: 8 comprises the sequence of SEQ ID NO: 34 and the sequence of SEQ ID NO: 35.
E84. The method of E83, wherein the functional portion of SEQ ID NO: 8 comprises the sequence of SEQ ID NO: 38.
E85. The method of E63, wherein the Myo15 promoter comprises the sequence of SEQ ID NO: 19.
E86. The method of E85, wherein the Myo15 promoter consists of the sequence of SEQ ID NO: 19
E87. The method of E63, wherein the Myo15 promoter comprises the sequence of SEQ ID NO: 21.
E88. The method of E87, wherein the Myo15 promoter consists of the sequence of SEQ ID NO: 21.
E89. The method of E63, wherein the Myo15 promoter comprises the sequence of SEQ ID NO: 22.
E90. The method of E89, wherein the Myo15 promoter consists of the sequence of SEQ ID NO: 22.
E91. The method of 63, wherein the Myo15 promoter comprises the sequence of SEQ ID NO: 42.
E92. The method of E91, wherein the Myo15 promoter consists of the sequence of SEQ ID NO: 42.
E93. The method of E63, wherein the Myo15 promoter comprises the sequence of SEQ ID NO: 43.
E94. The method of E93, wherein the Myo15 promoter consists of the sequence of SEQ ID NO: 43.
E95. The method of E62, wherein the Myo15 promoter comprises a first region having at least 85% sequence identity to SEQ ID NO: 23 or a functional portion or derivative thereof comprising the sequence of SEQ ID NO: 25 operably linked to a second region having at least 85% sequence identity to SEQ ID NO: 24 or a functional portion or derivative thereof comprising the sequence of SEQ ID NO: 26 and/or SEQ ID NO: 27, wherein the first region and the second region are directly fused or joined by linker comprising one to four hundred nucleotides between the first region and the second region.
E96. The method of E95, wherein the first region comprises the sequence of SEQ ID NO: 23.
E97. The method of E96, wherein the first region consists of the sequence of SEQ ID NO: 23.
E98. The method of E95, wherein the functional portion of SEQ ID NO: 23 comprises the sequence of SEQ ID NO: 25.
E99. The method of any one of E95-E98, wherein the second region comprises the sequence of SEQ ID NO: 24.
E100. The method of E99, wherein the second region consists of the sequence of SEQ ID NO: 24.
E101. The method of any one of E95-E98, wherein the functional portion of SEQ ID NO: 24 comprises the sequence of SEQ ID NO: 26.
E102. The method of any one of E95-E98, wherein the functional portion of SEQ ID NO: 24 comprises the sequence of SEQ ID NO: 27.
E103. The method of any one of E95-E98, wherein the functional portion of SEQ ID NO: 24 comprises the sequence of SEQ ID NO: 26 and the sequence of SEQ ID NO: 27.
E104. The method of E103, wherein the functional portion of SEQ ID NO: 24 comprises the sequence of SEQ ID NO: 28.
E105. The method of E103, wherein the functional portion of SEQ ID NO: 24 comprises the sequence of SEQ ID NO: 29.
E106. The method of E103, wherein the functional portion of SEQ ID NO: 24 comprises the sequence of SEQ ID NO: 30.
E107. The method of E95, wherein the Myo15 promoter comprises the sequence of SEQ ID NO: 31.
E108. The method of E107, wherein the Myo15 promoter consists of the sequence of SEQ ID NO: 31.
E109. The method of E95, wherein the Myo15 promoter comprises the sequence of SEQ ID NO: 32.
E110. The method of E109, wherein the Myo15 promoter consists of the sequence of SEQ ID NO: 32.
E111. The method of E62, wherein the Myo15 promoter comprises a sequence having at least 85% sequence identity to SEQ ID NO: 34.
E112. The method of E62, wherein the Myo15 promoter comprises a sequence having at least 85% sequence identity to SEQ ID NO: 38.
E113. The method of E62, wherein the Myo15 promoter comprises a sequence having at least 85% sequence identity to SEQ ID NO: 39.
E114. The method of E62, wherein the Myo15 promoter comprises a sequence having at least 85% sequence identity to SEQ ID NO: 40.
E115. The method of E61, wherein the promoter is a ubiquitous promoter.
E116. The method of E115, wherein the ubiquitous promoter is selected from the group consisting of a CAG promoter, a cytomegalovirus (CMV) promoter, a truncated CMV-chicken β-actin promoter (smCBA promoter), a chicken β-actin promoter, a CB7 promoter, a hybrid CMV enhancer/human β-actin promoter, or a human β-actin promoter.
E117. The method of E116, wherein the ubiquitous promoter is the smCBA promoter.
E118. The method of E117, wherein the smCBA promoter comprises the sequence of SEQ ID NO: 44.
E119. The method of E118, wherein the smCBA promoter consists of the sequence of SEQ ID NO: 44.
E120. The method of any one of E61-E119, wherein the first AAV vector and the second AAV vector comprise an Anc80 capsid, an AAV2 capsid, an AAVquad(Y-F) capsid, an AAV8 capsid, an AAV9 capsid, an AAV1 capsid, an AAV6 capsid, a DJ/9 capsid, a 7m8 capsid, an Anc80L65 capsid, or a PHP.B capsid.
E121. The method of E120, wherein the first AAV vector and the second AAV vector comprise an AAV1 capsid.
E122. The method of any one of E61-E121, wherein the subject has or is identified as having profound sensorineural hearing loss (e.g., the method comprises selecting a subject having biallelic OTOF mutations and profound sensorineural hearing loss).
E123. The method of any one of E61-E122, wherein the subject has or is identified as having present outer hair cell function (e.g., the method comprises selecting a subject having biallelic OTOF mutations and present outer hair cell function or selecting a subject having biallelic OTOF mutations, profound sensorineural hearing loss, and present outer hair cell function).
E124. The method of E123, wherein the subject is determined to have present outer hair cell function based on detectable otoacoustic emissions and/or a present cochlear microphonic.
E125. The method of any one of E1-E124, wherein the first and second recombinogenic regions are the same.
E126. The method of any one of E1-E125, wherein the first recombinogenic region and/or the second recombinogenic region is an AK recombinogenic region (e.g., an AK recombinogenic region comprising or consisting of the sequence of SEQ ID NO: 47).
E127. The method of any one of E1-E125, wherein the first recombinogenic region and/or the second recombinogenic region is an AP gene fragment.
E128. The method of E127, wherein the AP gene fragment comprises or consists of the sequence of any one of SEQ ID NOs: 48-53.
E129. The method of E128, wherein the AP gene fragment comprises the sequence of SEQ ID NO: 51.
E130. The method of E129, wherein the AP gene fragment consists of the sequence of SEQ ID NO: 51.
E131. The method of any one of E1-E130, wherein each of the first and second coding polynucleotides encode about half of the OTOF isoform 5 protein sequence.
E132. The method of any one of E1-E131, wherein the first and second coding polynucleotides are divided at an OTOF exon boundary.
E133. The method of E132, wherein the first and second coding polynucleotides are divided at the OTOF exon 20/exon 21 boundary.
E134. The method of any one of E1-E132, wherein the first coding polynucleotide consists of exons 1-20 of a polynucleotide encoding the OTOF isoform 5 protein and the second coding polynucleotide consists of exons 21-45 and 47 of a polynucleotide encoding the OTOF isoform 5 protein.
E135. The method of E132, wherein the first and second coding polynucleotides are divided at the OTOF exon 21/exon 22 boundary.
E136. The method of any one of E1-E132, wherein the first coding polynucleotide consists of exons 1-21 of a polynucleotide encoding the OTOF isoform 5 protein and the second coding polynucleotide consists of exons 22-45 and 47 of a polynucleotide encoding the OTOF isoform 5 protein.
E137. The method of any one of E1-E136, wherein the first and second coding polynucleotides that encode the OTOF isoform 5 protein do not comprise introns.
E138. The method of any one of E1-E137, wherein the OTOF isoform 5 protein is a human OTOF isoform 5 protein.
E139. The method of any one of E1-E138, wherein the OTOF isoform 5 protein comprises the sequence of SEQ ID NO: 1 or a variant thereof having one or more conservative amino acid substitutions.
E140. The method of E139, wherein no more than 10% of the amino acids in the OTOF isoform 5 protein variant are conservative amino acid substitutions.
E141. The method of E139, wherein the OTOF isoform 5 protein consists of the sequence of SEQ ID NO: 1.
E142. The method of any one of E1-E139 and E141, wherein the OTOF isoform 5 protein is encoded by the sequence of SEQ ID NO: 2 or SEQ ID NO: 3.
E143. The method of any one of E1-E134 and E137-E142, wherein the first coding polynucleotide encodes amino acids 1-802 of SEQ ID NO: 1 and the second coding polynucleotide encodes amino acids 803-1997 of SEQ ID NO: 1.
E144. The method of any one of E1-E134 and E137-E143, wherein the N-terminal portion of the OTOF isoform 5 protein consists of the sequence of SEQ ID NO: 58 or a variant thereof having one or more conservative amino acid substitutions.
E145. The method of E144, wherein no more than 10% (e.g., 10%, 9%, 8%, 7%, 6%, 5%, 4%, 3%, 2%, 1% or fewer) of the amino acids in the N-terminal portion of the OTOF isoform 5 protein variant are conservative amino acid substitutions.
E146. The method of E144, wherein the N-terminal portion of the OTOF isoform 5 protein consists of the sequence of SEQ ID NO: 58.
E147. The method of any one of E1-E134, E137-E144, and E146, wherein the N-terminal portion of the OTOF isoform 5 protein is encoded by the sequence of SEQ ID NO: 56.
E148. The method of any one of E1-E134 and E137-E147, wherein the C-terminal portion of the OTOF isoform 5 protein consists of the sequence of SEQ ID NO: 59 or a variant thereof having one or more conservative amino acid substitutions.
E149. The method of E148, wherein no more than 10% (e.g., 10%, 9%, 8%, 7%, 6%, 5%, 4%, 3%, 2%, 1% or fewer) of the amino acids in the C-terminal portion of the OTOF isoform 5 protein variant are conservative amino acid substitutions.
E150. The method of E148, wherein the C-terminal portion of the OTOF isoform 5 protein consists of the sequence of SEQ ID NO: 59.
E151. The method of any one of E1-E134, E137-E148, and E150, wherein the C-terminal portion of the OTOF isoform 5 protein is encoded by the sequence of SEQ ID NO: 57.
E152. The method of any one of E1-E151, wherein the first vector comprises a first inverted terminal repeat (ITR) sequence 5′ of the promoter and a second ITR sequence 3′ of the recombinogenic region, and the second vector comprises a first ITR sequence 5′ of the recombinogenic region and a second ITR sequence 3′ of the poly(A) sequence.
E153. The method of E152, wherein the ITRs in the first vector and second vector are AAV2 ITRs or have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.
E154. The method of any one of E1-E153, wherein the splice donor sequence in the first vector comprises the sequence of SEQ ID NO: 54.
E155. The method of E154, wherein the splice donor sequence in the first vector consists of the sequence of SEQ ID NO: 54.
E156. The method of any one of E1-E155, wherein the splice acceptor sequence in the second vector comprises the sequence of SEQ ID NO: 55.
E157. The method of E156, wherein the splice acceptor sequence in the second vector consists of the sequence of SEQ ID NO: 55.
E158. The method of any one of E1-E157, wherein the first AAV vector comprises a Kozak sequence 3′ of the promoter and 5′ of the first coding polynucleotide that encodes the N-terminal portion of the OTOF isoform 5 protein.
E159. The method of E1 or E62, wherein the first AAV vector comprises a polynucleotide sequence comprising the sequence of nucleotides 2272 to 6041 of SEQ ID NO: 60.
E160. The method of E1, E62, or E159, wherein the first AAV vector comprises a polynucleotide sequence comprising the sequence of nucleotides 2049 to 6264 of SEQ ID NO: 60.
E161. The method of E160, wherein the first AAV vector comprises a polynucleotide sequence consisting of the sequence of nucleotides 2049 to 6264 of SEQ ID NO: 60.
E162. The method of E1 or E62, wherein the first AAV vector comprises a polynucleotide sequence comprising the sequence of nucleotides 182 to 3949 of SEQ ID NO: 62.
E163. The method of E1, E62, or E162, wherein the first AAV vector comprises a polynucleotide sequence comprising the sequence of nucleotides 19 to 4115 of SEQ ID NO: 62.
E164. The method of E163, wherein the first AAV vector comprises a polynucleotide sequence consisting of the sequence of nucleotides 19 to 4115 of SEQ ID NO: 62.
E165. The method of E55 or E115, wherein the first AAV vector comprises a polynucleotide sequence comprising the sequence of positions 2267 to 6014 of SEQ ID NO: 64.
E166. The method of E55, E115, or E165, wherein the first AAV vector comprises a polynucleotide sequence comprising the sequence of positions 2049 to 6237 of SEQ ID NO: 64.
E167. The method of E166, wherein the first AAV vector comprises a polynucleotide sequence consisting of the sequence of positions 2049 to 6237 of SEQ ID NO: 64.
E168. The method of E55 or E115, wherein the first AAV vector comprises a polynucleotide sequence comprising the sequence of positions 177 to 3924 of SEQ ID NO: 65.
E169. The method of E55, E115, or E168, wherein the first AAV vector comprises a polynucleotide sequence comprising the sequence of positions 19 to 4090 of SEQ ID NO: 65.
E170. The method of E169, wherein the first AAV vector comprises a polynucleotide sequence consisting of the sequence of positions 19 to 4090 of SEQ ID NO: 65.
E171. The method of any one of E1, E55, E62, E115, E159-E161, and E165-E167, wherein the second AAV vector comprises a polynucleotide sequence comprising the sequence of nucleotides 2267 to 6476 of SEQ ID NO: 61.
E172. The method of any one of E1, E55, E62, E115, E159-E161, E165-E167, and E171, wherein the second AAV vector comprises a polynucleotide sequence comprising the sequence of nucleotides 2049 to 6693 of SEQ ID NO: 61.
E173. The method of any one of E172, wherein the second AAV vector comprises a polynucleotide sequence consisting of the sequence of nucleotides 2049 to 6693 of SEQ ID NO: 61.
E174. The method of any one of E1, E55, E62, E115, E162-E164, and E168-E170, wherein the second AAV vector comprises a polynucleotide sequence comprising the sequence of nucleotides 187 to 4396 of SEQ ID NO: 63.
E175. The method of any one of E1, E55, E62, E115, E162-E164, E168-E170, and E174, wherein the second AAV vector comprises a polynucleotide sequence comprising the sequence of nucleotides 19 to 4589 of SEQ ID NO: 63.
E176. The method of E175, wherein the second AAV vector comprises a polynucleotide sequence consisting of the sequence of nucleotides 19 to 4589 of SEQ ID NO: 63.
E177. The method of E1 or E62, wherein the first AAV vector comprises a polynucleotide sequence comprising the sequence of nucleotides 235 to 4004 of SEQ ID NO: 66.
E178. The method of E1, E62, or E177, wherein the first AAV vector comprises a polynucleotide sequence comprising the sequence of nucleotides 12 to 4227 of SEQ ID NO: 66.
E179. The method of E178, wherein the first AAV vector comprises a polynucleotide sequence consisting of the sequence of nucleotides 12 to 4227 of SEQ ID NO: 66.
E180. The method of E55 or E115, wherein the first AAV vector comprises a polynucleotide sequence comprising the sequence of nucleotides 230 to 3977 of SEQ ID NO: 68.
E181. The method of E55, E115, or E180, wherein the first AAV vector comprises a polynucleotide sequence comprising the sequence of nucleotides 12 to 4200 of SEQ ID NO: 68.
E182. The method of E181, wherein the first AAV vector comprises a polynucleotide sequence consisting of the sequence of nucleotides 12 to 4200 of SEQ ID NO: 68.
E183. The method of any one of E1, E55, E62, E115, and E177-E182, wherein the second AAV vector comprises a polynucleotide sequence comprising the sequence of nucleotides 229 to 4438 of SEQ ID NO: 67.
E184. The method of any one of E1, E55, E62, E115, and E177-E183, wherein the second AAV vector comprises a polynucleotide sequence comprising the sequence of nucleotides 12 to 4655 of SEQ ID NO: 67.
E185. The method of E184, wherein the second AAV vector comprises a polynucleotide sequence consisting of the sequence of nucleotides 12 to 4655 of SEQ ID NO: 67.
E186. The method of any one of E1-E185, wherein the subject is less than 18 years of age.
E187. The method of E186, wherein the subject is at least 7 years of age.
E188. The method of E186, wherein the subject is older than 24 months of age and younger than 7 years of age.
E189. The method of E186, wherein the subject is 24 months of age or younger.
E190. The method of any one of E1-E189, wherein the subject has or is identified as having profound sensorineural hearing loss (e.g., based on behavioral and/or physiological measurements of inner ear function, such as the absence of an ABR neural signal in response to a click stimulus at or below 85 dB normalized hearing level in the ear(s) to be treated or a ≥90 dB hearing level in the ear(s) to be treated).
E191. The method of any one of E1-E190, wherein the subject has or is identified as having behavioral open-set word detection scores consistent with the criteria from a cochlear implant label (e.g., has behavioral open-set word detection scores of <30% in the ear(s) to be treated).
E192. The method of any one of E1-E191, wherein the subject has or is identified as having congenital auditory neuropathy.
E193. The method of any one of E1-E192, wherein the subject has or is identified as having detectable otoacoustic emissions (e.g., presence of otoacoustic emissions (≥6 dB signal-to-noise ratio) at ≥ 3 frequencies from 1 to 8 kHz in the ear(s) to be treated).
E194. The method of any one of E1-E193, wherein the subject has or is identified as having a present cochlear microphonic in the ear(s) to be treated.
E195. The method of any one of E1-E194, wherein the administering to an inner ear comprises intracochlear injection via a catheter placed through the round window membrane of the cochlea.
E196. The method of any one of E1-E195, wherein the administering further comprises creating a fenestration in the lateral semicircular canal.
E197. The method of any one of E1-E196, wherein the injection is performed using a syringe and syringe pump.
E198. The method of any one of E1-E197, wherein the administering is at a rate of 0.8-1 mL/hr (e.g., 0.9 mL/hr).
E199. The method of any one of E1-E198, wherein the administering occurs once per ear.
E200. The method of any one of E1-E199, wherein the first vector and the second vector are administered at a ratio of about 1:1.
E201. The method of any one of E1-E200, wherein the dual vector system is formulated as an aqueous suspension.
E202. The method of E201, wherein the first vector and second vector are matched in titer and mixed at an approximately equal ratio.
E203. The method of E201 or E202, wherein the suspension comprises 10 mM sodium phosphate or disodium phosphate (also called sodium phosphate dibasic and having the formula Na₂HPO₄), 180 mM sodium chloride, 5% (w/v) sucrose, and 0.001% (w/v) poloxamer 188 at a pH of about 7.4.
E204. The method of E203, wherein the suspension comprises 10 mM sodium phosphate.
E205. The method of E203, wherein the suspension comprises 10 mM disodium phosphate.
E206. The method of any one of E1-E205, wherein the method further comprises administering a corticosteroid to the subject for the first four weeks after the administering of the OTOF dual vector system.
E207. The method of E206, wherein the method comprises administering 1 mg/kg of the corticosteroid daily during the first two weeks after the administering of the OTOF dual vector system, administering 0.5 mg/kg daily during the third week after the administering of the OTOF dual vector system, and administering 0.25 mg/kg daily during the fourth week after the administering of the OTOF dual vector system.
E208. The method of any one of E1-E207, wherein the OTOF dual vector system is administered in an amount of 1.0×10¹³ vg/mL to 5.0×10¹³ vg/mL in a volume of 200-250 μL.
E209. The method of E208, wherein the OTOF dual vector system is administered in an amount of 2.0×10¹³ vg/mL to 5.0×10¹³ vg/mL in a volume of 200-250 μL.
E210. The method of E209, wherein the OTOF dual vector system is administered in an amount of 3.0×10¹³ vg/mL to 5.0×10¹³ vg/mL in a volume of 200-250 μL.
E211. The method of E210, wherein the OTOF dual vector system is administered in an amount of 4.0×10¹³ vg/mL to 5.0×10¹³ vg/mL in a volume of 200-250 μL.
E212. The method of any one of E208-E211, wherein the OTOF dual vector system is administered in a volume of 210-250 μL.
E213. The method of E212, wherein the OTOF dual vector system is administered in a volume of 220-250 μL.
E214. The method of E213, wherein the OTOF dual vector system is administered in a volume of 230-250 μL.
E215. The method of E214, wherein the OTOF dual vector system is administered in a volume of 240-250 μL.
E216. The method of E215, wherein the OTOF dual vector system is administered in a volume of 240 μL.
E217. The method of E216, wherein the OTOF dual vector system is administered in an amount of 3.0×10¹³ vg/mL in a volume of 240 μL.
E218. The method of any one of E1-E207, wherein the OTOF dual vector system is administered in an amount of 5.0×10¹³ vg/mL to 1.0×10¹⁴ vg/mL in a volume of 200-250 μL.
E219. The method of E218, wherein the OTOF dual vector system is administered in an amount of 5.0×10¹³ vg/mL to 9.0×10¹³ vg/mL in a volume of 200-250 μL.
E220. The method of E219, wherein the OTOF dual vector system is administered in an amount of 5.0×10¹³ vg/mL to 8.0×10¹³ vg/mL in a volume of 200-250 μL.
E221. The method of E220, wherein the OTOF dual vector system is administered in an amount of 5.0×10¹³ vg/mL to 7.0×10¹³ vg/mL in a volume of 200-250 μL.
E222. The method of E221, wherein the OTOF dual vector system is administered in an amount of 5.0×10¹³ vg/mL to 6.0×10¹³ vg/mL in a volume of 200-250 μL.
E223. The method of any one of E218-E222, wherein the OTOF dual vector system is administered in a volume of 210-250 μL.
E224. The method of E223, wherein the OTOF dual vector system is administered in a volume of 220-250 μL.
E225. The method of E224, wherein the OTOF dual vector system is administered in a volume of 230-250 μL.
E226. The method of E225, wherein the OTOF dual vector system is administered in a volume of 240-250 μL.
E227. The method of E226, wherein the OTOF dual vector system is administered in a volume of 240 μL.
E228. The method of E227, wherein the OTOF dual vector system is administered in an amount of 7.3×10¹³ vg/mL in a volume of 240 μL.
E229. The method of any one of E1-E228, wherein the method further comprises identifying the subject as having a mutation in OTOF (e.g., a biallelic likely pathogenic or pathogenic OTOF mutation) prior to administering the dual vector system.
E230. The method of any one of E1-E229, wherein the subject has or is identified as having Deafness, Autosomal Recessive 9 (DFNB9).
E231. The method of any one of E1-E230, wherein the method further comprises evaluating the hearing of the subject prior to administering the dual vector system (e.g., the method further comprises identifying the subject as having profound sensorineural hearing loss prior to administering the dual vector system).
E232. The method of any one of E1-E231, wherein the method further comprises identifying the subject as having present outer hair cell function prior to administering the dual vector system.
E233. The method of any one of E1-232, wherein the method increases OTOF expression in a cochlear hair cell.
E234. The method of E233, wherein the cochlear hair cell is an inner hair cell.
E235. The method of any one of E1-E234, wherein the method further comprises evaluating the hearing of the subject after administering the dual vector system.
E236. The method of any one of E1-E235, wherein the method prevents or reduces hearing loss, delays the development of hearing loss, slows the progression of hearing loss, improves hearing, improves speech discrimination, or improves hair cell function.
E237. The method of any one of E1-E236, wherein the method improves one or more parameters selected from the subject's auditory brainstem response (ABR), behavioral audiometry, and score in one or more hearing questionnaires or behavioral tasks.
E238. The method of E237, wherein the hearing questionnaires and behavioral tasks include one or more of the Auditory Skills Checklist, Open & Closed set task, Early Speech Perception test, Pediatric Speech Intelligibility test, Lexical Neighborhood Test Multisyllabic Lexical Neighborhood Test, Consonant-Nucleus-Consonant test, Bamford-Kowal-Bench sentence test, LittIEARS® Auditory Questionnaire, MacArthur-Bates Communicative Development Inventories Words and Gestures, Quality of Life-Cochlear Implant, Pediatric Quality of Life Inventory, Hearing Environments and Reflection on Quality of Life (HEAR-QL)-26, HEAR-QL-28, The Health Utilities Index 3, Vanderbilt Fatigue Scales, and AzBio test.
E239. The method of E238, wherein the method improves the subject's score in the LittIEARS® Auditory Questionnaire.
E240. The method of E237, wherein the method improves hearing thresholds by at least 55 decibels hearing level with air conduction as assessed by behavioral pure tone audiometry.
E241. The method of E237, wherein the method results in a positive ABR wave V amplitude response
E242. A method of administering a nucleic acid vector to the inner ear of a human subject, the method comprising creating an opening in the round widow membrane of the cochlea and an opening in a second location in the inner ear and inserting a catheter through the opening in the round window membrane to infuse the nucleic acid vector into the perilymph.
E243. The method of E242, wherein the opening made in the second location in the inner ear is made in a semicircular canal.
E244. The method of E243, wherein the semicircular canal is a lateral semicircular canal.
E245. The method of E243, wherein the semicircular canal is a posterior semicircular canal.
E246. The method of E243, wherein the semicircular canal is a superior semicircular canal.
E247. The method of any one of E242-E246, wherein a surgical procedure is performed to access the middle ear before creating the opening in the round window membrane and lateral semicircular canal.
E248. The method of E247, wherein the surgical procedure involves a mastoidectomy and opening a facial recess.
E249. The method of E247 or E248, wherein the surgical procedure is a standard surgical procedure for cochlear implant surgery.
E250. The method of any one of E242-E249, wherein a syringe and/or a syringe pump are used in combination with the catheter for said administering.
E251. The method of any one of E242-E250, wherein the nucleic acid vector (e.g., a solution containing the nucleic acid vector) is administered at a rate of 0.5-1 mL/hr (e.g., 0.6-1 mL/hr, 0.7-1 mL/hr, 0.8-1 mL/hr, or 0.9-1 mL/hour, such as 0.9 mL/hr).
E252. The method of any one of E242-E251, wherein the nucleic acid vector is a viral vector.
E253. The method of E252, wherein the viral vector is a lentivirus vector, an adenovirus vector, or an adeno-associated virus (AAV) vector.
E254. The method of E253, wherein the viral vector is an AAV vector.
E255. The method of any one of E242-E254, wherein the human subject has or is at risk of developing sensorineural hearing loss.
E256. The method of E255, wherein the hearing loss is genetic hearing loss.
E257. The method of E256, wherein the genetic hearing loss is autosomal dominant hearing loss, autosomal recessive hearing loss, or X-linked hearing loss.
E258. The method of E256 or E257, wherein the nucleic acid vector comprises a polynucleotide encoding a protein that is absent or dysfunctional in the subject (e.g., a protein that is not produced or that does not function properly in the inner ear of the subject due to a genetic mutation associated with the hearing loss, e.g., the polynucleotide encodes a functional protein that is deficient in the subject due to a genetic mutation associated with the hearing loss) or a polynucleotide encoding an expression product designed to correct a genetic mutation in the subject (e.g., correct a genetic mutation associated with the hearing loss via gene editing).
E259. The method of E255, wherein the hearing loss is acquired hearing loss.
E260. The method of E259 wherein the acquired hearing loss is noise-induced hearing loss, age-related hearing loss, disease or infection-related hearing loss, head trauma-related hearing loss, or ototoxic drug-induced hearing loss.
E261. The method of E259 or E260, wherein the nucleic acid vector comprises a polynucleotide encoding an expression product that induces or increases cochlear hair cell regeneration, increases cochlear supporting cell numbers, prevents or reduces cochlear hair cell damage, prevents or reduces cochlear hair cell death, increases cochlear hair cell maturation, improves cochlear hair cell function, or promotes or increases cochlear hair cell survival.
E262. The method of any one of E242-E254, wherein the human subject has or is at risk of developing vestibular dysfunction.
E263. The method of E262, wherein the vestibular dysfunction is vertigo, dizziness, imbalance (e.g., loss of balance or a balance disorder), oscillopsia, or bilateral vestibulopathy.
E264. The method of E262 or E263, wherein the vestibular dysfunction is associated with a genetic mutation.
E265. The method of E264, wherein the nucleic acid vector comprises a polynucleotide encoding a protein that is absent or dysfunctional in the subject (e.g., a protein that is not produced or that does not function properly in the inner ear of the subject due to the genetic mutation associated with the vestibular dysfunction, e.g., the polynucleotide encodes a functional protein that is deficient in the subject due to the genetic mutation) or a polynucleotide encoding an expression product designed to correct a genetic mutation in the subject (e.g., correct a genetic mutation associated with the vestibular dysfunction via gene editing).
E266. The method of E262 or E263, wherein the vestibular dysfunction is associated with damage to or loss of vestibular cells (e.g., vestibular hair cells or supporting cells, such as damage to or loss of vestibular cells, related to disease or infection, head trauma, ototoxic drugs (e.g., vestibulotoxic drugs), or aging).
E267. The method of E266, wherein the nucleic acid vector comprises a polynucleotide encoding an expression product that induces or increases vestibular hair cell regeneration, increases vestibular supporting cell numbers, prevents or reduces vestibular hair cell damage, prevents or reduces vestibular hair cell death, increases vestibular hair cell maturation, improves vestibular hair cell function, or promotes or increases vestibular hair cell survival.
E268. An aqueous suspension comprising an OTOF dual vector system, 10 mM sodium phosphate or disodium phosphate, 180 mM sodium chloride, 5% (w/v) sucrose, and 0.001% (w/v) poloxamer 188 at a pH of 7.4, wherein the OTOF dual vector system comprises:
   a first AAV vector comprising a first ITR sequence; a promoter operably linked to a first coding polynucleotide that encodes an N-terminal portion of an OTOF isoform 5 protein; a splice donor sequence positioned 3′ of the first coding polynucleotide; a recombinogenic region positioned 3′ of the splice donor sequence; and a second ITR sequence; and a second AAV vector comprising a first ITR sequence; a second recombinogenic region; a splice acceptor sequence positioned 3′ of the second recombinogenic region; a second coding polynucleotide that encodes a C-terminal portion of the OTOF isoform 5 protein positioned 3′ of the splice acceptor sequence; a poly(A) sequence positioned 3′ of the second coding polynucleotide; and a second ITR sequence;
   wherein the first coding polynucleotide and the second coding polynucleotide that encode the OTOF isoform 5 protein do not overlap, and wherein neither the first nor second AAV vector encodes the full-length OTOF isoform 5 protein.
E269. The suspension of E268, wherein the suspension comprises 10 mM sodium phosphate.
E270. The suspension of E268, wherein the suspension comprises 10 mM disodium phosphate.
E271. The suspension of any one of E268-E270, wherein the ratio of the first vector to the second vector is about 3:1 to about 1:3.
E272. The suspension of any one of E268-E271, wherein the first vector and second vector are matched in titer and mixed at an approximately equal ratio.
E273. The suspension of E272, wherein the ratio of the first vector to the second vector is about 1:1.
E274. The suspension of any one of E268-E273, wherein the suspension has a titer of 1×10¹³ vg/mL to 1× 10¹⁴ vg/mL.
E275. The suspension of E274, wherein the suspension has a titer of 1×10¹³ vg/mL to 5×10¹³ vg/mL.
E276. The suspension of E275, wherein the suspension has a titer of 1×10¹³ vg/mL to 4×10¹³ vg/mL.
E277. The suspension of E276, wherein the suspension has a titer of 2×10¹³ vg/mL to 4×10¹³ vg/mL.
E278. The suspension of E277, wherein the suspension has a titer of 3×10¹³ vg/mL.
E279. The suspension of E274, wherein the suspension has a titer of 5×10¹³ vg/mL to 1×10¹⁴ vg/mL.
E280. The suspension of E279, wherein the suspension has a titer of 5×10¹³ vg/mL to 9×10¹³ vg/mL.
E281. The suspension of E280, wherein the suspension has a titer of 5×10¹³ vg/mL to 8×10¹³ vg/mL.
E282. The suspension of E281, wherein the suspension has a titer of 6×10¹³ vg/mL to 8×10¹³ vg/mL.
E283. The suspension of E282, wherein the suspension has a titer of 7.3×10¹³ vg/mL.
E284. The suspension of any one of E268-E283, wherein the suspension is formulated for intracochlear injection.
E285. The suspension of any one of E268-E284, wherein the promoter is a Myo15 promoter (e.g., a Myo15 promoter described hereinabove, such as a Myo15 promoter having at least 85% sequence identity to any one of SEQ ID NOs: 19, 21, 22, 31, 32, 34, 38, 39, 40, 42, or 43).
E286. The suspension of any one of E268-E284, wherein the promoter is a ubiquitous promoter.
E287. The suspension of E286, wherein the ubiquitous promoter is selected from the group consisting of a CAG promoter, a cytomegalovirus (CMV) promoter, a truncated CMV-chicken β-actin promoter (smCBA promoter), a chicken β-actin promoter, a CB7 promoter, a hybrid CMV enhancer/human β-actin promoter, or a human β-actin promoter.
E288. The suspension of E287, wherein the ubiquitous promoter is the smCBA promoter.
E289. The suspension of E288, wherein the smCBA promoter comprises the sequence of SEQ ID NO: 44.
E290. The suspension of any one of E268-E289, wherein the first AAV vector and the second AAV vector comprise an Anc80 capsid, an AAV2 capsid, an AAVquad(Y-F) capsid, an AAV8 capsid, an AAV9 capsid, an AAV1 capsid, an AAV6 capsid, a DJ/9 capsid, a 7m8 capsid, an Anc80L65 capsid, or a PHP.B capsid.
E291. The suspension of E290, wherein the first AAV vector and the second AAV vector comprise an AAV1 capsid.
E292. The suspension of any one of E268-E291, wherein the first and second recombinogenic regions are the same.
E293. The suspension of any one of E268-E292, wherein the first recombinogenic region and/or the second recombinogenic region is an AK recombinogenic region (e.g., an AK recombinogenic region comprising or consisting of the sequence of SEQ ID NO: 47).
E294. The suspension of any one of E268-E292, wherein the first recombinogenic region and/or the second recombinogenic region is an AP gene fragment (e.g., an AP gene fragment comprising or consisting of the sequence of any one of SEQ ID NOs: 48-53).
E295. The suspension of any one of E268-E294, wherein each of the first and second coding polynucleotides encode about half of the OTOF isoform 5 protein sequence.
E296. The suspension of any one of E268-E295, wherein the first and second coding polynucleotides are divided at an OTOF exon boundary.
E297. The suspension of E296, wherein the first and second coding polynucleotides are divided at the OTOF exon 20/exon 21 boundary.
E298. The suspension of any one of E268-E296, wherein the first coding polynucleotide consists of exons 1-20 of a polynucleotide encoding the OTOF isoform 5 protein and the second coding polynucleotide consists of exons 21-45 and 47 of a polynucleotide encoding the OTOF isoform 5 protein.
E299. The suspension of E296, wherein the first and second coding polynucleotides are divided at the OTOF exon 21/exon 22 boundary.
E300. The suspension of any one of E268-E296, wherein the first coding polynucleotide consists of exons 1-21 of a polynucleotide encoding the OTOF isoform 5 protein and the second coding polynucleotide consists of exons 22-45 and 47 of a polynucleotide encoding the OTOF isoform 5 protein.
E301. The suspension of any one of E268-E300, wherein the first and second coding polynucleotides that encode the OTOF isoform 5 protein do not comprise introns.
E302. The suspension of any one of E268-E301, wherein the OTOF isoform 5 protein is a human OTOF isoform 5 protein.
E303. The suspension of any one of E268-E302, wherein the OTOF isoform 5 protein comprises the sequence of SEQ ID NO: 1 or a variant thereof having one or more conservative amino acid substitutions.
E304. The suspension of E303, wherein no more than 10% of the amino acids in the OTOF isoform 5 protein variant are conservative amino acid substitutions.
E305. The suspension of E303, wherein the OTOF isoform 5 protein consists of the sequence of SEQ ID NO: 1.
E306. The suspension of any one of E268-E303 and E305, wherein the OTOF isoform 5 protein is encoded by the sequence of SEQ ID NO: 2 or SEQ ID NO: 3.
E307. The suspension of any one of E268-E298 and E301-E306, wherein the first coding polynucleotide encodes amino acids 1-802 of SEQ ID NO: 1 and the second coding polynucleotide encodes amino acids 803-1997 of SEQ ID NO: 1.
E308. The suspension of any one of E268-E298 and E301-E307, wherein the N-terminal portion of the OTOF isoform 5 protein consists of the sequence of SEQ ID NO: 58 or a variant thereof having one or more conservative amino acid substitutions.
E309. The suspension of any one of E268-E298 and E301-E308, wherein the N-terminal portion of the OTOF isoform 5 protein is encoded by the sequence of SEQ ID NO: 56.
E310. The suspension of any one of E268-E298 and E301-E309, wherein the C-terminal portion of the OTOF isoform 5 protein consists of the sequence of SEQ ID NO: 59 or a variant thereof having one or more conservative amino acid substitutions.
E311. The suspension of any one of E268-E298 and E301-E310, wherein the C-terminal portion of the OTOF isoform 5 protein is encoded by the sequence of SEQ ID NO: 57.
E312. The suspension of any one of E268-E311, wherein the first vector comprises a first inverted terminal repeat (ITR) sequence 5′ of the promoter and a second ITR sequence 3′ of the recombinogenic region, and the second vector comprises a first ITR sequence 5′ of the recombinogenic region and a second ITR sequence 3′ of the poly(A) sequence.
E313. The suspension of E312, wherein the ITRs in the first vector and second vector are AAV2 ITRs or have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.
E314. The suspension of any one of E268-E313, wherein the first AAV vector comprises a Kozak sequence 3′ of the promoter and 5′ of the first coding polynucleotide that encodes the N-terminal portion of the OTOF isoform 5 protein.
E315. A kit comprising the suspension of any one of E268-E314.
E316. The kit of E315, further comprising one or more of a syringe, syringe pump, and catheter.
E317. A syringe comprising the suspension of any one of E268-E314.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method of improving hearing in treating a human subject having biallelic otoferlin (OTOF) mutations and profound sensorineural hearing loss, the method comprising administering to an inner ear of the subject an amount of 1×10¹³ vg/mL to 1×10¹⁴ vg/mL of an OTOF dual vector system in a volume of 200-250 μL by intracochlear injection, wherein the OTOF dual vector system comprises:
a first adeno-associated virus (AAV) vector comprising a first inverted terminal repeat (ITR) sequence; a Myosin 15 (Myo15) promoter operably linked to a first coding polynucleotide that encodes an N-terminal portion of an OTOF isoform 5 protein; a splice donor sequence positioned 3′ of the first coding polynucleotide; a recombinogenic region positioned 3′ of the splice donor sequence; and a second ITR sequence; and
a second AAV vector comprising a first ITR sequence; a second recombinogenic region; a splice acceptor sequence positioned 3′ of the second recombinogenic region; a second coding polynucleotide that encodes a C-terminal portion of the OTOF isoform 5 protein positioned 3′ of the splice acceptor sequence; a poly(A) sequence positioned 3′ of the second coding polynucleotide; and a second ITR sequence;
wherein the first coding polynucleotide and the second coding polynucleotide that encode the OTOF isoform 5 protein do not overlap, wherein neither the first nor second AAV vector encodes the full-length OTOF isoform 5 protein, and wherein the first AAV vector and the second AAV vector are administered at a ratio of about 3:1 to about 1:3.

2. (canceled)

3. The method of claim 1, wherein the first AAV vector and the second AAV vector comprise an AAV1 capsid.

4. The method of claim 1, wherein the Myo15 promoter comprises a first region having at least 85% sequence identity to SEQ ID NO: 7 or a functional portion or derivative thereof comprising the sequence of SEQ ID NO: 9 and/or SEQ ID NO: 10 operably linked to a second region having at least 85% sequence identity to SEQ ID NO: 8 or a functional portion or derivative thereof comprising the sequence of SEQ ID NO: 14 and/or SEQ ID NO: 15.

5. The method of claim 1, wherein the Myo15 promoter comprises the sequence of SEQ ID NO: 21.

6. (canceled)

7. The method of claim 1, wherein the first recombinogenic region and the second recombinogenic region are each an AP gene fragment.

8-9. (canceled)

10. The method of claim 7, wherein the AP gene fragment comprises the sequence of SEQ ID NO: 51.

11. (canceled)

12. The method of claim 1, wherein the OTOF isoform 5 protein comprises the sequence of SEQ ID NO: 1 or a variant thereof having one or more conservative amino acid substitutions.

13-16. (canceled)

17. The method of claim 1, wherein the first coding polynucleotide encodes amino acids 1-802 of SEQ ID NO: 1 and the second coding polynucleotide encodes amino acids 803-1997 of SEQ ID NO: 1.

18-32. (canceled)

33. The method of claim 1, wherein:
(a) the first AAV vector comprises a polynucleotide sequence comprising the sequence of nucleotides 235 to 4004 of SEQ ID NO: 66 and the second AAV vector comprises a polynucleotide sequence comprising the sequence of nucleotides 229 to 4438 of SEQ ID NO: 67;
(b) the first AAV vector comprises a polynucleotide sequence comprising the sequence of nucleotides 2272 to 6041 of SEQ ID NO: 60 and the second AAV vector comprises a polynucleotide sequence comprising the sequence of nucleotides 2267 to 6476 of SEQ ID NO: 61; or
(c) the first AAV vector comprises a polynucleotide sequence comprising the sequence of nucleotides 182 to 3949 of SEQ ID NO: 62 and the second AAV vector comprises a polynucleotide sequence comprising the sequence of nucleotides 187 to 4396 of SEQ ID NO: 63.

34-43. (canceled)

44. The method of claim 1, wherein the subject has behavioral open-set word detection scores of <30% in the ear(s) to be treated, congenital auditory neuropathy, present outer hair cell function, or detectable otoacoustic emissions; or wherein a cochlear microphonic is present in the ear(s) to be treated.

45-48. (canceled)

49. The method of claim 1, wherein the administering to the inner ear comprises intracochlear injection via insertion of a catheter through the round window membrane into the inner ear perilymph.

50. The method of claim 49, wherein the administering further comprises creating a fenestration in the lateral semicircular canal.

51-53. (canceled)

54. The method of claim 1, wherein the first vector and the second vector are administered at a ratio of about 1:1.

55-61. (canceled)

62. The method of claim 1, wherein the OTOF dual vector system is administered in an amount of 1.0×10¹³ vg/mL to 5.0×10¹³ vg/mL in a volume of 200-250 μL.

63-66. (canceled)

67. The method of claim 1, wherein the OTOF dual vector system is administered in an amount of 5.0×10¹³ vg/mL to 1.0×10¹⁴ vg/mL in a volume of 200-250 μL.

68-72. (canceled)

73. The method of claim 1, wherein the method improves one or more parameters selected from the subject's auditory brainstem response (ABR), behavioral audiometry, and score in one or more hearing questionnaires or behavioral tasks.

74-77. (canceled)

78. An aqueous suspension comprising an OTOF dual vector system, 10 mM sodium phosphate or disodium phosphate, 180 mM sodium chloride, 5% (w/v) sucrose, and 0.001% (w/v) poloxamer 188 at a pH of 7.4, wherein the OTOF dual vector system comprises:
a first AAV vector comprising a first ITR sequence; a Myosin 15 (Myo15) promoter operably linked to a first coding polynucleotide that encodes an N-terminal portion of an OTOF isoform 5 protein; a splice donor sequence positioned 3′ of the first coding polynucleotide; a recombinogenic region positioned 3′ of the splice donor sequence; and a second ITR sequence; and
a second AAV vector comprising a first ITR sequence; a second recombinogenic region; a splice acceptor sequence positioned 3′ of the second recombinogenic region; a second coding polynucleotide that encodes a C-terminal portion of the OTOF isoform 5 protein positioned 3′ of the splice acceptor sequence; a poly(A) sequence positioned 3′ of the second coding polynucleotide; and a second ITR sequence;
wherein the first coding polynucleotide and the second coding polynucleotide that encode the OTOF isoform 5 protein do not overlap, and wherein neither the first nor second AAV vector encodes the full-length OTOF isoform 5 protein.

79-83. (canceled)

84. The suspension of claim 78, wherein the suspension has a titer of 1×10¹³ vg/mL to 1×10¹⁴ vg/mL.

85-89. (canceled)

90. A kit comprising the suspension of claim 78.

91. The kit of claim 90, further comprising one or more of a syringe, syringe pump, and catheter.

92. (canceled)


SEQUENCE LISTING (68 sequences)

SEQ ID NO: 1 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 2 - DNA, 5994 bp, Homo sapiens
  source 1..5994: mol_type = other DNA
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 3 - DNA, 5994 bp, synthetic construct
  source 1..5994: mol_type = other DNA
atggcactgctgatccacctgaaaaccgtctccgaactgagaggcagaggggacagaatcgctaaagtcaccttccggggacagagcttttacagcagggtgctggagaactgcgaggacgtggccgactttgacgagacattcaggtggcccgtggccagctccatcgatcgcaatgagatgctggagatccaggtgtttaactatagcaaggtgttctccaataagctgatcggcaccttccggatggtgctgcagaaggtggtggaggagtcccacgtggaggtgaccgacacactgatcgacgataacaatgccatcatcaagacatccctgtgcgtggaggtgcgctaccaggccaccgatggcacagtgggctcttgggacgatggcgacttcctgggcgatgagtccctgcaggaggaggagaaggactctcaggagacagatggcctgctgcctggctcccggccatctagccgcccccctggcgagaagtcttttaggagagccggcaggtccgtgttctctgccatgaagctgggcaagaacaggagccacaaggaggagcctcagaggcccgacgagccagccgtgctggagatggaggacctggatcacctggccatcagactgggcgatggcctggaccctgatagcgtgtccctggcctccgtgaccgccctgaccacaaacgtgtctaataagcggagcaagccagacatcaagatggagccatctgccggcaggcccatggattaccaggtgagcatcacagtgatcgaggccagacagctggtgggcctgaacatggaccccgtggtgtgcgtggaagtgggcgacgataagaagtacacctccatgaaggagtctacaaactgtccatactacaacgagtacttcgtgtttgatttccacgtgagccccgacgtgatgttcgataagatcatcaagatcagcgtgatccactccaagaatctgctgcggtctggcaccctggtgggaagctttaagatggacgtgggcacagtgtactctcagcctgagcaccagttccaccacaagtgggccatcctgagcgatccagacgatatctcctctggcctgaagggctatgtgaagtgcgacgtggcagtggtgggcaagggcgataacatcaagaccccacacaaggccaatgagacagacgaggacgatatcgagggaaacctgctgctgccagagggagtgccacccgagaggcagtgggccaggttctacgtgaagatctatagggcagagggcctgcctaggatgaacaccagcctgatggccaatgtgaagaaggccttcatcggcgagaacaaggacctggtggatccctacgtgcaggtgttctttgccggccagaagggcaagacctccgtgcagaagagctcctatgagcctctgtggaatgagcaggtggtgtttacagacctgttccctccactgtgcaagaggatgaaggtgcagatcagagactctgataaggtgaacgacgtggccatcggcacccactttatcgatctgaggaagatcagcaatgacggcgataagggcttcctgcccaccctgggccccgcctgggtgaacatgtacggcagcaccagaaattatacactgctggacgagcaccaggatctgaacgagggcctgggcgagggcgtgagctttagagccaggctgctgctgggcctggccgtggagatcgtggacacctccaatcccgagctgacctctagcacagaggtgcaggtggagcaggccacacctatctctgagagctgtgccggcaagatggaggagttctttctgtttggcgccttcctggaggcctccatgatcgaccggcgcaacggcgataagcctatcaccttcgaggtgacaatcggcaactacggcaatgaggtggacggcctgtctcggccccagcgcccaaggcccagaaaggagcctggcgacgaggaggaggtggatctgatccagaacgccagcgacgatgaggcaggcgacgcaggcgatctggcctccgtgtcctctaccccccctatgcggccacaggtgacagaccgcaattactttcacctgccttatctggagcgcaagccatgcatctacatcaagtcttggtggcccgatcagaggagacggctgtataacgccaatatcatggaccacatcgccgataagctggaggagggcctgaatgacatccaggagatgatcaagaccgagaagtcctatccagagcgcaggctgaggggcgtgctggaggagctgagctgtggctgctgtagattcctgtccctggccgacaaggatcaggggcactcatcacggacacggctggaccgggagcggctgaaatcatgtatgcgggagctggaaaatatgggacagcaggcaaggatgctgcgcgcccaggtgaagaggcacaccgtgagagacaagctgcggctgtgccagaacttcctgcagaagctgcgctttctggccgatgagccacagcacagcatccccgacatcttcatctggatgatgtccaacaataagagagtggcctacgcccgggtgccctctaaggatctgctgtttagcatcgtggaggaggagacaggcaaggactgtgccaaggtgaagaccctgttcctgaagctgcctggcaagagaggctttggcagcgccggatggaccgtgcaggcaaaggtggagctgtatctgtggctgggcctgtctaagcagcggaaggagttcctgtgcggcctgccctgtggctttcaggaggtgaaggcagcacagggactgggactgcacgccttcccccccgtgagcctggtgtacaccaagaagcaggcctttcagctgagggcccatatgtaccaggccaggtctctgttcgccgccgatagctccggactgagcgacccttttgccagggtgttctttatcaatcagagccagtgcacagaggtgctgaacgagaccctgtgcccaacatgggatcagatgctggtgttcgacaacctggagctgtacggagaggcacacgagctgagggacgatccacccatcatcgtgatcgagatctatgatcaggactccatgggcaaggccgatttcatgggcaggacctttgccaagcccctggtgaagatggccgacgaggcctactgccctccaagattcccccctcagctcgagtactatcagatctataggggaaatgcaaccgccggagacctgctggccgcctttgagctgctgcagatcggccccgccggaaaggcagacctgccacccatcaacggcccagtggatgtggacagaggccccatcatgcctgtgccaatgggcatcagaccagtgctgtccaagtacagggtggaggtgctgttctggggactgcgcgacctgaagagggtgaatctggcccaggtggataggcccagagtggacatcgagtgcgccggaaagggcgtgcagtctagcctgatccacaactataagaagaacccaaatttcaacaccctggtgaagtggtttgaggtggatctgcccgagaatgagctgctgcaccctccactgaacatccgggtggtggactgtagagccttcggcaggtacaccctggtgggcagccacgccgtgagcagcctgaggaggttcatctacaggccccctgacaggtccgccccttcttggaataccacagtgagactgctgcggcgctgcagggtgctgtgcaacggaggcagctcctctcactctaccggcgaggtggtggtgacaatggagcctgaggtacccatcaagaagctggagaccatggtgaagctggatgccacaagcgaggcagtggtgaaggtggacgtggcagaggaggagaaggagaagaagaagaagaagaagggaaccgccgaggagcctgaggaagaggagccagatgagagcatgctggactggtggtccaagtacttcgcctctatcgacacaatgaaggagcagctgagacagcaggagcctagcggcatcgatctggaggagaaggaggaggtggacaataccgagggcctgaagggctccatgaagggcaaggagaaggcaagggcagcaaaggaagagaagaagaagaagacccagagcagcggctctggacagggcagcgaggcaccagagaagaagaagcctaagatcgatgagctgaaggtgtacccaaaggagctggagtccgagttcgataattttgaggactggctgcacaccttcaacctgctgcgcggcaagacaggcgacgatgaggacggcagcaccgaggaggagagaatcgtgggccggtttaagggctccctgtgcgtgtacaaggtgccactgcctgaggacgtgagcagggaggccggatacgactctacctatggcatgttccagggcatcccctctaatgatcctatcaacgtgctggtgcgcgtgtatgtggtgagggccacagatctgcaccccgccgacatcaacggcaaggccgacccttacatcgccatccgcctgggcaagaccgatatcagggacaaggagaattatatctccaagcagctgaaccccgtgttcggcaagtcttttgacatcgaggccagcttccctatggagtccatgctgaccgtggccgtgtacgattgggacctggtgggcaccgacgatctgatcggcgagacaaagatcgatctggagaatcgcttttattctaagcacagggcaacctgcggaatcgcacagacctacagcacacacggctataacatctggcgcgaccccatgaagcctagccagatcctgacaaggctgtgcaaggatggcaaggtggacggaccacacttcggaccacccggcagagtgaaggtggccaatcgggtgtttacaggcccttccgagatcgaggatgagaacggccagcgcaagccaaccgacgagcacgtggccctgctggccctgaggcactgggaggatatcccaagggccggatgtaggctggtgcctgagcacgtggagaccagaccactgctgaatccagacaagccaggaatcgagcagggcaggctggagctgtgggtggatatgttcccaatggacatgccagccccaggaacacccctggatatctcccctagaaagccaaagaagtacgagctgagagtgatcatctggaacacagacgaggtggtgctggaggacgatgacttctttaccggcgagaagtctagcgatatctttgtgcgcggatggctgaagggacagcaggaggacaagcaggatacagacgtgcactaccactccctgaccggcgagggcaatttcaactggagatacctgttcccttttgattatctggccgccgaggagaagatcgtgatctctaagaaggagagcatgttttcctgggacgagacagagtataagatcccagccagactgaccctgcagatctgggatgccgaccacttcagcgccgatgactttctgggcgccatcgagctggacctgaaccggttcccaagaggcgccaagaccgccaagcagtgcacaatggagatggcaaccggagaggtggacgtgcctctggtgtctatcttcaagcagaagcgggtgaagggatggtggccactgctggccaggaacgagaatgatgagtttgagctgacaggcaaggtggaggcagagctgcacctgctgaccgccgaggaggcagagaagaacccagtgggcctggccaggaatgagcccgaccctctggagaagccaaacaggcccgatacagccttcgtgtggtttctgaatcctctgaagagcatcaagtacctgatctgtaccaggtataagtggctgatcatcaagatcgtgctggccctgctgggactgctgatgctgggcctgtttctgtactccctgcccggctatatggtgaagaagctgctgggcgcctga

SEQ ID NO: 4 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 5 - DNA, 5979 bp, Homo sapiens
  source 1..5979: mol_type = other DNA
atggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctga

SEQ ID NO: 6 - DNA, 5994 bp, synthetic construct
  source 1..5994: mol_type = other DNA
atggcactgctgatccacctgaaaaccgtctccgaactgagaggcagaggggacagaatcgctaaagtcaccttccggggacagagcttttacagcagggtgctggagaactgcgaggacgtggccgactttgacgagacattcaggtggcccgtggccagctccatcgatcgcaatgagatgctggagatccaggtgtttaactatagcaaggtgttctccaataagctgatcggcaccttccggatggtgctgcagaaggtggtggaggagtcccacgtggaggtgaccgacacactgatcgacgataacaatgccatcatcaagacatccctgtgcgtggaggtgcgctaccaggccaccgatggcacagtgggctcttgggacgatggcgacttcctgggcgatgagtccctgcaggaggaggagaaggactctcaggagacagatggcctgctgcctggctcccggccatctagccgcccccctggcgagaagtcttttaggagagccggcaggtccgtgttctctgccatgaagctgggcaagaacaggagccacaaggaggagcctcagaggcccgacgagccagccgtgctggagatggaggacctggatcacctggccatcagactgggcgatggcctggaccctgatagcgtgtccctggcctccgtgaccgccctgaccacaaacgtgtctaataagcggagcaagccagacatcaagatggagccatctgccggcaggcccatggattaccaggtgagcatcacagtgatcgaggccagacagctggtgggcctgaacatggaccccgtggtgtgcgtggaagtgggcgacgataagaagtacacctccatgaaggagtctacaaactgtccatactacaacgagtacttcgtgtttgatttccacgtgagccccgacgtgatgttcgataagatcatcaagatcagcgtgatccactccaagaatctgctgcggtctggcaccctggtgggaagctttaagatggacgtgggcacagtgtactctcagcctgagcaccagttccaccacaagtgggccatcctgagcgatccagacgatatctcctctggcctgaagggctatgtgaagtgcgacgtggcagtggtgggcaagggcgataacatcaagaccccacacaaggccaatgagacagacgaggacgatatcgagggaaacctgctgctgccagagggagtgccacccgagaggcagtgggccaggttctacgtgaagatctatagggcagagggcctgcctaggatgaacaccagcctgatggccaatgtgaagaaggccttcatcggcgagaacaaggacctggtggatccctacgtgcaggtgttctttgccggccagaagggcaagacctccgtgcagaagagctcctatgagcctctgtggaatgagcaggtggtgtttacagacctgttccctccactgtgcaagaggatgaaggtgcagatcagagactctgataaggtgaacgacgtggccatcggcacccactttatcgatctgaggaagatcagcaatgacggcgataagggcttcctgcccaccctgggccccgcctgggtgaacatgtacggcagcaccagaaattatacactgctggacgagcaccaggatctgaacgagggcctgggcgagggcgtgagctttagagccaggctgctgctgggcctggccgtggagatcgtggacacctccaatcccgagctgacctctagcacagaggtgcaggtggagcaggccacacctatctctgagagctgtgccggcaagatggaggagttctttctgtttggcgccttcctggaggcctccatgatcgaccggcgcaacggcgataagcctatcaccttcgaggtgacaatcggcaactacggcaatgaggtggacggcctgtctcggccccagcgcccaaggcccagaaaggagcctggcgacgaggaggaggtggatctgatccagaacgccagcgacgatgaggcaggcgacgcaggcgatctggcctccgtgtcctctaccccccctatgcggccacaggtgacagaccgcaattactttcacctgccttatctggagcgcaagccatgcatctacatcaagtcttggtggcccgatcagaggagacggctgtataacgccaatatcatggaccacatcgccgataagctggaggagggcctgaatgacatccaggagatgatcaagaccgagaagtcctatccagagcgcaggctgaggggcgtgctggaggagctgagctgtggctgctgtagattcctgtccctggccgacaaggatcaggggcactcatcacggacacggctggaccgggagcggctgaaatcatgtatgcgggagctggaaaatatgggacagcaggcaaggatgctgcgcgcccaggtgaagaggcacaccgtgagagacaagctgcggctgtgccagaacttcctgcagaagctgcgctttctggccgatgagccacagcacagcatccccgacatcttcatctggatgatgtccaacaataagagagtggcctacgcccgggtgccctctaaggatctgctgtttagcatcgtggaggaggagacaggcaaggactgtgccaaggtgaagaccctgttcctgaagctgcctggcaagagaggctttggcagcgccggatggaccgtgcaggcaaaggtggagctgtatctgtggctgggcctgtctaagcagcggaaggagttcctgtgcggcctgccctgtggctttcaggaggtgaaggcagcacagggactgggactgcacgccttcccccccgtgagcctggtgtacaccaagaagcaggcctttcagctgagggcccatatgtaccaggccaggtctctgttcgccgccgatagctccggactgagcgacccttttgccagggtgttctttatcaatcagagccagtgcacagaggtgctgaacgagaccctgtgcccaacatgggatcagatgctggtgttcgacaacctggagctgtacggagaggcacacgagctgagggacgatccacccatcatcgtgatcgagatctatgatcaggactccatgggcaaggccgatttcatgggcaggacctttgccaagcccctggtgaagatggccgacgaggcctactgccctccaagattcccccctcagctcgagtactatcagatctataggggaaatgcaaccgccggagacctgctggccgcctttgagctgctgcagatcggccccgccggaaaggcagacctgccacccatcaacggcccagtggatgtggacagaggccccatcatgcctgtgccaatgggcatcagaccagtgctgtccaagtacagggtggaggtgctgttctggggactgcgcgacctgaagagggtgaatctggcccaggtggataggcccagagtggacatcgagtgcgccggaaagggcgtgcagtctagcctgatccacaactataagaagaacccaaatttcaacaccctggtgaagtggtttgaggtggatctgcccgagaatgagctgctgcaccctccactgaacatccgggtggtggactgtagagccttcggcaggtacaccctggtgggcagccacgccgtgagcagcctgaggaggttcatctacaggccccctgacaggtccgccccttcttggaataccacagtgagactgctgcggcgctgcagggtgctgtgcaacggaggcagctcctctcactctaccggcgaggtggtggtgacaatggagcctgaggtacccatcaagaagctggagaccatggtgaagctggatgccacaagcgaggcagtggtgaaggtggacgtggcagaggaggagaaggagaagaagaagaagaagaagggaaccgccgaggagcctgaggaagaggagccagatgagagcatgctggactggtggtccaagtacttcgcctctatcgacacaatgaaggagcagctgagacagcaggagcctagcggcatcgatctggaggagaaggaggaggtggacaataccgagggcctgaagggctccatgaagggcaaggagaaggcaagggcagcaaaggaagagaagaagaagaagacccagagcagcggctctggacagggcagcgaggcaccagagaagaagaagcctaagatcgatgagctgaaggtgtacccaaaggagctggagtccgagttcgataattttgaggactggctgcacaccttcaacctgctgcgcggcaagacaggcgacgatgaggacggcagcaccgaggaggagagaatcgtgggccggtttaagggctccctgtgcgtgtacaaggtgccactgcctgaggacgtgagcagggaggccggatacgactctacctatggcatgttccagggcatcccctctaatgatcctatcaacgtgctggtgcgcgtgtatgtggtgagggccacagatctgcaccccgccgacatcaacggcaaggccgacccttacatcgccatccgcctgggcaagaccgatatcagggacaaggagaattatatctccaagcagctgaaccccgtgttcggcaagtcttttgacatcgaggccagcttccctatggagtccatgctgaccgtggccgtgtacgattgggacctggtgggcaccgacgatctgatcggcgagacaaagatcgatctggagaatcgcttttattctaagcacagggcaacctgcggaatcgcacagacctacagcacacacggctataacatctggcgcgaccccatgaagcctagccagatcctgacaaggctgtgcaaggatggcaaggtggacggaccacacttcggaccacccggcagagtgaaggtggccaatcgggtgtttacaggcccttccgagatcgaggatgagaacggccagcgcaagccaaccgacgagcacgtggccctgctggccctgaggcactgggaggatatcccaagggccggatgtaggctggtgcctgagcacgtggagaccagaccactgctgaatccagacaagccaggaatcgagcagggcaggctggagctgtgggtggatatgttcccaatggacatgccagccccaggaacacccctggatatctcccctagaaagccaaagaagtacgagctgagagtgatcatctggaacacagacgaggtggtgctggaggacgatgacttctttaccggcgagaagtctagcgatatctttgtgcgcggatggctgaagggacagcaggaggacaagcaggatacagacgtgcactaccactccctgaccggcgagggcaatttcaactggagatacctgttcccttttgattatctggccgccgaggagaagatcgtgatctctaagaaggagagcatgttttcctgggacgagacagagtataagatcccagccagactgaccctgcagatctgggatgccgaccacttcagcgccgatgactttctgggcgccatcgagctggacctgaaccggttcccaagaggcgccaagaccgccaagcagtgcacaatggagatggcaaccggagaggtggacgtgcctctggtgtctatcttcaagcagaagagggtgaagggctggtggccactgctggccagaaacgagaatgatgagtttgagctgacaggcaaggtggaggcagagctgcacctgctgaccgccgaggaggcagagaagaacccagtgggcctggccaggaatgagcccgaccctctggagaagccaaacaggcccgacaccagcttcatctggtttctgaatcctctgaagtccgcccggtacttcctgtggcacacctatcgctggctgctgctgaagctgttattactgttattactgctgctgctgctggccctgtttctgtacagcgtgcccggctatctggtgaagaagatcctgggcgcctga

SEQ ID NO: 7 - DNA, 454 bp, Mus musculus
  source 1..454: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 8 - DNA, 1157 bp, Mus musculus
  source 1..1157: mol_type = genomic DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 9 - DNA, 76 bp, Mus musculus
  source 1..76: mol_type = genomic DNA
cccatgtcagctgcttgtgctttccagagacaaaacaggaataatagatgtcattaaatatacattgggccccagg

SEQ ID NO: 10 - DNA, 95 bp, Mus musculus
  source 1..95: mol_type = genomic DNA
agcctgagcctcctttccatctctgtggaggcagacataggacccccaacaaacagcatgcaggttgggagccagccacaggacccaggtaaggg

SEQ ID NO: 11 - DNA, 171 bp, synthetic construct
  source 1..171: mol_type = other DNA
cccatgtcagctgcttgtgctttccagagacaaaacaggaataatagatgtcattaaatatacattgggccccaggagcctgagcctcctttccatctctgtggaggcagacataggacccccaacaaacagcatgcaggttgggagccagccacaggacccaggtaaggg

SEQ ID NO: 12 - DNA, 171 bp, synthetic construct
  source 1..171: mol_type = other DNA
agcctgagcctcctttccatctctgtggaggcagacataggacccccaacaaacagcatgcaggttgggagccagccacaggacccaggtaagggcccatgtcagctgcttgtgctttccagagacaaaacaggaataatagatgtcattaaatatacattgggccccagg

SEQ ID NO: 13 - DNA, 184 bp, Mus musculus
  source 1..184: mol_type = genomic DNA
cccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggg

SEQ ID NO: 14 - DNA, 82 bp, Mus musculus
  source 1..82: mol_type = genomic DNA
tgaggtgggagctgggctctccctgatgtattattcagctccctggagttggccagctcctgttacactggccacagccctg

SEQ ID NO: 15 - DNA, 106 bp, Mus musculus
  source 1..106: mol_type = genomic DNA
cacaggcctcccagtctaccctcactagaaaacaacacccaggcactttccaccacctctcaaaggtgaaacccaaggctggtctagagaatgaattatggatcct

SEQ ID NO: 16 - DNA, 188 bp, synthetic construct
  source 1..188: mol_type = other DNA
tgaggtgggagctgggctctccctgatgtattattcagctccctggagttggccagctcctgttacactggccacagccctgcacaggcctcccagtctaccctcactagaaaacaacacccaggcactttccaccacctctcaaaggtgaaacccaaggctggtctagagaatgaattatggatcct

SEQ ID NO: 17 - DNA, 188 bp, synthetic construct
  source 1..188: mol_type = other DNA
cacaggcctcccagtctaccctcactagaaaacaacacccaggcactttccaccacctctcaaaggtgaaacccaaggctggtctagagaatgaattatggatccttgaggtgggagctgggctctccctgatgtattattcagctccctggagttggccagctcctgttacactggccacagccctg

SEQ ID NO: 18 - DNA, 430 bp, Mus musculus
  source 1..430: mol_type = genomic DNA
tgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcct

SEQ ID NO: 19 - DNA, 1611 bp, synthetic construct
  source 1..1611: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactatggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 20 - DNA, 1611 bp, synthetic construct
  source 1..1611: mol_type = other DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 21 - DNA, 965 bp, synthetic construct
  source 1..965: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 22 - DNA, 520 bp, synthetic construct
  source 1..520: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 23 - DNA, 235 bp, Homo sapiens
  source 1..235: mol_type = genomic DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttcc

SEQ ID NO: 24 - DNA, 688 bp, Homo sapiens
  source 1..688: mol_type = genomic DNA
tgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 25 - DNA, 77 bp, Homo sapiens
  source 1..77: mol_type = genomic DNA
cccctgtcagctgctcactcctttccagagacaaaacaggaataatagacatcattaaatatacatagggccccagg

SEQ ID NO: 26 - DNA, 82 bp, Homo sapiens
  source 1..82: mol_type = genomic DNA
tgagccggcagcctggctccccaccccatgtattattcagctcctgagagccagccagctcctgttacactgaccgcagccc

SEQ ID NO: 27 - DNA, 111 bp, Homo sapiens
  source 1..111: mol_type = genomic DNA
cacaggcctcataggcaggcttccccatcctggtaaacaacacccacacactttctactactgctctagggtgaaacccaaggcgctctagaggagatgaattatggatcc

SEQ ID NO: 28 - DNA, 193 bp, synthetic construct
  source 1..193: mol_type = other DNA
tgagccggcagcctggctccccaccccatgtattattcagctcctgagagccagccagctcctgttacactgaccgcagccccacaggcctcataggcaggcttccccatcctggtaaacaacacccacacactttctactactgctctagggtgaaacccaaggcgctctagaggagatgaattatggatcc

SEQ ID NO: 29 - DNA, 193 bp, synthetic construct
  source 1..193: mol_type = other DNA
cacaggcctcataggcaggcttccccatcctggtaaacaacacccacacactttctactactgctctagggtgaaacccaaggcgctctagaggagatgaattatggatcctgagccggcagcctggctccccaccccatgtattattcagctcctgagagccagccagctcctgttacactgaccgcagccc

SEQ ID NO: 30 - DNA, 499 bp, Homo sapiens
  source 1..499: mol_type = genomic DNA
tgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatcc

SEQ ID NO: 31 - DNA, 1262 bp, synthetic construct
  source 1..1262: mol_type = other DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttagcctagagtccttggggtctgtcactggccaccctcgcatcccaggctgcaggaaactgaggcccagagaggacaaggactttcctggacccacacagccagtcagtgacagagcctagggtctgagccaggcctgacccaacctccatttctgcctctctacccctgcccccgccccaacacacacacacacacaagtggagttccactgaaacgcccctccttgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 32 - DNA, 649 bp, synthetic construct
  source 1..649: mol_type = other DNA
gcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcgggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgc

SEQ ID NO: 33 - DNA, 240 bp, Mus musculus
  source 1..240: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 34 - DNA, 95 bp, Mus musculus
  source 1..95: mol_type = genomic DNA
tttatgaggtgggagctgggctctccctgatgtattattcagctccctggagttggccagctcctgttacactggccacagccctgggcatccgc

SEQ ID NO: 35 - DNA, 185 bp, Mus musculus
  source 1..185: mol_type = genomic DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 36 - DNA, 335 bp, synthetic construct
  source 1..335: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 37 - DNA, 425 bp, synthetic construct
  source 1..425: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 38 - DNA, 280 bp, synthetic construct
  source 1..280: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 39 - DNA, 520 bp, synthetic construct
  source 1..520: mol_type = other DNA
tttatgaggtgggagctgggctctccctgatgtattattcagctccctggagttggccagctcctgttacactggccacagccctgggcatccgcctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 40 - DNA, 520 bp, synthetic construct
  source 1..520: mol_type = other DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 41 - DNA, 520 bp, synthetic construct
  source 1..520: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 42 - DNA, 964 bp, synthetic construct
  source 1..964: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 43 - DNA, 519 bp, synthetic construct
  source 1..519: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 44 - DNA, 943 bp, synthetic construct
  source 1..943: mol_type = other DNA
ggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 45 - DNA, 548 bp, synthetic construct
  source 1..548: mol_type = other DNA
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 46 - DNA, 425 bp, synthetic construct
  source 1..425: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 47 - DNA, 77 bp, synthetic construct
  source 1..77: mol_type = other DNA
gggattttgccgatttcggcctattggttaaaaaatgagctgatttaacaaaaatttaacgcgaattttaacaaaat

SEQ ID NO: 48 - DNA, 865 bp, Homo sapiens
  source 1..865: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 49 - DNA, 437 bp, Homo sapiens
  source 1..437: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 50 - DNA, 428 bp, Homo sapiens
  source 1..428: mol_type = other DNA
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 51 - DNA, 287 bp, Homo sapiens
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 52 - DNA, 290 bp, Homo sapiens
  source 1..290: mol_type = other DNA
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 53 - DNA, 288 bp, Homo sapiens
  source 1..288: mol_type = other DNA
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 54 - DNA, 84 bp, synthetic construct
  source 1..84: mol_type = other DNA
gtaagtatcaaggttacaagacaggtttaaggagaccaatagaaactgggcttgtcgagacagagaagactcttgcgtttctga

SEQ ID NO: 55 - DNA, 49 bp, synthetic construct
  source 1..49: mol_type = other DNA
taggcacctattggtcttactgacatccactttgcctttctctccacag

SEQ ID NO: 56 - DNA, 2406 bp, Homo sapiens
  source 1..2406: mol_type = other DNA
atggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctg

SEQ ID NO: 57 - DNA, 3588 bp, Homo sapiens
  source 1..3588: mol_type = other DNA
gaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatga

SEQ ID NO: 58 - protein, 802 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMREL

SEQ ID NO: 59 - protein, 1195 aa, Homo sapiens
ENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 60 - DNA, 10032 bp, synthetic construct
  source 1..10032: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 61 - DNA, 10461 bp, synthetic construct
  source 1..10461: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 62 - DNA, 6678 bp, synthetic construct
  source 1..6678: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccgaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 63 - DNA, 7152 bp, synthetic construct
  source 1..7152: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 64 - DNA, 10005 bp, synthetic construct
  source 1..10005: mol_type = other DNA
tcgcgcgtttcggtgatgacggtgaaaacctctgacacatgcagctcccggatagaggtcatccttcctgaccatttccatcattccagtcgaactcacacacaacaccaaatgcatttaagtcgcttgaaattgctataagcagagcatgttgcgccagcatgattaatacagcatttaatacagagccgtgtttattgagtcggtattcagagtctgaccagaaattattaatctggtgaagttattcctctgtcattacgtcatggtcgatttcaatttctattgatgctttccagtcgtaatcaatgatgtattttttgatgtttgacctctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaattaaggccactatcaggcagctttgttgttctgtttaccaagttctctggcaatcattgccgtcgttcgtattgcccatttatcgacatatttcccatcttcctatacaggaaacatttcttcaggcttaaccatgcattccgattgcagcttgcatccattgcatcgcttgaattgtccacaccattgatttttatcaatagtcgtagtttaacggatagtcctggtattgttccatcacatcctgaggatgcccttcgaactcttcaaattcttcttcctaatatcaccttaaatagtggattgcggtagtaaagattgtgcctgtcttttaaccacatcaggctcggtggttctcgtgtacccctacagcgagaaatcggataaactattacaacccctacagtttgtagagtatagaaaatgatccactcgttattctcggacgagtgttcagtaatgaacctctggagagaaccatctatatgatcgttatctgggtttgacttctgcttttaagcccagataacttgcctgaatatgttaatgagagaatcggtattcctcatgtgtggcatgttttcgtctttgctcttgcattttcactagcaattaatgtgcatcgattatcagctattgccagcgccagatataagcgatttaagctaagaaaacgcattaaggtgcaaaacgataaagtgcgatcagtaattcaaaaccttacaggagagcaatctatggttttgtgctcagcccttaatgaaggcaggtagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgttactctgcgcgacgttcgcggcggcgtgtttgtgcatccatctggattctcctgtcagttagctttggtggtgtgtggcagttgtagtcctgaacgaaaaccccccgcgattggcacgttggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcaggcacgatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaaatccacatctgtatgttttttatattaatttattttttgcaggggggcattgtttggtaggtgagagttctgaattgctatgtttagtgagttgtatctatttatttttcaataaatacaattagttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcaggtagccgcttatgctggaaagaagcaataacccgcagaaaaacaaagctccaagctcaacaaaactaagggcatagacaataactacctatgtcatatacccatactctctaatcttggccagtcggcgcgttctgcttccgattagaaacgtcaaggcagcaatcaggattgcaatcttggttcctgcataggatgacaatgtcgccccaagaccatctctatgagctgaaaaagaaacacaaggaatgtagtggcggaaaaggagatagcaaatgcttacgataacgtaaggaattattactatgtaaacaccaggcaagattctgttccgtataattactcctgataattaatccttaactttgcccacctgccttttaaaacattccagtatatcacttttcattcttgcgtagcaatatgccctctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttctgccgatgaaatgctaatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgtagctttagagcgatttatcttctgaaccagactcttgtcatttgttttggtaaagagaaaagtttttccatcgattttatgaatatacaaataattggagccaaccttcaggtgatgattatcagccagcagagaattaaggaaaacagacaggtttattgagcacttatctttccctttatttttgctgcggtaagtcgcataaaaaccattcttcacaattcaatccatttactatgttatgttctgaggggagtgaaaattcccctaattcgatgaagattcttgctaaattgttatcagctatgcgccgaccagaacaccttgccgatcagccaaacgtctaatcaggccactgactagcgataactttccccacaacggaacaactctcattgcatgggataattgggtactgtgggtttagtggttgtaaaaacacctgaccgctatccctgatcagtttcttgaaggtaaactcatcacccccaagtctggctatacagaaatcacctggctcaacagcctgctcagggtcaacgagaatttacattccgtcaggatagcttggcttggagcctgttggtgcggtcacggaattaccttcaacctcaagccagaatgcagaatcactggcttttttggttgtgcttacccatctctccgcatcacctttggtaaaggttctaagctaaggtgagaacatccctgcctgaacatgagaaaaaacagggtactcatactcacttattagtgacggctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 65 - DNA, 6653 bp, synthetic construct
  source 1..6653: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgca

SEQ ID NO: 66 - DNA, 6986 bp, synthetic construct
  source 1..6986: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 67 - DNA, 7414 bp, synthetic construct
  source 1..7414: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaacggccgctatgctagcttggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 68 - DNA, 6959 bp, synthetic construct
  source 1..6959: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg